(19) United States
(12) Patent Application Publication
(10) Pub. No.: US 2025/0263810 A1
(43) Pub. Date: Aug. 21, 2025

(54) COMPOSITIONS, SYSTEMS, AND METHODS FOR EXTRACTION OF METALS FROM MINERALS

(71) Applicant: Maverick Labs, Inc., San Antonio, TX (US)

(72) Inventors: Eric HERRERA, San Antonio, TX (US); Jesse M. EVANS, San Antonio, TX (US); Antoine James Jean-Pierre Marie GUÉGUEN, San Antonio, TX (US); Daniel GONZALEZ, San Antonio, TX (US); David Gustavo Murcia RINCON, San Antonio, TX (US); Jose Juan Morales CORONA, San Antonio, TX (US); Noushaba Nusrat MAFY, San Antonio, TX (US)

(21) Appl. No.: 19/188,951

(22) Filed: Apr. 24, 2025

Related U.S. Application Data
(63) Continuation of application No. PCT/US2024/046780, filed on Sep. 13, 2024.
(60) Provisional application No. 63/583,201, filed on Sep. 15, 2023.

(51) Int. Cl. C22B 3/18; C12N 9/88

(52) U.S. Cl. CPC C22B 3/18; C12N 9/88; C12Y 402/01001

(57) ABSTRACT

Provided herein are methods, systems, and compositions for degrading minerals. The methods, systems, and compositions provided herein involve the use of enzymes having silicase activity and an increased ability to degrade minerals such as silicate materials. The methods, systems, and compositions provided herein may be used to release metal from the amorphous silica. The methods, systems, and compositions provided herein may further involve collecting, extracting, and/or purifying the metal released from the minerals.

### CROSS-REFERENCE

This application is a continuation of International Application No. PCT/US2024/046780, filed Sep. 13, 2024, which claims the benefit of U.S. Provisional Application No. 63/583,201, filed Sep. 15, 2023, each of which is incorporated herein by reference in its entirety.

### SEQUENCE LISTING

The instant application contains a Sequence Listing which has been submitted electronically in XML format and is hereby incorporated by reference in its entirety. Said XML copy, created on Oct. 14, 2024, is named 66122_702_301.xml and is 614,155 bytes in size.

### BACKGROUND

Metals such as lithium, aluminum, iron, nickel, cobalt, strontium, and rare earth elements have vast industrial applications and are in high demand across various industries. For example, lithium is widely used for energy storage, rechargeable batteries, electronic motors, electric vehicles, air mobility, clean energy, energy storage from solar panels, and other applications. Lithium has pharmaceutical applications, such as in lithium-based bipolar disorder treatments. Currently available sources and technologies for obtaining metals for use in industrial applications and products are limited, inefficient, costly, energy-intensive, and harmful to the environment.

### SUMMARY

There is a significant unmet need for compositions, methods, and systems that facilitate access to existing sources of metals, such as to extract and/or collect the metals from their sources, separate them, process them, and make them available for use in various industrial applications and/or products, in a manner that is industrially scalable, efficient, and inexpensive. This is at least in part to meet the demand for such metals in the industrial applications and products in need thereof. Many of the currently available methods and techniques for doing so are limited with respect to efficiency, scalability, and high cost. In many cases, such existing technologies may require performing processes and reactions at high temperatures and pressures that are energy-intensive, costly, and harmful to the environment. The compositions, methods, and systems of the present disclosure address the aforementioned needs and shortcomings, in some aspects, by providing compositions, methods, and systems for extracting, separating, and/or collecting metals from mineral sources, such as mineral materials including solid mineral materials, natural mineral materials, man-made mineral materials, rocks, ores, deposits, and/or other sources in an efficient, inexpensive, and scalable manner. In some cases, the disclosure further provides methods and systems for processing the extracted metals and/or using them in a product (e.g., rechargeable batteries). The extracted metal may be processed and turned into an industrial grade metal, battery grade metal, pharmaceutical grade metal, or other useful forms of metals.

In an aspect, provided herein is a method of extracting a metal from a mineral, the method comprising: (a) contacting the mineral material with an enzyme having silicase activity under reaction conditions such that the metal contained within the mineral material is solubilized and released; and (b) collecting the released metal, thereby extracting the metal from the mineral material. In some embodiments, the mineral material comprises an ore, a rock, a natural mineral material, a man-made mineral material, or any combination thereof. In some embodiments, the mineral material comprises a silicate. In some embodiments, the mineral material comprises an inosilicate, a phyllosilicate, an amorphous silicate, a tectosilicate, or any combination thereof. In some embodiments, the amorphous silicate is selected from the group consisting of: obsidian, coal fly ash, pumice, glass, and any combination thereof. In some embodiments, the tectosilicate comprises quarts, sand, or both. In some embodiments, the enzyme having silicase activity has a sequence identity of at least about at least about 30%, at least about 40%, at least about 50%, at least about 60%, at least about 70%, at least about 80%, at least about 90%, or more identity with a carbonic anhydrase. In some embodiments, the enzyme having silicase activity has a sequence identity of at least about 30%, at least about 400%, at least about 50%, at least about 60%, at least about 70%, at least about 80%, at least about 90%, or more identity with an alpha carbonic anhydrase. In some embodiments, the enzyme having silicase activity has a sequence identity at least at least about 30%, at least about 40%, about 50%, at least about 60%, at least about 70%, at least about 80%, at least about 90%, or more identity with a gamma carbonic anhydrase. In some embodiments, the enzyme having silicase activity is derived from an organism selected from the group consisting of: of: Methanosarcina thermophila, Bacillus licheniformis CG-B52, Pelobacter carbinolicus, Syntrophus aciditrophicus, Methanosarcina barkeri, Methanosarcina mazei, Bacillus halodurans, Alkalihalobacillus clausii (strain KSM-K16) (Bacillus clausii), Methanosarcina acetivorans, Kofleriaceae bacterium SLC26A/SulP, Thermodesulfitimonas autotrophica, Fischerella thermalis/Mastigocladus laminosus, Thermosynechococcus vestitus BP-1/(Thermosynechococcus elongatus BP-1) carboxysome assembly protein CcmM, Methanothrix thermoacetophila, Thermosyntropha lipolytica, isoleucine patch superfamily, Desulfofundulus thermobenzoicus, Archaeoglobus veneficus, Suberites domuncula, and any combination thereof. In some embodiments, the enzyme having silicase activity has a sequence identity of at least about 30%, at least about 40%, at least about 50%, at least about 60%, at least about 70%, at least about 80%, at least about 90% or more, with a Methanosarcina thermophila gamma carbonic anhydrase, Bacillus licheniformis CG-B52 gamma carbonic anhydrase, Pelobacter carbinolicus gamma carbonic anhydrase, Syntrophus aciditrophicus gamma carbonic anhydrase, Methanosarcina barkeri gamma carbonic anhydrase, Methanosarcina mazei carbonic anhydrase, Bacillus halodurans alpha carbonic anhydrase, Alkalihalobacillus clausii (strain KSM-K16) (Bacillus clausii) alpha carbonic anhydrase, Methanosarcina acetivorans carbonate dehydratase, Kofleriaceae bacterium SLC26A/SulP transporter domain-containing protein, Thermodesulfitimonas autotrophica carbonic anhydrase/acetyltransferase-like protein (Isoleucine patch superfamily), Fischerella thermalis/Mastigocladus laminosus JSC-11 carboxysome assembly protein CcmM, Thermosynechococcus vestitus BP-1/(Thermosynechococcus elongatus BP-1) carboxysome assembly protein CcmM, Methanothrix thermoacetophila carbonate dehydratase, Thermosyntropha lipolytica carbonic anhydrase or acetyltransferase, isoleucine patch superfamily, Desulfofundulus thermobenzoicus transferase, Archaeoglobus veneficus carbonate dehydratase, Suberites domuncula carbonic anhydrase. In some embodiments, the enzyme having silicase activity has an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, or at least 99.9%, or more sequence identity to an amino acid sequence of any one of SEQ ID NOS: 1-18. In some embodiments, the enzyme having silicase activity is an engineered enzyme, and wherein the engineered enzyme has the sequence of any one of SEQ ID NOS: 19-402, or an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, or at least 99.9%, or more sequence identity to any one of SEQ ID NOS: 19-402. In some embodiments, the enzyme having silicase activity is an enzyme having at least one amino acid variation as compared to a wild-type enzyme. In some embodiments, the enzyme having silicase activity has a pKd of at least about 5, at least about 6, at least about 7, at least about 8, at least about 9, at least about 10, at least about 11, or higher. In some embodiments, the enzyme having silicase activity has a Kcat value of at least about 2 mol per second (mol/s), at least about 10 mol/s, at least about 50 mol/s, at least about 100 mol/s, at least about 200 mol/s, at least about 300 mol/s, at least about 400 mol/s, at least about 500 mol/s, at least about 600 mol/s, at least about 700 mol/s, at least about 800 mol/s, at least about 900 mol/s, at least about 1000 mol/s, or higher. In some embodiments, the reaction conditions comprise a temperature from about 23 to about 85 degrees Celsius (C). In some embodiments, the reaction conditions comprise a temperature from about 45 to about 50 degrees Celsius (C). In some embodiments, the reaction conditions comprise a temperature of about 50 degrees Celsius (C). In some embodiments, the reaction conditions comprise a pH from about 4 to about 11. In some embodiments, the reaction conditions comprise a pH of about 5. In some embodiments, the reaction conditions comprise a pH of about 10. In some embodiments, the reaction conditions comprise contacting the enzyme having silicase activity with a co-factor. In some embodiments, the co-factor is selected from the group consisting of: iron, zinc, copper, nickel, and cobalt. In some embodiments, the co-factor is iron. In some embodiments, the enzyme having silicase activity depolymerizes silicate mineral in the mineral material. In some embodiments, the enzyme having silicase activity cleaves one or more Si—O bonds in the mineral material to generate silicic acid (Si(OH)4). In some embodiments, the metal is lithium, aluminum, iron, nickel, cobalt, strontium, or a rare earth element. In some embodiments, the metal is lithium. In some embodiments, the metal is iron. In some embodiments, the metal is aluminum. In some embodiments, the metal is strontium. In some embodiments, the metal is released into a solution. In some embodiments, the method further comprises extracting the metal from the solution. In some embodiments, the method further comprises purifying the metal from the solution, thereby generating a purified metal. In some embodiments, the purified metal has a purity of at least about 80%. In some embodiments, the purified metal has a purity of at least about 90%. In some embodiments, the purified metal has a purity of at least about 95%. In some embodiments, the purified metal has a purity of at least about 99%. In some embodiments, the purified metal has a purity of at least about 99.99%. In some embodiments, the purified metal has a purity of at least about 99.999%. In some embodiments, the purified metal is purified lithium. In some embodiments, the purified lithium is industrial grade, battery grade, or pharmaceutical grade. In some embodiments, the method is performed in situ or ex situ. In some embodiments, the enzyme having silicase activity has a catalytic efficiency of at least about 80%, at least about 85%, at least about 90%, at least about 91%, at least about 92%, at least about 93%, at least about 94%, at least about 95%, at least about 98% at least about 99%, at least about 99.5% or higher. In some embodiments, the method has a maximum metal extraction rate of at least about 80%, at least about 85%, at least about 90%, at least about 91%, at least about 92%, at least about 93%, at least about 94%, at least about 95%, at least about 98% at least about 99%, at least about 99.5% or higher. In some embodiments, the enzyme having silicase activity is recombinantly produced in a host cell or in a cell-free production system. In some embodiments, the host cell is a bacterial cell or yeast cell. In some embodiments, the bacterial cell is Escherichia coli or the yeast cell is Pichia pastoris or Saccharomyces cerevisiae. In some embodiments, the reaction conditions comprise a rock to liquid ratio from about 1-40% (w/v). In some embodiments, the reaction conditions comprise a rock to liquid ratio of about 30% (w/v). In some embodiments, the reaction conditions comprise a buffer. In some embodiments, the buffer is TRIS, PBS, citrate, monosodium glutamate, or a combination thereof.

In an aspect, provided herein is a method of extracting a metal from a mineral. The method comprises: extracting a metal from a mineral material, the method comprising: contacting the mineral material with an enzyme having silicase activity under reaction conditions such that the metal contained within the mineral material is solubilized and released, wherein the reaction conditions comprise a temperature from about 23-85 degrees Celsius (C), a pH from about 4-11, a co-factor, and a rock to liquid ratio from about 1-40% (w/v), and further comprises collecting the released metal, thereby extracting the metal from the mineral material. In some embodiments, the reaction conditions proceed for about 1-48 hours. In some embodiments, the reaction conditions proceed for about 48 hours. In some embodiments, the reaction conditions comprise a temperature of about 50 degrees Celsius (C). In some embodiments, the reaction condition comprises a pH of about 10. In some embodiments, the co-factor is zinc, iron, copper, cobalt, or any combination thereof. In some embodiments, the co-factor is iron. In some embodiments, the rock to liquid ratio is about 30% (w/v).

In an aspect, provided herein is a non-naturally occurring enzyme having silicase activity, the enzyme comprising at least one amino acid variation relative to a wild-type enzyme and having increased ability to release metals from mineral materials as compared to the wild-type enzyme. In some embodiments, the wild-type enzyme is selected from the group consisting of: Methanosarcina thermophila gamma carbonic anhydrase, Bacillus licheniformis CG-B52 gamma carbonic anhydrase, Pelobacter carbinolicus gamma carbonic anhydrase, Syntrophus aciditrophicus gamma carbonic anhydrase, Methanosarcina barkeri gamma carbonic anhydrase, Methanosarcina mazei carbonic anhydrase, Bacillus halodurans alpha carbonic anhydrase, Alkalihalobacillus clausii (strain KSM-K16) (Bacillus clausii) alpha carbonic anhydrase, Methanosarcina acetivorans carbonate dehydratase, Kofleriaceae bacterium SLC26A/SulP transporter domain-containing protein, Thermodesulfitimonas autotrophica carbonic anhydrase/acetyltransferase-like protein (Isoleucine patch superfamily), Fischerella thermalis/Mastigocladus laminosus JSC-11 carboxysome assembly protein CcmM, Thermosynechococcus vestitus BP-1/(Thermosynechococcus elongatus BP-1) carboxysome assembly protein CcmM, Methanothrix thermoacetophila carbonate dehydratase, Thermosyntropha lipolytica carbonic anhydrase or acetyltransferase, isoleucine patch superfamily, Desulfofundulus thermobenzoicus transferase, Archaeoglobus veneficus carbonate dehydratase, Suberites domuncula carbonic anhydrase, and any combination thereof. In some embodiments, the non-naturally occurring enzyme comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, or at least 99.9%, or more sequence identity to an amino acid sequence of any one of SEQ ID NOS: 1-18. In some embodiments, the wild-type enzyme is a carbonic anhydrase. In some embodiments, the carbonic anhydrase is a gamma carbonic anhydrase or alpha carbonic anhydrase. In some embodiments, the mineral materials comprise rock, ore, natural mineral, man-made mineral, or any combination thereof. In some embodiments, the mineral materials comprise a silicate. In some embodiments, the mineral materials comprise inosilicates, phyllosilicates, amorphous silicates, tectosilicates, or any combination thereof. In some embodiments, the inosilicate is selected from the group consisting of: spodumene, wollastonite, balangeroite, eveslogite, holmquistite, jadeite, shattuckite, augite, tremolite, and any combination thereof. In some embodiments, the phyllosilicate is selected from the group consisting of: lepidolite, hectorite, kaolinite, vermiculite, muscovite, montmorillonite, and any combination thereof. In some embodiments, the amorphous silicate is selected from the group consisting of obsidian, coal fly ash, pumice, and any combination thereof. In some embodiments, the tectosilicate comprises sand, glass, quartz, or any combination thereof. In some embodiments, the non-naturally occurring enzyme has increased ability to depolymerize silicate mineral in the mineral material as compared to the wild-type enzyme, increased selectivity or specificity toward a mineral structure in the mineral material, or both. In some embodiments, the non-naturally occurring enzyme has increased ability to cleave one or more Si—O bonds in the mineral material to generate silicic acid (Si(OH)4) as compared to the wild-type enzyme. In some embodiments, the metal comprises lithium, aluminum, iron, strontium, or any combinations thereof. In some embodiments, the metal comprises lithium. In some embodiments, the metal comprises iron. In some embodiments, the metal comprises aluminum. In some embodiments, the metal comprises strontium. In some embodiments, the non-naturally occurring enzyme is recombinantly produced in a host cell or in a cell-free production system. In some embodiments, the host cell is a bacterial cell or yeast cell. In some embodiments, the bacterial cell is Escherichia coli or the yeast cell is Pichia pastoris or Saccharomyces cerevisiae.

In an aspect, provided herein is a reaction mixture comprising a mineral material and a non-naturally occurring enzyme having silicase activity, wherein the non-naturally occurring enzyme comprises at least one amino acid variation relative to a wild-type enzyme and has increased ability to release metals from the mineral material as compared to the wild-type enzyme. In some embodiments, the mineral material comprises an ore, a rock, a natural mineral material, a man-made mineral material, or any combination thereof. In some embodiments, the reaction mixture has a pH from about 4 to about 11. In some embodiments, the reaction mixture has a pH of about 5. In some embodiments, the reaction mixture has a pH of about 10. In some embodiments, the reaction mixture has a temperature from about 23 to about 85 degrees Celsius (C). In some embodiments, the reaction mixture has a temperature from about 45 to about 50 degrees Celsius (C). In some embodiments, the reaction mixture has a temperature of about 50 degrees Celsius (C). In some embodiments, the reaction mixture further comprises a co-factor of the non-naturally occurring enzyme. In some embodiments, the co-factor is selected from the group consisting of: iron, zinc, copper, nickel, and cobalt. In some embodiments, the co-factor is copper. In some embodiments, the co-factor is iron. In some embodiments, the reaction mixture further comprises a buffered saline solution. In some embodiments, the reaction mixture further comprises an activator co-factor of the non-naturally occurring enzyme. In some embodiments, the activator co-factor is glycine. In some embodiments, the wild-type enzyme is selected from the group consisting of: Methanosarcina thermophila gamma carbonic anhydrase, Bacillus licheniformis CG-B52 gamma carbonic anhydrase, Pelobacter carbinolicus gamma carbonic anhydrase, Syntrophus aciditrophicus gamma carbonic anhydrase, Methanosarcina barkeri gamma carbonic anhydrase, Methanosarcina mazei carbonic anhydrase, Bacillus halodurans alpha carbonic anhydrase, Alkalihalobacillus clausii (strain KSM-K16) (Bacillus clausii) alpha carbonic anhydrase, Methanosarcina acetivorans carbonate dehydratase, Kofleriaceae bacterium SLC26A/SulP transporter domain-containing protein, Thermodesulfitimonas autotrophica carbonic anhydrase/acetyltransferase-like protein (Isoleucine patch superfamily), Fischerella thermalis/Mastigocladus laminosus JSC-11 carboxysome assembly protein CcmM, Thermosynechococcus vestitus BP-1/(Thermosynechococcus elongatus BP-1) carboxysome assembly protein CcmM, Methanothrix thermoacetophila carbonate dehydratase, Thermosyntropha lipolytica carbonic anhydrase or acetyltransferase, isoleucine patch superfamily, Desulfofundulus thermobenzoicus transferase, Archaeoglobus veneficus carbonate dehydratase, Suberites domuncula carbonic anhydrase, and any combination thereof. In some embodiments, the non-naturally occurring enzyme comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, or at least 99.9%, or more sequence identity to an amino acid sequence of any one of SEQ ID NOS: 1-18. In some embodiments, the wild-type enzyme is a carbonic anhydrase. In some embodiments, the carbonic anhydrase is a gamma carbonic anhydrase or alpha carbonic anhydrase. In some embodiments, the mineral material comprises an inosilicate, a phyllosilicate, an amorphous silicate, a tectosilicate or any combination thereof. In some embodiments, the inosilicate is selected from the group consisting of: spodumene, wollastonite, balangeroite, eveslogite, holmquistite, jadeite, shattuckite, augite, tremolite, and any combination thereof. In some embodiments, the phyllosilicate is selected from the group consisting of: lepidolite, hectorite, kaolinite, vermiculite, muscovite, montmorillonite, and any combination thereof. In some embodiments, the amorphous silicate is selected from the group consisting of: obsidian, coal fly ash, pumice, glass, and any combination thereof. In some embodiments, the non-naturally occurring enzyme has increased ability to depolymerize silicate in the mineral material as compared to the wild-type enzyme. In some embodiments, the non-naturally occurring enzyme has increased ability to cleave one or more Si—O bonds in the mineral material to generate silicic acid (Si(OH)4) as compared to the wild-type enzyme. In some embodiments, the metal comprises lithium. In some embodiments, the metal comprises aluminum. In some embodiments, the metal comprises iron. In some embodiments, the metal comprises strontium. In some embodiments, the non-naturally occurring enzyme is recombinantly produced in a host cell. In some embodiments, the host cell is a bacterial cell or a yeast cell. In some embodiments, the bacterial cell is Escherichia coli or the yeast cell is Pichia pastoris or Saccharomyces cerevisiae. In some embodiments, the reaction mixture has a rock to liquid ratio from about 1-40% (w/v). In some embodiments, the reaction mixture has a rock to liquid ratio of about 30% (w/v). In some embodiments, reaction mixture has a buffer. In some embodiments, the buffer is TRIS, PBS, citrate, monosodium glutamate, or a combination thereof. In some embodiments, the reaction mixture proceeds for about 1-48 hours. In some embodiments, the reaction mixture proceeds for about 48 hours.

In an aspect, provided herein is a polynucleotide comprising a nucleotide sequence encoding the non-naturally occurring enzyme disclosed herein.

In an aspect, provided herein is a vector comprising the polynucleotide disclosed herein.

In an aspect, provided herein is a method of increasing silicase activity of an enzyme, the method comprising contacting the enzyme with a non-natural co-factor, wherein the non-natural co-factor increases silicase activity of the enzyme as compared to the enzyme in the presence of a natural co-factor. In some embodiments, the non-natural co-factor is copper. In some embodiments, the natural co-factor is zinc. In some embodiments, the natural co-factor is iron. In some embodiments, the method is performed in the absence of the natural co-factor. In some embodiments, the non-natural co-factor does not act as a co-factor for the enzyme having silicase activity in nature. In some embodiments, the method further comprises contacting the enzyme and the non-natural co-factor with a mineral material under reaction conditions such that a metal contained within the mineral material is solubilized and released from the mineral material. In some embodiments, the amount of metal solubilized and released from the mineral material is greater than an amount of metal solubilized and released from the mineral material when the enzyme is contacted with the natural co-factor.

In some embodiments, the mineral material comprises a rock, an ore, a natural mineral material, a man-made mineral material, or any combination thereof. In some embodiments, the mineral material comprises a silicate. In some embodiments, the mineral material comprises an inosilicate, a phyllosilicate, an amorphous silicate, a tectosilicate, or any combination thereof. In some embodiments, the inosilicate is selected from the group consisting of: spodumene, wollastonite, balangeroite, eveslogite, holmquistite, jadeite, shattuckite, augite, tremolite, and any combination thereof. In some embodiments, the phyllosilicate is selected from the group consisting of: lepidolite, hectorite, kaolinite, vermiculite, muscovite, montmorillonite, and any combination thereof. In some embodiments, the amorphous silicate is selected from the group consisting of: obsidian, coal fly ash, pumice, glass, and any combination thereof. In some embodiments, the enzyme having silicase activity is a carbonic anhydrase. In some embodiments, the carbonic anhydrase is a gamma carbonic anhydrase or alpha carbonic anhydrase. In some embodiments, the enzyme having silicase activity is derived from an organism selected from the group consisting of: Methanosarcina thermophila, Bacillus licheniformis CG-B52, Pelobacter carbinolicus, Syntrophus aciditrophicus, Methanosarcina barkeri, Methanosarcina mazei, Bacillus halodurans, Alkalihalobacillus clausii (strain KSM-K16) (Bacillus clausii), Methanosarcina acetivorans, Kofleriaceae bacterium, Thermodesulfitimonas autotrophica, Fischerella thermalis/Mastigocladus laminosus JSC-11 carboxysome assembly protein CcmM, Thermosynechococcus vestitus BP-1/(Thermosynechococcus elongatus BP-1) carboxysome assembly protein CcmM, Methanothrix thermoacetophila, Thermosyntropha lipolytica, Desulfofundulus thermobenzoicus, Archaeoglobus veneficus, Suberites domuncula, and any combination thereof. In some embodiments, the enzyme having silicase activity comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, or at least 99.9%, or more sequence identity to an amino acid sequence of any one of SEQ ID NOS: 1-18. In some embodiments, the enzyme having silicase activity is an engineered enzyme, optionally wherein the engineered enzyme has the sequence of any one of SEQ ID NOS: 19-402. In some embodiments, the enzyme having silicase activity is an enzyme having at least one amino acid variation as compared to a wild-type enzyme. In some embodiments, the enzyme having silicase activity has a pKd of at least about 5, at least about 6, at least about 7, at least about 8, at least about 9, at least about 10, at least about 11, or higher. In some embodiments, the enzyme having silicase activity has a Kcat value of at least about 2 mol per second (mol/s), at least about 10 mol/s, at least about 50 mol/s, at least about 100 mol/s, at least about 200 mol/s, at least about 300 mol/s, at least about 400 mol/s, at least about 500 mol/s, at least about 600 mol/s, at least about 700 mol/s, at least about 800 mol/s, at least about 900 mol/s, at least about 1000 mol/s, or higher. In some embodiments, the method is performed under reaction conditions. In some embodiments, the reaction conditions comprise a temperature from about 23 to about 85 degrees Celsius (C). In some embodiments, the reaction conditions comprise a temperature from about 45 to about 50 degrees Celsius (C). In some embodiments, the reaction conditions comprise a temperature of about 50 degrees Celsius (C). In some embodiments, the reaction conditions comprise a pH from about 4 to about 11. In some embodiments, the reaction conditions comprise a pH of 5. In some embodiments, the reaction conditions comprise a pH of 10. In some embodiments, the enzyme having silicase activity depolymerizes silicate mineral in the mineral material. In some embodiments, the enzyme having silicase activity cleaves one or more Si—O bonds in the mineral material to generate silicic acid (Si(OH)4). In some embodiments, the metal is selected from the group consisting of: lithium, aluminum, iron, nickel, cobalt, strontium, and rare earth metals. In some embodiments, the metal is lithium. In some embodiments, the metal is iron. In some embodiments, the metal is aluminum. In some embodiments, the metal is strontium. In some embodiments, the metal is released into a solution. In some embodiments, the method further comprises extracting the metal from the solution. In some embodiments, the method further comprises purifying the metal from the solution, thereby generating a purified metal, a solid metal complex, a metal precipitate, or any combination thereof. In some embodiments, the purified metal has a purity of at least about 80%, at least about 90%, at least about 95%, at least about 99%, at least about 99.99%, at least about 99.9999% or greater. In some embodiments, the method is performed in situ or ex-situ. In some embodiments, the enzyme having silicase activity has a catalytic efficiency of at least about 80%, at least about 85%, at least about 90%, at least about 91%, at least about 92%, at least about 93%, at least about 94%, at least about 95%, at least about 98% at least about 99%, at least about 99.5% or higher. In some embodiments, the method has a maximum metal extraction rate of at least about 80%, at least about 85%, at least about 90%, at least about 91%, at least about 92%, at least about 93%, at least about 94%, at least about 95%, at least about 98% at least about 99%, at least about 99.5% or higher. In some embodiments, the enzyme having silicase activity is recombinantly produced in a host cell or in a cell-free production system. In some embodiments, the host cell is a bacterial cell or a yeast cell. In some embodiments, the bacterial cell is Escherichia coli or the yeast cell is Pichia pastoris or Saccharomyces cerevisiae. In some embodiments, the reaction conditions comprise a rock to liquid ratio from about 1-40% (w/v). In some embodiments, the reaction conditions comprise a rock to liquid ratio of about 30% (w/v). In some embodiments, the reaction conditions comprise a buffer. In some embodiments, the buffer is TRIS, PBS, citrate, monosodium glutamate, or a combination thereof.

In an aspect, provided herein is a reaction mixture comprising an enzyme having silicase activity, and a non-natural co-factor.

In some embodiments, the non-natural co-factor is bound to the enzyme having silicase activity. In some embodiments, the non-natural co-factor increases a function of the enzyme having silicase activity as compared to a reaction mixture comprising the enzyme having silicase activity and a natural co-factor. In some embodiments, the non-natural co-factor is copper. In some embodiments, the natural co-factor is zinc. In some embodiments, the natural co-factor is iron. In some embodiments, the reaction mixture does not contain the natural co-factor. In some embodiments, the non-natural co-factor does not act as a co-factor for the enzyme having silicase activity in nature. In some embodiments, the reaction mixture further comprises a mineral material and reaction conditions such that a metal contained within the mineral material is solubilized and released from the mineral material. In some embodiments, the enzyme having silicase activity has increased ability to release metals from the mineral material in the presence of the non-natural co-factor as compared to the enzyme having silicase activity in the presence of the natural co-factor. In some embodiments, the mineral material comprises an inosilicate, a phyllosilicate, an amorphous silicate, or any combination thereof. In some embodiments, the inosilicate is selected from the group consisting of: spodumene, wollastonite, balangeroite, eveslogite, holmquistite, jadeite, shattuckite, augite, tremolite, and any combination thereof. In some embodiments, the phyllosilicate is selected from the group consisting of: lepidolite, hectorite, kaolinite, vermiculite, muscovite, montmorillonite, and any combination thereof. In some embodiments, the amorphous silicate is selected from the group consisting of: obsidian, coal, pumice, glass, and any combination thereof. In some embodiments, the enzyme having silicase activity has a sequence identity of at least about 30%, at least about 40%, at least about 50%, at least about 60%, at least about 70%, at least about 80%, at least about 90%, or more identity with a carbonic anhydrase. In some embodiments, the enzyme having silicase activity has a sequence identity of at least about 30%, at least about 40%, at least about 50%, at least about 60%, at least about 70%, at least about 80%, at least about 90%, or more identity with an alpha carbonic anhydrase or a gamma carbonic anhydrase. In some embodiments, the enzyme having silicase activity is derived from an organism selected from the group consisting of: Methanosarcina thermophila, Bacillus licheniformis CG-B52, Pelobacter carbinolicus, Syntrophus aciditrophicus, Methanosarcina barkeri, Methanosarcina mazei, Bacillus halodurans, Alkalihalobacillus clausii (strain KSM-K16) (Bacillus clausii), Methanosarcina acetivorans, Kofleriaceae bacterium SLC26A/SulP, Thermodesulfitimonas autotrophica, Fischerella thermalis/Mastigocladus laminosus, Thermosynechococcus vestitus BP-1/(Thermosynechococcus elongatus BP-1) carboxysome assembly protein CcmM, Methanothrix thermoacetophila, Thermosyntropha lipolytica, isoleucine patch superfamily, Desulfofundulus thermobenzoicus, Archaeoglobus veneficus, Suberites domuncula, and any combination thereof. In some embodiments, the enzyme having silicase activity comprises an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, or at least 99.9%, or more sequence identity to an amino acid sequence of any one of SEQ ID NOS: 1-18. In some embodiments, the enzyme having silicase activity is an engineered enzyme, optionally wherein the enzyme has the sequence of any one of SEQ ID NOS: 19-402. In some embodiments, the enzyme having silicase activity is an enzyme having at least one amino acid variation as compared to a wild-type enzyme. In some embodiments, the enzyme having silicase activity has a pKd of at least about 5, at least about 6, at least about 7, at least about 8, at least about 9, at least about 10, at least about 11, or higher. In some embodiments, the enzyme having silicase activity has a Kcat value of at least about 2 mol per second (mol/s), at least about 10 mol/s, at least about 50 mol/s, at least about 100 mol/s, at least about 200 mol/s, at least about 300 mol/s, at least about 400 mol/s, at least about 500 mol/s, at least about 600 mol/s, at least about 700 mol/s, at least about 800 mol/s, at least about 900 mol/s, at least about 1000 mol/s, or higher. In some embodiments, the reaction mixture has a temperature from about 23 to about 85 degrees Celsius (C). In some embodiments, the reaction mixture has a temperature from about 45 to about 50 degrees Celsius (C). In some embodiments, the reaction mixture has a temperature of about 50 degrees Celsius (C). In some embodiments, the reaction mixture has a pH from about 4 to about 11. In some embodiments, the reaction mixture has a pH of 5. In some embodiments, the reaction mixture has a pH of 10. In some embodiments, the reaction mixture further comprises a buffered saline solution. In some embodiments, the reaction mixture further comprises an activator co-factor of the non-naturally occurring enzyme. In some embodiments, the activator co-factor is glycine or an iron ion. In some embodiments, the metal is lithium, aluminum, iron, nickel, cobalt, strontium, or a rare earth element. In some embodiments, the metal is lithium. In some embodiments, the metal is iron. In some embodiments, the metal is aluminum. In some embodiments, the metal is strontium. In some embodiments, the enzyme having silicase activity is recombinantly produced in a host cell. In some embodiments, the host cell is a bacterial cell or yeast cell. In some embodiments, the bacterial cell is Escherichia coli or the yeast cell is Pichia pastoris or Saccharomyces cerevisiae. In some embodiments, the reaction conditions comprise a rock to liquid ratio from about 1-40% (w/v). In some embodiments, the reaction conditions comprise a rock to liquid ratio of about 30% (w/v). In some embodiments, the reaction conditions comprise a buffer. In some embodiments, the buffer is TRIS, PBS, citrate, monosodium glutamate, or a combination thereof. In some embodiments, the reaction conditions proceed for about 1-48 hours. In some embodiments, the reaction conditions proceed for about 48 hours.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows an example workflow according to the methods of the present disclosure;

FIG. 2 presents the rate of production of Si(OH)₄ corresponding to reaction rates of degrading silicate minerals(Alpha Spodumene and Beta Spodumene) using an enzyme having silicase activity according to the embodiments of the present disclosure;

FIG. 3 presents the rate of production of Si(OH)4 corresponding to reaction rates of degrading silicate minerals (iron ore, platinum group metal (PGM) tailing, and Bauxite) using an enzyme having silicase activity according to the embodiments of the present disclosure;

FIG. 4 presents the rate of production of Si(OH)₄ corresponding to reaction rates of degrading silicate minerals (Rhyolite and Olivine) using an enzyme having silicase activity according to the embodiments of the present disclosure;

FIG. 5 presents the rate of production of Si(OH)₄ corresponding to reaction rates of degrading silicate minerals (Hectorite mix, Clay, a silicate named Maverick source, and a Lepidolite) using an enzyme having silicase activity according to the embodiments of the present disclosure;

FIG. 6 presents the rate of production of Si(OH)4 corresponding to reaction rates of degrading silicate minerals (crushed glass and Perlite) using an enzyme having silicase activity according to the embodiments of the present disclosure; and

FIG. 7 presents the rate of production of Si(OH)₄ corresponding to reaction rates of degrading silicate minerals (Oil Shale and Fly Ash) using an enzyme having silicase activity according to the embodiments of the present disclosure.

### DETAILED DESCRIPTION

Metals such as lithium, aluminum, iron, nickel, cobalt, strontium, and rare earth elements have vast applications across various industries and in different products. The demand for such metals continues to increase, and there is an unmet need for efficient technologies to facilitate access to metal sources, and to extract and collect the metals for use in products and industries in need thereof and to meet demand. As an example, lithium is highly in demand for rechargeable batteries which can be used in a variety of products such as electronics, electric motors and electric vehicles, clean energy industry, solar panels, and beyond. As these industries advance and become more prominent in global markets, so does the demand for lithium. Lithium can be found in a number of sources, including in brine, for example, in brine deposits generated as a result of accumulations of saline groundwater enriched in dissolved lithium. However, brine sources of lithium are limited in abundance and can only be found in limited geographical locations, mostly located in South America.

Another prominent and abundant source of metals are mineral materials, such as natural minerals (e.g., rock, ore, clay) and man-made minerals that are commonly available across the world in a diverse range of geographical areas, constituting a major primary source of metals. The currently available technologies for extracting metals from solid mineral materials/sources, rocks, and ores are limited, inefficient, costly, and environmentally harmful. An example of such process is acid leaching or acid roasting which involves contacting a mineral with a strong acid (e.g., sulfuric acid) to extract a metal, such as lithium, from the mineral. Acid leaching/roasting usually requires reaction conditions involving highly acidic pH, high temperatures (e.g., 200 degrees Celsius (C) and above) and significant energy consumption (e.g., over 6000 megajoules (MJ) per ton of Li₂O extracted). This process is expensive, energy-inefficient, and harmful to the environment. Therefore, there is an unmet need for improved compositions, methods, and systems to address these shortcomings.

Provided herein are compositions, methods, and systems that can efficiently extract metals from minerals (e.g., natural minerals (e.g., rock, ore, clay), man-made minerals) in an efficient, industrially scalable, inexpensive, and environmentally friendly fashion. For example, in some cases, the methods may avoid reaction conditions requiring substances (e.g., highly acidic solvents), high temperatures and pressures, and the like, which may cause harm to the environment and/or increase the cost, energy demands, and/or environmental footprint of the process. In some cases, this is accomplished by performing an enzymatic reaction on a mineral material, to extract and separate a metal (e.g., metal ion/atom) therefrom. The enzymatic reaction may have improved features such as higher reaction rate, specificity toward degrading a mineral material, acting on a certain substrate in the mineral material with high/improved substrate specificity, such that performing the reaction can be industrially scaled and implemented for releasing and collecting the metal. For example, in some cases, the reaction may not require temperatures that are significantly higher than room temperature, pressures significantly higher than atmospheric pressure, highly acidic or highly basic pH conditions, and other conditions that are environmentally harmful and costly. Instead, in many cases, the reactions of the present disclosure may be efficiently performed in near-ambient temperature, near-atmospheric pressure, and/or near-neutral pH conditions, reducing their cost, energy demand, and environmental footprint. The details of such reaction conditions are further elaborated on herein.

In some cases, the enzymatic reaction may comprise using one or more enzymes and/or co-factors. The enzymatic reaction may comprise an enzymatic degradation/digestion reaction performed with the aid of one or more enzymes and/or co-factors. The enzymes may catalyze the reaction and facilitate the extraction of the metal from the mineral material encasing it. For example, enzymes can be used to degrade, dissolve, and/or depolymerize silicates, and liberate metals (e.g., metal ions) therefrom at near-ambient temperatures without the need for an energy-intensive, high temperature acid separation process. This process significantly decreases the environmental impact of refining lithium and other metals deposited in mineral materials. Provided herein are also enzymes and co-factors with enhanced features and capabilities for performing such reactions. Such enzymes may be semi-synthetic and/or engineered enzymes with features, capabilities, sequences, methods of generation, and methods of use that are presented and further elaborated on in the present disclosure.

The term “sequence identity” as used herein generally refers to an exact nucleotide-to-nucleotide or amino acid-to-amino acid correspondence of two polynucleotides or polypeptide sequences, respectively. Typically, techniques for determining sequence identity include determining the nucleotide sequence of a polynucleotide and/or determining the amino acid sequence encoded thereby, and comparing these sequences to a second nucleotide or amino acid sequence. Two or more sequences (polynucleotide or amino acid) can be compared by determining their percentage (%) of “sequence identity”. The % of sequence identity of two sequences, whether nucleic acid or amino acid sequences, is the number of exact matches between two aligned sequences divided by the length of the longer sequence and multiplied by 100. Percent identity may also be determined, for example, by comparing sequence information using the advanced BLAST computer program, including version 2.2.9, available from the National Institutes of Health. The BLAST program is based on the alignment method of Karlin and Altschul, Proc. Natl. Acad. Sci. USA, 87:2264-2268 (1990) and as discussed in Altschul, et al., J. Mol. Biol., 215:403-410 (1990); Karlin And Altschul, Proc. Natl. Acad. Sci. USA, 90:5873-5877 (1993); and Altschul et al., Nucleic Acids Res., 25:3389-3402 (1997). The program may be used to determine percent identity over the entire length of the proteins being compared. Default parameters are provided to optimize searches with short query sequences in, for example, with the blastp program. The program also allows use of an SEG filter to mask-off segments of the query sequences as determined by the SEG program of Wootton and Federhen, Computers and Chemistry 17:149-163 (1993). Ranges of desired degrees of sequence identity are approximately 50% to 100% and integer values therebetween. In general, this disclosure encompasses sequences with at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, or at least 99.9% sequence identity with any sequence provided herein.

The terms “variant enzyme”, “enzyme variant”, “modified enzyme”, “synthetic enzyme”, “truncated enzyme”, and “engineered enzyme” are used interchangeably throughout to generally refer to non-naturally occurring polypeptides. The non-naturally occurring polypeptides have been designed and sequences included herein.

The term “about” or “approximately” generally means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

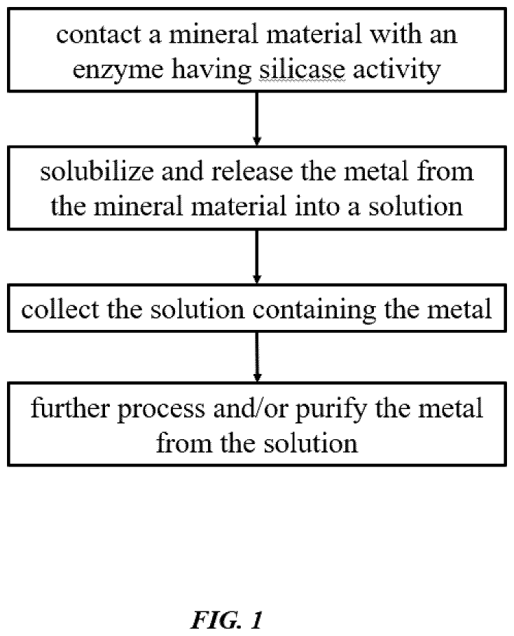

In an aspect, provided herein is a method of extracting a metal from a mineral material (e.g., natural mineral material, man-made mineral material, rock, ore, clay and/or other kinds of mineral material). The method may comprise contacting the mineral material with an enzyme having silicase activity under reaction conditions such that the metal contained within the mineral material is solubilized and released. The method may further comprise collecting the released metal, thereby extracting the metal from the mineral material. In some cases, the mineral material comprises or is a natural mineral material or a man-made mineral material. In some cases, the natural mineral material comprises or is a rock, an ore, or a clay. In some cases, the metal is a metal ion or metal atom. FIG. 1 shows an example workflow according to the embodiments of the present disclosure.

In some cases, the enzyme comprises silicase activity such as the capability to digest or degrade a silicate. In some cases, the mineral material (e.g., rock/ore/clay) comprises a silicate. Silicate may comprise any kind of silicate. In some cases, the mineral material comprises an inosilicate, a phyllosilicate, an amorphous silicate, or any combination thereof. In some cases, the mineral material comprises similar or near-similar unit cell geometries. In some cases, the inosilicate is selected from the group consisting of: spodumene, wollastonite, balangeroite, eveslogite, holmquistite, jadeite, shattuckite, augite, tremolite, and any combination thereof. In some cases, the phyllosilicate is selected from the group consisting of: lepidolite, hectorite, kaolinite, vermiculite, muscovite, montmorillonite, and any combination thereof. In some cases, silicate may be an amorphous silicate. In some cases, the amorphous silicate is selected from the group consisting of a tectosilicate, obsidian, coal fly ash, pumice, glass, and any combination thereof.

In some cases, the enzyme having silicase activity disclosed herein is an engineered enzyme with improved characteristics compared to a wild-type enzyme, such that the modified enzyme exhibits improved ability (e.g., increased reaction rate, increased efficiency, increased ability to degrade silicates, increased specificity for a silicate type) to release metals from mineral materials. The enzyme used in the methods of the present disclosure are generally capable of degrading silicates. In some cases, the enzyme having silicase activity comprises or is a carbonic anhydrase, such as a gamma carbonic anhydrase, or an alpha carbonic anhydrase. In some cases, a wild-type enzyme may be used to perform the methods of the present disclosure. Alternatively or in addition, a modified, mutated, and/or engineered enzyme may be used to perform the methods of the present disclosure. In some cases, an enzyme used in a reaction/process of the present disclosure is a synthetic or semi-synthetic engineered enzyme. In some cases, an enzyme of the present disclosure may be a variant of a carbonic anhydrase, such as a gamma carbonic anhydrase, or alpha carbonic anhydrase. In some cases, an enzyme of the present disclosure may share some similarities (e.g., sequence identity, enzymatic activity) with a carbonic anhydrase. Any combination of wild-type and engineered/modified enzymes may be used.

In some cases, the enzyme having silicase activity is an engineered enzyme. In some cases, the enzyme having silicase activity is an enzyme having at least one amino acid variation as compared to a wild-type enzyme. In some cases, the enzyme having silicase activity may comprise an amino acid sequence having at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, at least about 99.5%, at least about 99.5%, or greater, sequence identity to an amino acid sequence of a wild-type silicase enzyme. In some cases, the enzyme having silicase activity comprises an amino acid sequence having at most about 95%, at most about 90%, at most about 80%, at most about 70%, at most about 60%, at most about 50%, at most about 40%, at most about 30%, at most about 20%, or at most about 10% sequence identity to an amino acid sequence of a wild-type silicase enzyme. In some cases, the enzyme having silicase activity comprises an amino acid sequence having about 95%, about 90%, about 85%, about 80%, about 75%, about 70%, about 65%, about 60%, about 55%, about 50%, about 45%, about 40%, about 35, about 30%, about 25%, about 20%, about 15%, or about 10% sequence identity to an amino acid sequence of a wild-type silicase enzyme.

In some cases, the enzyme having silicase activity is derived from an organism selected from the group consisting of: Methanosarcina thermophila, Bacillus lichenmformis CG-B52, Pelobacter carbinolicus, Syntrophus aciditrophicus, Methanosarcina barkeri, Methanosarcina mazei, Bacillus halodurans, Alkalihalobacillus clausii (strain KSM-K16) (Bacillus clausii), Methanosarcina acetivorans, Kofleriaceae bacterium SLC26A/SulP, Thermodesulfitimonas autotrophica, Fischerella thermalis/Mastigocladus laminosus, Thermosynechococcus vestitus BP-1/(Thermosynechococcus elongatus BP-1) carboxysome assembly protein CcmM, Methanothrix thermoacetophila, Thermosyntropha lipolytica, isoleucine patch superfamily, Desulfofundulus thermobenzoicus, Archaeoglobus veneficus, Suberites domuncula, and any combination thereof.

In some cases, the enzyme having silicase activity has a sequence identity of at least about 10%, at least about 20%, at least about 30%, at least about 40%, at least about 50%, at least about 60%, at least about 70%, at least about 80%, at least about 90% or more with a Methanosarcina thermophila gamma carbonic anhydrase, Bacillus lichenmformis CG-B52 gamma carbonic anhydrase, Pelobacter carbinolicus gamma carbonic anhydrase, Syntrophus aciditrophicus gamma carbonic anhydrase, Methanosarcina barkeri gamma carbonic anhydrase, Methanosarcina mazei carbonic anhydrase, Bacillus halodurans alpha carbonic anhydrase, Alkalihalobacillus clausii (strain KSM-K16) (Bacillus clausii) alpha carbonic anhydrase, Methanosarcina acetivorans carbonate dehydratase, Kofleriaceae bacterium SLC26A/SulP transporter domain-containing protein, Thermodesulfitimonas autotrophica carbonic anhydrase/acetyltransferase-like protein (Isoleucine patch superfamily), Fischerella thermalis/Mastigocladus laminosus JSC-11 carboxysome assembly protein CcmM, Thermosynechococcus vestitus BP-1/(Thermosynechococcus elongatus BP-1) carboxysome assembly protein CcmM, Methanothrix thermoacetophila carbonate dehydratase, Thermosyntropha lipolytica carbonic anhydrase or acetyltransferase, isoleucine patch superfamily, Desulfofundulus thermobenzoicus transferase, Archaeoglobus veneficus carbonate dehydratase, Suberites domuncula carbonic anhydrase.

In some cases, the enzyme having silicase activity has a sequence identity of at most about 80%, at most about 70%, at most about 60%, at most about 50%, at most about 40%, at most about 30%, at most about 20%, at most about 10% or less with a Methanosarcina thermophila gamma carbonic anhydrase, Bacillus lichenmformis CG-B52 gamma carbonic anhydrase, Pelobacter carbinolicus gamma carbonic anhydrase, Syntrophus aciditrophicus gamma carbonic anhydrase, Methanosarcina barkeri gamma carbonic anhydrase, Methanosarcina mazei carbonic anhydrase, Bacillus halodurans alpha carbonic anhydrase, Alkalihalobacillus clausii (strain KSM-K16) (Bacillus clausii) alpha carbonic anhydrase, Methanosarcina acetivorans carbonate dehydratase, Kofleriaceae bacterium SLC26A/SulP transporter domain-containing protein, Thermodesulfitimonas autotrophica carbonic anhydrase/acetyltransferase-like protein (Isoleucine patch superfamily), Fischerella thermalis/Mastigocladus laminosus JSC-11 carboxysome assembly protein CcmM, Thermosynechococcus vestitus BP-1/(Thermosynechococcus elongatus BP-1) carboxysome assembly protein CcmM, Methanothrix thermoacetophila carbonate dehydratase, Thermosyntropha lipolytica carbonic anhydrase or acetyltransferase, isoleucine patch superfamily, Desulfofundulus thermobenzoicus transferase, Archaeoglobus veneficus carbonate dehydratase, Suberites domuncula carbonic anhydrase.

In some embodiments, the enzyme having silicase activity comprises an amino acid sequence having at least about 50%, at least about 60%, at least about 70%, at least about 80%, at least about 85%, at least about 90%, at least about 91%, at least about 92%, at least about 93%, at least about 84%, at least about 95%, at least about 96%, at least about 97%, at least about 98%, at least about 99%, at least about 99.5%, at least about 99.9%, or more sequence identity sequence identity to an amino acid sequence of any one of SEQ ID NOS: 1-18 provided in Table 1.

TABLE 1
Library of Carbonic Anhydrase Enzymes

| | | Uniprot | |
|---|---|---|---|
| SEQ ID | | Accession | |
| NO: | Name Enzyme | number | SEQUENCE |
| 1 | γ Carbonic Anhydrase | P40881 | MMFNKQIFTILILSLSLALAGSGCI |
| | | | SEGAEDNVAQEITVDEFSNIRENP |
| | | | VTPWNPEPSAPVIDPTAYIDPQAS |
| | | | VIGEVTIGANVMVSPMASIRSDE |
| | | | GMPIFVGDRSNVQDGVVLHALE |
| | | | TINEEGEPIEDNIVEVDGKEYAVY |
| | | | IGNNVSLAHQSQVHGPAAVGDD |
| | | | TFIGMQAFVFKSKVGNNCVLEPR |
| | | | SAAIGVTIPDGRYIPAGMVVTSQ |
| | | | AEADKLPEVTDDYAYSHTNEAV |
| | | | VYVNVHLAEGYKETS |
| 2 | γ Carbonic Anhydrase | T5H8M4 | MKLSSKLILGLTVSSLAGKFLEKL |
| | | | LIQDNVSPNITASFNQEADIPDIDA |
| | | | SSYIHHFASVIGSVVIGRNVFIGPF |
| | | | SSIRGDVGLKIFISHDCNIQDGVV |
| | | | LHGLKNYEYNSPVTEHSVFKDRE |
| | | | SYSIYIGEKVSLAPQCQIYGPVRID |
| | | | KNVFVGMQSLVFDAYIQEDTVIE |
| | | | PGAKIIGVTIPPKRFVSAGRVISNQ |
| | | | EDANRLPEITDSYPYHDLNSKMT |
| | | | SVNLELAKGYKKEERQWKL |
| 3 | γ Carbonic Anhydrase | Q3A3H4 | MIEKNVVTDFCSEASEPVIDASTY |
| | | | VHPLAAVIGNVILGKNIMVSPTA |
| | | | VVRGDEGQPLHVGDDSNIQDGV |
| | | | VIHALETEMNGKPVAKNLYQVD |
| | | | GRSYGAYVGCRVSLAHQVQIHG |
| | | | PAVVLDDTFVGMKSLVFKSFVG |
| | | | KGCVIEPGSIVMGVTVADGRYVP |
| | | | AGSVIRTQEDADALPEIGADYPFR |
| | | | AMNPGVVHVNTALAKGYMVKQ |
| | | | GN |
| 4 | γ Carbonic Anhydrase | | MIGKNVLTDFSARASEPVIGSFTF |
| | | | VHPLAAVIGNVILGDNIMVSPGA |
| | | | SIRGDEGQPLYVGSDSNVQDGVV |
| | | | IHALETELDGKPVEKNLVEVDGK |
| | | | KYAVYVGNRVSLAHQVQVHGP |
| | | | AVIRDDTFVGMKSLVFKSYVGSN |
| | | | CVIEPGVLLMGVTVADGRYVPA |
| | | | GSVVKTQEQADALPVITDDYPM |
| | | | KEMNKGVLHVNKALARGYLAA |
| | | | GS |
| 5 | γ Carbonic Anhydrase | Q2LUP7 | MRFNKQTFTILILSLSLALLGSGCI |
| | | | SEGEGAEGNVTQGITESEFSNIRE |
| | | | NPVTPWNPVPVAPVIDPTAFIDPQ |
| | | | ASVIGNVTIGASVMVSPMASIRSD |
| | | | EGMPIFVGDRSNVQDGVVLHAL |
| | | | ETIDEEGEPVENNIVEVGGKKYA |
| | | | VYIGENVSLAHQAQVHGPASVG |
| | | | NDTFIGMQAFVFKSKIGNNCVLE |
| | | | PTSAAIGVTVPDGRYIPAGMVVT |
| | | | SQAEADNLSEITDDYAYKHTNEA |
| | | | VVYVNVHLAEGYNKA |
| 6 | Carbonic Anhydrase | Q467M8 | MALLLSLAITLAGSGCVSQGEGA |
| | | | EEGENIEAEEVEANVEESNIRANP |
| | | | VTPWNPEPTEPVIDPTAYIHPQAS |
| | | | VIGDVTIGASVMVSPMASVRSDE |
| | | | GMPIFVGDECNIQDGVILHALET |
| | | | VNEEGEPVEENQVEVDGKKYAV |
| | | | YIGERVSLAHQAQVHGPSLVGND |
| | | | TFIGMQTFVFKAKIGNNCVLEPTS |
| | | | AAIGVTVPDGRYIPAGTVVTSQD |
| | | | EADKLPEVTDDYAYKHTNEAVV |
| | | | YVNTNLAEGYNA |
| 7 | α Carbonic | Q8PSJ1 | MKKYLWGKTCLVVSLSVMVTA |
| | Anhydrase | | CSSAPSTEPVDEPSETHEETSGGA |
| | | | HEVHWSYTGDTGPEHWAELDSE |
| | | | YGACAQGEEQSPINLDKTEAIDT |
| | | | DTEIHVHYEPSSFTIKNNGHTIQA |
| | | | ETTSDKNTIEIDGKEYTLVQFHFH |
| | | | IPSEHEMEGKNLDMELHFVHKNE |
| | | | NDELAVLGVLMKAGEENEELAQ |
| | | | LWSKLPAEETEENISLDESIDLNV |
| | | | LLPESKEGFHYNGSLTTPPCSEGV |
| | | | KWTVLSEPITVSQEQIDAFAEIFP |
| | | | DNHRPVQPWNDRDVYDVITE |
| 8 | α Carbonic | A0A0N9WRG3 | MKRSHLFTSITLASVVTLATAPA |
| | Anhydrase | | ASAASFLSPLQALKASWSYEGET |
| | | | GPEFWGDLDEAFAACSNGKEQSP |
| | | | INLFYDREQTSKWNWAFSYSEAA |
| | | | FSVENNGHTIQANVENEDAGGLE |
| | | | INGEAYQLIQFHFHTPSEHTIEETS |
| | | | FPMELHLVHANHAGDLAVLGVL |
| | | | MEMGNDHEGIEAVWEVMPEEEG |
| | | | TAAYSISLDPNLFLPESVTAYQYD |
| | | | GSLTTPPCSEGVKWTVLNDTISIS |
| | | | ETQLDAFRDIYPQNYRPVQELGD |
| | | | REIGFHYH |
| 9 | Carbonate | Q5WD44 | MKINRIFLALLFSLALTLAGSGCV |
| | dehydratase | | SQGEGAEDGESADTEVESEVSNI |
| | | | RANPVTPWNPEPTEPVIDSTAYIH |
| | | | PQAAVIGDVTIGASVMVSPMASV |
| | | | RSDEGTPIFVGDETNIQDGVVLH |
| | | | ALETVNEEGEPVESNLVEVDGEK |
| | | | YAVYVGERVSLAHQSQIHGPAY |
| | | | VGNDTFIGMQALVFKANVGDNC |
| | | | VLEPKSGAIGVTIPDGRYIPAGTV |
| | | | VTSQAEADELPEVTDDYGYKHT |
| | | | NEAVVYVNVNLAAGYNA |
| 10 | Kofleriaceae | Q8TMW3 | MRTNRVRTAGASKWSGVSDIRT |
| | bacterium Carbonic | | TLRERWSEIAAQGLSYHDVLAGL |
| | Anhydrase | | TVATVAIPLNVALAISAGLPPSAG |
| | | | LLAGAVGGLFAAAFGGSNFQVS |
| | | | GPAAALNVMVFGVVAKFGLGGA |
| | | | AAAALVCGIVGIALGVSGLGKYS |
| | | | NLMPKLVLAGFTTGVGLKLLDQ |
| | | | QIPILLGSDLALWHMLSNFWAME |
| | | | WLREVEWFSVVCGLLVAWITVG |
| | | | LAHLKSFPSALLGIVLATLIAYEL |
| | | | DWNVARVGEVDLSDLALALPSIA |
| | | | DGTSWFALIAVALPLAVLSSVES |
| | | | LISAKAVDAMANGKSGYSANTE |
| | | | LFGQGVGSIASALVGGMPLAGV |
| | | | VVRSSVNQQSGARTRLAAMCHA |
| | | | VFLGIVAYFFGGLLGVIPVAALA |
| | | | GLLVVIATRLMKLSYFFSALREN |
| | | | KLHALAFLAAAIGTLLGYLISGLA |
| | | | LGCALVYIAHKLAHRPVKDAPVL |
| | | | RPSPTIRAVISQAGERAQDHTPSI |
| | | | DEQAKWSRHVRTRPKIHPTAYV |
| | | | HPTASVIGWVELGREVNIAADTS |
| | | | VRADEGAPFYVGDRSNVQDGVV |
| | | | IHALKDKWVMVDGRRWAVWIG |
| | | | SDVSLAHQALVHGPSMIGSRSFIG |
| | | | FKAIVHDSVVGEGCFIGLGAVVV |
| | | | GVEIPAGKRVPNGWIVDSPEKVR |
| | | | ELPDVEHAHAHFNEDVVQVNRG |
| | | | LVVAYSRHVPTEELPQRTPSDSPL |
| | | | FHLKPL |
| 11 | Thermodesulfitimonas | A0A7Y6PMB4 | MRLPKMLTVVAVGATLCFTAGC |
| | autotrophica | | ASTQTTATKEPAKPANIRPNVVT |
| | Carbonic Anhydrase | | TFNPTTETPVIAKDAYIDPLASVI |
| | | | GNVEIGSKVYVAPFASVRGDEGQ |
| | | | PIYVGEGSNVQDGVVLHALETED |
| | | | NGKPVEKNLVEYGGKKYAVYIG |
| | | | KHVSLAHQAQVHGPALVDDGTF |
| | | | VGMQALVFKAQVGKNCVIEPGA |
| | | | KLLNGVKVPDGRYVPAGTVVTT |
| | | | QAQADKLPVITDAYPLKNLNKG |
| | | | VLHVNEQLAEGYLKAQEGATGE |
| | | | TKSH |
| 12 | Fischerella | A0A3N5BJ34 | MAVRSIAEAAPPTPWSRNLAEPTI |
| | thermalis/ | | HPSAFLHSFSNIIGDVRIGANVIIA |
| | Mastigocladus | | PGTSVRADEGTPFYIGENTNLQD |
| | laminosus JSC-11 | | GVVVHGLEKGRVIGDDRQEYSV |
| | Carbonic Anhydrase | | WIGKNNCITHMALIHGPCYIGDD |
| | | | CFIGFRSTVFNARVGAGCIVMMH |
| | | | ALIQDVEIPPGKYVPSGAIITNQQ |
| | | | QADRLPDVQADDKEFAHHVVGI |
| | | | NQALRAGYLCAADSKCIRAIRDE |
| | | | LNNSYTSIEVDVLERSDEVSSNSL |
| | | | GAETVEQVRYLLQQGYHIGTEH |
| | | | VDQRRFRTGSWTSCKPIEARSLG |
| | | | EAIAALEACLRDHSGEYVRLFGI |
| | | | DPKGKRRVLENIIQRPDGVVQAS |
| | | | SSLKAPAYSSNNGSYNGNGSSRL |
| | | | SSETIDQIRQLLAGGYKIGTEHVD |
| | | | ERRFRTGSWQSCKPIESSSPGDVV |
| | | | AALEDCMDNHQGEYVRLIGIDPK |
| | | | AKRRVLESIIQRPNGPVSTPSSKST |
| | | | ATTTSYAASGTTATATSSKLSSEA |
| | | | IEQLQQLLAGGFKISAEHVDGRR |
| | | | FRTGSWASCGQIQANSIREAIAAL |
| | | | EGYMNEYQGEYVRLIGIDPKVKR |
| | | | RVLELIVQRP |
| 13 | Thermosynechococcus | G6FUV4 | MLRKNPRTSWNSQESMPSVATT |
| | vestitus BP- | | AYVDETAVVIGDVRIGERVYVGP |
| | 1/(Thermosynechococcus | | CASIRADEATPIVISEECNVQDGA |
| | elongatus BP-1 | | IFHGLKGSSIKLGKKVSVAHGAV |
| | Carbonic Anhydrase | | VHGPMTIGDESFIGFNAVVHAST |
| | | | VGERCFIGHRALVMGVKLKDGS |
| | | | FVPHGSVIDTQDKADALGPVPDS |
| | | | LKGFNAEVVEVNCEFAKGYRSL |
| | | | R |
| 14 | Methanothrix | Q8DKB5 | MSENLRLNPQGDKPVIDPSSYVD |
| | thermoacetophila | | PTAVIIGPVTIGKNCYIGPHTVIRA |
| | Carbonic Anhydrase | | DEVDEKTGKVAPVIIGDNVNLQD |
| | | | GVIIHALAGTSVEVGSNTSLAHG |
| | | | CVVHGPCKIEAGCFIGFRAVVFK |
| | | | TVIGSGSMVKHGAIVEGVNIPSG |
| | | | KLVPTGEIITSEDHLVKLKEVGQA |
| | | | EKEFMQEVVHVNMELAHGYKK |
| 15 | Thermosyntropha | A0B700 | MSENLRLNPQGDKPVIDPSSYVD |
| | lipolytica Carbonic | | PTAVIIGPVTIGKNCYIGPHTVIRA |
| | Anhydrase | | DEVDEKTGKVAPVIIGDNVNLQD |
| | | | GVIIHALAGTSVEVGSNTSLAHG |
| | | | CVVHGPCKIEAGCFIGFRAVVFK |
| | | | TVIGSGSMVKHGAIVEGVNIPSG |
| | | | KLVPTGEIITSEDHLVKLKEVGQA |
| | | | EKEFMQEVVHVNMELAHGYKK |
| 16 | Desulfofundulus | A0A1M5PQH8 | MSENLRLNPQGDKPVIDPSSYVD |
| | thermobenzoicus | | PTAVIIGPVTIGKNCYIGPHTVIRA |
| | Carbonic Anhydrase | | DEVDEKTGKVAPVIIGDNVNLQD |
| | | | GVIIHALAGTSVEVGSNTSLAHG |
| | | | CVVHGPCKIEAGCFIGFRAVVFK |
| | | | TVIGSGSMVKHGAIVEGVNIPSG |
| | | | KLVPTGEIITSEDHLVKLKEVGQA |
| | | | EKEFMQEVVHVNMELAHGYKK |
| 17 | Archaeoglobus | A0A6N7IXF4 | MLQKSPAVSWKPAGYPRISSLAF |
| | veneficus Carbonic | | VHPTAVLIGEVVIHDGAIIFPLAII |
| | Anhydrase | | RADEGFPIIVGENTNIQDGVIIHCL |
| | | | KGGRVEIGRRVSLAHGAVIHGPC |
| | | | VIGDETFVGFRAMVINSRIGRGCF |
| | | | IDHGALIEGVEIPDGKYIPGLTRV |
| | | | SSQEQVSRLAGITEQQKDFAAEV |
| | | | LAVNGELKEAMQVIITSRDDAYP |
| | | | GQ |
| 18 | γ Carbonic Anhydrase | F2KNT3 | MRWAIILTTVLFAALLLGCAAEK |
| | | | GAIEPLETPEEKASNIHANPITEW |
| | | | NDEQTMPDIDPTAFVHPYATVIG |
| | | | DVHIGKYVCISPHASVRGDEGMP |
| | | | IYVGDYSNIQDCVVIHALETRDA |
| | | | EGNPIEKNLVVGDDGKKYAVYIA |
| | | | DHVSLAHQSQVHGPAYVGSGTFI |
| | | | GMQALVFKAKVGKNCVIEPGAK |
| | | | VIGVTIPDGRYVPAGMAVTNQSV |
| | | | ADNLPEITEDYPFKHTNEAVVHV |
| | | | NIELAKGYNAMFGGESTEGTEGE |
| | | | GGH |

In some embodiments, an enzyme of the present disclosure is an engineered enzyme. In some cases, the engineered enzyme may have the sequence of any one of SEQ ID NOS: 19-402 provided in Table 2, or an amino acid sequence having at least about 50%, at least about 60%, at least about 70%, at least about 80%, at least about 85%, at least about 90%, at least about 91%, at least about 92%, at least about 93%, at least about 84%, at least about 95%, at least about 96%, at least about 97%, at least about 98%, at least about 99%, at least about 99.5%, at least about 99.9%, or more sequence identity to any one of SEQ ID NOS: 19-402.

In some embodiments, an enzyme of the present disclosure is an engineered enzyme.

In some cases, the engineered enzyme may have the sequence of any one of SEQ ID NOS: 403-464 provided in Table 2, or an amino acid sequence having at least about 50%, at least about 60%, at least about 70%, at least about 80%, at least about 85%, at least about 90%, at least about 91%, at least about 92%, at least about 93%, at least about 84%, at least about 95%, at least about 96%, at least about 97%, at least about 98%, at least about 99%, at least about 99.5%, at least about 99.9%, or more sequence identity to any one of SEQ ID NOS: 403-464.

TABLE 2
Library of modified or engineered enzymes

| SEQ.ID. NO | Sequence |
|---|---|
| 19 | MQEITVTRFENIRPSPVTPWNPEPRYPEIHPTAYIDPAAVVQGD |
| | VKIGANVLVMANAVIRADEGYPIYIGDNSSVQDNVVLHALETR |
| | DADGRDLEENIVRVGDERYAVYVGDNVVLAHNAQVHGPAAV |
| | GDNTFVGMNALVFRSRVGADCVLAPLAAAIGVTVPDGRYVPA |
| | GTVVTTQAAAAALPAVTPDHPFAGLNARVVAVNVALAKGYL |
| | ALS |
| 20 | MSKIYLAFVCGPEQWHRDFPTANGLRQSPIDIIPSKAVYDPKLR |
| | PLELKYDPSTCLHILNNGHSFQVEFDDSQDKSVLKGGPLDGIY |
| | RLIQFHFHWGSVDGQGSEHTVDKKKYAAELHLVHWNTKYGD |
| | FGKAVQQPDGLAVLGIFLKVGRHKPELQKLVDALSSIKHKDTL |
| | VDFGNFDPSCLMPTCPDYWTYSGSLTTPPLSESVTWIIKKQPVE |
| | VDHDQLEQFRSLLFTSEGEKEKRMVDNFRPLQPLMNRTVRSSF |
| | R |
| 21 | MKRSLVATIFGYCPEWNDHQSEWGYGETNGPKTWGKHFPEA |
| | NGLLQSPIDIKTEETQHDPNLRPLTLKYDPSTAKEILNNGHSFQ |
| | VTFVDDTDSSTLTDGPITGTYRLKQFHFHWGSSDDKGSEHTVD |
| | GAKYPAELHLVHWNTKYASFGEAASKPDGLAVVGVFLKIGKE |
| | HPGLKKLTDALYMVRFKGTKAQFTNFNPKCLLPTSLDYWTYP |
| | GSLTTPPLSECVTWIVLKEPISVSSAQMEKFRNLLFTSEGEKAC |
| | CMVDNYRPPQPLKG |
| 22 | MQEITVTNYNNIRPSPVTSWNPTPKLPKIHPTAYIDPAAVVQGD |
| | VTIGENVMVSANASIRSDEGYPIYIGDNSNVQDNVVLHALETV |
| | DADGNVLEENVVTVGDKKYAVYIGKNVSLAHQAQVHGPAAV |
| | GDNTFIGMQAFVFNSVVGKDCVLMPLAAAIGVTIPDGKYIPAG |
| | TVVTTQEEADKLPEVTPDHPFANTNKAVVAVNVELAKGYLAL |
| | A |
| 23 | MRFFECSCSPFPSQLSSFLTHLLILYTLSSSVEASSRNNYQWSYD |
| | SDVFGGPDFWGLVEKDWWMCRKGRLQSPIDIQPDRLLFDASV |
| | KPVRLDKLPVLSEFVNTGQMVRIRIGYSTKKPSVNITNGPLYG |
| | YRYRVQRIDFHMGRGKENGSEHTINGRRFPMEVQLVAFNTDL |
| | YPNFTAASKSPHGIAILSVLVDFGAQTNQELTKLTIATASISYKD |
| | QRVQMADFEPWRLLPFTRDIITYEGSLTSPGCHETVTWIILNQPI |
| | FITREHFEEWSHLYHTMEGAEKVPVAPNYRKIQETNNRLVRTN |
| | IQHKV |
| 24 | MQEITVLEFSNVTKNEVTPWNPKPVTPVIDPTAYIDPTATVIGD |
| | VTIGANCYIAASAVIRADEGKPIVIGDRSNVQDGVVLHALESV |
| | DDGGKVREDNVVIHGDNWYAVYIGENVSLAHQSQVHGPAYV |
| | GDDSFVGMKSLVFKSIVGSNCVIEPEAAAIGVTIPDGKYIPAGT |
| | VVTTQAEADKLPEVTPDYAFYTQVAAVVTVNVNLCRAYRNL |
| | S |
| 25 | MQEITVAEYSNITKNEVTPWNPKPSTPVIDPTSYVDPNATVIGD |
| | VTIGKNCYIAASAVIRADEGKPIVIGDRSNVQDGVVLHALESV |
| | DDGGMIIGDNVVVEGDKYYAVYIGNNVKLAHQSQVHGPAMV |
| | GDDSFVGMQSFVFNSIVGSNCVIEPEAAAIGVTVPDNKYIPAGT |
| | VVTTQAEADKLPEVTPDDAAFTKNAAVVNVNVGLAKAYREK |
| | A |
| 26 | MQEITVTNYNNIRPSPVTSWNPEPKLPEIHPTAYIDPAAVVQGD |
| | VTIGENVLVMANAVIRADEGYPIVIGDNSAVQDNVVLHALETV |
| | DENGNRIEENIVKVGDEEYAVYIGKNVVLAHNAQVHGPAIVG |
| | DNTFVGMNALVFRSRVGKNCVLEHNAAAIGVTVPDGKYIPAG |
| | TVVTTQEEADKLPEVTPDHPHYKLNERVVKVNVELAKGYLAL |
| | K |
| 27 | MQEITVTRYENIQPSPVTPWNPTPKRPQIHPTAYVHPLAYVQG |
| | DVTIGANVMISPNASIRSDEGYPIKIGDNSNVQDNVVLHALETV |
| | DADGKRIEENIVKVGDEEYAVYIGDNVSLAHQAQVHGPAAVG |
| | DNTFIGMQAFVFRSIVGKNCVLEPLAAAIGVTIPDGTYIPAGTV |
| | VTTQEEADKLPKVTPDHPFAKTNAAVVAVNVALAKGYLALA |
| 28 | MLRKNPSGHIPQVAETAFIDPTAIICGKVIIEDYVFIGPYAVIRA |
| | DEVNEQGDMEAIVIKRDTNIQDGVVIHSKAGAAVTIGERSSIAH |
| | RSIIHGPCWVGDDVFIGFNSVVFNAKIGKGCVIRHNSVVDGLD |
| | LPENFHVPPMTNIGPGFDLESISKVPPEYSAFSESVVSANHELV |
| | QGYRRIANEL |
| 29 | MGRSCLTLSRYQAKVSANFLKNRVMASWGYKTDNGPSQWHI |
| | GYPVAKTGTRQSPVNIVPSTVTRDDLLKALKYEYTPSMIKMIN |
| | TGSSWRMDFSPEGSNLSGGPLGDDYKVLQMHAHWGDKAGR |
| | GSEHTMDGKMFDAELHIVHYNSKYGEPAIALDKPDGLAVLGM |
| | FIKTGWRSHPEFDKLCDNLKLIEMKGESLQLQEYLNPANCLPN |
| | NKTFVTYPGSLTTPPLFESVTWIVFLEPIEMSSKQLDSMRALKI |
| | GDTADCGCMVNNYRPPCALGNRKIRVKV |
| 30 | MSLVPIERETARRGRPPVAPRALGALLALASAVAATPAIAWQS |
| | GIAVPDPNAMPQWRYTGERGPEHWSELDPSYGACAHTDTQSP |
| | VALTESMAVAVACEPLRFRYRSGPLYVINDGRALRLGYDRGS |
| | HLLVEGLSYELVELRFHAPAEHVINGSRADAELQLIHANNRGD |
| | IAVVAVALMPGPRANSMLQRLLKHAPRLSGESFYGRNVGVNP |
| | LFLLPGRKDYFAYRGSVTRPPCTEGVRWYVLRTPLEVADADL |
| | QRLVGFMEPNARPLQPLGGRRVTKACGP |
| 31 | MQEITVTRYENIRPSPVTPWNPEPKLPKIHPTAYIDPAAVVQGD |
| | VTIGANVMVSANASIRSDEGYPIYIGDNSNVQDNVVLHALETV |
| | DENGNVIEENVVTVGDKKYAVAIGDNVSLAHQAQVHGPAIVG |
| | DNTFIGMQAFVFRSRVGKNCVLAPLAAAIGVTVPDGTYIPAGK |
| | VVTTQEEAAKLPKVTPDHPFANTNAAVVKVNVALAKGYLAL |
| | A |
| 32 | MQEITVHHYSNVTKNEVTPTNPKPVTPVIDPTSYVDPNATVIG |
| | DVTIGKNVLIAANAVIRADEGAPIVIGDRSTVQDGVVLHALES |
| | VDDAGKVREDNVVIEGDEEYAVYIGKDVSLAHQSQVHGPAR |
| | VGDHSFVGMKSLVFKSIVGSNCVLEPEAAAIGVTVPDGKYIPA |
| | GTVVTTQEEAAKLPEITPDYPLYHANQVVVNVNVLLCQAYKA |
| | LS |
| 33 | MAKTSFFPVVLSFIFILSYTMCINANATGKHEVDDEEPFSYLLG |
| | TAEGPYKWGTLKPDWEICNTGLFQSPINFRNKTVKVTKHIPHF |
| | TPNYKIASATIMNRGHDIKLQWEGDAGSITLNGTVYKLIQCHW |
| | HTPSEHKVDGQSLAMEAHLIHQSVNGKLIAVIGILFNIGPPDPFL |
| | NELIHHAKKVDHKGKKVGLVDPNKLGVKAEPFYRYIGSLTIPP |
| | CTEGIVWNVLHQPRTVSMDQMMALRNAVNDGFQANARPAQ |
| | GLRRRPVYLVM |
| 34 | MQEITVTTYNNIRPSPVTSWNPEPRLPKIHPTAYIDPAAVVTGD |
| | VTIGANVLVMANAVIRADEGYPIVIGDNSSVQDNVVLHALETV |
| | DADGNVLEENVVLVGDERYAVYVGDNVVIAHNAQVHGPAIV |
| | GDNTFVGMNALVFRSRVGANCVLAPLAAAIGVTIPDGTYIPAG |
| | KVVTTQEEAAKLPRVTPDHPFADLVARVVKVNVELAKGYLAL |
| | S |
| 35 | MQEITVTDYSNITKNEVTSTNPKPTTPVIDPTSYVDPNATVTGD |
| | VTIGKNVMISDSASIRSDEGRPIVIGDRSNVQDGVVLHALESVD |
| | DDGEILEDNVVEVGDENYAVYVGKNVSLAHQSQVHGPAAVG |
| | DDSFIGMQAFVFKSKVGSNCVIEPDAAAIGVTVPDGKYIPAGT |
| | VVTTQEEAAKLPEITPDYEYSDTVEAVVEVNVALREAYKEKS |
| 36 | MLAFVALVSLIFLGVQAQHGADWTYSEGMLDETHWPEEYPD |
| | CGGQRQSPIDLQRRKVRFNPDLQPLELTGYGDSQGSPFLMTNN |
| | GHTVQITLPPTMQLTAPDGAVYKATQMHYHWGGASYELSGS |
| | EHTIDGIRRVIEMHLVHYNAKYESYDVAKDKPDGLAVMAAFV |
| | EIEEYAENTHYSSLISHLANIRYPGQTTYLTDFDILDMLPGDMY |
| | HYYTYNGSLTTPPCTQNVRWFVMSDSVKISKAQVIKLENSVM |
| | NHQNQTLHNGYRKTQPLHSRVVEANFPYFPNTMPGEGSGLRA |
| | KDPAREFGSRRHCYAWRGWQPAAAAALEGHGEPRRRWRPLE |
| | EASTPPP |
| 37 | MQEITVTRYNNIRPSPVTPWNPEPKLPEIHPTAYIDPAAVVQGD |
| | VTIGANVMVSANASIRSDEGYPIYIGDNSNVQDNVVLHALETV |
| | DADGKRIEENVVKVGDKDYAVYVGDNVSLAHQAQVHGPAA |
| | VGDNTFIGMQSFVFRSIVGKNCVLEPLAAAIGVTVPDNTYIPAG |
| | KVVTTQEEADKLPKVTPDHPFANTNAAVVKVNVALAKGYLA |
| | LA |
| 38 | MKNRRIKPEIMKTKFLFAILTLFFFSGCQFFDKNKSTEIESKPSS |
| | HEKWSYTGESGPEHWAELEDQAVCDGQHQSPVNISDIDIKPGK |
| | LIQESLDLSYQEVTTIKSITNNGHTIQYNFDANSNLVSLHDKQY |
| | KLKQFHFHSPSEHTINGTHSPLEIHLVHHSEATNSYIVIAILVQQ |
| | GEPDDAFDFLEKYLPINVGETKEINSKYYFGSTFPEMYGKDTL |
| | NIYTYEGSLTTPPCTESVLWVVIKDPAYASSSQIVMLQKLMPK |
| | DNYREVQSLNGRLIYNEIIEDDISVLNH |
| 39 | MTKLSFAVIGPENWHRYCDQAQGDQQSPINIQTRDVKHDPTL |
| | RPLTLRYDPSTAREIVNNGHSFNVEFEDSTDRSVLRGGPLTDRY |
| | RLTQFHFHWGSSDDHGSEHTVDGVKYAAELHLVHWNTKYGD |
| | FGEAASKPDGLAVVGVFLKVGRHNPRLQKILDALHAIKTKGK |
| | RASFTNFDPSVLLPGCLDYWTYSGSLTTPPLSESVTWIVLREPIS |
| | VSPSQMAKFRSLLFTS |
| 40 | MQEITVTTYNNIQPSPVTPWNPEPKLPKIHPTAYIHPKAVVQGD |
| | VTIGKNVMVSANASIRSDEGYPIVIGDNSNVQDNVVLHALETV |
| | DENGNEIEENIVTVGDKKYAVYIGKNVSLAHQAQVHGPAIVG |
| | DNTFIGMQAFVFNSNVGSNCYLAPLAAAIGVTVPDGTYIPAGK |
| | VVTTQEEAAKLPKITPDHPFYNTNAAVVKVNVALAKGYLALS |
| 41 | MQEITVDEFSNITKNEVTPFNPKPTIPVIDPTAYVDPNATVIGDV |
| | TIGKNCYIAPFASIRADEGKPIVIGDNSNVQDGVVLHALESIDD |
| | GGKLIEENVVVEGEKRYAVYIGKNVSLAHQSQVHGPARVGDD |
| | SFVGMNSLVFNSKVGSNCVIEPFAAAIGVTVPDGKYVPAGTVV |
| | TTQEEADKLPEITDDYAFAGTNEAVVKVNVKLCKAYREKA |
| 42 | MQEITVLEYSNVTKNEVTSQNPKPVTPVIDPTSYVDPNATVVG |
| | DVTIGENCLVWPTAVIRADEGRPIVIGNESSVQDGVVLHALES |
| | VDDGGELVEDNVVVVGDKNYAVYVGKNVSLAHQAQVHGPA |
| | RVGDDSFVGMKSLVFKSDVGSNCVIEPFAAAIGVTVPDGKYIP |
| | AGTVVTTQEEAAQLPEVTPDHAEYTTQATVVTVNVELNEAYR |
| | NQR |
| 43 | MTEKLWGYDSHNGPARWFQICVPAQGKRQSPIDIQPDKAVLD |
| | STLKPLELKYDPSTARRIVNVGHSFHVEFEDSTDKSVLQGGPLT |
| | GSYRLRQFHFHWGKKDDVGSEHVLDGVKYSAELHVVHWNA |
| | DKYSSFVEAAHEPDGLVVLGVFLQIGDQHPGLQRLTDALYAV |
| | RFKGTKAQFACFNPKCLLPTSRHYWTYPGSLTTPPLSESVTWI |
| | VLREPISVSERQMEKFRSLLFTSEDDERIHMVNNFRPLQPLMNR |
| | TVRSSF |
| 44 | MYHNALFLTPITVFYVAAHKFGYDAEDGPSTWRGVCQTGKR |
| | QSPVDIRAFEIEIAPLDPLQFLNYDLTGHIHLANNGHTVVGSGF |
| | ERWGEKRPYISGGGLNGTYQLSQFHFHWSQQNDTGSEHTIASL |
| | HYPGELHLVHIKKEPSPDEVNTIAVVAAFIKLDDHAGSLHNLK |
| | PYVHNIRMPNTELVVPGFSVSSLLPEHRENFYRYEGSLTTPGCD |
| | EVVVWTLMADPIAVTPSQMGAFHQVHFASGKTGHNWRPTQP |
| | LNGRKILFRPSITLRTFKSGGAMLKPVFQPFISIWLYGIYHIISVF |
| 45 | MQEITVTKYNNIRPSPVTPWNPEPKLPEIHPTAYIDPAAVVRGD |
| | VKIGENVLVMANAVIRADEGYPIYIGNNSSVQDNVVLHALETV |
| | DENGNRIEENIVLVGDKEYAVYIGDNVVIAHNAQVHGPAAVG |
| | DNTFIGMNSLVFRSRVGSNCVLAPLAAAIGVTVPDGTYIPAGK |
| | VVTTQEEAAKLPKITPDHPFANLNDRVVKVNVALAKGYLAQA |
| 46 | MKMFPLDCLILPCCYFFFISTPHFANADVHIADWDHDHHHTHP |
| | DNWEGMCKEGQRQSPIDIITNETTKEKWGQPFIFHGYERKLSM |
| | NVKNNRHSMVVEFDNDKKYEDIWIRGGGLGESKFRFAQLHFH |
| | WGSTNDQGSEHTIDGKASPMEMHIVHWNLDVGKDVKEATEK |
| | DAYNSLEVLGVLFKLGKFNKDYDAIFNAARKVEKENTNATLE |
| | KDVRLRDLLPEDTNAFYRYVGSLTTPPCNQIVMWTIFKDPIEIS |
| | QEQLDIMRKGSYRLEGENDVRYIANNYRSTQTLYERDVLDIDT |
| | HIVHLACNSKGSTRYHFEEGSEGFVHNTGNSLNSPIVTCMLFY |
| | LSIFVISMRLLH |
| 47 | MQEITVTRYENIRPSPVTPWNPEPRRPVIHPTAYVDPLAYVQG |
| | DVTIGANVMVSANASIRSDEGYPIVIGDNSNVQDNVVLHALET |
| | RDADGRVLEENVVVVGDERYAVYVGDNVSLAHQAQVHGPA |
| | AVGDNTFIGMQAFVFRSRVGKNCVLEPLAAAIGVTVPDGTYV |
| | PAGKVVTTQEEAAKLPKVTPDHPFATTNAAVVAVNVALAKG |
| | YLALA |
| 48 | MQEITVLEFSNITKNEVTSFNPEPVTPVIDPTAYIDPNATVIGDV |
| | TIGANVLIWPTAVIRADEGKPIVIGDRSNVQDGVVLHALESVD |
| | DGGKVREDNVVIEGDEEYAVYIGKNVTLAHQSQVHGPARVG |
| | DDSFVGMKSLVFNSDVGENCVIEPFAAAIGVTVPDGKYIPAGT |
| | VVTTQAEAATLPEVTPDYAFYTQVAAVVSVNVGLCQAYKNE |
| | A |
| 49 | MQEITVDEFSNVTKNEVTPWNPKPTTPVIDPTSYIDPEATVIGD |
| | VTIGKNCYIAPFAVIRADEGSPIVIGDDSTIQDGVVLHALESVD |
| | DGGKLIEDNVVLEGDQYYAVYIGRNVVLAHQSQVHGPAWVG |
| | DDSFVGMKSLVFKSTVGSNCVLEPNAAAIGVTVPDGKYIPAGQ |
| | VVTTQAEADNLPEVTADDAYYTKVAAVVKVNVALCEAYREQ |
| | S |
| 50 | MQEITVTKYNNIRPSPVTPWNPEPKLPEIHPTAYIDPAAVVQGD |
| | VTIGANVMVSANASIRSDEGYPIKIGDNSNVQDNVVLHALETV |
| | DADGKELTENVVTVGDEKYAVYVGDNVSLAHQAQVHGPAA |
| | VGDNTFIGMQAFVFRSTVGKNCVLAPLAAAIGVTVPDGRYIPA |
| | GLVVTTQEEADKLPKVTPDHPFYNTNAAVVAVNVALAKGYL |
| | AQA |
| 51 | MSRPVALTIFGYEDKNQWHCCYPSAQGNRQSPINIDIKKTVYD |
| | PKLKPLELSYDPATAKGILNNGHSFNVEFEDSQDKSVLKGGPL |
| | TGTYRLIQFHFHWGATDDKGSEHTVDGVKYPSELHLVHWNA |
| | VKYSSFAEAASKPDGLAVLGVFLKVGDHNAALQKLTDALYM |
| | VRFKGTKAQFTGFNPKCLLPASLDYWTYSGSLTTPPLLESVTW |
| | IVLKEPISVSSEQMAKFRSLLFTSEGEAECCMVDNYRPPQPLKG |
| | R |
| 52 | MQEITVTTYTNIRKSPVTSWNPTPKYPKIHPTAYIDPAAVVQGD |
| | VTIGENVMVSANASIRSDEGYPIYIGDNSNVQDNVVLHALETV |
| | DANGNVIEENVVTVGDKKYAVYVGNNVSLAHQAQVHGPAA |
| | VGDNTFIGMQAFVFNSRVGKNCVLEPLAAAIGVTVPDGTYIPA |
| | GEVVTTQEAADKLPKVTPDHPFANTNAAVVKVNIELAKGYLA |
| | QA |
| 53 | MQEITVTVYTNIQPSPVTSWNPTPKLPKIDETAYVHPQAVVQG |
| | DVTIGKNVMISANASIRSDEGYPIVIGDNSNVQDNVVLHALET |
| | VDADGKEIEENVVTVGDKKYAVYIGDNVSLAHQAQVHGPAA |
| | VGDNTFIGMQAFVFRSNVGKDCVLEPLAAAIGVTVPDGTYIPA |
| | GKVVTTQEEAAKLPKVTPDHPFYKTNAAVVKVNVELAKGYL |
| | ALA |
| 54 | MLRKNPSGHIAVIDQTAYIDETAIICGKVIIEANVFVGPYAVIRA |
| | DEVNEQGDMEPIVIKRDTNIQDGVVIHSKAGAAVTIGERSSIAH |
| | RSIIHGPCWVGDDVFIGFNSVVFNAKIGKGCVIRHNSVVDGLD |
| | LPENFHVPPMTNIGPDFDLNSISKVPPEYSAFSESVVSANHELV |
| | QGYRRIANEL |
| 55 | MQEITVLEFSNITKNEVTPWNPKPSTPVIDPTSYVDPNATVIGD |
| | VTIGKNCYIAASAVIRADEGKPIVIGDRSNVQDGVVLHALESV |
| | NDGGKIREDNVVLEGDKYYAVYIGKNVVLAHQSQVHGPAAV |
| | GDDSFVGMKSLVFKSIVGSNCVIEPEAAAIGVTVPDGKYIPAGT |
| | VVTTQAEADKLPEITPDYAFYTQVAAVVKVNVDLCEAYRNKA |
| 56 | MQEITVTTYTNIRPSPVTPWNPEPKLPEIHPTAYVDPAAVVQGD |
| | VTIGKNVMVSANASIRSDEGYPIKIGDNSNVQDNVVLHALETV |
| | DEDGNVIEENVVTVGDEKYAVYVGDNVSLAHQAQVHGPAAV |
| | GDNTFIGMQAFVFRSNVGKNCYLAPLAAAIGVTIPDGTYIPAG |
| | TVVTTQEEAAKLPKMTPDHPGYNTNAAVVKVNIALAKGYLAL |
| | S |
| 57 | MQEITVDNFSNITKNEVTPTNPKPSTPVIDPTSYVDPNATVTGD |
| | VTIGKNCLIAANAKIRADEGKPIVIGDRSSVQDGVVLHALESVN |
| | DDGKVLEDNVVLEGDEYYTVYIGKNVVLAHQAQVHGPAAVG |
| | DDSFVGMKALVFKSKVGKNCVIEPGAAAIGVTVPDGKYIPAG |
| | TVVTTQEEADKLPEITPDYPLSDANEAVVKVNVGLCEAYRNK |
| | S |
| 58 | MQEITVTKYENIRPSPVTSWNPTPKLPKIHPTAYIDPLAYVQGD |
| | VTIGENVMVSANASIRSDEGYPIYIGNNSNVQDNVVLHALETV |
| | DKNGKVLEENVVTVGDKKYAVYVGDNVSLAHQAQVHGPAA |
| | VGDNTFIGMQAFVFRSTVGKNCYLAPLAAAIGVTVPDGTYIPA |
| | GKVVTTQEEAAKLPKMTPDHPGYKTNEAVVEVNVELAKGYL |
| | ALA |
| 59 | MASKLLRRNLLFTIQKRAVKSSVTRNSIPWLRKSAPSSNWGYN |
| | GSELDPEDWPKEYQCGNCQSPIDIDLSKVTYSSELSPLEYSYPD |
| | NFKYMVNDGKNIRIHWRGETALSGGPLKGTYELVQLHFHWGS |
| | AEGKGAEHLVNGESVEGEAHLVHWNPKYGSIREALKHQDGIA |
| | VVGVFLKEADDGAESPLSSILNRFPTLSKFNEKYIFENDVFNVG |
| | NLIPKNSDFICYDGGLTTPPLTECVQWIVLLKPLVVTKREMDIF |
| | RSLEGSFGNNFTDNFRPCQPVGDRVVSSSFEPEK |
| 60 | MKITALSVFCWGPNYEDHRQKWSNLYPIAKGNRQSPINIVPGS |
| | AVYDSSLKPLKLKYDPSTCLEIWNNGHSFQVTFEDTDDKSVLS |
| | GGPLTDKYKLKQFHFHWGKTDDHGSEHTVDGVKYAAELHLV |
| | HWNAKYGSFGEAADKPDGLAVVGIFLKIGREKGEFKLILDALD |
| | SIKTKGKQTTFTNFDPSCLFPSCPDYWTYSGSLTTPPLSESVTWI |
| | ILKQPIEVDHDQLEKFRTLLFTSEGEKEKRMVDNFRPLQPLMN |
| | RTVRSSFR |
| 61 | MQEITVTVYNNIRPSPVTPWNPEPKLPKIHPTAYVHPLADVTG |
| | DVTIGANVMVSAHASIRSDEGYPIYIGDNSNVQDNVVLHALET |
| | VDAAGKEIEKNIVTVGDKKYAVYIGDNVSLAHQAQVHGPAAV |
| | GNNTFIGMQAFVFNSVVGENCVLEPLAAAIGVTVPDGTYIPAG |
| | KVVTTQEEAAKLPKVTPDHPFYNTNKAVVAVNVALAKGYLA |
| | LA |
| 62 | MQEITVMEYSNVVKNEVTSTNPKPTTPKIDPTSYVDPNATVIG |
| | DVEIGKNVLIAPFAVIRADEGSPIVIGDNSNVQDGVVLHALESV |
| | DDGGKINEDNVVVKGDKYYAVYIGKNVHLAHQAQVHGPAY |
| | VGDDSFVGMKALVFKAKVGNNCVIEPNAAAIGVTVPDGKYVP |
| | AGTVVTTQEEADKLPEITEDYPFSTANEVVVKVNVNLAKAYR |
| | NLA |
| 63 | MQEITVTKFENIRESPVTPWNPEPKKPEIHPTAYIDPAAVVIGD |
| | VTIGANVLVAARAVIRADEGYPIVIGDNSSVQDNVVLHALETV |
| | DEDGNIIEENVVEVGDKRYAVYIGDNVVLAHNAQVHGPAAVG |
| | DNTFVGMNSLVFRSRVGANCVLEPLAAALGVEVPDGRYVPAG |
| | KVVTTQEEAARLPAVTPDHPFADLVARVVAVNVALAKGYLA |
| | LS |
| 64 | MAAHGAHWGYSGEAGPENWAKLTPEYGACTGKNQSPINLTG |
| | FIEAELKPIKIAYKAGAKEIVNNGHTVQVNYQPGSFITIDGQQFE |
| | LKQFHFHAPSENTIEGKSFPLEAHFVHANSKGELAVVAVMYEE |
| | GKENPLIAKAWQQMPEKAGEKNELKSTISAESLLPKDKDYYRF |
| | SGSLTTPPCSEGVRWIVLKNYSTVSKEQVEQFLHTMHHANNRP |
| | VQPVNARKVLK |
| 65 | MKSTLIAGFVCEQNPDHWYRQYPVAKGHHQSPIDIISHTAKYD |
| | PSLKPLSISYDPSTSLEILNNGHSFQVTFEDSNDKSVLKGGPLDG |
| | VYRLKQFHFHWGKKHSVGSEHTVNGKSFPSELHLVHWNAVK |
| | YESFGEAALEENGLAVVGVFLELGEHNAELQKITDALYMVRF |
| | KGTKTTFSCFNPKCLLPSSLDYWTYSGSLTTPPLSESVTWIVLR |
| | EPISISPSQLAKFRSLLFTSEGEKAVCMVDNFRPLQPLMNRSVR |
| | SSFR |
| 66 | MQEITVTRYENIRPSPVTPWNPEPKLPEIHPTAYIDPKAVVQGD |
| | VRIGANVMVSAHASIRSDEGYPIVIGDNSNVQDNVVLHALETV |
| | NENGEVIEENVVEVGDERYAVYVGDNVSLAHQAQVHGPAAV |
| | GDNSFIGMQAFVFRSRVGKNCVLAPLAAAIGVEVPDGTYIPAG |
| | KVVTTQEEAAKLPKVTPDHPFANTNAAVVKVNVALAKGYLA |
| | LS |
| 67 | MSQIWSYTGDTGPEFWPELCEEFYTAAQFPLQSPIALSYEETQA |
| | LEEALKFTYVEQNIYVQKVNETMHFVPVDAASFVEFAQNRYY |
| | LTDIHFHMPSEHVINKQQAPLEFHLVHKDEGGNPLVCAVLFDL |
| | VENEDKKCNKDKLILEADKDKEQLLNPEIFLPENITYFHYEGSL |
| | TTPPTQGPVQWFVFDQIGVMSRSFIEDFKTSLLPNNRPLQNKN |
| | QRPIFYKK |
| 68 | MKGLTPSIAVFCYRQENWDHIFPIAAGNRQSPINIDTRKAKYDS |
| | SLKPLNLKYDPSTSLEILNNGHSFQVNFEDTDNKSVLKGGPLT |
| | GSYRLRQFHFHWGASDDKGSEHTVDGVKYASELHVVHWNA |
| | VKYSSFAEAASKPDGLAVVGVFLKVGQHNPQLQKITDALSSIK |
| | HKDTQALFSNFDPSSLLPSCPDYWTYSGSLTTPPLSESVTWIVL |
| | KQPINVSPAQLAQFRSLLFTSEGEKACCMVDNYRPLQPLKGRQ |
| | VRASF |
| 69 | MSARLVTWGYKEDNGPHQWCIFFPEANGECQSPIDIITSETK |
| | HDPSLKPLSLSYNPATSKEIINVGHSFHVNFEDNDNRSVLKG |
| | GPLTDSYRLTQFHFHWGKKNDRGSEHTIDKKKYSSELHLV |
| | HWNTKYGDFGKAVQQPDGLAVLGIFLKVGKHNPSLQKVL |
| | DTLNSIKTKGKQTTFTNFDPSTLLPGCLDYWTYSGSLTTPPL |
| | LESVTWIILKEPISVSSEQMAKFRSLLFTSEGEKACCMVDNY |
| | RPLQPLMNRTVRSSF |
| 70 | MQEITVSAFSNIRKNEVTPWNPEPSTPVIDPTAYVDPQATVIGD |
| | VTIGANVLVSASASIRADEGRPIVVGDRSNVQDGVVLHALESV |
| | DDGGEVIEDNVVLEGGELYAVYVGENVSLAHQSQVHGPALV |
| | GDDSFVGMKSLVFKSKVGSNCVLEPGAAAIGVTIPDGKYIPAG |
| | TVVTSQAEADNLPEVTPDYAYYTTNEAVVKVNVALAEAYRN |
| | LS |
| 71 | MRLSAIFVTGWCPEKQDHNYYQWGYGKHNGPEHWKDHFPA |
| | ANGLQQSPIDIQISKVQHDPALKPLSLSYDPATARRILNNGHSF |
| | NVEFDDSQDKAVLKGGPLTGSYRLIQFHFHWGSADGQGSEHT |
| | VDKKKYAAELHLVHWNAVKYESFAEAAKQENGLAVLGVFLK |
| | VGEHNAQLQKLLDALSAIKHKGKQTAFTNFDPSCLLPACPDY |
| | WTYSGSLTTPPLSESVTWIVLKEPISVSSEQMAKFRSLLFTSEGE |
| | TACCMVDNYRPPQPLKGRKVRASF |
| 72 | MQEITVTRFENIRPSPVTSWNPEPKRPVIDPTAYIDPAAVVQGD |
| | VTIGKNVMISANASIRSDEGYPIYIGDNSNVQDNVVLHALETV |
| | DADGKRLEENVVKVGDKEYAVAIGDNVSLAHQAQVHGPAIV |
| | GDNSFIGMQAFVFRSRVGKNCVLAPLAAAIGVEVPDGKYIPAG |
| | KVVTTQEEADKLPEVTPDHPYATTNAAVVKVNVELAKGYLAL |
| | S |
| 73 | MARTVLGIFCSPNKEWQYDHAKNGPEVWKEYFPIADGDQQSP |
| | IEIKTKEVKHDSSLKPLSISYNPATAKEILNVGHSFHVNFEDND |
| | NRSVLKGGPLSDSYRLSQFHFHWGSSDDHGSEHTVDGVKYAS |
| | ELHLVHWNAKYGKFGEASKKPDGLAVVGIFLKVGSAKPGLQ |
| | KVVDALGSIKTKGKQASFTNFDPSVLLPGCLDYWTYDGSLTTP |
| | PLLESVTWIVLKEPISVSPSQMAKFRSLLFSSEGEAACCMVDNY |
| | RPPQPLKGRQVK |
| 74 | MPLPNARERRRDWRAVVTAAAVFGIVVPIGTGLHAEDWGYS |
| | GTHGPRFWAKTPGWEACAGTAATERQSPIDIDEVVADKELTR |
| | LQADLKETPVAVVNNGHTIEEEYRLGSSLTLAGVRYDLKQFHF |
| | HTPSEHTVRGAHAAMEMHVVFKDAGSDKLVVIGVLFEVGKA |
| | NAFLSALMADGLPGKRGEEVDAHSRPVNVAQALTDTSQYYT |
| | YPGSLTTPPCSENVTWFVLKGRPEMSAEQLAAFHRVLGDNAR |
| | PVQKLNHRVAHETVSGAR |
| 75 | MTSLQYNNIRPNLAGDYPQIDPTALIDPSAQIIGNVKIDRDVFV |
| | GPLTVIRADQRGPNGKVSPIQIDREANIQDGVIIHTDPGASVIIGS |
| | KTTVAHGAIIHGPCTIGQECFIAIRASLYKVTLEDHVWLGIGAIA |
| | KLVTLHSFTRVPAGAVIRDSPEVLPLRLITDKERKYMEEVWAA |
| | NSLLRTDYLELRDKVESIRSTAKKKG |
| 76 | MQEITVTRYENIRPSPVTPWNPEPKRPKIHPTAYIDPAAVVTGD |
| | VTIGENVLVMANAVIRADEGYPIVIGDNSSVQDNVVLHALETV |
| | DENGNRIEENVVRVGDEDYAVYVGKNVVLAHNAQVHGPAAV |
| | GDNTFVGMNALVFRSRVGKNCVLAPNAAAIGVTVPDGTYVP |
| | AGLVVTTQEEAAKLPKVTPDHPFANLNARVVKVNVALAKGY |
| | LALA |
| 77 | MQEITVTKFENIRPSPVTPWNPTPKRPEIHPTAYVDPLAYVQGD |
| | VTIGANVMISANASIRSDEGYPIVIGDNSNVQDNVVLHALETV |
| | DANGNEIKENIVTVGDEKYAVYVGDNVSLAHQAQVHGPAAV |
| | GDNTFIGMQAFVFRSVVGKNCVLEPLAAAIGVTIPDGTYIPAG |
| | KVVTTQEEADKLPKMTPDHPFYNTNKAVVAVNIALAKGYLAL |
| | S |
| 78 | MQEITVHHHHHITKNEVTPTNPKPTTPVIDPTSYIDPNATVIGD |
| | VTIGKNVLIAPFASIRADEGSPIVIGDRSNVQDGVVLHALETVD |
| | DGGKVIEDNVVLEGDKYYAVYIGRNVTLAHQAQVHGPAAVG |
| | DDSFVGMKALVFKAKVGKNCVIEPDAAAIGVTVPDGKYIPAG |
| | TVVTTQEEADKLPEITPDYAFYTQNETVVKVNVALCEAYREK |
| | A |
| 79 | MLSLSALLAATAFSASASAPHWEYSGEAGPAHWASLTPEFGA |
| | CTGKNQSPVNLTGFVDAKLKPIKFAYQAGGKSIVNNGHTVQV |
| | NYQPGSSITLDGVTFELKQFHFHAPSENQIDGQSYPLEAHLVHA |
| | DKEGNLAVVALMFKQGEANPELAKLWQAMPEKANQSQPLKA |
| | SIRADQLLPENRDYYRFSGSLTTPPCSEGVRWIVMKQPITASAA |
| | QIEEFEHVMHHPNNRPVQPLNGKTIVTGLSS |
| 80 | MQEITVTVFSNVEKNEVTSQNPRPTTPVIDPTSYIDPNATVIGD |
| | VTIGKNCYIAASASIRGDEGRPIHIGDRSNVQDGVVLHALESVD |
| | TDGEVLEDNVVLEGDEDYAVYIGKNVSLAHQSQVHGPARVG |
| | DDSFIGMKSLVFKSIVGSNCVLEPDAAAIGVTVPDGKYIPAGTV |
| | VTTQAEADKLPEVTPDYAYYTKNAAVVAVNVALCEAYRNQS |
| 81 | MLKIVSAFTGYCPENWHRQFDKAAGSQQSPIDIQTKDIQHDPC |
| | LQPLKLSYDPSTCLEIWNNGHSFLVQFEDSGDKSVIEGGPLEGV |
| | YRLKQFHFHWGAKDSEGSEHTVDGVKFPCELHLVHWNAKYG |
| | SFAEAASKPDGLAVVGVFLKIGKEHAEFQKLLDALDAIKTKGK |
| | QTTFTNFDPSCLLPACRDYWTYDGSLTTPPLLESVTWIVLKEPI |
| | SVSPGQMAKFRSLLFTSEGEAACCMVDNYRPPQPLKGRHVRA |
| | SF |
| 82 | MQEITVLSFSNVQKNKVTPTNPKPTTPVIDPTAYIDPDATVIGD |
| | VTIGKNCFIGAFAVIRADEGKPIVIGDRSNVQDGVVLHALESVD |
| | DEGKVIEDNVVVKGDKEYAVYIGKNVSLAHQSQVHGPARVG |
| | DDSFVGMNATVFNSIVGSNCVIEPFAAAIGVVVPDNTYIPAGT |
| | VVTSQEEADKLPEVTPDYAYYTQVAAVVKVNVKLCEAYREK |
| | A |
| 83 | MKITFLSAVCGWNYQDPERWHDDFPIAKGERQSPIDIDLSKVQ |
| | RDPSLKPLSFKYDPSTSRRILNNGHSFNVEFEDSEDKSVLKGGP |
| | LTGSYRLKQFHFHWGATDDKGSEHTVDGVKYASELHLVHWN |
| | AKYGDFGEAASKPDGLAVVGVFLKIGRHHEEFQKLLDALPAIK |
| | HKDTLVDFGSFDPSCLMPTCPDYWTYSGSLTTPPLLESVTWIV |
| | LKQPIEVDHDQLEQFRTLLFTSEGEKEKRMVDNFRPLQPLMNR |
| | TVRSSFR |
| 84 | MQEITVDEFSNVTKNEVTPWNPKPSTPVIDPTSYIDPDATVIGD |
| | VTIGANVLIGPNAVIRADEGKPIVIGDNSNVQDGVVLHALESV |
| | DDAGKVIEDNVVKVGNNSYAVYVGKNVVLAHNAQVHGPAA |
| | VGDDSFVGMNAFVFNSIVGSNCVIEPNAAAIGVTVPDGKYIPA |
| | GTVVTTQEEADKLPEITEDYEYYTKVAEVVEVNVALCEAYKE |
| | KA |
| 85 | MQEITVLLFSNVTKNEVTTTNPKPTTPVIDPTSYVDPNATVTGD |
| | VTIGANVMISANASIRSDEGRPIVIGDRSNVQDGVVLHALESVD |
| | DDGKIIEENVVIHGDEDYAVFIGKDVSLAHQAQVHGPAYVGD |
| | DSFIGMQSFVFKSKVGSNCVIEPEAAAIGVTVPDGKYIPAGTVV |
| | TTQAEAEKLPDVTPDHAQYTTQAAVVTVNVQLTKAYRNLK |
| 86 | MQEITVLKFSNVTKNEVTVTNPKPTTPVIDPTSYVDPKATVTG |
| | DVTIGKNVLIAANATIRADEGKPIVVGDRSTVQDGVVLHALES |
| | VDDTGKVIEENVVIKGNEDYAVYIGNNVSLAHQAQVHGPAHV |
| | GDDSFVGMKALVFKSKVGKNCVIEPDAAAIGVTVPDGKYIPA |
| | GTVVTTQEEAAKLPEITPDYPFYTTNAEVVSVNVKLCEAYKGE |
| | A |
| 87 | MIVRILVVTLLVLSGFPALSTSGTLQDKKAASECSDQPFSYDHG |
| | ASGQQSWCGRCNESGALPLPQAPINIPKIAESAQPAIVENGYNE |
| | NTSLVIYPHNPYNLKVDYKSSSNPVATIDIGSSANSRFKLLEFHF |
| | HRPSEEAIDNRRFPMVLHLVHLREVEGCEPGKPGCVAAVAILI |
| | KEGTPSQQTTDLLNALFSHFPPPDKPKDVEINLEGLLPPDHVNA |
| | GYWSYGGSLTTPPCTENITFYLLKPMLTFSAAQIAEFERRYPTP |
| | NARDIQPLHDRHRVVNRH |
| 88 | MIKSNPRGDLPQVHETAFVDPTAILCGYVIVEENVFIGPYAVIR |
| | ADETDADGRIAPIVIGAHSNIQDGVVIHSKSGAWVTIGQRTSIA |
| | HRAIVHGPCTVGDGVFIGFNSVLFNCTIDDGCVVRYNAVVDG |
| | CHLPPGFYVRSTERIGPETDLAALPQVTADASDFSEDVARTNN |
| | ALVLGYKHIQNEF |
| 89 | MQEITVFEFSNVEKNEVTSTNPKPTTPKIDPTSYVDPNATVIGD |
| | VTIGENCMISATASIRSDEGRPIVIGDRSNVQDGVVLHALESVD |
| | DQGMVREDNVVLEGDEYYAVYVGDRVSLAHQSQVHGPAKV |
| | GDDSFIGMQSFVFKSTVGSNCVIEPGAAAIGVTVPDGKYIPAGT |
| | VVTTQEEADKLPEITDDYPFYSTQAAVVEVNVGLCEAYRGKA |
| 90 | MQEITVFDFSNVRKNKVTGTNPKPVTPVIDPTSYVDPNATVIG |
| | DVKIGKNCLIGASAVIRADEGHPIVIGDRSNVQDGVVLHALES |
| | VNDDGKILEENVVIEGDEYYAVYVGKNVVLAHQSQVHGPAA |
| | VGDDSFVGMKSLVFRSIVGSNCVIEPNAAAIGVTVPDNKYIPA |
| | GTVVTTQEEADNLPEITPDYPYYDTVEQVVKVNVNLCEAYRE |
| | KE |
| 91 | MQEITVTRYENIRPSPVTPWNPEPRRPEIHPSAYIDPAAVVQGD |
| | VTIGANVYVAANAVIRADEGYPIVIGDNSSVQDNVVLHALETV |
| | DADGRRIEENIVEVGGERYAVYVGANVVLAHNAQVHGPAIVG |
| | DNTFVGMNALVFRSRVGANCVLMHLAAAIGVTVPDGTYVPA |
| | GKVVTTQEEAAKLPKVTPDHPFYKLNARVVAVNVALAKGYL |
| | ALS |
| 92 | MQEITVTKFENIQPSPVTPWNPEPKKPEIDPTAYIHPAAVVQGD |
| | VKIGKNVMISALASIRSDEGYPIVIGDNSNVQDQVVLHALETV |
| | DENGNVIEENVVTVGDEKYAVYIGKNVSLAHQAQVHGPAIVG |
| | DNTFIGMQAFVFRSKVGENCVLEPLAAAIGVTIPNNTYIPAGKV |
| | VTTQEEAAKLPKITPDHPFANTNAAVVKVNVALAKGYLALS |
| 93 | MKSILAVFTGCYQPNDEHWRYEDENGPEKWAEIEKNSDCGGK |
| | HQSPINIIHKETDSVHGPLDLQINYEPSTLITEVRNNGHSIQFDFE |
| | KGDSINYKNETYYLKQIHFHEPSEHKINGIIYPIEMHLVHMNKS |
| | GKITVLGILGEEGEESQLFEFFESFLPLKNGETKDIHQKIDLSSLF |
| | LEDKHYYSYDGSLTTPPCSENVNWIVFKEPIVLSVEEVIKLRNN |
| | MPLNNYRNEQP |
| 94 | MQEITVSLFSNVTKNEVTSWNPKPTTPVIDPTSFIDPNATVTGD |
| | VTIGKNCLIGPNAVIRADEGSPIVIGDRSNVQDGVVLHALESVN |
| | DEGKIIEENVVLYGSKLYAVYIGKNVSLAHQSQVHGPARVGD |
| | DSFVGMNSLVFNSIVGSNCVIEPNAAAIGVTVPDGKYIPAGTV |
| | VTSQAEADKLPEITPDHAYYTQNFAVVNVNVNLCRAYRNKS |
| 95 | MASSAFAAEGAHWGYTGHGGPAHWGDLSADYATCKLGKHQ |
| | SPIDIRGAKEADLPAIQFDYKASPLKILNNGHTVQVNYAPGSGI |
| | VVDGKPYELVQFHFHKPSEEKIDGKAYPMVAHLVHRDAAGH |
| | LAVVAVLIKEGKENPLIKTLWPHLPAEEGPEQAVAGATINAAD |
| | LLPADRGYYAFDGSLTTPPCSEGVRWHVLKQPITMSKAQIDAF |
| | QKLYKPNARPLQPLNGRIM |
| 96 | MQEITVTRYENIRASPVTPWNPEPKLPKIHPTAYIDPAAVVQGD |
| | VTIGENVLVMANAVIRADEGYPIVIGNNSSVQDNVVLHALETV |
| | DENGNEIEENVVTVGDKKYAVYVGDNVVLAHNAQVHGPAAV |
| | GDNTFVGMNALVFRSRVGKNCVLAPNAAAIGVTVPDGKYIPA |
| | GKVVTTQEEADKLPEITPDHPFANLNARVVKVNIALAKGYLA |
| | QA |
| 97 | MQEITVLIFSNITKNEVTSTNPKPKTPKIDPTSYIDPNAKVIGDV |
| | TIGKNVLIAAFAVIRADEGKPIVIGDRSNVQDGVVLHALESIND |
| | DGKIIEDNVVIEGNNHYAVYVGNNVSLAHQSQVHGPAHVGND |
| | SFVGMKSLVFKSDVGDNCVIEPEAAAIGVTVPDGKYIPAGTVV |
| | TTQEEAAKLPEITEDYPFYTKVAEVVKVNVDLCLAYRNKQ |
| 98 | MQEITVHHFSNVRKNEVTPTNPKPTTPVIDPTSYIDPNATVIGD |
| | VTIGKNCYVAHSAVIRADEGHPIVIGDRSNVQDGVVLHALESV |
| | DDGGEIREDNVVEVGDESYAVYVGKNVVLAHQSQVHGPAAV |
| | GDDSFVGMKSLVFQSTVGSNCVIEPEAAAIGVTVPDGKYIPAG |
| | TVVTSQAEAAKLPEVTPDHADYTTQAAVVTVNVALCEAYKA |
| | QA |
| 99 | MGSYKTLTDIGKMMLKTLLLASTVSAWTYSDQTAWGGECKT |
| | SKSQSPINIVTSSAVCKNSKDDPIKADSFVAEKLGGKHAMTLN |
| | NVTSSGTHSATWTFKTMPENSQLKCAQHHCHFDVAEHSMDG |
| | EKHFGECHVVCMQAKYADLGKALESKATDALAVFGFLLAKG |
| | TATTADHAVTKQMIDAKKNYAEGKEYEMEIPATTQLADGYY |
| | RYNGGLTTPGCNEAVTWTVFKNVQYVSVAQYNEIMTWKDGN |
| | LRGNDRKVQPMNGRSLTFYKSSASKMMASLAIIGVMFMF |
| 100 | MRFNRFVTTLLAACLMPLMTQAAPWGYTGETGPAQWGKISK |
| | EYATCQTGINQSPVDIQTATTSKLGLPALNTQYIDNPTRFRSIN |
| | YTLRATMSSYSSNFIEIEGRLYYLKHFDFHAPSEHTLNGKTYPL |
| | ELQLVHKNQHGDIAIVAVMFDVGEPNQAIQNLWESFPTMVDN |
| | SMPIFSDVDINQLLPDNKAYWLYSGSLTTPPCTEGVTWVVLKK |
| | PVALSAEQLDNFHYIVGPANNRPPQPLNARTITDSHSGNTEILY |
| 101 | MQEITVTRFENIQPSPVTPWNPEPKLPEIHPTAYIHPAAVVQGD |
| | VTIGENVMVSANASIRSDEGYPIVIGDNSNVQDNVVLHALETV |
| | GEDGEVLEENVVVVGDERYAVYVGKNVSLAHQAQVHGPAA |
| | VGDNTFIGMQAFVFRSRVGKDCVLEPLAAAIGVTIPDGTYIPA |
| | GLVVTTQEEAAKLPKVTPDHPFANTNAAVVKVNVALAKGYL |
| | AQA |
| 102 | MQEITVLEFSNVTKNEVTPFNPKPETPVIDPTSYIDPNATVIGDV |
| | TIGKNCMISANASIRSDEGKPIVIGDRSNVQDGVVLHALESVDD |
| | GGMVLGDNVVVHGNSWFAVYVGKNVSLAHQAQVHGPAAV |
| | GDDSFIGMQSFVFNSIVGSNCVIEPNAAAIGVTVPDNKYIPAGT |
| | VVTSQEEADKLPEVTEDDKYFTTQEAVVKVNVNLAEAYRGLA |
| 103 | MLKRISAFTVCGWNYEDQHPEKWASLFPLCAGKRQSPINIVTS |
| | EVVYDPKLKPLKLSYEPATCREIVNNGHSFNVEFDDSQDKSVL |
| | KGGPLSGIYRLKQFHFHWGAADDKGSEHTVDGAKYSAELHLV |
| | HWNAKYGDFAEAASKPDGLAVVGVFLKVGKANPELQKLLDA |
| | LGSIKTKGKQTRFTNFDPSTLLPSSLDYWTYDGSLTTPPLLESV |
| | TWIVLKEPISVSPAQMEQFRSLLFTSEGETACCMVDNYRPPQPL |
| | KGRQVRASF |
| 104 | MIKTNPRGDLPQVHESAFVDPTAILCGWVIVEEYVFIGPYAVIR |
| | ADELNADGDMEPIVIGAHSNIQDGVVIHSKSGAAVTIGRHTSIA |
| | HRAIVHGPCRVGDGVFIGFNSVLFNCTIDDGCVVRYNAVVDG |
| | CHLPPGFYVRSTERIGPETDLAALPQVTADASDFSEDVARTNN |
| | ALVLGYKHIQNEF |
| 105 | MQEITVTVFENIRPSPVTPWNPEPRLPEIHPTAYIDPAAVVQGD |
| | VTIGENVMISANASIRSDEGYPIYIGNNSNVQDNVVLHALETVD |
| | ENGKRIEENIVTVGDKEYAVYIGDNVSLAHQAQVHGPAAVGD |
| | NTFIGMQAFVFASRVGKNCVLAPLAAAIGVTVPDGTYVPAGK |
| | VVTTQEEADKLPKMTPDHPFYKTNDAVVKVNVALAKGYLAQ |
| | A |
| 106 | MKAISLVFTCGYWRENDPHQWHLTFPAAKGERQSPIDIQPAKA |
| | KYDPGLKPLKLSYDPATARRILNNGHSFNVEFEDSQDKAVLKG |
| | GPLTGSYRLLQFHFHWGSTDDHGSEHTVDGVKYASELHLVH |
| | WNAVKFSSFAEAASKADGLAVIGVFLKVGEPHAEMEKLLNAL |
| | HAIKTKGKEAPFTNFDPSCLLPTCLDYWTYSGSLTTPPLLECVT |
| | WIVLKQPISVSSEQMAKFRSLLFTSEGEKEKRMVDNFRPLQPL |
| | MNRTVRSSFR |
| 107 | MTRRAVLNRRGALAALALLAVAGCAGSDPTAAAPHWDYDHE |
| | GPDHWADLGKQYATCRNGHAQSPIDLPDAGEAHPTDDIDIVY |
| | RRIRTATLTNNGHAIQVGVPADSGNRIVVDGTSFTLTQYHFHL |
| | PSEHTVAGAETAMELHLVHTDAHGRLAVLAVLLRAQEAPAPL |
| | SAILAAAPDRVGATRTLSNIDPRAFLPDNRAQFRYEGSLTTPPC |
| | TEGVAWIVLREPSPVAVADVDRYRRLFPHSNRPTQPRNDRPVI |
| | LAGTN |
| 108 | MKIISWLFIFLLGACATDWSYSGRGSPQNWAEISESNKFCKIGY |
| | NQSPIDINLSMNKDFILNDLKFDYKISEIEKVNEKYYQKINFYSK |
| | SFVLRGKKKYWLKYIEFRHPSEHFLDSSPHSLEMQIYHKSEDE |
| | QWLATSYFLEIPAMNNNENLYFNNLIDFLKSKKIEDKFDLSKII |
| | DETSLSFFYEGSFTTPPCTEGVKWYIMKNPIFISKEQMNTIIKSTI |
| | FVKSNARGIQKFNPEKF |
| 109 | MQPSAFHKLLLLLPLAYHRTPNVGDDKDEHWNYETNGKNWG |
| | GICASGERQSPISLSVQKSYIISIPRIVFGNYDIKLRGPLTITNNGH |
| | TAHMDIPETTNGKKPFITEGMLNGRYVAESLHFHWGSPGSRGS |
| | EHAINKQRYDVEMHIVHRNAKYKDMSEAVGKKDGLAVIGVM |
| | LKIVKNPKLMFLGLHNVLGAVSRITKTKAKTYVPGSFSLGQVL |
| | GIVNPRSYFTYRGSLTTPFCQEAVTWTVFTQVLPVSYTLVSKL |
| | WRLRDSEGHRLINNFRDIQPTNRRAVFYRP |
| 110 | MQEITVTKYENIQASPVTPWNPEPKLPEIHPTAYIHPAAVVQGD |
| | VTIGANVLVMANAVIRADEGYPIVIGDNSSVQDNVVLHALETV |
| | DADGKVIEENVVTVGDKKYAVYVGDNVVLAHNAQVHGPAA |
| | VGDNTFVGMNALVFNSVVGKNCVLAPLAAAIGVTVPDGKYIP |
| | AGKVVTTQEEAAKLPEVTPDHPFYNLVDRVVAVNVALAAGY |
| | LAQA |
| 111 | MQEITVTRFENIQPSPVTPWNPEPKLPEIDETAYVHPAAVVQGD |
| | VKIGKNVLIMANAVIRADEGYPIVIGDNSSVQDNVVLHALETV |
| | DENGNVIEENVVTVGDEKYAVYVGDNVVIAHNAQVHGPAAV |
| | GDNTFVGMNALVFRSTVGKNCYLAPNAAAIGVTVPDGTYVPA |
| | GTVVTTQEEAAKLPKITPDHPFANLNKRVVKVNVALAKGYLA |
| | LS |
| 112 | MSYSRVSTYLLALSVLCFSVFVVVPTGVCQAINPPEKVQPGQH |
| | VHNGQHHMLHMMLGEEKCGPTYTYEEGVKGPSHWPEVCTTG |
| | KMQAPIDIQSTQKLPINNLKFNYQPADLDILNDCNQYRVLVKF |
| | PDNYWLMVGKKPYNLAEIHFREPGETAVNGKRPKMSIEFLHFS |
| | PEGVFLVIEVPVVAGKENPTMQAILQNVPAPGKEEKVAGVKIN |
| | PTDLLPIDRRSFYRYPGSLTTPDCTEVVTWYVMKTPIEMSEAQI |
| | AEYSKHYHDTARPLQPVNGRPVVEDQ |
| 113 | MTCTGKSTDYWNYDNPSEWGTHFPAANGLCQSPIDIDSHKTIR |
| | HVYPKFQFSKKYHSSELFKLINQTYQVTATLADRTYGQNDND |
| | LWFTGGGLEGTFYFVNFHLHWGRDDRHGSEHEIDGHQFPAEG |
| | HVVFQNRQTKQAAVFAFLFTVADRFHKENKEWCKYADAASQ |
| | LTNDEDSIQCLFNLHDLMNVNDRLFYRYTGSLTTPPCTEGIVW |
| | TIFSQKIAIKQESLQKLRKNILTKVYRPVQPLNDRIVYKNH |
| 114 | MKLSNQIRFYGVATCPEWHDYYPIADGDRQSPINIISSQARYDP |
| | SLRPLELKYDPSTSLEILNNGHSFQVTFADDSDSSTLKDGPISGV |
| | YRLKQFHFHWGAADDKGSEHTVDGVKYPAELHLVHWNAVK |
| | YSSFAEAASKENGLAVIGVFLKIGQHNANLQKIVDALNAIKTK |
| | GKQTTFTNFDPSTLLPGCLDYWTYDGSLTTPPLLESVTWIVCK |
| | EPISVSSEQMAKFRSLLFS |
| 115 | MRIMTRGALTGVLWMLSVVGLQAAEPGSIPWGYEGDLGPNH |
| | WGSLGSEFALCEKGMSQSPIDLVQTHKLALTDIQFSYRDAPFH |
| | VINTGHTLEELEPLSETVKSRYPKHGQTVLHFQKDSTIVFDDDL |
| | YLLEQFHFHSPSEHTLHEKHYPMELHLVHHNERHEAAVVAVF |
| | MKEGKHNPFFETFLDHAPKTVGEFVEDRERVINPVNLLPKNHT |
| | YYRYFGSYTTPPCHEGVIWAVMHDPIEVSREQVQRFRSLVGH |
| | DNARPTQPLHKRFVLESNDVRAPGKLK |
| 116 | MSLKEWGYDAHNGPQTWCRVFIAAEGKRQSPIDIQTKEVESD |
| | LTLKPLKLNYEPASSLRILNNGHSFQVEFDDSTDKSVLTGGPLT |
| | GTYRLRQFHFHWGSCDDHGSEHTVDGVKYASELHLVHWNAK |
| | YESFAEAAKQPDGLAVVGVFLKIGKENPKLQRVLDALNAIKT |
| | KGKQTTFTNFDPSTLLPPCLDYWTYHGSLTVPPLLESVTWIILK |
| | EPISVSPSQMSKFRSLLFT |
| 117 | MQEITVLNYSNIVKNEVTSTNPKPEVPVIDPTSYVDPNATVIGD |
| | VTIGKNCYIAAFARIRADEGKPIVIGDRSNVQDGVVLHALESID |
| | DTGEVNKDNVVIEGNELYAVYIGDNVSLAHQSQVHGPARVGD |
| | DSFVGMKSLVFKSDVGDNCVIEPEAAAIGVTVPDNKYIPAGTV |
| | VTSQEEAAKLPEVTPDYAYYTTQEAVVEVNVALTEAYKGKM |
| 118 | MQEITVMDFSNITKNEITSWNPEPSTPKIDPTSYIDPNATVIGDV |
| | TIGKNCYIGPFAVIRADEGAPIVIGDESNVQDGVVLHALESVDA |
| | GGKIREDNVVLHGDKLYAVYIGKNVSLAHQAQVHGPAYVGD |
| | DSFVGMNSLVFKSKVGSNCVIEPFAAAIGVTVPDGKYIPAGTV |
| | VTTQAEADKLPEVTPDHAEYTKNAAVVNVNVALCEGYKSLA |
| 119 | MRRKRVSRFNAPQRLPYMHKLAIAAALFLAAAIPSFAADDCPV |
| | PWGYTVDNGPATWGRYSAICASGLSQSPVKINNLLPSPATNLP |
| | TLSFQGGPSRFRVKNNQHDLEVYPVNQWTLQPFGARLTKFHF |
| | HVPAEHLDGNTRHDAEAHFVYELGNRIFAIAVWIDQVNQGGN |
| | AALQKIAAVQRPGLCLMSPLSLPAATLNILDFLPDRNNYAAYH |
| | GSLTTPPCTENVTFFIMRTPITATATQINALTLVAPAPPGNARPV |
| | QQTKWRR |
| 120 | MQEITVTNYNNIRPSPVTPWNPEPKLPEIHPTAYIDPKAVVQGD |
| | VTIGKNVLVMANAVIRADEGYPIVIGDNSSVQDNVVLHALETV |
| | DENGNVLEENVVEVGDKRYAVYIGDNVVLAHNAQVHGPAAV |
| | GDNTFVGMNALVFRSRIGKNCVLAPLAAAIGVEVPDGTYIPAG |
| | TVVTTQEEAAKLPKVTPDHPFANLNERVVKVNVALAKGYLAL |
| | A |
| 121 | MQEITVLLFSNIRKNEVTPTNPKPTTPVIDPTSYIDPNATVIGDV |
| | TIGANCFVGPFAVIRADEGAPIVIGDRSNVQDGVVLHALESVD |
| | DGGKVREDNVVVHGDEWYAVYIGRNVSLAHQSQVHGPAHV |
| | GDDSFVGMKSLVFKSKVGSNCVIEPGAAALGVTVPDGKYIPA |
| | GTVVTTQAEADTLPEVTPDYAFYTTQAAVVSVNVNLCEAYRA |
| | QA |
| 122 | MKRSLAFVTIGCQHNPEDWYPFIEGDEFGYSDSLQREWVMCK |
| | SGRMQSPIDISPENLLFDPNLRSLQIDKHKVSATLENLGQLPLLT |
| | INDSKIRPDSINISGGPASPYKYRLHHIIIHFGRSIDEEKGSEHTID |
| | HIRFPAELQLLAYNTDLYSNFSEAMTQPRGLLAISIIVDIGKITN |
| | TELRKLTVASQSITYKGQKTILKRFNAYGLLPETEDYITYEGSL |
| | TFPGCYETVTWVIMNNPIYITKEDLHIWNDLQQTEFKQPNPVF |
| | MFPNYRPLKPLNGRLLRTNINIKYK |
| 123 | MQEITVLEFSNIKKNEVTSYNPKPKTPVIDPTSYIDPNATVIGDV |
| | TIGKNCYIGPFAVIRADEGAPIHIGDNSNVQDGVVLHALESVDD |
| | GGKVREDNVVLYGDKYYAVYIGKNVSLAHQSQVHGPARVGD |
| | DSFVGMNSLVFNSIVGNNCVIEPNAAAIGVTVPDNKFIPAGTV |
| | VTSQAEADKLPEITPDHAFYTDIAKVVSVNVKLCKAYLEKQ |
| 124 | MQEITVLTYSNVTKNEVTSTNPKPTTPVIDPSSYVDPNATVTGD |
| | VTIGKNCLIGANAVIRADEGAPIVIGDNSSVQDGVVLHALESVD |
| | DGGKVIEDNVVLHGDNWYAVYVGKNVVLAHNAQVHGPAYV |
| | GDDSFVGMKSLVFKAIVGSNCVIEPDAAAIGVTVPDGKYIPAG |
| | TVVTTQEEADKLPEITPDDAKYTKVAEVIAVNVALCKAHREK |
| | A |
| 125 | MRKISFLVAGCTPENWHDYQPVAGGERQSPINIITKEAKYDPSL |
| | KPLSFTYDPSTSLEILNNGHSFQVTFADNSDSSTLTGGPLTDKY |
| | RLTQFHFHWGSTDDHGSEHTVDGVKYASELHLVHWNADKYS |
| | SFAEAASKPDGLAVLGVFLKVGEHNPSLQKLTDALYSVRFKG |
| | TKAQFTNFNPKCLLPSSLDYWTYSGSLTTPPLLESVTWIVLKEP |
| | ISVSSEQMEKFRSLLFTSEGETACCMVDNYRPLQPLKGRKVRA |
| | SF |
| 126 | MVFSIATFGLLLLLGFCLGDDFGYDGNHGPSHWGEEYHTCIGK |
| | HQSPINIEEHNVKNVSLPPLKLIGIDDPYQSFVTNNGHTVMLKI |
| | NESKVIMLSGGPLGNKVYVFEQLHFHWGQNDFEGSEDLINNH |
| | SFPMEMHAVFYKEDYKSMNEALNHSDGLAILAYLYEVSPNPN |
| | VMYEPIVEVLPDIETVGSEKVLREPLMLRKLFISDITTMQDYFT |
| | YNGSLTTPPCLEVAIWIDFKDHLRLSHEQIAAFRNLRSTEGDKL |
| | THNFRPVQSLEDRIVLHNIPREQNIPRNIPPKTYHRFDEHSGQH |
| | NVEMPLSIIALAVLFAVILFAI |
| 127 | MTLFSKIRENVYGHAPQCDWCYEGEGAPESWGRLRPEFATCA |
| | VGRRQSPIDIRDGIAVDLEPIRFDYRPTSFRIVDTGNTIQVNVAP |
| | GNTIEVMGRRYELVQFHFHRPSEERIDGRQFDMVAHLVHKDG |
| | EGRLAVVAVLLERGDDQPLVRTVWNNLPLEKGDEVAARTPID |
| | LNALLPEDRRYYTYMGSLTTPPCSEGVLWMVMKQPVQLS |
| 128 | MQEITVTTYNNIRPSPVTPWNPEPKLPKIHPTAYVDPAAVVQG |
| | DVTIGKNVMISALASIRSDEGYPIYIGDNSNVQDNVVLHALETV |
| | DENGNVIEENVVTVGDKKYAVYVGDNVSLAHQAQVHGPAAV |
| | GDNTFIGMQAFVFRSNVGKDCVLEPLAAAIGVTVPDGTYVPA |
| | GKVVTTQEEADKLPKVTPDHPFYKTNEAVVKVNVALAKGYL |
| | ALS |
| 129 | MQEITVLEFSNITKNEVTPWNPKPKTPVIDPTSYIDPDATVIGDV |
| | TIGANCYIGASAVIRADEGKPIVIGDDSNVQDGVVLHALESIND |
| | EGKVIEDNVVIHGNKRYAVYVGKNVSLAHQSQVHGPAAVGD |
| | DSFVGMQSLVFNSKVGSNCVIEPNAAAIGVTIPDGRYIPAGTVV |
| | TSQAEADKLPEITPDYAKSNAVAAVVNVNVGLCEAYREEA |
| 130 | MQEITVGEFSNVTKNEVTTTNPKPETPVIDPSSYVDPSSTVIGD |
| | VTIGKNCYIAANAVIRADEGAPIVIGDNSNVQDGVVLHALESV |
| | NDGGKLREDNVVLEGDEYYAVYVGKNVHLAHQAQVHGPAA |
| | VGDDSFVGMKSLVFNSIVGSNCVIEPNAAAVGVVVPDGKFIPA |
| | GTVVTTQEEADNLPDITPDHAAYTTQAAVVKVNVALCEAYKA |
| | EA |
| 131 | MQEITVTKFENIRPSPVTPWNPTPKLPKIHPTAYIDPAAVVQGD |
| | VTIGENVMVSANASIRSDEGYPIYIGNNSNVQDNVVLHALETV |
| | DENGKRIEENIVRVGDKDYAVYIGDNVSLAHQAQVHGPAAVG |
| | DNTFIGMQAFVFRSRVGKNCVLEPLAAALGVEVPDGTYIPAGE |
| | VVTTQEAAAKLPKITPDHPFANTNAAVVKVNVALAKGYLALS |
| 132 | MRARAVTGGIHGRIRSTLGAALLAPLFLAAGCGGEGGGGTGE |
| | ESGLAETHLAAWSHAGADGPDEWASLDPAYATCGTGERQSPI |
| | DIVGAKRRPFPPVELDYAPVRATLIDNGHAIEAELEDSGSSARI |
| | GGDEFTLEQFHFHMPAEEVVGGKSFAASIHLVHLDEDGGAAV |
| | VGLLVEPGPENPVIERLAEEVPEETDEPVEVEGELDLAGLVPDG |
| | DAFRYEGSLTTPPCTEGITWTVFEDPVTMSPEQLEAFAGAYDA |
| | NARPVQARNGREISVGPGLG |
| 133 | MQEITVTVFENIRPSPVTPWNPTPRRPEIHPTAYVDPLATVVGD |
| | VRIGANVMVSANASIRSDEGYPIVIGDNSNVQDNVVLHALETV |
| | DAAGRRLEENVVEVGDELYAVYVGANVSLAHQAQVHGPAAV |
| | GDNTFIGMQAFVFRSRVGANCVLEPLAAAIGVTIPDGTYIPAG |
| | KVVTTQEEAAKLPKITPDHPFANTNAAVVAVNVALAAGYRAL |
| | A |
| 134 | MQEITVLTFSNVTKNDVTATNPKPVTPVIDPTSYVDPNATVTG |
| | DVTIGKNCLIEANATIRADEGHPIVIGDRSSVQDGVVLHALESV |
| | DDGGELIEDNVVLEGDEEYAVYIGKNVHLAHQSQVHGPAKVG |
| | DDSFVGMKSTVFKSIVGSNCVIEPDAAAIGVTVPDGKYIPAGT |
| | VVTTQEEADKLPEITPDHAKYTANAAVVTVNVALCEAYRNEA |
| 135 | MQEITVLEFSNITKNEVTPWNPKPKTPEIDPTSYIDPQATVIGDV |
| | TIGKNCYIGPFAVIRADEGAPIVIGDDSNVQDGVVLHALESINE |
| | KGEIIEDNVVIKGNKRYAVYIGKDVSLAHQSQVHGPARVGDH |
| | SFVGMNSLVFNSIVGDNCVIEPNAAAIGVTVPDGKYIPAGTVV |
| | TSQAEADKLPEITPDHAYYTKNAAVVNVNVALCRAYKSKE |
| 136 | MSTIPWRLGAVFCNYQKHEDAVEEKEFSYDEGSERGPSRWGEI |
| | RPEWRTCGNGEMQSPIDLLNQRVEIVSKLGKLKRDYKPSNATL |
| | KNRGHDISLEWKGGAGSIEINGTEYVLQQCHWHSPSEHTINGR |
| | RFDMELHMVHESRDGKVAVVGIVYKLGRPDSFLSSLMDHLEA |
| | ISDTKDRERAVGVIDPRHIKFGSRKYYRYMGSLTVPPCTENVI |
| | WTIVKRVRTVSREQLK |
| 137 | MKYGVLVLILSFIQFTYAQNKKDWGYKDSGAPQYWANINPLY |
| | LGCTEGNQQSPINIITKNVNKGAAHFELKYSVAKGVNLILSHNT |
| | FKMVYPQGNFLEMNGNRYQLKEIYFKTPGENAIDSLRGMLEA |
| | QLLHEDSKGNKVILAVFFIEGRSNPIIDMLVKNLPTQPDKANFI |
| | ANVDVHQLLPSDLASYQFDGSLTMPPCSQGVRWIVLKQTMTI |
| | TQSQVDSMRDITGVNSRPTQEIFNRLIVK |
| 138 | MQEITVLIFSNIRKNEVTPTNPKPVIPVIDPTSYVDPNATVIGDV |
| | TIGKNCYIAHSAVIRADEGKPIVIGDRSNVQDGVVLHALESVN |
| | DGGKIREDNVVIEGDEEYAVYIGKDVSLAHQSQVHGPARVGD |
| | HSFVGMKSLVFNSIVGSNCVIEPDAAAIGVTVPDNKYIPAGTV |
| | VTSQEEADKLPEITPDHAKYTAIAAVVNVNVALCQAYKEKS |
| 139 | MKRTFLIAVSLCPGLIQYNHWDEWWTYEGISGPAYWGLINPA |
| | WSLCNKGRRQSPVDIDPEKLLFDPNLKSLHLDKHKVSGTLENT |
| | GQSLVFRVDKDTKHHVNISGGPLAYKYQFQEIYFHWGVHDGL |
| | GSEHTINHQSFPAELQLYGFNSELYSNMSEAQEKPHGVVGISLL |
| | VQIGKTPNPELKILTSQLENIRYKGQSAPIKNFSLRGLLPNTEHY |
| | VTYEGSTTHPGCWETTVWVVLNKPVYITKQELYALRRLMQGS |
| | KEHPKAPLGNNARPTQDLHHRTVRTNI |
| 140 | MQEITVTRYENIRESPVTPWNPTPRRPEIHPTAYVDPAAVVVG |
| | DVTIGENVMISANASIRSDEGYPIYIGDNSNVQDNVVLHALETV |
| | DAAGKRITENIVTVGDKEYAVYIGKNVSLAHQAQVHGPAAVG |
| | DNTFIGMQAFVFRSRVGKNCVLEPLAAAIGVTVPDGTYIPAGK |
| | VVTTQEEAAKLPKITPDHPFAKTNAAVVAVNVALAAGYRALA |
| 141 | MKLTSIAVFCGYPEQNRDHWGYQDHNGPEMWKEKFPSAGGK |
| | KQSPIDIQTAETTFDPKLKPLELKYDPSTAKEILNNGHSFQVTFV |
| | DDTDSSTLKDGPISGIYRLKQFHFHWGASDDHGSEHTVDGVK |
| | YAAELHLVHWNAKYGKFGEAASQPDGLAVVGIFLKIGRHHEE |
| | FQKLLDALDSIKTKGKQTTFTNFDPSTLLPGCLDYWTYFGSLT |
| | TPPLLESVIWIVLKEPISVSSEQLAKFRSLLFTSEGEKEKRMVDN |
| | FRPLQPLMNR |
| 142 | MQEITVAEFSNVTKNEVTPYNPKPVTPVIDPTAYVDPEATVIGD |
| | VTIGKDCMISANASIRSDEGHPIVIGDRSNVQDGVVLHALESVN |
| | DGGEVIEDNVVVEGNELYAVYVGKNVSLAHQAQVHGPAAVG |
| | DDSFIGMQSFVFNSIVGSNCVIEPEAAAIGVIVPDNKYIPAGTVV |
| | TTQEEADKLPEITPDYAYYTTVAAVVNVNVALCKAYRRLM |
| 143 | MPAPAPKAAPKAGHGAKKAAPAPKAAPKAAPKAAPRAKVVK |
| | AAPAPPPPEPAHAHWSYEGEGAPARWGQLKPEWKQCAVGTR |
| | QSPIDIRDGIKVDLDPIQFDYKASGFSVIDNGHTVQVNLAPGNFI |
| | TVLGRRYELVQFHFHKPSEERINGKPYDMVAHLVHKDAEGRL |
| | AVVAVLLRPGEANPLIEKVWTYMPLDAGDRVRMPTELIDLNQ |
| | LLPADRAYFTYMGSLTTPPCSEGVLWMVMKQPVPVSADQIAIF |
| | ARLYPMNARPLQAVSGKIIKETLM |
| 144 | MNRKKLNSLIAAVIVFFATSAFSESPHWDHAEQSTWWAIEDTT |
| | QTYPPKRFPFAVCGVGQHQSPIDLAAAEVIDTIQINPLEILYDVD |
| | HAPVFFNSGHGIQVNTSIEYSGKLKVGEELFPLIQFHFHAPGEH |
| | VIGDTKFPAELHYVHIQADGKIAVLAVAINIGDENSAFQTILEN |
| | VPSVSGGKNENSGLQFDPAALLPPLDHPIKYYTVAGSLTTPPCS |
| | EGVQWYFLPTAITISEAQLNQLRSLYADNNRLPQDVNGRSLLT |
| | Q |
| 145 | MQEITVTRYENIRESPVTPWNPTPRRPKIHPTAYVDPAAVVVG |
| | DVTIGANVMVSANASIRSDEGYPIYIGDNSNVQDNVVLHALET |
| | VDADGKTLEENVVTVGDKKYAVYVGDNVSLAHQAQVHGPA |
| | AVGNNTFIGMQAFVFRSTVGENCVLEPLAAAIGVTVPNGKYIP |
| | AGKVVTTQEEADKLPEVTPDHPFANTNAAVVKVNVALAAGY |
| | RALA |
| 146 | MQEITVDEFSNITKNEVTGTNPEPSTPVIDPTAYVDPNATVIGD |
| | VTIGANVLVAANAVIRADEGRPIVVGDRSSVQDGVVLHALES |
| | VDDEGEVREDNVVLVGDENYAVYVGKNVSLAHQSQVHGPA |
| | AVGDDSFVGMKANVFRSTVGSNCVIEPDAAAIGVTVPDGKYIP |
| | AGTVVTTQAEADKLPDVTPDHAKSNDVAAVVAVNVALCEAY |
| | REQS |
| 147 | MQEITVLLFSNVTKNEVTPINPKPTTPVIDPTSYIDPNATVTGDV |
| | TIGKNCMISANASIRSDEGKPIVIGDRSNVQDGVVLHALESVDD |
| | GGMIIGDNVVVEGDEYYAVYVGDNVSLAHQAQVHGPAYVGD |
| | DSFIGMQSFVFKSIVGSNCVIEPEAAAIGVTVPDGKYIPAGTVV |
| | TTQEEADKLPEITEDDADYTTNVAVVNVNVALCKAYREKA |
| 148 | MQEITVTRFENIRPSPVTPWNPEPKRPVIHPTAYVHPAAVVEGD |
| | VTIGENVMVSANASIRSDEGYPIYIGDNSNVQDNVVLHALETV |
| | DENGKVLEENVVTVGDKKYAVYVGKNVSLAHQAQVHGPAA |
| | VGDNTFIGMQAFVFRSTVGKDCVLEPLAAAIGVTVPDGTYIPA |
| | GKVVTTQEEAAKLPKITPDHPFANTNAAVVKVNVALAKGYLA |
| | LA |
| 149 | MQEITVAVFSNVTKNEVTGTNPKPRTPVIDPTSYVDPNATVIG |
| | DVTIGANCYIAHSAVIRADEGRPIHVGDNSNVQDGVVLHALES |
| | VDADGERLEDNVVIEGDKRYAVYIGKNVSLAHQAQVHGPAR |
| | VGDDSFVGMKSLVFNSVVGSNCVIEPNAAAIGVTVPDGKYIPA |
| | GTVVTTQAEADKLPEVTPDHAAYTEIAKVVTVNVNLCRAYRE |
| | QA |
| 150 | MYPIIHIKEGKYKMNYFFLFTILSSLTLSACSNSKIVQEVHPNKS |
| | IVSAARNEDWSYTGKTGPNYWSSINKKYALCSTGKQQSPVNID |
| | QAIKKSLPLGINYHNDLFKIERSQYTVKFIPVNHSNSINLNGTN |
| | YTLLQFHFHTPSEHTLNGKQSDLEIHFINENSNKSIITIGVLVDR |
| | GRLNKEFQKILNANPMDEDLEGKVVKINLQSFIPYTSKKFSYT |
| | GSFTTPPCTEGIKWIIFNKPIQFSEEQIHSYQNYFEPNSRPVQPLN |
| | GRDLFESW |
| 151 | MQEITVFVFSNVEKNEVTSTNPKPTTPVIDPTSYVDPNATVTGD |
| | VTIGANVMISASASIRSDEGKPIVIGDRSNVQDGVVLHALESVD |
| | DEGEVIEDNVVIHGDKNYAVYVGKNVSLAHQAQVHGPARVG |
| | DDSFIGMQSFVFKSDVGSNCVIEPFAAAIGVTVPDGKYIPAGTV |
| | VTTQEEADKLPEVTDDYAYSDTNEAVVKVNVNLCEAYKGKA |
| 152 | MNPTGHMPVVSETAFIDPTAIICGKVIIEDNVFIGPYAVIRADEV |
| | NEQGDMEAIVIKRDTNIQDGVVIHSKAGAAVTIGERSSIAHRSII |
| | HGPCQVCDDVFIGFNSVVFNAVIGKGCVIRHNSVVDGLDLPEN |
| | FHVPPMTNIGADFDLNSISKVPPEYSSFSESVVSANHTLVKGYK |
| 153 | MLSRKPGAVTFCIWNHQEYDVKMASWGYTKENGPATWYKD |
| | FPVANGPRQSPINIDPGSAKYDPGLKALTLKYDPSTSLEILNNG |
| | HSFQVTFADDSDSSTLTDGPISGVYRLKQFHFHWGASDDKGSE |
| | HTVDGVKYAAELHLVHWNAVKYSSFGEAASKEKGLAVLGVF |
| | LKVGEHNANLQKVLDALDSIKTKGKQAPFTNFDPSTLLPASLD |
| | YWTYHGSLTTPPLLESVTWIVLKEPISVSPAQMAKFRSLLFSSE |
| | GEKEKRMVDNFRPLQPLMNRTVRSSF |
| 154 | MKKGLVLICLSLSLLGAFGGEHWGYSKGVGPRYWGKLSRDY |
| | EICKSGKTQSPINIQHYYHSPDKEDLSFEYENTKPLSIAYSHYTL |
| | VAQFNEPGNAVIFRDHEYSLVNLHFHIPMEFAIHGKKQPLSMH |
| | LVHRDKEGDLLVVGIGFSIGKKNPFFTPILNAYKYHTEPKLLAL |
| | KTLLPDTIHYYHENGSLTTPPCSEGVTWFIIEETLSISKEQFDEM |
| | QQIMHHQSNQRPLQKDYNRVIVKSSAIVREH |
| 155 | MQEITVDEFSNVTKNEVTATNPKPTTPVIDPTSYVDPEATVTG |
| | DVTIGKNCLIAANAKIRADEGKPIVIGDRSSVQDGVVLHALESV |
| | DDGGKVIEDNVVLEGNELYAVYVGENVVLAHNSQVHGPARV |
| | GDDSFVGMKSLVFKSDVGSNCVIEPFAAAIGVTVPDGKYIPAG |
| | TVVTTQEEAAKLPEVTEDYPFYTKQAEVVKVNVGLCEAYRNK |
| | A |
| 156 | MIGVKPKSYPAQNKKKKWSYDGDNGPQNWGDLSADYLSCEV |
| | GLNQSPVDFSKSFSSPDLHSIRFNYGISAGRVYLARGSITFKVIP |
| | GNVAYFKGKQWVLERVVLRTPSEHSIEGHSFDGELQLYHSYK |
| | GESFLWVSVLLEAGSALKPFRQIVDKAKGISGASKLMGVDLRL |
| | LIPRRKNYYFYPGSDTIPPCKEGRSWVVLRQPVGASIKYIEHLE |
| | SQVGKGARPTQPLYARVPLRYD |
| 157 | MKGLCVMAIVALIGLQTATGYTRQDLSCGGRMDYSKPVCHNI |
| | PKHAHKKCDVYNQWSHHLFGTSQKCWGKLNPACRGMRQSPI |
| | NINEHKVEPNHNYGDLCITGPHHLKVHIHNTGHDLQAKLDESS |
| | SRATLVTGGPLGNKKYRVLQFHFHFASHPGGKGSEHSINCHFS |
| | DIEMHIVLQNVAYGSFDVAKDHRDGLSVIAVMLSEDVRPNTV |
| | SNAMRNNPSWSQYYINTLIYYASLRKHCDLHEVPGNTRFSLFH |
| | LLPSDYARNYYAYGGSLTTPPCSESVSWIIMRTRFHINRYHLNL |
| | LKDVSLLSRYHRGFEPMSQNKRNLQLLNNRKVYYPAGHGRCP |
| | SRG |
| 158 | MRLKSNIFVAGTCQPEDYHWGYEDHNGPATWAKHFPAAKGE |
| | KQSPIDIQLSNVKNVSFPPLVFNYKDSTLKEIINVGHSVQVNLE |
| | DSDNRSVLKGGPLSGPYRLKQFHFHWGKTNDVGSEHTIDGKS |
| | FPSELHLVHWNAKKYASFGEAASKPDGLAVVGVFLEIGDEHP |
| | EMNRLTDALYMVRFKGTKAQFSCFNPKCLLPASRHYWTYPGS |
| | LTTPPLSENVTWIVLREPISISERQMEKFRSLLFTSEGEKEKRMV |
| | DNFRPLQPLMNR |
| 159 | MKFLTPSIFFTSLRVASAATGVKFYYNDQSQWPAVPATPEGTN |
| | VCDGQQQSPINIDTGDFSCQADAQGYSFYTGDCTLGDYEFTM |
| | NDHGLKASVEKSNCEKPKMIIPGTGKVYEVLQFHIHTGCENKF |
| | NNTGCDAELHLVHIAKTDIALPAATTSADLPDLAVLGLMMYG |
| | VDEKHASVDALIDSWSEVSCSNQKCMTVSDELKSQKFSPYSLI |
| | PSDTSIYNFQGSLTTPPCWEVVTWNVAEKPIKMSFKQVLAITN |
| | LINKYSGYRAEDGSCVADSTVADDAGITSRDVQSLNGRQIVKN |
| | CEPLTVMSDFPAAAQEQSPMTASSESSAQFLNIKNIFGVVSVLA |
| | SIVLF |
| 160 | MQEITVTRYENIRPSPVTPWNPEPKLPKIHPTAYVDPAAVVQG |
| | DVTIGENVMVSANASIRSDEGYPIYIGDNSNVQDNVVLHALET |
| | VDEDGKVLEENVVTVGDKKYAVYVGKNVSLAHQAQVHGPA |
| | AVGDNTFIGMQAFVFRSVVGKDCVLEPLAAAIGVTVPDGTYIP |
| | AGKVVTTQEEAAKLPKITPDHPFANTNAAVVKVNVALAKGYL |
| | ALA |
| 161 | MIVLIIPLLFIVQSQTTTTTNTTTTTATTISYASQGSDWTSGVCSS |
| | STSQSPINLEVSSGTCDNSMVLDIQFKKDAMQIVMERVQYTIQS |
| | KAAVSNLYATDINGNLYGYTATSFMFHSPSEHTIEGTRYDLEM |
| | QIVHDLKSEFSATITKAIVSILFEVSSTDQPFFTTYDFALVASAST |
| | NTTTTNTTNSTNTTTAASVTSTIASINFNDLLGSQLDANPAYYT |
| | YVGSLTIPDCDENVNWYILDSILPITQTQLDAFNTYFLSNSTFAS |
| | GNGNNRAIQSTNDRTIKKGGVACEEQFVYFFSFFILYIFINYFIF |
| | KLL |
| 162 | MQEITVLEFSNITKNEVTATNPKPTTPVIDPTSYVDPQATVIGD |
| | VTIGKNCYIAASAVIRADEGKPIYIGDRSNVQDGVVLHALESV |
| | NDGGKIREDNVVVHGDEYYAVYIGKNVVLAHQAQVHGPAAV |
| | GDDSFVGMKSLVFKSIVGSNCVIEPDAAAIGVTVPDNKYIPAG |
| | TVVTTQEEADNLPEITPDYAYYNDVAAVVKVNVDLCKAYREK |
| | A |
| 163 | MQEITVLEFSNVTKNEVTSTNPKPVTPVIDPTSYVDPNATVIGD |
| | VTIGKNCYIAASAVIRADEGKPIVIGDRSNVQDGVVLHALESV |
| | DDGGKVREDNVVLEGDEYYAVYIGKNVVLAHQAQVHGPAA |
| | VGDDSFVGMKSLVFNSIVGSNCVIEPEAAAIGVTVPDGKYIPA |
| | GTVVTTQAEADKLPEITPDYAKSNAIAAVVKVNVALCEAYRN |
| | QS |
| 164 | MLKTNPRGDWPQVHASAFIEPTAILCGYVIVEENVFIGPYAVIR |
| | ADETDADGRIAPIVIGAHSNIQDGVVIHSKSGASVTIGRHTSIAH |
| | RAIVHGPCKVGDGVFIGFNSVLFNCTIDDGCVVRYNAVVDGC |
| | HLPPGFYVRSTERIGPETDLAALPQVTADASDFSEDVARTNNA |
| | LVLGYKHIQNEF |
| 165 | MKRNFLAISTVCGYDPEQWHMDYPIANGNRQSPINIITKDAKY |
| | DPNLKPLTLSYDPATAKEIVNVGHSFNVEFEDTDNKSVLKGGP |
| | LTGSYRLTQFHFHWGSVDGQGSEHTVDNVKYASELHLVHWN |
| | SVKFSSFAEAALKDNGLAVLGIFLKVGEHNPHLQKITDILNSIK |
| | TKGKQTTFTNFDPSCLLPASLDYWTYHGSLTVPPLLESVTWIV |
| | LKEPISVSSEQLAKFRSLLFTSEGET |
| 166 | MQEITVTRYENIRPSPVTPWNPTPKLPKIDPTAYVDPLAYVQG |
| | DVTIGKNVMISAHASIRSDEGYPIVIGDNSNVQDNVVLHALET |
| | VDANGNPLEANIVKVGDKDYAVYIGDNVSLAHQAQVHGPAA |
| | VGDNTFIGMQAFVFNSRVGKDCYLAPLAAAIGVTVPDGTYIPA |
| | GKVVTTQEEAAKLPKMTPDHPFYNTNAAVVKVNVALAKGYL |
| | ALA |
| 167 | MQEITVLVFSNIRKNEVTPENPEPVTPVIDPTSYIDPKATVIGDV |
| | TIGKNCYIGASAVIRGDEGYPIVVGDESNVQDGVVLHALESVD |
| | EQGKVIEDNVVRKGDKLYAVYVGKNVVLAHQSQVHGPAMV |
| | GDDSFVGMNSLVFKSRVGSNCVIEPYAAAIGVTVPDGKYIPAG |
| | TVVTTQAEADKLPEVTDDYKFYTQVAAVVTVNVKLCEAYRE |
| | QM |
| 168 | MGSSLILLPRVQTSRNGMKLFGILLSFGSVLGALAGNSWNYAG |
| | HGEYWPTSHAKSSTGAESYWDCDGIRQSPIDINSSMVQDVYF |
| | WNQLNLANYAASYAGKFKNNGHTLQFDLDDAETSGATLPTFS |
| | SPFMCTGCSYELQQFHFHWGSTAYQGSEHTKEGIAFPMELHLV |
| | HKKTSYSSVTASLSYNDGLAVIGIMFQLADTSDAGLTEIINAAV |
| | AIKNAADQHTHKEAKSIDMSTFLYQTGRSYYYYKGSLTTPPCT |
| | ETVDWHLMEGAIRITEADLEKLRDLTYTDDAPLVDNYRLPMP |
| | LNNRIIKRVFN |
| 169 | MQEITVTNYNNIQPSPVTSWNPTPKLPDIHPTAYIHPKAVVQGD |
| | VTIGKDVMISANASIRSDEGYPIVIGDNSNVQDNVVLHALETV |
| | DANGNRIEENIVTVGDEEYAVYIGKDVSLAHQAQVHGPAAVG |
| | DNTFIGMQSFVFRSRVGKDCVLEPLAAAIGVTVPDGTYVPAGK |
| | VVTTQEEAAKLPKITPDHPFANTNAAVVKVNVELAKGYLALA |
| 170 | MGLLDSKVWFVTQVLAAVPLVYSGPYPRTTSSYYDPNGFFQF |
| | ADKTHHRFYNFYQHVTEAARAEKLISNNEVWPSDKSISQMAS |
| | GSFSYREEDDYGPSNWGALNATCEGMYQSPINLIANRSVIVQQ |
| | KRALELKGSRNVPMAMVVENEGGAAAFFPEFRTNEQPRLRGG |
| | PLRGEYLFYQFHYHLGSEHTFDKKRYSAEMHLVFYNELYGSF |
| | KAARDQANGVAVIALTFDVLKSRRINSLNKWTRSLAEVVEAE |
| | SEYSIPRQELFSVSDVLGDMEWPYFAYEGSLTTPPCSETVQWIV |
| | ASERQLLTRSELKTMRMLKGRGGDWVQTARPTQALNFRRVFI |
| | Y |
| 171 | MQEITVLEFSNVTKNEVTPWNPKPKTPVIDPTSYIDPQATVIGD |
| | VTIGKNCYIAASAVIRADEGKPIVIGDRSNVQDGVVLHALESV |
| | DDGGEIREENVVIEGDEEYAVYIGKNVSLAHQSQVHGPARVG |
| | DDSFVGMKSLVFNSDVGSNCVIEPFAAAIGVTVPDGKYIPAGT |
| | VVTTQEEAAKLPEVTEDYPFYTAIQEVVKVNVKLCEAYREQK |
| 172 | MQEITVFEFSNVRKNEVTAWNPKPSVPVIDPTAYIDPNATVIGD |
| | VTIGKDCYIAASAVIRADEGSPIFIGDRSNVQDGVVLHALESVN |
| | PDGMYREENVVLKGNNLYAVYVGRNVSLAHQAQVHGPAAV |
| | GDDSFVGMNSLVFNSKVGSNCVIEPNAAAIGVTVPDGKYIPAG |
| | TVVTTQEEADKLPEITPDYAFYTQVAAVVQVNVELCRAYRGK |
| | A |
| 173 | MQEITVTRYENIRPSPVTPWNPTPRLPKIHPTAYVDPLAYVQGD |
| | VTIGDNVMISPHASIRSDEGYPIVIGNNSNVQDNVVLHALETVD |
| | ADGNEIEENIVTVGDEKYAVYIGDNVSLAHQAQVHGPAAVGD |
| | NTFIGMQAFVFKSRVGKNCVLEPLAAAIGVTVPDGTYIPAGKV |
| | VTTQEEADKLPKVTPDHPFYNTNAAVVKVNVALAKGYLALK |
| 174 | MQEITVTRYENIQPSPVTPWNPEPKLPEIDPTAYIHPAAVVQGD |
| | VTIGKNVMVSALASIRSDEGYPIVIGDNSNVQDNVVLHALETV |
| | DADGKRLTENIVTVGDEEYAVYVGDNVSLAHQAQVHGPAAV |
| | GDNTFIGMQAFVFRSTVGKNCVLEPLAAAIGVTVPDGKYVPA |
| | GKVVTTQEEAAKLPEVTPDHPFANTNAAVVKVNVALAKGYL |
| | ALA |
| 175 | MQEITVTVFENIRESPVTPWNPTPRRPKIHPTAYVDPQAVVQG |
| | DVTIGANVMISANASIRSDEGYPIVIGDNSNVQDNVVLHALET |
| | VDENGKTLEENVVTVGDKKYAVYIGDNVSLAHQAQVHGPAA |
| | VGDNTFIGMQAFVFRSTVGKNCVLEPLAAAIGVTIPDGKYIPA |
| | GTVVTTQEEADKLPEVTPDHPFANTNAAVVKVNVALAKGYL |
| | AQA |
| 176 | MGSCQVHAGGSRHFSFVLTFGRAYNGVVMLVPTSYLLLLLVT |
| | LLTPTFCADWSYKLGDQSGPDHWEYECKKEYQSPVNIPKGET |
| | TSTVFPALSFWNYELQPATATIENNGHTVKLATEPHRPKETPLL |
| | SGGGLLHSYKFAQIHFHWGAEDFKGSEHLVGDTQYPMEMHL |
| | VHYKAVHDTIKDALAEGAYDSLAVIGIFFEVSEQRNPALDLLM |
| | PYLAKIKAAHSEAAATPFPISSFLWGGDMSSFYRYNGSLTTPTC |
| | NEIVQWSVMKVPVPVTVDQLEVFRQLMTKDYEPLVDNFRPPQ |
| | ALGGRDVLDVMTVEMLRKGSHSGCEILAGPAALLASLILILCC |
| | RTWDL |
| 177 | MQEITVTVYNNIRPSPVTPWNPEPRLPKIHPSAYIDPAAVVQGD |
| | VTIGENVMVSPNASIRSDEGYPIYIGNNSNVQDNVVLHALETV |
| | DENGKEIEENIVTVGDKKYAVAVGDNVSLAHQAQVHGPAIVG |
| | DNTFIGMQAFVFRSKVGKNCVLEPLAAAIGVTVPDGTYIPAGT |
| | VVTTQEEAAKLPKVTPDHPFANTNAAVVKVNVALAKGYLAL |
| | K |
| 178 | MSTLVKAIRENPGCDWQYHFNPKISLIGRGQHQSPIDIHTKDAL |
| | FDPSLKPLSVSYDPATARLVNNGHTIQVEFEDSTDKSVVEGGP |
| | LEGPYRLKQFHFHWGKKDGVGSEHTVDGKSFPSELHLVHWN |
| | AEKYASFGEAAAAPDGLAVLGVFLQVGEHHPSMNRLTDALY |
| | MVRFKGTKAQFSCFNPKCLLPASRHYWTYPGSLTTPPLSESVT |
| | WIVLREPISVSERQMEKFRSLLFTSEGEKEKRMVDNFRPLQPL |
| | MNRTVRSSF |
| 179 | MSEKGPAYWGEIKEEWAACSNGTMQSPIDLLNERVEVVPGLG |
| | ELKRNYKPSNATLKNRGHDIALEWNGEAGSILINGTPYFLKQC |
| | HWHSPSEHSINGRRYDMELHLVHQSPENKIAVIGILYEIGPPDT |
| | FLSSLMDHIKAVTDTTEAERSVGVINPREIKRGSRKYYRYIGSL |
| | TVPPCTESVIWTVLAEIKKKIN |
| 180 | MLMRSFLIPTIVLSLILVSSNFAAEQDGADFDYNERGPEHWSQL |
| | DAKYKLCKDGERQSPINFITSIDLAIKLPNVNFIKQNTPNVKFPT |
| | ISMKKEGHATKFLPQQLIGSSFDSVRYIFNQVHFHTPSEHRFDGI |
| | HTDLEAHFVFEDSVTKKYSVIGVLYEVDCAVGSSSFFDSIIKLY |
| | NQDPNAKDNVPVDINSEVFSHIKEVYKYFGSLTTPNCTEDVTW |
| | WVVNKPLLISTSQLVKLRKHIGFNSRPTQPRNGRKESNRLILFH |
| | VKRFLLNYQVTVFAVFGLVGGITIE |
| 181 | MKLYTVIRGNPACSFHEDQWFAPSVQPGGHQSPINIVTSQTKY |
| | DPNLKPLTISYDPATSLEILNNGHSFQVTFDDTQDKSVLRGGPL |
| | DGVYRLVQFHFHWGSSDEQGSEHTVDKKKYAAELHLVHWN |
| | AVKYETFAEAAQEPDGLAVLGIFLKVGEHNAELQKITDILDSIK |
| | HKGKQTRFTNFDPICLLPPCPDYWTYPGSLTTPPLSESVTWIVL |
| | KQPIEVSPSQLAKFRSLLFTSEGETACCMVDNYRPLQPLMNRT |
| | VRSSF |
| 182 | MQEITVSFFSNVSKNEVTSTNPKPVTPVIDPTSYVDPKATVIGD |
| | VHIGKNCYIAASAVIRADEGAPIYIGDRSNVQDGVVLHALESV |
| | ADGGKVLEDNVVLEGDENYAVYVGKDVTLAHQAQVHGPAA |
| | VGDHSFVGMKALVFNAKVGKNCVIEPEAAAIGVTVPDNKYIP |
| | AGTVVTTQEEADKLPEITPDHENYTKVAEVVAVNVKLCEAYK |
| | SKA |
| 183 | MLSVPVSIAIATRAPDAVDASAPGEWGYADSSNGPARWSDILD |
| | ADGKASYPACGCAACQQSPIDLVRTAAKGNVRVGSLADRLVA |
| | PAKPVTLAVSQKHGTPNYVATDQNNDAAVVAPDGVRYTENS |
| | LHFHTPAENTVDGVANAMEMHMVHLSEAGDIAVLGVLFRHA |
| | DADLPANAEVTKLLRKIDADGGKTKVAVDLGGLYDGGAGFW |
| | EWTGSLTTPPCSGNVRWLLQKEVRGVDARQAEAFKKHVGGF |
| | PGNARPTQPLNGRAVLSFDPTGV |
| 184 | MQEITVFEFSNITKNEVTSTNPKPVTPVIDPTSYIDPNATVIGDV |
| | TIGKNVMIWPTAVIRADEGKPIVIGDNSNVQDGVVLHALESVN |
| | DGGKIREDNVVIEGNELYAVYIGKNVTLAHQSQVHGPARVGD |
| | DSFVGMKSLVFKSDVGSNCVIEGNAAAIGVTVPDGKYIPPGTV |
| | VTTQAEAEKLPEITEDYPFSDANQAVVEVNVKLCKAYRGLQ |
| 185 | MLAAGAHWEYSGEAGPANWAKLTPEFGACSGKNQSPINLTGF |
| | IEAELEPLAFAYQASATQVLNNGHTVQVNYAEGSTLTLDGQTF |
| | TLKQFHFHSPSENRIEGKSFPLEAHFVHASEQGALAVVALMFQ |
| | EGAANPELEKAWRVMPAHADQPVALPRPLDVQALLPKDHAY |
| | YRFNGSLTTPPCSEGVRWLVLKQPVEASKAQIEKFQKIMGYPN |
| | NRPVQPVNARTVLSS |
| 186 | MQEITVLEYSNVTKNEVTSTNPKPTTPVIDPTSYVDPNATVTG |
| | DVTIGKNVLIGPNAVIRADEGAPIVIGDNSSVQDGVVLHALESV |
| | DDDGEVIEDNVVLYGNKDYAVYVGKNVVLAHQAQVHGPAA |
| | VGDDSFVGMKALVFKSIVGSNCVIEPDAAAIGVTVPDGKYIPA |
| | GTVVTSQEEAANLPEITPDHEDYTTQEAVVKVNVGLCEAYRN |
| | QA |
| 187 | MMSVATALLLLSAVGTLAADWRYPTPGPDGSVGSPENWGGS |
| | CDHGRRQSPIDIAYAASVRGSYPEFIFDSYDSLPDSAYIVNNGH |
| | TVQINLDSSASSSVYGGGFRSKYVLEQLHFHWSSEHTIEDRRY |
| | ALEMHLVHRQSRYASVEQASSHKAGIAVLAVLFHVDEHPNEA |
| | IQLILNSTSPIKAKVDDRQPLRGSLHLNDLLPKDRTVYFRYEGS |
| | LTTPVCAESVVWTVFPESLPISLGQVQDFMTIHDADNRTLVVN |
| | YRPVQPLNTRVLVLVSDTEVEASGARRIASGMFAAVLLSLAIS |
| | LF |
| 188 | MKILVTFASCGYEPRNDHWQEPWSYEGISGPDHWGELNPEYS |
| | LCSTGKEQSPIDIDHTIKAQLPALKFDYKSEPLKYVINNGYTIRV |
| | NYHDAPGSGNFLIVDDTRYQLTQFHFHRPSEEYIHGKPYDMEL |
| | HLMHQSSDGKVAGVTVFIKTGRANSTTQKIWEHMPKTEGQQE |
| | VAGVEINPADMLPHDTGYYVYMGSVTAPPCTEGVNWFVLKT |
| | PVEISADQIEAFAKLYPHDVRPLQPLNGR |
| 189 | MKNSLFATIVGYCPEWRDHQPVAPLGQRQSPIDIVPADAQYDS |
| | SLKPLKLQYDPSTCLDILNNGHSFQVTFVDDTDSSTLTDGPISG |
| | VYRLKQFHFHWGASDDKGSEHTVNGAKYAAELHLVHWNAV |
| | KYASFAEAALEPNGLAVLGVFLKVGEHNPALQKLTDILPSIKH |
| | KDTQASFGKFDPSCLMPTCPDYWTYAGSLTTPPLSESVTWIVL |
| | KEPIEVSEEQLGKFRSLLFTSEGEKEKRMVDNFRPLQPLMNR |
| 190 | MQEITVLTYSNVTKNEVTATNPKPTTPVIDPTSYVDPNATVTG |
| | DVTIGKNVLIAANAKIRADEGKPIVIGDRSTVQDGVVLHALES |
| | VDDDGKVIEENVVLEGDEYYAVYVGKNVVLAHNAQVHGPAA |
| | VGDDSFVGMNSLVFNSVVGSNCVIEPNAAAIGVTVPDGKYIPA |
| | GTVVTTQEEADKLPEVTEDYEFYTQIAEVVKVNVNLCEAYRK |
| | QA |
| 191 | MKFIATTTALVAACALAISTVSAVSAVSEAGVKGAPWGYKPD |
| | DTTQASPVQWADHYPDCNGTHQSPIDLVTADVKQQTKAQNT |
| | LRFRGDCASFNLTQSAEGYKAEVVGGSCQVRGNKARYDLLQF |
| | HVHAPSEHTLNGEPLDGEVHFVHSNKDGSALLVVGLFMEIDPS |
| | GNTDPWLETLIDGIDDVSPTKEVMLDLTSYSALVKKTVRGGSL |
| | FNYPGSLTTPGCSEIVDWWVVEKPMKISAKDLTRIRENQGEID |
| | LNYKSESARPVQPLNDRIVKSFQ |
| 192 | MINSRPFLIFAVYDHGTAICGPADWKNVSAHCVENTQSPINIKT |
| | DKIFMHMFPYFDGFHFIVDNVVGSVSGVLVNNGHAPTLVIDQF |
| | ETPAILTGGPWANKVYRLNQIHFHFGCDASKGSEHTVDGRVY |
| | SGEIHFVTYNTKYFDFHAAADKPDGLSVVAVFLLDNGDKSNW |
| | KQLTDEMKKIIKADSFTKVPMYYINLYKMVPELRALFRAPFYT |
| | YKGSLTTPPCYQSVKWVVLQNPVSTSRELMTAMRSLKNHEGH |
| | SLCNNFRPTQPLNGRILAKHLKY |
| 193 | MSLKRIFGVATPEDQHNYCWCYEEENGPSEWKEHFPIANGPR |
| | QSPIDIKTSETKYDSSLKPLSVSYDPSTAREILNVGHSFHVTFED |
| | SENKSVLKDGPITGVYRLKQFHFHWGAADDKGSEHTVDGAK |
| | YAAELHLVHWNAVKYKSFEEAALEENGLAVIGVFLKLGKHHE |
| | ELQKLVDTLPAIKHKDALVTFGSFDPSCLMPTCPDYWTYPGSL |
| | TTPPLSESVTWIIKKQPVEVDHDQLEQFRTLLFTSEGE |
| 194 | MQEITVTRYENIQPSPVTSWNPEPKLPEIHPTAYIHPAAVVQGD |
| | VTIGKNVLVMANAVIRADEGYPIYIGDNSSVQDNVVLHALETV |
| | DKNGNTIEENVVTVGDEKYAVYVGDNVVLAHNAQVHGPAAV |
| | GDNTFVGMNALVFRSRVGKNCVLEPLAAAIGVTVPDNKYVPA |
| | GTVVTTQEEADKLPEITPDHPFANLNKRVVEVNVELAKGYLAL |
| | S |
| 195 | MKLIRSAVTGFCPEWNDHYQYDGISGPAYWGLINPDWTLCNS |
| | GKRQSPIDIDPNKLLFDPNLKSLHIDKHKVSGVLENTGQSLVFR |
| | VDKDTKQHVNISGGPLAYKYQFHEIFIHYGLEDSNGSEHSVDG |
| | YSFPAEIQLYGYNSDLYSNMSEAQEKSQGLVGISLLVQIGDMS |
| | NPELRVLTTALEKVKYKGQTTRIRKENVRGLLPDTQHYMTYE |
| | GSTTHPGCWETTVWIILNKPIYITKQELYALRRLMQGSKEHPK |
| | APLGNNARPTQPLHHRTVRTNIDFKHKKDG |
| 196 | MSQDEQKWSYAQDYLWKSPSCTGSKQSPINIDTSQIQRCGVLC |
| | DLKLYLKSEKPSVEFTSQNDVILSFVNSQSSITFNNRYFNLRSIR |
| | VHVPSLHTIDNSKTDMEVVCLFDSGNNNETSSNDSLQNVAKG |
| | VQLCFMMNQSNNEYGNIEQFFNQFIHKIPTVQDELPIEVNVSSS |
| | WSPELLIPNKQNFYYYEGSLAYPPCSEMYINIVYEEIGNIGVSNF |
| | RILKKYIRNNTRALKPKNNRVVYYSVDETNSASIQSNSVDKISD |
| | DRFLQCERRNNVVKTKKQVIASETIPEDN |
| 197 | MWLFSFLFYVAVHKNSGKNLHIKVLRAPKMIALLSHTQGAQP |
| | SLPDRTYMPIAKKLPYTKHHRLAAVILLIGMFVYHSALSEEQP |
| | WHFTTPAKADDCSQQSPEGAPCGCGELQSPINIKHSLRAHLPEL |
| | VTRYSPGPATVKHIGHTLEVRTEMKGHLTLGAKSYDFVQLHF |
| | HLPGVDLIKGRSYPLVAHLVHRSSTGEVAVVAIVFKRGQENAN |
| | LAQLLAVMPRHKGDAFVLGKFDIAQLLPQQRKYYAYKESMS |
| | AQPGIEGINWHILKTPMEVSDAQLHAFQLILPAHRRPAHPARN |
| | RSVRVGG |
| 198 | MQEITVLTFSNITKNEVTSTNPKPKTPIIDPTSYVHPLATVIGDV |
| | TIGKNCMISASASIRSDEGRPIYIGDNSNVQDGVVLHALESVDD |
| | GGKIIEENVVLEGNEYYAVYIGKNVSLAHQSQVHGPARVGDD |
| | SFIGMQSFVFNSIVGSNCVIEPNAAAIGVTVPDNKYIPAGTVVT |
| | TQEEADNLPEITPKHAAFTTQEAVVKVNVNLCRAYRNLA |
| 199 | MTTATDHIDYGYGPTNGPHTWCITCRTAAGTHQSPINIITHNCH |
| | FDPTLTPFKVFVSHHGHQILSRKQHNFQVSFKTDRPTYVEGGP |
| | LKNKYNLLQLHFHWGCYDEWGSEHHIDGHSYAGELHLVFMN |
| | EKYANINQAFNDPEGLCVIGIFLKPSVEGCSAMAPMMAAMKS |
| | SKPGCETSVKGEIDINGLIPNNSRYFTYEGSLTTPPCVECVRWIV |
| | CAKPLRLSKDQLAALRSMHCCETCYTNENFRPPVPVGDRVVV |
| | CSFPQSIRPQKCDT |
| 200 | MQEITVTTFNNIQPSPVTPWNPEPRLPEIHPTAYVHPAAVVQGD |
| | VTIGANVLVMANAVIRADEGYPIYIGDNSSVQDNVVLHALETV |
| | DADGRRIEENVVTVGDKEYAVYIGDNVVLAHNAQVHGPAAV |
| | GDNTFVGMNALVFNSRIGANCVLEPNAAAIGVTVPDGRYIPAG |
| | TVVTTQAAAAALPAVTPDHPFATLNARVVAVNNALAAGYLA |
| | LA |
| 201 | MVAILGVLMSSCEEEEVTVERHSPHWDYESTMWQNIGYTDCG |
| | GIVQTPINIETANTIKSADLSDVTFNYNAFDIKIVDNGHTVQVN |
| | RDATKTNNMVIDGVTYDFLQFHYHTHSEHEIDGATDEMEIHL |
| | VHQDPITKNLAVVSVMLNANGTTPNDFIESYLENFPSTEENEV |
| | ATTTSIDLDDLLPSNHNYYTYTGSLTTPPCSQGLKWIVLKDKV |
| | DVSVEQMHKFEETHGVNARPIQPLNGRLVLEKI |
| 202 | MQEITVLDFSNVTKNEVTSWNPKPTVPVIDPTSYVDPNATVIG |
| | DVTIGANCYIGPSASIRADEGRPIVIGDRSNVQDGVVLHALESV |
| | DDGGKIREDNVVVHGDEYYAVYIGKNVVLAHQAQVHGPAAV |
| | GDDSFVGMKSLVFKSDVGSNCVIEPFAAAIGVTVPDGKYIPAG |
| | TVVTTQAEAAKLPEITPDHANYTQQEAVVKVNVKLCRGYRRL |
| | Q |
| 203 | MQEITVLIYSNVEKNEVTSTNPKPKTPVIDPTSYVDPQATVIGD |
| | VTIGKNCYIAASAVIRADEGKPIVIGDNSNIQDGVVLHALESVD |
| | DGGKVLEDNVVIKGNKLYAVYIGKNVALAHQSQVHGPARVG |
| | DDSFVGMNSLVFNSIVGSNCVIEPFAAAIGVTVPDNKYIPAGTV |
| | VTTQAEADQLPEVTDDHPFYTEVAAVVKVNVALCQAHKGLS |
| 204 | MLTPARSIFCVGWKEQDHNYSLSPTLRPLVDGDRQSPINIVPGN |
| | AVYDPRLKPLTLSYDPATSLEILNNGHSFQVTFDDSQDKSVLK |
| | GGPLDGVYRLKQFHFHWGASDDHGSEHTVDGVKYPSELHLV |
| | HWNAKYGDFGEAASKPDGLAVVGVFLKIGHEKPHMQKVLDA |
| | LDAIKTKGKQTTFTNFDPSTLLPGCLDYWTYDGSLTTPPLLESV |
| | TWIVLKEPISVSPAQMAKFRSLLFTSEGETACCMVDNYRPPQP |
| | LKGRQVRASF |
| 205 | MSLKNIFTAVCGPEQWHDYFRKANGNFQSPINIDTKETKYDSS |
| | LKPLTLSYDPATAKEILNNGHSFQVTFDDTDNKSVLKGGPLTG |
| | SYRLRQFHFHWGATDEKGSEHTVDGVKYASELHLVHWNAVK |
| | YASFAEAASKPDGLAVLGVFLKIGKHHEELQKITDTLNSIKTKG |
| | KQTTFTNFDPSCLLPSCLDYWTYFGSLTTPPLYESVTWIVCKQP |
| | ISVSSEQLAQFRSLLSNAEGEKACCMVDNYRPPQPLKGRKVR |
| 206 | MQEITVTNYNNIRQSPVTSWNPTPKLPKIHPTAYIDPAAVVTGD |
| | VTIGKNVMVSANASIRSDEGYPIYIGDNSNVQDNVVLHALETV |
| | DANGKEIEENIVVVGDKKYAVYIGDNVSLAHQAQVHGPAAVG |
| | DNTFIGMQAFVFRSVVGKNCVLAPLAAAIGVTVPDNTYIPAGK |
| | VVTTQEEAAKLPKITPDHPFANTNKAVVAVNVELAKGYLALS |
| 207 | MRSIKLLCLPALLATTIANAGASLDQWDYSSHGERYWRSHFPA |
| | CQGMQQSPINISTKRALKHPKAFPSLKPGPVLDFSPPAAPVHIE |
| | NNGHSIQFEYRGNYSLLHRGESYQLKQFHFHHASETTIDGKHS |
| | PLEVHFVHKSQQGHTLVIAVLLDSGRAENILISSFKAADSSPQN |
| | GVNKSSFNPKKLLPKEKDFYYFEGSLTTPPCTEGVHWAVMKH |
| | KGLVSEQDVRYFAKFDYPANFRHTQPINGRSTYYFSDIDRSED |
| | DIKNSSGR |
| 208 | MQEITVSNFSNVTKNEVTPYNPKPVTPVIDPTSYIDPNATVIGD |
| | VTIGANCMVSANASIRSDEGKPIVIGDRSNVQDGVVLHALESV |
| | NDEGKILEENVVTVGDRNYAVYVGKNVSLAHQSQVHGPAAV |
| | GDDSFIGMQSFVFRSKVGSNCVIEPQAAAIGVTIPDGKYIPAGT |
| | VVTTQAEADKLPEITPDYAQSNTQAAVVTVNVKLCEAYRNKQ |
| 209 | MQEITVTNYTNIQPSPVTPWNPEPKLPEIHPTAYIHPAAVVQGD |
| | VKIGENVLVMANAVIRADEGYPIYIGNNSSVQDNVVLHALETV |
| | DENGNRIEENIVKVGDKEYAVYIGDNVVIAHNAQVHGPAAVG |
| | DNTFVGMNSLVFRSRIGKNCVLEPLAAAIGVEVPDGKYIPAGT |
| | VVTTQEEAAKLPEVTPDHPFANLNERVVKVNIALAKGYLALA |
| 210 | MRKTLAVSIFCGWNYDPEHQRWDYDDQENGPHRWPKLYPEC |
| | GGNAQSPIDIKTKETKYDPNLKPLTLVGYDKNGLEFSMTNNGH |
| | TVQISLPSSMYLKDSDGTVYIAKQMHFHWGGDSSEISGSEHTID |
| | GMRYLIEIHVVHYNSKYKSYDVAQDAPDGLAVLAAFVEVKD |
| | YAENTYYSNFISHLENIKYPGQSTVLRGLDIQDMLPKNLHHYY |
| | SYLGSLTTPPCTENVHWFVLADSVKLSKTQVWKLENSLLDHQ |
| | NKTIHNDYRKTQPLNHRVVEANF |
| 211 | MQEITVMEFSNVTKNAVTPTNPKPTTPVIDPTSYVDPEATVIGD |
| | VTIGENCMISAFASIRSDEGKPIHIGNRSNVQDGVVLHALESVN |
| | PTGMVNEENVVVAGDELYAVYVGKNVSLAHQAQVHGPAMV |
| | GDDSFIGMQSFVFKSIVGSNCVIEPNAAAIGVTVPDNKYIPAGT |
| | VVTTQAEADKLPDITPDYAYYTTVAAVVSVNVNLCKAYREQA |
| 212 | MKILTFASVCYGPENWHRDFQAAKGKRQSPIDIVPASAKYDSS |
| | LKPLTFTYEAGTSRCIVNNGHSFNVEFDDSQDKSVLSGGPLTD |
| | KYRLTQFHFHWGKTDDEGSEHTVDGHSYPAELHLVHWNADK |
| | FASFGEAASKPDGLAVVGVFLKVGDEHPGLKKVTDALYSVKF |
| | KGTKAEFKNFNPKCLLPASLDYWTYDGSLTTPPLSECVTWIVL |
| | KEPISVSSGQMGKFRSLLFTSEGETECCMVDNYRPPQPLKGR |
| 213 | MQRKLPSVAIFCTGYENHDWGYEDHNGPEHWHELFPIANGDN |
| | QSPIELHTKEVKYDSSLQPWSASYDPGSAKTILNNGKTCRVVF |
| | DDTYDRSMLRGGPLTGPYRLRQFHLHWGSSDDHGSEHTVDG |
| | VKYAAELHLVHWNAVKFESFEAAALEENGLAVIGVFLKIGRH |
| | NPELQKLVDVLPAIKHKDTLVEFGSFDPSCLMPTCPDYWTYPG |
| | SLTTPPLSESVTWIILKQPIEVDHDQLEQFRTLLFTSEGEKEKRM |
| | VDNFRPLQPLMNRTVRSSFRH |
| 214 | MLNSPSAANDERPEVEDGAWIHPSAALVGNVSIGSRAYVGPQ |
| | ASIRADEPGPDGSVAPVVIESEANVQDGAVLHALGGTSVVVRS |
| | RTSVAHGAVVHGPCQVGPGCFIGFNTVVYDAELGEQVVVMH |
| | GAVVENVEIPDGLIVPSRAAVCCQEDVRALDEASESALAFADE |
| | VSRTNVHLAEVKNSEQQTGYYE |
| 215 | MQEITVLEFSNVRKNEVTPTNPKPTTPVIDPTSYVDPNATVIGD |
| | VTIGANVLIWPTAVIRADEGRPIVIGDRSNVQDGVVLHALESV |
| | DDGGEIREDNVVRVGDENYAVYVGKNVSLAHQSQVHGPAAV |
| | GDDSFVGMKSLVFKSKVGSNCVIEPDAAAIGVTVPDGKYIPAG |
| | TVVTTQEEAAKLPEVTPDYAYYTTVEEVVTVNVALCEAYREE |
| | A |
| 216 | MQEITVTRYENIRESPVTPWNPEPRRPEIHPTAYIDPKAVVQGD |
| | VTIGANVLVMANAVIRADEGYPIVIGDNSSVQDNVVLHALETV |
| | DENGNPIKENIVKVGDKDYAVYIGDNVVIAHNAQVHGPAAVG |
| | DNTFIGMNALVFRSVVGKNCVLEPLAAAIGVTVPDGRYIPAGT |
| | VVTTQEEADKLPKVTPDHPFANLNARVVKVNVALAKGYLAQ |
| | A |
| 217 | MQEITVTVYNNIQPSPVTPWNPEPKLPKIDPTAYIHPKAVVIGD |
| | VTIGKNVMISANASIRSDEGYPIYIGDNSNVQDNVVLHALETV |
| | DENGNRIEENIVVVGDKEYAVYVGDNVSLAHQAQVHGPAAV |
| | GDNTFIGMQAFVFRSVVGKNCYLAPLAAAIGVTVPDGKYIPAG |
| | KVVTTQEEAAKLPEMTPDHPFYKTNEAVVKVNIALAKGYLAL |
| | K |
| 218 | MQEITVTRFENIQPSPVTPWNPEPRRPEIHPTAYIHPLAYVQGD |
| | VTIGENVLVAAHAVIRADEGYPIVVGNNSSIQDNVVLHALETV |
| | DENGNRIEENIVTVGDEEYAVYVGDNVVIAHNAQVHGPAAVG |
| | DNTFVGMNSLVFRSRVGKNCVLEPLAAAIGVTVPDGTYVPAG |
| | TVVTTQEEAAKLPKITPDHPFANLNARVVKVNVALAKGYLAL |
| | S |
| 219 | MKIGTVLSIFLGMAHAAVDDHSSPWNYNTWGSDWGSLTAIAG |
| | NECGNRNQSPIDLPSSVDSSQIYASKSDNFNKMYTDQTNAKIY |
| | WDGHTSKITIVNPGEDLQKFSSSFAKDYLQGPERFSGVQFHFH |
| | HGSEHTIDGERHDLEMHTVHVPDEGAKGGIKYAAMGIMFSVD |
| | KHTANAEEWEVKIIDDFFENLQWSETTTDPIVDLVSYGKVMM |
| | MVDTDNRWVYKGSVTTPPCATLVYWNVVRKIYPLKQKYLDQ |
| | FKNQLKRGSLTGNYREIQAYDDHDLHII |
| 220 | MITFLVSFLAALVCEFVHSDNLPVAWCYNNPACNFPNWPNIAP |
| | QYCNGSSQSPIDIVTAQVQGNPNLTQFILTGFDANTTFTSITNSG |
| | TSVVVSLDEDIMSVQGGDLPGLYVSVQFHLHWGSSSSLPGSEH |
| | TVDGKQYAMELHIVNLHSTYDGNVSAALAANDSSALAVLGFF |
| | IEGTDEADKTNSWDIFTSFLSNIPNSGNTYTDIMDQITMNSLLE |
| | GVNKTKYYRYQGSLTTPPCNEDVIWTVFKEPIKVNNNLINRFC |
| | TKVFAKTAKASDLNVNNFRGVQPLNGRVVTSQVEQTGSSAAP |
| | SLVPTSISSLSLILLLTSLSCL |
| 221 | MQEITVYDYSNVTKNEVTSTNPKPTTPVIDPTSYVDPKATVTG |
| | DVTIGKNVLIGPFAVIRADEGAPIVIGDRSNVQDGVVLHALESV |
| | DDGGKVREDNVVRHGDELYAVYVGRNVSLAHQAQVHGPAR |
| | VGDDSFVGMKSLVFRAKVGKNCVIEPGAAAIGVTVPDGKYIP |
| | AGTVVTTQAEAAKLPEITPDHPNYSKVDEVVAVNVGLCEAYR |
| | ERA |
| 222 | MRSALVIPFYKGHQEDWNTCKNGGMQSPIDLLHERVEVVSHL |
| | GRLQKSYKPSNATLKNRGHDMMLRWGDAGGYLEINGTEYVL |
| | QQCHWHSPSEHTINGRRFDMELHMVHQSRDNKIAVIGIMYKIG |
| | RPDSFLSKLMDHISAIADTTEEEKAVGVIDPRNIKIGSRKYYRYI |
| | GSLTVPPCTQNVVWTIVRKVRTVTREQVRLLRVAVHD |
| 223 | MRPPPQRQGKTHREGKEMTTAAWKAIFAMVLASVLLVDADD |
| | AHVKFGYSGSIGPEKWASLSPGYQMCSKGERQSPVNIDKSKLA |
| | YNPGLAALERNYVPANATLVNKGYQIALLFDKNVGTLVVDGK |
| | NYSLKSVHWHSPSEHTINGKRFAVELHMVHMSDNGRIAVVAI |
| | LYQIGRRDPFVVQIERKLKELAEEACKGDEEAYVPVGVVHTRS |
| | LKRHSSKYFRYSGSLTTPPCTENVIWSILGKVREMAEEQLAAL |
| | QAPLSQENRNNARPTQPLNYRAVQLYHESRKHDEYSR |
| 224 | MQEITVLEFSNVTKNEVTPTNPKPTTPVIDPTSYVDPNATVTGD |
| | VTIGKNVMISDSASIRSDEGKPIVIGDRSNVQDGVVLHALESVD |
| | DDGEVIEDNVVIYGDENYAVYVGENVSLAHQAQVHGPAAVG |
| | DDSFIGMQAFVFKSTVGSNCVIEPEAAAIGVTVPDGKYIPAGTV |
| | VTTQEEAAKLPEVTPDYPFYTTQAAVVTVNVALCEAYRAER |
| 225 | MKKPKLFNLLGTFAATFAYEYNPGNSDYRPENWAQMDSPNNI |
| | QCDWANQSPIDLQTQFVLIQSRANSLSITNLRTMPENVVLTNV |
| | GHGAEISFEFANNDNMVVTGGPLNDQFIVAQAQWHWGTADC |
| | AGSEHMLNSQRYSAEVHIVTYNSKYASLEDAADKYDGLAVLG |
| | FLYEVDEAANSDFPQSVQTSLGGITFGCDSTTVSPFPLIDLFRTE |
| | FFDYIAYSGSLTTPPCYQTVQWMVSTKPLKIWSSDLDALRSIN |
| | DVNGSPLLRNFRPCQNSYSRALNGYYL |
| 226 | MQEITVLDFSNITKNEVTSWNPKPTTPVIDPTSYVDPNATVIGD |
| | VTIGENCLIGASAVIRADEGHPIVIGDRSNVQDGVVLHALESVD |
| | DEGEYIEDNVVVKGDEEYAVYIGKNVSLAHQSQVHGPARVGD |
| | DSFVGMKSLVFKSDVGSNCVIEPFAAAIGVTVPDGKYIPAGTV |
| | VTSQEEAAKLPEVTDDYPFSTANEAVVKVNVALCEAYREQK |
| 227 | MQEITVTRYENIRPSPVTPWNPEPKLPKIHPTAYVDPAAVVQG |
| | DVTIGANVLIMANAVIRADEGYPIVIGDNSSVQDNVVLHALET |
| | RDENGNLLEENVVKVGDELYAVYVGDNVVLAHNAQVHGPA |
| | AVGDNTFVGMNALVFRSVVGKNCVLEPLAAAIGVTVPDGTYI |
| | PAGKVVTTQEEADKLPKITPDHPHYNLNERVVKVNVALAKGY |
| | LAQA |
| 228 | MQEITVTVFNNIRPSPVTPWNPEPKLPKIDPTAYIDPAAVVQGD |
| | VTIGKNVMVSANASIRSDEGYPIYIGDNSNVQDNVVLHALETV |
| | DENGKVLEENVVTVGDKKYAVYIGDNVSLAHQAQVHGPAAV |
| | GDNTFIGMQAFVFKSVVGKDCVLEPLAAAIGVTVPDGTYIPAG |
| | KVVTTQEEAAKLPKITPDHPFANTNAAVVKVNVALAKGYLAL |
| | K |
| 229 | MKNSWRITAVLFGCYHQPEDYFSYDGISGPAYWGEINPEWSL |
| | CNQGKMQSPIDLLNERVEVVSKLERIKKNYKPSNATLKNRGH |
| | DMMLKWESGAGSIHINGTEYVLKQCHWHSPSEHTINGRRYDM |
| | ELHMVHQSADNKTAVIGVTYKLGRPDSFLSSIMKHIKAISDTTE |
| | AEKAVGVIDPRHIKFGSRKYYRYMGSLTVPPCTEGVVWTIVK |
| | KVRTVSREQLRLLREAV |
| 230 | MELKVLSAHLFSWILVGPLFVVHIKAAEWSYADTSKWPKDYP |
| | SCSGYYQSPIDLTYKDSVYAPQLGQITITNLSKVEQTTYKVINN |
| | GHTVEVSFNEKQWKISLGSDEDPYYPIQMHFHWGGPTREGSE |
| | HLIGDLRHAMETHIVCYNGRLYKSKEEATSSPNGLAVVGILHE |
| | EDKLAQTEQTEFGKMGEFETALASITTTKESKNIAAFDLAGLL |
| | GQVDTTQYFRYQGSLTTPPCTQNVMWTVFTTFVPVTPAQLEL |
| | LRGLRTSSSTPLQDNYRPVQPLNDPHSPLPRTVYRTISAANRFT |
| | HSWWSFVMLSFLACCSHGIL |
| 231 | MQEITVTEFENIQPSPVTSWNPTPKKPVIHPTAYVHPAAVVQG |
| | DVTIGKNVMISPLASIRSDEGYPIYIGDNSNVQDNVVLHALETV |
| | DANGNTIEENVVTVGDKKYAVYIGDNVSLAHQAQVHGPAAV |
| | GDNTFIGMQAFVFRSIVGKNCVLEPLAAAIGVTIPDGTYIPAGT |
| | VVTTQEEADKLPKITPDHPFANTNKAVVKVNVELAKGYLALS |
| 232 | MTLSLFAAAAFADVQECPPRYSYCGYSGPEQWKNIVFKDKRN |
| | ECNGTTQSPINLGTPTPTSGPTIHVEYVGNVAGNATIRNTGHDI |
| | EVTPMRGNNKIKVGSRVYTLLQLHFHVPNEHHVPRIGKAVAE |
| | MHILHQLDGGTDYAVIGVMLTIGTPTDSALAPVFENLPKEACA |
| | PPKPLEINFKKLLPEELTGYYTYVGSLTTPPCTEKEKTVTWYVL |
| | DAPREIPASDLLKLGALGKNARPIQTNPLTVTYVSPTPTPK |
| 233 | MQEITVAEFSNVVKNEVTPTNPKPTTPEIDPTSYVDPNATVEGD |
| | VTIGANVLIHAFAVIRADEGRPIVIGDRSNVQDGVVLHALESVD |
| | DGGEIREDNVVLHGDDLYAVYVGKNVHLAHQAQVHGPARVG |
| | DDSFVGMKSLVFKSDVGSNCVIEPFSAAIGVTIPDGKYIPAGTV |
| | VTTQEEADKLPEVTEDYAFSGQNEAVVTVNVDLNEAYRQQR |
| 234 | MQEITVTNFNNIRPSPVTPWNPEPKLPKIDPTAYVDPLATVTGD |
| | VTIGKNVMISANASIRSDEGYPIYIGDNSNVQDNVVLHALETV |
| | DENGNRIEENVVTVGDKEYAVYVGDNVSLAHQAQVHGPAAV |
| | GDNTFIGMQAFVFRSNVGKNCVLEPLAAAIGVTVPDNKYIPAG |
| | KVVTTQEEAAKLPEVTPDHPFYKTNAAVVKVNVALAKGYLA |
| | LS |
| 235 | MLATFSRPNQGHVEIDYCKWTKYVSISGSSTWKDQFPIANGNR |
| | QSPIDIKTSETKYDSSLKPLSVSYDPSTSLEILNNGHSFQVTFAD |
| | DSDSSTLKDGPITGVYRLKQFHFHWGASDDHGSEHTVDGVKY |
| | PAELHLVHWNTKYGDFGEAASKPDGLAVVGVFLKIGREKPEF |
| | QLVLDALESIKTKGKQASFTNFDPSTLLPGCLDYWTYDGSLTT |
| | PPLLESVTWIVLKEPISVSPAQMAKFRSLLFTSEGETACCMVDN |
| | YRPPQPLKGRQVRASF |
| 236 | MIKTNPRGDLPQVHESAFVDPTAILCGLVIVEEYVFIGPYAVIR |
| | ADETDAAGRIAPIVIGAHSNIQDGVVIHSKSGASVWIGQRTSIA |
| | HRAIVHGPCRVGDGVFIGFNSVLFNCTIDDGCVVRYNAVVDG |
| | CHLPPGFYVRSTERIGPETDLAALPQVTADASDFSEDVARTNN |
| | ALVLGYKHIQNEF |
| 237 | MQEITVTNFNNIAESPVTPWNPEPKKPKIHPTAYIHPLAYVQGD |
| | VTIGENVMVSANASIRSDEGYPIYIGNNSNVQDNVVLHALETV |
| | DENGNEIEENIVKVGDKKYAVYVGDNVSLAHQAQVHGPAAV |
| | GDNTFIGMQAFVFRSNVGKNCVLEPLAAAIGVTIPDNTYIPAG |
| | KVVTTQEEAAKLPKITPDHPFYKTNEAVVKVNVALAKGYLAL |
| | A |
| 238 | MAAWAGGGPHWSYEGAGGPANWARLTPEFGACAGRNQSPID |
| | LTGFIEAELPPLAFAYRAGGRSIVDNGHTVQVTYAPGSVLEVG |
| | GRRFELQQFHFHTPSEERINGRSYPLVAHLVHRDAAGHLAVVA |
| | VLFKQGAENPALAPLWAAMPGKAGETRALKAPLDAGALLPA |
| | RRDYFTYMGSLTTPPCSEGVRWMVLRQPLEVSAAQVARFREV |
| | MGENARPVQPLNGRTVLHRVM |
| 239 | MQEITVTVFENIRPSPVTPWNPEPKLPKIHPTAYIDPAAVVQGD |
| | VTIGKNVMVSANASIRSDEGYPIYIGDNSNVQDNVVLHALETV |
| | DENGNVIEENVVTVGDKKYAVYVGDNVSLAHQAQVHGPAAV |
| | GDNTFIGMQAFVFRSTVGKDCVLEPLAAAIGVTVPDGTYVPA |
| | GKVVTTQEEAAKLPKMTPDHPFYKTNEAVVKVNVALAKGYL |
| | ALS |
| 240 | MQEITVTRYENIRESPVTPWNPTPKRPEIHPTAYVDPLAYVQG |
| | DVTIGANVMVSAHASIRSDEGYPIVIGDNSNVQDNVVLHALET |
| | VDENGNEITENIVTVGDKKYAVYVGDNVSLAHQAQVHGPAA |
| | VGDNTFIGMQAFVFNSVVGKNCFLAPLAAAIGVTVPDGTYIPA |
| | GKVVTTQEEAAKLPKITPDHPFANTNAAVVKVNVALAKGYLA |
| | LS |
| 241 | MQEITVTLFNNIRPSPVTPWNPEPKLPEIHPTAYIDPAAVVQGD |
| | VTIGKNVLIMANAVIRADEGYPIKIGDNSSVQDNVVLHALETV |
| | DENGNVIEENVVVVGDERYAVYVGDNVVLAHQAQVHGPAA |
| | VGDNTFVGMQALVFRSRVGKNCVLAHQAAAIGVEVPDGRYIP |
| | AGLVVTTQEEAARLPEVTPDHPFANLVDRVVKVNVALAKGYL |
| | ALK |
| 242 | MVPERCRRTPTLLLFVSLAMAVAVSGACADDDKATAMRTAM |
| | PKPVSRRASTASSAETVVTPSVKEKRNTTPSPHHGTQDSWSYD |
| | NVAAWPATCAGNQQSPMPLRHTPADAHGRGSIRTLLTAATTL |
| | RLRAVRDGSSIALLCNGYCGVVKVHGVTHMIKNMHWHTPSE |
| | HTIDGRRLDAELHMVAFAGGKIAVLSSLFKVANKNVLVDRTIR |
| | AMSGMRSMSATRKEVKDYFFSGAVKVSAAVYKGSLTTPPCTE |
| | GLSWVVNAKVSTMSKKQLSKIRELLGGHDNARPLQAPKGRV |
| | VEWMDVP |
| 243 | MKQNLFAITVSCYWREPGDHLVDKSAPSHWNKLYPIAQGNRQ |
| | SPINIITSQAVYSPSLKPLELSYDAATSLSITNNGHSVQVDFNDS |
| | DDRTVLKGGPLTGPYRLKQFHLHWGKKDAVGSEHTVDGVKY |
| | ASELHLVHWNAKYGKFGEAVKQPDGLAVLGIFLKVGREKGEF |
| | QIFLDALDKVKTKGKEAPFTKFDPSCLFPACRDYWTYHGSFTT |
| | PPCEECIVWLLLKEPMTVSSDQMAKLRSLYSSAENEPPVPLVS |
| | NWRPPQPIKG |
| 244 | MIKKISLVLSIAALVLTGCNYSEGGKPKANVSAGYKKNWNYG |
| | TNNGPTHWEEFSSTCGKGIHQSPVNIIPGKTLKMNHAYDLSMH |
| | DDITGLAKVIDNGHSIKVTPEHGGHIKLHGEIFDLLQYHFHGKS |
| | EHTIDGKRFDMVAHMVHQNPKTKQLAVVAVFFEEGAKNKVL |
| | EKIINHVGSTVQLDAQDFVPLQTEHYYHYIGSLTTPPCSENVQ |
| | WYLLKQPQEASEEQIKHFRKFYVDNERPVQELHDRFIEVN |
| 245 | MKITFLAVSCQHNEPDYWGRIKEDWKICKTGKMQSPIDLSNQ |
| | RVKIISHLGDLKMNYKPSNATLKNRGHDIELEWKGGAGSIEIN |
| | GTEYVLQQCHWHSPSEHTINGRRYDLELHMVHESRDGKIAVI |
| | GILYKIGRPDSFLSKLMKNIKSISDTKDEERAVGVIDPRHIKIGS |
| | RKYYRYIGSLTTPPCSQNVIWTIVKKV |
| 246 | MQEITVLEFSNVTKNEVTSWNPKPTTPVIDPTSYVDPNATVIGD |
| | VTIGKNCLIAASAVIRADEGAPIVIGDRSNVQDGVVLHALESVN |
| | DGGKIREENVILHGDEEYAVYVGKDVSLAHQAQVHGPARVG |
| | DDSFVGMKSLVFKSDVGSNCVIEPFAAAIGVTVPDGKYIPAGT |
| | VVTTQAEAEKLPEVTEDYPFYTTQEEVVKVNVNLCEAYREQA |
| 247 | MSTPLVKWGYDEQNGAHIWCRFFPAANGKRQSPIDIDINTVKH |
| | DPSLKPLSVSYDPSTAKEILNVGHSFHVNFEDSDNRSVLKGGPL |
| | TGSYRLRQFHLHWGSADDHGSEHTVDGVKYAAELHLVHWNP |
| | KYNTFAEALKQPDGIAVVGVFLKIGREKGEFQILLDALDKIKTK |
| | GKEAPFTKFDPSCLFPACRDYWTYHGSLTVPPLLESVTWIILKQ |
| | PISVSSEQLAKFRSLLCTSEGETAVFMLRNHRPPQPL |
| 248 | MQEITVLEFSNVRKNEVTPWNPKPSTPVIDPTAYIDPQATVIGD |
| | VTIGANVLIGPMAVIRADEGAPIVIGDRSNVQDGVVLHALESIN |
| | EEGEVREDNVVEVGDENYAVYIGKNVSLAHQSQVHGPARVG |
| | DDSFVGMKSLVFKSDVGSNCVLEPGAAAIGVTVPDGKYIPAGT |
| | VVTTQAEAAKLPEVTADYPFYTAQEAVVEVNVALCQAYNEQS |
| 249 | MQEITVYEFSNVTKNEVTPTNPKPSVPVIDPTSYIDPNATVIGD |
| | VTIGKNVLIAAFAVIRADEGRPIVVGDRSTVQDGVVLHALESV |
| | DDDGKVIEDNVVIHGNKDYAVYIGKNVVLAHQSQVHGPARV |
| | GDDSFVGMNSLVFNSVVGNNCVIEPNAAAIGVTVPDGKYIPAG |
| | TVVTTQEEADKLPEVTPDYAFYTKNEVVVNVNVDLCKAYKE |
| | KA |
| 250 | MQEITVHHFSNVTKNEVTSWNPKPSTPVIDPTSYVDPNATVIG |
| | DVTIGENVLIGANAVIRADEGAPIVIGDRSNVQDGVVLHALES |
| | VDDGGEEIEDNVVIEGDEEYAVYIGKDVSLAHQAQVHGPAAV |
| | GDDSFVGMKSTVFNSTVGENCVIEPDAAAIGVTVPDGKYIPAG |
| | TVVTTQEEAAKLPEVTPDHPSHDEIEAVVEVNVALNEAHREQA |
| 251 | MCDLNCIMTKMDNSDYMIVVCCVLLSIFLLFEIVEWIFKVFTW |
| | TDNDVCLPPTFSFGYAHKNGPHTWKDLYPESAGSNQSPINITT |
| | RYAIVVQPSEPLRWINYNSVPLSTTLSNDGHTVILRGFWDQSS |
| | WPQLQGGPLSDKYDFFNILFHWGPSNQEGSEHTLDYIRYPMEL |
| | QVIHMKHGLKSPKDAIILGARDGIVIVSFFLQINAMDNPYLDHI |
| | VSNLWKISNPSHYKTNIPPFPLEWIFAPFDRDYYTYSGSLSQPPC |
| | NEVVTWIIQKEPIVISALQVEKFREICSVDGPLLLNCRPVQPLNE |
| | RDVYFYEESKL |
| 252 | MQEITVTVYNNIQPSPVTSWNPTPKLPEIHPTAYVHPAAVVIGD |
| | VKIGENVMISPHASIRSDEGMPIYIGDNSNVQDGVVLHALETV |
| | DANGNTIEENVVTVGDKKYAVYVGKNVSLAHQSQVHGPAAV |
| | GDNTFIGMQSFVFKSVVGKNCVLEPLAAAIGVTVPDGTYVPA |
| | GKVVTTQEEAAKLPKVTPDHPYANTNAAVVYVNVELAKGYL |
| | ALA |
| 253 | MQEITVLVYSNVQKNEVTSQNPKPVVPVIDPTSYVDPKATVIG |
| | DVTIGKNCMISASASIRSDEGHPIVIGDRSNVQDGVVLHALESV |
| | NDGGMILEENVVLAGGEDYAVYVGKNVSLAHQSQVHGPAKV |
| | GDDSFIGMQSFVFNSIVGSNCVIEPNAAAIGVTVPDNKYIPAGT |
| | VVTTQEEADKLPEITPDHAYYTTVAAVVNVNVGLCRAYKNEA |
| 254 | MTWAVPLVLLPLVLASALGAVVEVPETCGAEAGACVDEESA |
| | MVQVKTQPSQRAPSAATASGDVDYQGFQLGDWPEIAPLCAGG |
| | STTGFQAPINIAVEGADYEKMPQASWPKFYAKEGGCDEAHFV |
| | EKGTAWQVDFMNPKINLDCKNLEMEWKGKVYALVQFHFHTL |
| | SEDTVDFQPTAMQMHMVHLAADGSFAVVGVLIKTDGFFKNG |
| | FLEGIFETGFESDRMVTLLAKHRFNPYAGVLSKHGEFWHYEGS |
| | FTTPPCTEGVDFLIAQSPVVTSKSYVTSYMEYLKGNGKGNSYG |
| | QNHRPIQPLNGREITTGRFLEVCPKKPAPDCGKLDPKKVQFCEE |
| | SA |
| 255 | MLSGPQTWYKRFAINCEDVHQSPINIVTKKTIPDPNLKPLELTY |
| | DATTTRTIVNNGHSVQVDFEDSSNRTVITGGPLTGPYRLKQFH |
| | FHWGASDDKGSEHTVDGVKYASELHLVHWNAEKYSSFVEAA |
| | HEPDGLVVLGVFLKIGEHNPNLQKLTDALYSVRFKGTKAQFT |
| | NFNPKCLLPPSLDYWTYPGSLTTPPLLESVTWIVLKEPISVSPSQ |
| | LAKFRSLLFTSEGETACCMVDNYRPLQPLMNRKVRASF |
| 256 | MQEITVTRFENIAPSPVTPWNPEPKLPKIHPTAYVHPLAYVQGD |
| | VTIGENVLIAPLASIRADEGYPIYIGDNSSVQDNVVLHALETVD |
| | ENGNVLEENVVTVGDKKYAVYIGKNVVIAHLAQVHGPAAVG |
| | DNTFVGMLALVFKSNVGKNCVLEPLAAAIGVTIPDGKYIPAGK |
| | VVTTQEEAAKLPEVTPDHPFYKLNERVVKVNVALAKGYLAQA |
| 257 | MLGMKNTENHSAVLVQGHPAPANGGQIKVINNQEEGPSTWA |
| | ESYPDYCNGSSQSPIDIDDWEVSPNPCDLSFVNYDLPFTGYWK |
| | NNGHALQFTLDDGSGAVVSGPCLGNSTYQLLQVHFHWGSAK |
| | GQGSEHTIEGKQHDLEMHMVHTNTAYETDEAANYKDGYLVV |
| | GVLFDEAKQNKIRGFERTFRNFVKKSSKLQDSDEGTLTAMFDV |
| | SDILRKSGVARSHFQYSGSLTTPSCNEVVTWILATKILKEKRSE |
| | LNALRSLQTHDDEALVDNFRPTQELNGRKIMMF |
| 258 | MQEITVAEYSNVTKNEVTPTNPKPTTPVIDPSSYVDPNATVTG |
| | DVTIGKNCLIGASAVIRADEGAPIVIGDNSSVQDGVVLHALESV |
| | DDDGEVIEDNVVLYGNEDYAVYVGKNVVLAHQAQVHGPAA |
| | VGDDSFVGMKALVFKSIVGSNCVIEPDAAAIGVTVPDNKYIPA |
| | GTVVTTQEEAAKLPEVTPDHEYYTEVAEVVKVNVALCEAHLA |
| | KA |
| 259 | MQEITVTRFENIQPSPVTPWNPTPKLPEIHPTAYIHPAAVVQGD |
| | VTIGANVLVMANAVIRADEGYPIVIGDNSSVQDNVVLHALETV |
| | DANGNVIEENVVVVGDERYAVYVGDNVVLAHLAQVHGPAA |
| | VGDNTFVGMLSLVFRSRVGKNCFLAPLAAAIGVTVPDGKYIPA |
| | GKVVTTQEEADKLPEITPDHPGANLNARVVAVNVALAAGYLA |
| | QA |
| 260 | MQEITVTEFSNITKNEVTATNPEPVTPVIDPTSYVDPNATVTGD |
| | VTIGKNCLIAANAVIRADEGKPIVIGDRSSVQDGVVLHALESVD |
| | DEGMPIEENVVLEGDKYYAVYIGENVTLAHQSQVHGPARVGD |
| | DSFVGMKSLVFKSDVGSNCVIEPFAAAIGVTVPDGKYIPAGTV |
| | VTTQAEADKLPEVTDDYPFSTAQEAVVEVNVNLCEAYRNKA |
| 261 | MQEITVLEFSNVRKNEVTPTNPKPTTPVIDPTSYVDPNATVIGD |
| | VTIGENCYIAPFASIRADEGSPIVIGNNSNVQDGVVLHALESVN |
| | DGGKLIEDNVVLEGNEYYAVYIGNNVKLAHQSQVHGPAYVG |
| | DDSFVGMKSLVFKSKVGSNCVIEPEAAAIGVTVPDGKYIPAGT |
| | VVTTQAEADKLPEVTPDYAKSNAQEAVVKVNVALCEAYKKL |
| | S |
| 262 | MQEITVTKYENIRPSPVTPWNPEPKLPEIHPTAYVDPAAVVQG |
| | DVTIGANVMVSAHASIRSDEGYPIYIGDNSNVQDQVVLHALET |
| | VDEAGNVIEENVVTVGDKKYAVYVGDNVSLAHQAQVHGPAA |
| | VGDNTFIGMQAFVFKSVVGKNCVLEPLAAAIGVTVPDGKYIPA |
| | GTVVTTQEEAAKLPEVTPDHPFYNTNAAVVKVNVALAKGYL |
| | ALS |
| 263 | MCQLENAIEDIFELKEDIVQCQWTVPLVTITIVNEGTGEIEAQPN |
| | KIELQKVQNLCTIVKTEWMYQGADNQNDKWPQNCPSCDASL |
| | EGNERQSPIDLNPQMTNMVTKTLPKLTFTPNPNGDTLGKFENK |
| | VNTIQFTANDLSQNKMHGGPLSGEYSFWQMHCHWGKTNYEP |
| | GTTEPTKVEQHGSEHWIDGKQYDAECHWVHFNNKYATVGDA |
| | IASGDADALSVIGVMLEIDETNGQDEVEWIGTVKDAASALVTP |
| | DDGPAEDAPFNVYGFLDQLGDQSQCFGYYNYLGGLTTPGCNQ |
| | LVSFIIIDTPIRINMAQVKNVKYNKIESFCSIYVRQY |
| 264 | MQEITVLRFSNVTKNAVTATNPKPTTPVIDPTAYVDPNATVIG |
| | DVTIGKNCYIAAFARIRADEGRPIVIGDNSNVQDGVVLHALESI |
| | DDAGKVIEDNVVVEGNKLYAVYVGRNVSLAHQSQVHGPARV |
| | GDDSFVGMKSLVFKSDVGSNCVIEPFAAAIGVTVPDGKYIPAG |
| | TVVTTQAEADALPEVTPDYAFYTQVAAVVTVNVALCEKYKA |
| | QA |
| 265 | MKLINFVGTACSPYEQWRDHYPIADGNRQSPIDIVPGSASYDS |
| | GLKPLTLKYDPSTSLEILNNGHSFQVTFVDDSDSSTLKGGPISG |
| | VYRLKQFHFHWGSSDDHGSEHVVDGVKYAAELHVVHWNAA |
| | KYSSFVEAAHEPDGLAVLGVFLKVGEHNSQLQKITDILNSIKEK |
| | GKQTRFTNFDPICLLPPCPDYWTYPGSLTVPPLLESVTWIVLKQ |
| | PISVSSQQLAAFRNLLFTSEGEKACCMVNNYRPLQPLMNRTVR |
| | SSFR |
| 266 | MQEITVLVYSNVTKNERTSYNPKPTVPVIDPTSYVDPNATVIG |
| | DVKIGKNCYVAAFAVIRADEGKPIVIGDRSNVQDGVVLHALES |
| | VDAGGKLIEDNVVIHGDNWFAVYVGKNVVLAHRAQVHGPAA |
| | VGDDSFVGMNSLVFNSKVGSNCVIEPEAAAIGVTVPDGKYIPA |
| | GTVVTSQAEADKLPEITPDYAYYTQNAAVVNVNIGLCRGYKR |
| | LA |
| 267 | MQEITVTVFENIRESPVTPWNPTPKRPVIHPTAYIDPAAVVQGD |
| | VTIGANVMVSANASIRSDEGYPIYIGDNSNVQDNVVLHALETV |
| | DANGKRIEENVVRVGDKDYAVYIGDNVSLAHQAQVHGPAAV |
| | GDNTFIGMQAFVFRSRVGANCVLEPLAAAIGVTVPDGTYVPA |
| | GKVVTTQEEADKLPKVTPDHPFATTNAAVVAVNVALAKGYL |
| | AQK |
| 268 | MQEITVLEFSNVTKNEVTSWNPKPVTPVIDPTSYVDPDATVIG |
| | DVTIGENVLIAAGATIRADEGKPIYIGDRSSVQDGVVLHALESR |
| | DDGGMENGDNVVIHGNTLYAVYVGNNVSLAHQSQVHGPAA |
| | VGDDSFVGMNSLVFNSKVGSNCVIEPNAAAIGVTVPDGKYIPA |
| | GTVVTSQAEADKLPEITPDYEYYTAVAKVVGVNVALCEAYQE |
| | LQ |
| 269 | MTAALLSASAWAAPHWEYSGEAGPANWAKLTPEFGACAGKN |
| | QSPINLTGFTQAQLKPLKFNYQADAKSILNNGHTVQVNFKPGN |
| | YLELDGQRFELKQFHFHAPSENLIEGKSFPLEAHFVHANAQGE |
| | LAVLALMFKPGKANPELAKAWQQMPEKAGEETVLKAPINAQ |
| | DLLPKNLEYYRFSGSLTTPPCSEGVRWLVMKQPVELSQEQIDA |
| | FKEIMHHPNNRPLQPLNGRPVLTS |
| 270 | MQEITVTVYNNIRESPVTSWNPEPKKPEIDPTAYIDPKAVVRGD |
| | VKIGKNVMVSANASIRSDEGYPIYIGDNSNVQDNVVLHALETV |
| | DEDGNEIEENIVTVGDEKYAVYVGENVSLAHQAQVHGPAAVG |
| | DNTFIGMQAFVFRSVVGKNCVLEPLAAAIGVTVPDGTYIPAGK |
| | VVTTQEEADKLPKVTPDHPFYKTNEAVVKVNIELAKGYLAQS |
| 271 | MQEITVTRYENIRESPVTPWNPTPRRPQIHPTAYIDPAAVVQGD |
| | VTIGANVMVSPLASIRSDEGYPIVIGDNSNVQDQVVLHALETV |
| | DAAGKTLEENVVTVGDEKYAVYVGDNVSLAHQAQVHGPAA |
| | VGDNTFIGMQAFVFKSTVGKNCVLAPLAAAIGVTVPDGTYVP |
| | AGTVVTTQEEAAKLPKVTPDHPFATTNAAVVAVNVALAAGY |
| | RALA |
| 272 | MRSSAFAVPAVAALAVAGLSAALAFAAQANEPAKPAAAGHH |
| | EVYDYDHQEAWQALHDKSQSPIDIVTAGAAAADPAEPRAIEFS |
| | HTHGAIDKIEDNGHAVQVDTHATEATIRGRHFKLAQFHFHAQS |
| | EHTLDGKHFPLEGHFVFKAQDGRLAVVGVMYEQGKANAVAQ |
| | EVLDDLKPGKAKPAQPEIDIEGLLPKAHGYYHYLGSLTTPPLTE |
| | NVEWYVMPTPVTMSKQQIDGFLSHYRRNNRNIQPLNGRPLIRY |
| | EG |
| 273 | MARKPSGHLTIYEQDVNCFWSFIEPIEGTGQSPIDLHTKEIKYDS |
| | SLKPLSVKYDPSTAKEISNTGHSFQVTFEDNDNKSVLRGGPLT |
| | DSYRLSQFHFHWGSSDEHGSEHVVDGVKYAAELHLVHWNAA |
| | KYSSFAEAAHEPDGLAVLGVFLKVGEHNPQLQKVIDALNSIKT |
| | KGKRAPFTNFDPSTLLPSSLDYWTYDGSLTTPPLLESVTWIVLK |
| | EPISVSSEQMSKFRSLLFTSEGETACCMVDNYRPPQPLKGRQV |
| | R |
| 274 | MQEITVTRFENIRESPVTSWNPTPKKPKIHPTAYVDPLASVIGD |
| | VTIGENVMISPHASIRSDEGMPIYIGDNSNVQDGVVLHALETVD |
| | DNGNVIEENVVVVGDEKYAVYIGENVSLAHQSQVHGPAIVGD |
| | NTFIGMQSFVFKSKVGKNCVLMPLAAAIGVEVPDNKYIPAGK |
| | VVTTQEEADKLPEITPDHPYYNTNKAVVYVNVELAKGYLALS |
| 275 | MQEITVTVYNNIQASPVTPWNPTPKLPEIHPTAYVHPAAVVQG |
| | DVTIGENVYIAANAVIRADEGYPIVIGDNSSVQDNVVLHALET |
| | VDEDGNVIEENVVKVGDKDYAVYVGKNVVLAHNAQVHGPA |
| | AVGDNTFVGMNALVFRSTVGKNCFLAPNAAAIGVTVPDGTYI |
| | PAGKVVTTQEEAAKLPKITPDHPGANLNERVVKVNVALAKGY |
| | LAQA |
| 276 | MQEITVTKYENIRESPVTPWNPTPKKPEIHPTAYIDPAAVVQGD |
| | VTIGKNVMVSANASIRSDEGYPIKIGDNSNVQDNVVLHALETV |
| | DENGKEIEENIVVVGDEKYAVYIGDNVSLAHQAQVHGPAAVG |
| | DNTFIGMQAFVFRSRVGKNCVLEPLAAAIGVTVPDGTYIPAGK |
| | VVTTQEEADKLPKITPDHPFANTNAAVVKVNVALAKGYLAQA |
| 277 | MQEITVFIFSNVEKNEVTETNPKPVVPKIDPTSYIDPNATVIGDV |
| | TIGENCYIAPFASIRADEGKPIVIGDRSNIQDGVVLHALESVDDN |
| | GEIIEENVVLEGDEYYAVYIGKNVSLAHQSQVHGPAKVGDDSF |
| | VGMKSLVFNSIVGSNCVIEPNAAAIGVTVPDGKYIPAGTVVTT |
| | QEEADSLPEVTPDHAAYTKIAAVVTVNVSLCLAYLGES |
| 278 | MSAPVIRWTYEGDKGPHFWNQLCEEYEIAKTGKNQSPIDIHME |
| | KVMEVQGAPPLELNYKPTKYTVRRVENSVHLFPKDKEQGLTF |
| | NGKRYNLIAFHGHIPSEHTLNEHYFAIEWHLVHMNEAGERLVL |
| | GIWMEKELEGSDFGELAEIFPEVFADFGIEKEISLDVSGFLPEER |
| | AYFTYQGSLTTPPTFEGVTWIVLRNATSIS |
| 279 | MVASSLSSLCLVLASLVGQTLATSPCDVDKTSPECDKTGVNRA |
| | SWGYAASNGPATWAANYPDFCAGDMQSPIDLDSSKAVTMDP |
| | GPITMVGYNLKQAGKIENNGHTLGFAFASGSTPYIMGGRLPAG |
| | DRFDFVQLHWHWGSDSSKGSEHTMNGKEYPIEVHLVHANTK |
| | YYVNGAPSNDNLVMPDGLAVLGIFYEVSTEDNANLTNIVSKV |
| | NEVAVEQRRRRKQGRAGSNEVDLDMTLALDSFLPADTTqyyyy |
| | qggLTTPSCNEAVLWTNMKSTQTISEAQLEVFRSMTDSDGITLN |
| | NNYRPPQPLNNRTIYTTGTSTTAGSSNMFTELLNTAFTAAVVT |
| | GLVGIVAPLFAPPPSQQRSDAASARAEQALRAGRDQWGGYW |
| | G |
| 280 | MQEITVLTFSNVTKNEVTATNPKPTTPVIDPTSYVDPNATVTG |
| | DVTIGKNCFIGANAVIRADEGKPIVIGDNSNVQDGVVLHALES |
| | VDDGGKVREDNVVVHGNEWYAVYIGKNVSLAHQSQVHGPA |
| | YVGDDSFVGMKSLVFKSIVGSNCVIEPEAAAIGVTVPDGKYIP |
| | AGTVVTTQEEADKLPEITPDYAFYTQIAEVVKVNVGLCKAYRE |
| | KA |
| 281 | MQEITVTRYTNIRPSPVTPWNPEPKLPEIHPTAYIDPLAYVQGD |
| | VTIGENVMISANASIRSDEGYPIVIGNNSNVQDNVVLHALETVD |
| | ENGNRLEENVVKVGDEEYAVYVGDNVSLAHQAQVHGPAAV |
| | GDNTFIGMQAFVFRSRVGKNCYLAPLAAALGVTVPDGKYIPA |
| | GKVVTTQEEAAKLPEITPDHPFANTNAAVVKVNVALAKGYLA |
| | QA |
| 282 | MFKSSLILLATLSVVLCGDDAKSWGYRNKGRNIVPEKWGEMQ |
| | PKCLGSVQSPINVDFASTQFDANLGKLNIKKHGNETEQWDVK |
| | NNGHSVVFTPVNTDFSFVIYPQKEEFKLLQLHFHWRGSEHFVN |
| | GIKYAGELHLVHQSKTNPNQFSVIGFLLQLVNADNLKMKAVID |
| | VLADVTEYEATKKIDNFELNDMVPFEVENFFRYSGSLTTPGCD |
| | EFVEWNLADKPVIGLSENQILEFQSLLDNHKYPILSNSRPVQEI |
| | NDRIVKRSFYPFEAKARTHGASGYSVSGANKFQFTSSVFFTLIA |
| | SAFCFYSL |
| 283 | MQEITVLEFSNVTKNEVTPTNPKPVTPVIDPTSYVDPDATVVG |
| | DVTIGANVLIWPKAVIRADEGKPIVIGDRSNVQDGVVLHALES |
| | VDDGGEIREDNVVLVGDENYAVYVGNNVSLAHQSQVHGPAR |
| | VGDDSFVGMKSLVFKSDVGSNCVLEPNAAAIGVTVPDGKYIP |
| | AGTVVTTQEEAAKLPEVTDDYPFYTAQDAVVEVNVDLCEAY |
| | KGQA |
| 284 | MQEITVDTFSNVTKNEVTSTNPEPVTPVIDPTSYVDPNATVTGD |
| | VTIGKNCYIGANAVIRADEGAPIVIGDRSNVQDGVVLHALESV |
| | DDEGEIREDNVVVHGDENYAVYIGEDVSLAHQSQVHGPARVG |
| | DDSFVGMKSLVFNSTVGENCVIEPEAAAIGVTVPDGKYIPAGT |
| | VVTTQAEADKLPEVTPDYAFYTEVAEVVTVNVALCEAHREQK |
| 285 | MIGRSSLRARLATASAGLVLSAVPVAAPVTAAAAATPVMSIM |
| | AGETAEWNHDPASPIGPTHWGELDPAWSACRSVQDQSPIAVT |
| | PTREADRPVLLVDYPRTPLVVRNTGHVIEVPAPPGGGGTLLVG |
| | GHSYRLLQWHTHVPSEHVVNGHRADLEIHLVHQDEQGEIAVL |
| | AVFADVVSLGEAAPRMPAADLLRTTVQAAPSTAGEEIDLDQK |
| | VSAAALLGATVEDGEQRRAITNYLSYTGSLTTPPCTGGVRWFL |
| | LPGIIGVDPASVQPLHALIASFPGYDGYPDNNRPVQPVGSRMV |
| | ERRVGWPSVGGVTSGAA |
| 286 | MSPLCWGYEKDNGAHVWRQTFIAAEGPRQSPIDIQTSKAVPDL |
| | TLKPLTLSYDPATSLEILNNGHSFQVTFADDSDSSTLTEGPVSGI |
| | YRLKQFHFHWGASDDKGSEHTVDGVKYPAELHLVHWNAVKF |
| | KSFGEAALEENGLAVVGVFLKIGKHHPELQKLVDALPAIKHKD |
| | TLVKFGSFDPSCLMPTCPDYWTYPGSLTTPPLSESVTWIVLREP |
| | ISVSPEQL |
| 287 | MAAPSASKGHDVHWSYEGDNGPANWGKIKPEWAKCSTGNR |
| | QSPIDIRDGMKVELDQIQFDYRPSSFSVIDNGHTVQVGVSGGN |
| | YITVQNRMYELQQFHFHRPSEERINGKAFEMVIHLVHKDAEGR |
| | LAVLAVLLERGAPQPVIQTVWNHLPLEKFETMQPTILLDPAEL |
| | LPARRDYFTYMGSLTTPPCTEGVLWMVMREPIQASSEQIAIFA |
| | RLYPMNARPIQETNGRMIWKSKYLS |
| 288 | MRLSTIFVAGYCPEKWDHQNPITGGEHQSPINIISSQTKYDPNL |
| | KPLNISYDPSTSLEILNNGHSFQVTFKDNDNRSVLKGGPLDDV |
| | YRLEQFHFHWGKKDAEGSEHTVDGVKYSSELHLVHWNAVKY |
| | SSFEEAASKENGLAVLGVFLKVGEHNPKLQKIIDALNSIKTKGK |
| | QTTFTNFDPSTLLPSSLDYWTYSGSLTTPPLSECVTWIVLKEPIS |
| | VSPAQMAKFRSLLFTSEGEKACCMVDNYRPPQPLKGRKVRAS |
| | F |
| 289 | MQEITVLFFSNVTKNEVTATNPKPVTPVIDPTSYVHPEATVIGD |
| | VTIGENCYIAPFASIRADEGSPIVIGNDSNVQDGVVLHALESVD |
| | DGGKLIEDNVVLEGHKNYTVYIGKNVSLAHQSQVHGPARVGD |
| | DSFVGMNSFVFNSKVGSNCVIEPNAAAIGVTIPDGKYIPAGTVV |
| | TSQAEADNLPEITEDYKYYTQIAAVVNVNVGLCRAYREKA |
| 290 | MKNVTGHLSARCFQIEDYPWSYDNDLLGGPDFWGLINKHWK |
| | LCAIGKMQSPIDIDPNILLYDPNLKPIHIDKHKVSGTLENTGQSL |
| | VFRVDKETKHHVNISGGPLAYKYQFHEFYIHFGLHDHLGSEHS |
| | IDRYSFPAEIQLYGFNSDLYNNMSEAQEKSQGLVGVSLMVQIG |
| | ETPNPELRIITSTFNKVIYRGKSAPVKRLSVRSLLPDTKDYVTYE |
| | GSTTHPGCWETTVWIILNKPIYITKQELYALRKLMQGSKSHPK |
| | APLGNNARPIQDLHGRTVRTNI |
| 291 | MQEITVTRYENIRPSPVTPWNPTPRRPRIHPTAYVDPAAVVQG |
| | DVTIGANVLVMANAVIRADEGYPIVIGDNSSVQDNVVLHALET |
| | VDAAGRRLEENVVRVGDEDYAVYVGANVVLAHNAQVHGPA |
| | AVGDNTFVGMNALVFRSRVGANCVLAPNAAAIGVTVPDGTY |
| | VPAGLVVTTQEEAARLPRVTPDHPFANLNARVVAVNVALAAG |
| | YRALA |
| 292 | MQEITVTKYENIRPSPVTPWNPEPKLPEIHPTAYIDPAAVVQGD |
| | VTIGANVMVSANASIRSDEGYPIVIGDNSNVQDNVVLHALETV |
| | DADGKVLEENVVKVGDERYAVYVGDNVSLAHQAQVHGPAA |
| | VGDNTFIGMQSFVFRSVVGKNCVLEPLAAAIGVTVPDGTYVPA |
| | GKVVTTQEEAAKLPKITPDHPFANTNAAVVAVNVALAKGYLA |
| | QS |
| 293 | MAGSSARALAALVALVLVAVAVIAEPRQQQLKTALLRIEGGP |
| | ADTFAAAAEAEAAAAKAAEAAAACPWSYEGANGPANWGTIC |
| | GKIFSECATGMQQSPINIKLLRMHQGPGQSMIGWKIPSDAYNK |
| | FVTFGGGGDYLESYDGHSFVVSHADALFPFGGVTYKLQSFHT |
| | HTVSEHTIDGEHYDMEMQFVHKTVDGAKFSTGLKGELGQTLI |
| | VSVMFQVGKGQGSPHWLRQLAKAVPSVTNESAQVIPLDFTEV |
| | AQSVMVGTLPQDARFKDFKPNYNHYYGYTGSLTAPPCTQGV |
| | QWLVLANPIYAEAEDIQAFKDLEGDNFRPVQRINGRIVTQRYC |
| | GLSCE |
| 294 | MAFTRLSISLLLSGLILSAGMPAQAAPETVMVPEVTAMALEGK |
| | WPADWSYQGENGPAHWGELHPSYSKCARGRVQSPVDLGKAT |
| | TRSRRSTVRVAFHPIRYEIFNDGRGIRAVPLEAQHPIRIDRHDYT |
| | LKHIVFRAPSEHTFQGRHYPLEAQLVYEADDGALAVLATVFSP |
| | GHSNPSLAALTRQPLAEGQLDKPMGTRVLLPRRLPHLRLNGSL |
| | TTPPCTEGVNWVVFTQPVQATRAQIDAMTRLIGHPNNRPVQP |
| | AHRRLMVEEMR |
| 295 | MKRTSLFAVIGECPQWNYDHQEEWKIVFPQANGDQQSPINIEP |
| | SSAVYDSALKPLELKYDPSTSLEILNNGHSFQVTFVDDSDSSTL |
| | KDGPITGVYRLKQFHFHWGAADDKGSEHTVDGVKYPAELHL |
| | VHWNAKYGSFGEAASKPDGLAVVGVFLKIGKHHPGLQKLTD |
| | ALYSIRFKGTKAEFSGFNPKCLLPASLDYWTYPGSLTTPPLSES |
| | VTWIVLKEPISVSPEQMAKFRSLLFTSEGETACCMVDNYRPLQ |
| | PLKGRKVRASF |
| 296 | MQEITVLEFSNITKNEVTSWNPKPKTPVIDPTSYIDPNATVIGDV |
| | TIGKNCYIGASAVIRADEGRPIVIGDNSNVQDGVVLHALESVN |
| | DDGKVIEDNVVLEGNKYYAVYIGKNVVLAHQSQVHGPAAVG |
| | DDSFIGMNSLVFRSIVGSNCVIEPNAAAIGVTVPDNKYIPAGTV |
| | VTTQEEADKLPEITEDYAYWNTIAEVVKVNVNLCEAYKNEA |
| 297 | MQEITVLEYSNVRKNEVTTTNPKPKTPVIDPTSYVDPKATVIGD |
| | VTIGENVLIGPFAVIRADEGRPIVIGDRSNVQDGVVLHALESVD |
| | DDGKIIEDNVVVEGDEYYAVYIGKNVVLAHQAQVHGPARVG |
| | DDSFVGMNALVFNSIVGDNCVIEPNAAAIGVTVPDGKYIPAGT |
| | VVTTQEEADTLPEITPDDEYYTKIAEVVKVNVALCEAYREKA |
| 298 | MQEITVTNYNNIQASPVTSWNPEPKLPEIHPTAYIHPKAVVQG |
| | DVTIGANVMISANASIRSDEGYPIVIGDNSNVQDNVVLHALET |
| | VDENGNVIKENVVKVGDKDYAVYIGKNVSLAHQAQVHGPAA |
| | VGDNTFIGMQAFVFRSNVGKNCVLMPLAAAIGVTIPDGTYIPA |
| | GKVVTTQEEADKLPKVTPDHPFYKTNEAVVKVNVELAKGYL |
| | AMA |
| 299 | MQEITVLFFSNVRKNEVTPQNPKPVTPVIDPTSYVDPNATVIGD |
| | VTIGENCLIGASAVIRADEGHPIVIGDRSSVQDGVVLHALESVD |
| | DGGKIIEDNVVLEGDEYYAVYVGRNVVLAHQSQVHGPAAVG |
| | DDSFVGMKSLVFRSIVGSNCVIEPEAAAIGVTVPDGKYIPAGTV |
| | VTTQEEADKLPEVTPDHADYTKQAEVVKVNVGLCEAYRELS |
| 300 | MKNSCATYSVVVMTLSLILVLTISLGYQNPFAMGQDTINDTSII |
| | SKQWPNIMSNVNTFVVENVTSPRIDDTAYIHPFAIIIGDCSIGKK |
| | VLVAPTAVCRADEGIPIHIGDYSNIQDGVILHALDAVRDGTNV |
| | DNKRFSQEGDRLLGNDTRFDEGYAIYLSGNVSLAHDSLIHGPV |
| | WIGNNTLIGVKSAVLDSKIGNNVVIRVGSIITGVEIPDNTLVPPG |
| | SVLTNQSQVATLPSVIGSPSQNLNQGDLKNSQALATAYDNTNI |
| | ER |
| 301 | MQEITVLSFSNVTKNEVTSTNPKPTTPKIDPTSYVDPKATVTGD |
| | VTIGKNCLIGPFAVIRADEGAPIVIGDNSNVQDGVVLHALESVD |
| | DGGKIIEENVVLYGNKYYAVYIGKNVVLAHQAQVHGPARVG |
| | DDSFVGMNSLVFNSIVGSNCVIEPNAAAIGVTVPDNKYIPAGT |
| | VVTTQAEADNLPEITPDHPYYTAVEKVVEVNVNLCKAYRNKE |
| 302 | MQEITVTKYENIQESPVTPWNPTPKRPVIDPTAYVHPAAVVQG |
| | DVTIGKNVMISANASIRSDEGYPIYIGDNSNVQDNVVLHALET |
| | VDADGNEIEENIVTVGDKKYAVYIGDNVSLAHQAQVHGPAAV |
| | GDNTFIGMQAFVFRSNVGKNCVLAPLAAAIGVTVPDGTYVPA |
| | GTVVTTQEEADKLPKVTPDHPFANTNAAVVKVNVALAKGYL |
| | ALS |
| 303 | MRGCLEKTGEGAIAGILVMAGPPQIQGSNFHSGFIFLKNFIMNS |
| | TQHCTLLAISFCFALLLLFACNGANKEQQQSSTSNISKDHPKAD |
| | TLLGDNEVKAEAPDANSKAEQGYALPQHTDRLAQSPIDIISVK |
| | ADKTVKEQISFAFHSDINAAKNLGHTIELEFKEGSTCKVNGKD |
| | YASRQFHFHTPSEHLVDGITFPMEMHIVNILADSVNTNKPSYLV |
| | LAVLFKIGTENKFIKEFFNKIPNKEGEENTLQTGDVRLDDLLSQ |
| | FTPNDIKSYYTYQGSLTTPPFTESVQWVILKHIVEASEEQIMAIE |
| | KMEGNNARHVQAINDRKIYSH |
| 304 | MSRPGTVLAIFCWNHQEKYDMKKVLAVAAALLALGGVAAEA |
| | SHWGYEGEGAPEHWGALDEAYKACQAGKNQSPINIEHALKA |
| | HHGQLDLAFKPGAQQIVNNGHTIQVNVSAGNTLTLDGDTFTL |
| | QQFHFHAPSENEIDGKQFPLEAHFVYKDKDGALVVLALMFQQ |
| | GKANPQLAQAWQQMPAAIDQVATLNQPVDIKALLPKEFNFYR |
| | FSGSLTTPPCSEGVRWLVLDQPVSASAEQIQQFRAVVHHANNR |
| | PVQPLHGRVIVD |
| 305 | MQEITVLDFSNITKNEVTPTNPEPITPVIDPTSYIDPNATVTGDV |
| | TIGKNVLIGPNAVIRADEGRPIVVGDRSNVQDGVVLHALESVD |
| | DEGEPIEDNVVEKGDELYAVYIGKNVSLAHQSQVHGPARVGD |
| | DSFVGMKSLVFKSDVGENCVIEPESAAIGVTIPDGKYIPAGTVV |
| | TSQAEAAKLPEVTPDYAYSDTNEAVVKVNVGLCEAYKEQA |
| 306 | MQEITVTRFENIRPSPVTPWNPTPKRPVIHPTAYVDPAAVVQG |
| | DVTIGANVMISALASIRSDEGYPIYIGDNSNVQDNVVLHALETV |
| | DADGKRIEENVVKVGDKDYAVYIGDNVSLAHQAQVHGPAAV |
| | GDNTFIGMQAFVFRSRIGKNCYLAPLAAAIGVEVPDGTYIPAG |
| | KVVTTQEEAAKLPKMTPDHPFYKTNEAVVAVNVALAKGYLA |
| | LA |
| 307 | MKRSLVATIFGCYEPNDHQWGYTKDNGPATWAKSFPAANGP |
| | RQSPIDIKPSETKYDSSLKPLSLKYDPSTALEILNNGHSFQVTFK |
| | DSENKSVLQGGPLEGTYRLEQFHFHWGSSDEHGSEHVVDGVK |
| | YASELHVVHWNAKYGDFGEAVKHPDGLAVLGIFLKVGKHHP |
| | EFQKLLDALNSIKNKGKQASFTNFDPSVLLPACLDYWTYSGSL |
| | TTPPLLESVTWIVLKEPISVSPAQMEQFRSLLFTSEGEKEKRMV |
| | DNFRPLQPLMNRTVRSSFR |
| 308 | MTLSRAFIVGCPQEDHKNWYNQYPIAKGNRQSPINIETKQAQY |
| | DSSLKPLTFSYDPSTAKEIVNVGHSFHVNFEDNDNQSVLSGGPL |
| | TGSYRLKQFHFHWGASDEHGSEHTVDGLKYPAELHLVHWNA |
| | KYGSFSEAASQPDGLAVVGVFLKIGDENPKLQKIIDALESIKTK |
| | GKQTRFTNFDPSTLLPSCLDYWTYHGSLTTPPLLESVTWIICKE |
| | PISVSPSQMEKFRSLLFTSEGEKECCMVDNYRPLQPLMNRTVR |
| | SSFR |
| 309 | MQEITVTRYENIRPSPVTPWNPEPKRPKIHPTAYIDPAAVVVGD |
| | VTIGENVLVMANAVIRADEGYPIVIGDNSVVQDNVVLHALETV |
| | DENGNRIEENVVRVGDEDYAVYVGKNVVLAHNAQVHGPAAV |
| | GDNTFVGMNALVFRSRVGKDCVLMPNAAAIGVTVPDGTYVP |
| | AGLVVTTQEEAAKLPKVTPDHPFANLNARVVKVNVALAKGY |
| | LALA |
| 310 | MQEITVTRYENIRPSPVTPWNPTPKLPKIHPTAYVDPAAVVQG |
| | DVTIGANVMVSANASIRSDEGYPIVIGDNSNVQDNVVLHALET |
| | VDAAGRRLEENVVRVGDEEYAVYVGDNVSLAHQAQVHGPA |
| | AVGDNTFIGMQAFVFRSRVGKNCVLEPLAAAIGVTVPDGRYV |
| | PAGTVVTTQAEADKLPKVTPDHPFYKTNAAVVKVNVALAKG |
| | YLAQA |
| 311 | MGSHAHWSYSGASGPEHWASLTPEYGACAGRNQSPVDLAGFI |
| | EADLAPIAFHYQAGGTEVVNNGHTVQVNYAPGSAIELDGHRF |
| | ELKQFHFHAPSENLIDGKSYPLEMHLVHADEAGHLAVVALMF |
| | KAGAENAALAKLWKAMPEQPGETVHLAPLVSAEALLPKDRD |
| | YYRFNGSLTTPPCSEGVRWLVMKEPVSASAEQIAAFEKRLPHP |
| | NNRPLQPTNARLVLK |
| 312 | MQEITVLFFSNVTKNEVTPTNPKPSTPVIDPTSYVDPNATVTGD |
| | VTIGANVLVAANATIRADEGKPIVIGDRSSVQDGVVLHALESV |
| | DDEGEIKEDNVVVVGNKNYAVYIGKNVSLAHQSQVHGPAHV |
| | GDDSFVGMKSLVFKSDVGSNCVIEPFAAAIGVTVPDGKYIPAG |
| | TVVTTQEEADKLPEVTPDYAESNTNEAVIKVNVGLCEAYKNK |
| | S |
| 313 | MAWNRRNFLGSFACFGLSLATGRALAVTSCKPEEQPCWGYH |
| | GDEGPDHWGRLHPDWVACAEGSEQSPIALAGEEVKPTAERFA |
| | LHYQPTTARLSDNVHTVRIDMEPGSQLLLGDRTFSLRQFHFHT |
| | PSEHLWSDTADLGELHLVHVADSREIAVLGVALRPDAAQAFP |
| | DSFWNWLQAAEAGESLTLDPAGLVPRQGRLVGYRGSFTTPPC |
| | TEGVNWLLAMEPMAMGPEEQRWLEQRMGRNARPVQPLGSR |
| | TVHTVLREGS |
| 314 | MQEITVTRYENIRESPVTSWNPEPRRPEIHPTAYVDPAAVVQG |
| | DVTIGENVMVSAHASIRSDEGYPIVIGNNSNVQDNVVLHALET |
| | VDENGNEIEENIVTVGDKKYAVYVGDNVSLAHQAQVHGPAA |
| | VGDNTFIGMQAFVFRSRVGKDCYLAPLAAAIGVTVPDGTYIPA |
| | GKVVTTQEEAAKLPKMTPDHPFYKTNAAVVKVNVELAKGYL |
| | AQA |
| 315 | MQEITVTVFNNIQPSPVTPWNPEPKLPEIHPTAYVHPLAYVQGD |
| | VKIGENVMISALASIRSDEGYPIVIGNNSNVQDNVVLHALETVD |
| | ENGNEIEENIVTVGDEKYAVYIGDNVSLAHQAQVHGPAAVGD |
| | NTFIGMQAFVFNSIVGKNCVLEPLAAAIGVTIPDGTYIPAGKVV |
| | TTQEEADKLPKVTPDHPFANTNAAVVKVNVALAKGYLALS |
| 316 | MIRKMFCTIIAVALAGLFASSEQLSQQNPLASKSPEPAKTESNT |
| | KPAEAAKQEEHAKHWDYTENGPDKWAGLDPQNKLCSEGKM |
| | QSPIDITNPKPDQLPEVSIEFPPAVFSMTHNEHVKDIENNGHTIQ |
| | VDFDEKNTDTLKIGNAKYSLSQFHFHSSSEHTVNGKSFPMEMH |
| | LVHKAGDNFAVLGIFIEEGPEDNKAFEPIWSKLPQKGKTEENIN |
| | IDINQFLPKSRTTYRYEGSLTTPKCGEAVKWIVFAEPIRMSSGQI |
| | AKFRSIVKKNNRPTQPLNERVVQTDIIEEKDSK |
| 317 | MLTSLFLLSALFSTAWSCPKHDNYQSHPHLGRRQIRVDKGREP |
| | KDWNYDVSADWATINPEYVLCQSGTHQSPINIAQQDLSTLHKP |
| | NFEGYQSVKIPGNFFNWQFAPAWTPHHPEGDVTGLPSFNEDGE |
| | EVFNIGWHIHAPSEHLIDGKRSRAEIHMVHVTAEEHEAAVIGIR |
| | LAVGPQESAFIKQLGPMIHYNDTAQLEGLEVNLRLAIDEVGGV |
| | EEFWTYKGSLTTPPCSEGLRWFLPKQELIVSEQQMVEILAASRF |
| | SHRVEQPVWLHDINL |
| 318 | MRLSVFATICGYQPEHWNDKYKMASGKRQSPIDIQPKDTKYD |
| | SSLKPLTISYDPSTSKEILNNGHSFQVTFEDSNNKSVLKGGPLTD |
| | SYRLTQFHFHWGASDEHGSEHVVDGAKYSAELHLVHWNSDK |
| | YSSFAEAASKPDGLAVLGVFLKVGKEHAELQKLTDALPSIKHK |
| | DTLAKFGNFDPSCLMPSCPDYWTYAGSLTTPPLLESVTWIILKE |
| | PISVSPSQMAKFRSLLFTSEGEKEKRMVDNFRPLQPLMNRTVR |
| | SSF |
| 319 | MQEITVTKYENIQPSPVTPWNPEPKKPEIHPTAYIHPAAVVIGD |
| | VKIGENVMVSANASIRSDEGYPIVIGDNSNVQDNVVLHALETV |
| | DENGNEIEENIVTVGDEKYAVYVGKNVSLAHQAQVHGPAAV |
| | GDNTFIGMQAFVFRSRVGKNCVLEPLAAAIGVTVPDGTYVPA |
| | GKVVTTQEEAAKLPKVTPDHPFYNTNAAVVKVNVALAKGYL |
| | ALS |
| 320 | MQEITVDEYSNVTKNEVTPYNPKPTTPVIDPTSYVDPNATVTG |
| | DVTIGKNVLIGAFARIRADEGQPIVIGDRSNVQDGVVLHALESV |
| | DDDGEVLEDNVVLHGDEDYAVYVGKDVSLAHQSQVHGPAR |
| | VGDDSFVGMKSLVFNSDVGDNCVIEPFAAAIGVTVPDGKYIPA |
| | GTVVTTQEEAAKLPEITPDHAASNAQAAVVEVNVQLNQAYRG |
| | QA |
| 321 | MQEITVTKFENIQPSPVTSWNPEPKLPKIDPTAYIHPLATVVGD |
| | VTIGKNVMISANASIRSDEGYPIYIGDNSNVQDNVVLHALETV |
| | DENGNRIEENIVVVGDKEYAVYIGKNVSLAHQAQVHGPAAVG |
| | DNTFIGMQAFVFRSVVGKNCVLEPLAAALGVTVPDGRYIPAG |
| | KVVTTQEEADKLPKITPDHPFATTNAAVVKVNVELAKGYLAL |
| | K |
| 322 | MKRISGVLATFCWPEQHNDYLLLHLLYVLKMNSWGYNESNG |
| | PATWHEHYPIANGDRQSPIDIKTKEVKYDSSLRPLSIKYDPSTA |
| | KEILNNGHSFNVEFEDSQDKSVLKGGPLTGSYRLRQFHFHWGS |
| | ADDHGSEHTVDGVKYPSELHLVHWNAVKFSSFGEAALEENGL |
| | AVIGVFLKLGRHHGEFDKIVDALDSIKTKGKQASFTNFDPSCLL |
| | PPCPDYWTYSGSLTTPPLSESVTWIILKQPISVDSEQLAKFRSLL |
| | SSSEGEKASFMLSNHRPLQPLKGRKVRSSF |
| 323 | MQEITVFNYSNVTKNEVTPENPKPTTPEIDPTAFVDPDATVIGD |
| | VTIGKNVMISASASIRSDEGKPIVIGDRSNVQDGVVLHALESVD |
| | ENGKVLEDNVVLEGDEWYAVYVGKNVSLAHQSQVHGPAMV |
| | GDDSFIGMQSFVFKSHVGSNCVIEPDAAAIGVTVPDGKYIPAGT |
| | VVTTQEEADKLPEVTPDYAYYTTVAAVVEVNVNLAQAYKSK |
| | A |
| 324 | MQEITVTRYENIRPSPVTPWNPTPKRPKIHPTAYIDPAAVVQGD |
| | VTIGANVLVMANAVIRADEGYPIVIGDNSSVQDNVVLHALETV |
| | DAAGRVLEENVVKVGDERYAVYVGANVVLAHQAQVHGPAA |
| | VGDNTFVGMQALVFRSTVGANCVLAPLAAAIGVTVPDGTYVP |
| | AGLVVTTQAEAAALPRVTPDHPFADLNARVVAVNVALAAGY |
| | RALA |
| 325 | MQEITVTRFENIRPSPVTPWNPEPKLPEIHPTAYIDPAAVVQGD |
| | VTIGENVLVMANAVIRADEGYPIYIGDNSVVQDNVVLHALETV |
| | DENGNEIEENVVVVGDKKYAVYIGKNVVIAHNAQVHGPAIVG |
| | DNTFVGMNALVFRSEVGKDCYLAPNAAAIGVKVPDGTYIPAG |
| | KVVTTQEEAAKLPKMTPDHPGYKLNERVVKVNIALAKGYLAL |
| | S |
| 326 | MQEITVTKYNNIRPSPVTPWNPEPKLPEIHPTAYIDPAAVVQGD |
| | VTIGENVLVMANAVIRADEGYPIVIGDNSSVQDNVVLHALETV |
| | DENGNRIEENIVKVGDEEYAVYIGKNVVIAHLAQVHGPAAVG |
| | DNTFIGMLALVFNSIVGKNCVLAPLAAAIGVTIPDGKYIPAGKV |
| | VTTQEEADKLPEVTPDHPLYNLNARVVKVNVALAKGYLALS |
| 327 | MKLIFTVASGQEYDPNHWCRGYPIAKGNRQSPIDIDTKSAKYD |
| | SSLKPLTVSYDPATAREIVNVGHSFNVTFDDSQDKSVLRGGPL |
| | TGVYRLRQFHFHWGSSDDHGSEHVVDGVKYSAELHLVHWNA |
| | KYGSFAEAARHPDGLAVVGVFLKIGREKGEFQILLDALDAIKT |
| | KGKQTRFTNFDPSCLFPPCRDYWTYSGSLTTPPLSESVTWIVLK |
| | QPIEVDHDQLEKFRTLLFTSEGE |
| 328 | MITKLFAGSVQRCYEPDNWHRYYPVANGNSIDIEAAETQYDSS |
| | LKPLTISYNPATAKEILNVGHFFTVHFEDKDNQAQEKGGPLDG |
| | VYRLIQFHFHWGSIDGQGSEHTVDKKKYAAELHLVHWNAKY |
| | GDFGEAAQQPDGLAVLGIFLKVGSAKPGLQKVVDALNSIKTK |
| | GKSADFTNFDPSTLLPGCLDYWTYDGSLTTPPLLESVTWIVLK |
| | EPISVSSEQMSKFRSLLFTSEGEAACCMVDNYRPPQPLKGR |
| 329 | MDNNLAAAVRIVVEVLLFVFICILIWVVIQSKREEATLQVKLA |
| | GAQVQINTATEKLATTEAAFAEAEHKLDKAAKGALWEYEGRF |
| | GPDFWGKVFPTCGIGKSQAPLDIRGPFGKAKAKIQVDYKLSGL |
| | KLIHNGHTVQVNVAPGSRLLVDGVAYELLQFHFHRPSEEWIEG |
| | KPSDMSLHLVHKSADGKLAVLGVLLQAMAADNQGLVPIWTH |
| | LPSAEGPEQSFPETNVDPAKLIPSNLAYYQYEGSLTTPPCTEGV |
| | TFFILKTKMPISKGQLDAFPIPHSNARPVQPLNGRTIYSSS |
| 330 | MRIKRLGLSRLGIGLLSITVVGTASAEGVLATEAGPPAQGATLA |
| | WQYEGEQGPSHWGTLAPTTASCEKGTHQSPINIRTASHPHGHD |
| | GMLIQYRAASGHVGTSHHTVEVDFQSGGTLELSGRSYSLKEFH |
| | FHEPSEHQLNGRIYPMEAHLVHRDESGHLVVLAILMELGTETA |
| | PLADVWERIPSGKQEEVRDLLFNPQDLLPKDLHHYAYDGSLTT |
| | PPCTEGVHWIVLKEPIHITAAHLERFVSLIGHNARPVQPLNERE |
| | VDEE |
| 331 | MQEITVTRFENIRPSPVTPWNPEPRLPRIHPTAYIDPAAVVQGD |
| | VTIGANVMVSANASIRSDEGYPIVIGDNSNVQDNVVLHALETR |
| | DAAGRELEENVVTVGDEKYAVYVGANVSLAHQAQVHGPAA |
| | VGDNTFIGMQAFVFNSRVGANCVLMPLAAAIGVTVPDGTYIP |
| | AGTVVTTQEEAAKLPKVTPDHPFATTNAAVVKVNVALAAGY |
| | RALA |
| 332 | MQEITVLVFSNVRKNEVTPWNPKPETPKIDPTSYIDPEATVIGD |
| | VTIGKNCYIAASAVIRADEGRPIVIGDRSNVQDGVVLHALESV |
| | DDGGKVREENIVLEGGKYYAVYVGKNVVLAHQAQVHGPAW |
| | VGDDSFVGMKSLVFKAIVGSNCVLEPEAAAIGVTVPDGKYIPA |
| | GTVVTTQAEADKLPEVTPDDAKYTANVAVVNVNVNLAKAYR |
| | ELA |
| 333 | MLNSAGIFPKRVTQECYHWDYGKHMEWEKTFPSAAGNRQSPI |
| | NIQPREAQFDPSLKPLTLKYDPSTSLEILNNGHSFQVTFVDDTD |
| | SSTLTGGPITGTYRLKQFHFHWGAADDKGSEHTVDGVKYPCE |
| | LHLVHWNAVKYASFAEAAAEPDGLAVVGVFLKIGQHHEELQ |
| | KLVDALPSIKHKDTLVTFGSFDPSCLMPTCPDYWTYSGSLTTPP |
| | LSESVTWIIKKQPVEVDHDQLEQFRTLLFTSEGE |
| 334 | MKSLIAVFGTCHWNYDEQRPEEWHNDYPVANGLRQSPIDIKP |
| | AETQYDSTLRPLSFKYDPSTAKEILNNGHSFQVTFDDSSDKSVL |
| | SGGPLTGTYRLKQFHFHWGASDEHGSEHTVDGVKYAAELHL |
| | VHWNSDKYASFAEAAAKPDGLAVVGVFLKIGEANPALQKLL |
| | DALSSIKTKGKQTTFTNFDPSTLLPSSLDYWTYLGSLTVPPLLE |
| | SVTWIVLKEPISVSPAQLAKFRSLLCSGEGEAACCMVDNYRPP |
| | QPLKGR |
| 335 | MQEITVTRFENIRPSPVTPWNPTPKLPKIHPTAYIDPAAVVQGD |
| | VTIGENVLVMANAVIRADEGYPIYIGDNSSVQDNVVLHALETV |
| | DENGNRIEENIVTVGDKEYAVYIGKNVVIAHNAQVHGPAIVGD |
| | NTFIGMNALVFRSKVGKNCVLEPLAAAIGVTIPDGTYIPAGKV |
| | VTTQEEADKLPKVTPDHPFYKLNERVVKVNIALAKGYRALS |
| 336 | MLRSAPGVFICTNWQEKYDHVAEGSEQSPINIVTDKAVYDSTL |
| | KPLELKYDASTALEIVNNGHSVQVKFDDSSDKAVLKGGPLTGP |
| | YRLKQFHFHWGKKDDVGSEHTVDGVKYASELHLVHWNAKY |
| | GSFGEAASQPDGLAVVGVFLKIGSAKPGLQKVVDALNSIKTKG |
| | KSADFTNFDPSTLLPSSLDYWTYDGSLTTPPLLESVTWIVLKEPI |
| | SVSSEQMSKFRSLLFTSEGEAACCMVDNYRPPQPLKGRQVRAS |
| | F |
| 337 | MQEITVLEFSNITKNEVTPWNPEPVTPVIDPTAYIDPQATVIGD |
| | VTIGANCYIAASAVIRADEGKPIVIGDRSNVQDGVVLHALESIN |
| | DGGMVREDNVVEVGDENYAVYVGKNVVLAHQSQVHGPAAV |
| | GDDSFVGMKSLVFKSIVGSNCVIEPEAAAIGVTVPDGKYIPAGT |
| | VVTTQAEAAKLPEVTPDHAAYSQIAAVVAVNVALCQAYRDQ |
| | A |
| 338 | MVLFFLLSSSYLISASTAHGEVEDESEFTYDEGSEKGPKNWGKI |
| | KPQWKACSTGKLQSPIDLLDQRVQVLPNLGELKREYKPAPAVI |
| | KNRGHDITIKWKGDAGKIKINGTDFKLQQCHWHSPSEHTFNGS |
| | RYNLEMHIVHLSAQNKIAVIAILYKYGRPDPFLSRLFHHIKTVG |
| | TEERDIGIINPGEIKFGSRKYYRYIGSLTTPPCTEGVIWTVFK |
| 339 | MQEITVLLFSNVQKNEVTTTNPKPTTPVIDPTSYVDPNATVVG |
| | DVTIGKNVLIWATAVIRADEGKPIVIGDRSNVQDGVVLHALES |
| | VDDGGKIRTDNVVLHGDELYAVYIGNNVSLAHQAQVHGPAH |
| | VGDDSFVGMKSLVFKSKVGSNCVIEPFAAAIGVTIPDGKYVPS |
| | GTVVTTQEEAAKLPEITADHAQYTQQAQVVSVNVRLCKAYRE |
| | QK |
| 340 | MQEITVTLYENIRPSPVTSWNPTPKRPVIDPTAYIDPAAVVQGD |
| | VTIGKNVLVMANAVIRADEGYPIVIGDNSSVQDNVVLHALETV |
| | DENGNVIEENVVEVGDKKYAVYVGDNVVLAHLAQVHGPAAV |
| | GDNTFVGMLALVFRSRVGKNCVLEHLAAAIGVTVPDNTYVPA |
| | GTVVTTQEEAAKLPKMTPDHPHYNLVERVVKVNVELAKGYL |
| | ALS |
| 341 | MKLNSFVAIGCTPREQYHWDEVIKGGPNSWAEYFPLANGDKQ |
| | SPIDIVPGSAKYDSGLKPLTLKYDPSTSLEILNNGHSFQVTFSDD |
| | TDSSTLTEGPISGVYRLKQFHFHWGASDDKGSEHTVDGVKYA |
| | AELHLVHWNAVKYSSFGEAASKPDGLAVLGVFLKVGKHHGE |
| | FEKIVNALGSIKHKDTLATFENFDPSCLMPACPDYWTYDGSLT |
| | TPPLLESVTWIVLKEPISVSPSQMAKFRSLLFTSEGEKACCMVD |
| | NYRPPQPLKGRQVRASF |
| 342 | MQEITVTVFENIQPSPVTPWNPEPKRPEIHPTAYIHPAAVVQGD |
| | VTIGANVLVMANAVIRADEGYPIVVGDNSAVQDNVVLHALET |
| | VDASGKELEENIVTVGDEKYAVYVGDNVVLAHNAQVHGPAA |
| | VGDNTFVGMNALVFRSRVGANCVLEHLAAALGVTVPDGRYV |
| | PAGRVVTTQEEAARLPAVTPDHPHADLNARVVAVNVALAKG |
| | YLALA |
| 343 | MKKTIMLVPVLFVFIAIFMTCDNKTNNHKDVKHSKETKEEMK |
| | KETAKKDCDQVHWSHHKGEHGPENWANLCEGFKDCNGEKQ |
| | SPIDIKEAVKGEDLKPLEFEYGKTKVNIINNGHTVQFNIDKGSS |
| | MMVDGKKYDLLQFHYHATSEHTIKGEYSPLEVHFVHRHADD |
| | DFAVLGIMYEEGEANDLFNKYLKHFPADKGEYTSDEKFDLDA |
| | LLPDNLSYYHYGGSLTTPPCSEVVSWYLLQNPLRASQEQIKDF |
| | SEILDKNFRPIQELNGRTIYKFGE |
| 344 | MKRLASVFTICGWNYQDPEHWHELFPTAKGNHQSPINIETRKT |
| | IYDSTLKPLTFSYEAATSRRIVNNGHSFQVEFEDTDNKSVLKGG |
| | PLTDRYRLTQFHFHWGSSDDHGSEHTVDGLKYPAELHLVHW |
| | NAKYGSFGEAASKPDGLAVVGVFLKIGRENAEFQLVLDALDSI |
| | KTKGKQTPFTNFDPSCLFPACRDYWTYSGSLTTPPLSESVTWIV |
| | LKQPIEVSPRQLSKFRSLLFTSEGEKEKRMVDNFRPLQPLMNRS |
| | VRSSF |
| 345 | MITRLPAVTAVLAMFVVCSMAHDPWNTDYTSQRGPLFWGQR |
| | PEFKMCGLGREQSPINIRRSSTIYQDFPPLAFELKSPIVHSNIENK |
| | GSATAAFPLSDIPILSGGPLGNRKYRVYNVHLHFGNYSFRAAE |
| | HAFDGVRTTGEFHIVTYDSRYPHIKAALGSGRRGALAVLGVM |
| | FEARNVSNIDMGVTNLIELSSNVTYKGDHYMTGIDFSNLVSEV |
| | DMGYYYAYNGSLTTPTCNEVVQWMVIDRIHYVLPETADLLLE |
| | LKTGYRREHSIPIFGNTRPLQPLYGRKVLRSFGPVVTDVHDQG |
| | EEIVYSSADLVGPLGRVMLLALAAIATFVIKA |
| 346 | MKLTSAFIVCGYQNHEPRDWHEVAPSAKGNRQSPINIQWRDS |
| | VYDPGLKPLTISYDPATCLHIWNNGYSFLVEFEDSTDKSVIKGG |
| | PLENNYRLKQFHFHWGATDDHGSEHTVDGVKYSAELHLVHW |
| | NADKFDSFVEAAHEKDGLAVLGVFLKIGEHNAQLQKITDILDSI |
| | KEKGKQTRFTNFDPVCLLPPCPDYWTYPGSLTVPPLSESVTWII |
| | LKQPINISSQQLAKFRTLLFTSEGETA |
| 347 | MNNRPIQPLNDRSIWINRIKTEKCEFGWCPPVEEEPEKASKkvek |
| | dddskasskqgksdkkgkssgdkksgkksgksnkkekePPQWNYASVQRWED |
| | DYSMCGGKKQSPVNANTSKIQSVQGPGAGLVSRMAYSAVGP |
| | NAGFQFKNNGKSLVLEGNWGTLRLPDGDYIAKSIKFHFPSEHA |
| | VDGVLAAGEMHIVHQRSDATGTDGLAVIAILLRDSDLLGQAG |
| | PVGFFDRLGFSSRLPVEGETVILGADTVLDIGAIFAPQLGGKYW |
| | HYEGSLTTPPCSETVHWYLMQTPAGINKAMVNNFKSLFPSPAN |
| | NRPVQGMYGRAIVVTELSVSSKEFD |
| 348 | MKFAAAVASIVFAVSGAAAIAAPEGAVDWVYGDGDLPEKWSI |
| | TNEAYGACDAGNMQSPIDLDLANTRGEIEFASSYEETTGELKT |
| | GPSKVQVDVAPGMGMISGQHLFSLVQFHFHTPSEHRLHGQRY |
| | PLTVHLVHGTATGDFAVLGVMFEEGDENPALARILSGIDGGSK |
| | NVAVDVRELVPENIDVYRYMGSLTTPPCTEGVLWLILKQPASI |
| | SAEQLRLFSQLYPNNARPVQSLNGRPVRDALLIAPGGRP |
| 349 | MQEITVTKYENIRPSPVTPWNPEPKLPKIHPTAYIDPAAVVQGD |
| | VTIGANVMVSANASIRSDEGYPIYIGDNSNVQDNVVLHALETV |
| | DENGKPLEENIVKVGDKDYAVYVGDNVSLAHQAQVHGPAAV |
| | GDNTFIGMQAFVFRSRVGKNCVLEPLAAAIGVTVPDGTYVPA |
| | GTVVTTQEEAAKLPKVTPDHPFYKTNEAVVKVNIALAKGYLA |
| | LK |
| 350 | MQEITVLTYSNVTKNEVTSTNPKPTTPVIDPTSYVDPNATVTG |
| | DVTIGKNCFIGAFAVIRADEGKPIVIGDRSNVQDGVVLHALESV |
| | DDGGEIREDNVVVHGDEYYAVYIGKNVSLAHQAQVHGPAHV |
| | GDDSFVGMKSLVFNSIVGDNCVIEPDAAAIGVTVPDGKYIPAG |
| | TVVTTQEEAAKLPEITPDHEFYTQIAAVVQVNVDLCKAYRDK |
| | K |
| 351 | MLKEWGYASHNGPDTWVQIFRCARGNNQSPIELKTKDIKHDP |
| | SLQPLSVSYDPGTAKEIVNVGHSFHVNFEDSDNRSVLKDGPITG |
| | SYRLRQFHFHWGASDDHGSEHVVDGVRYAAELHVVHWNAD |
| | KYPSFVEAAHEPDGLAVLGVFLKIGEHNPHLQKITDVLYAVKF |
| | KGTKAQFTNFNPKCLLPASLDYWTYPGSLTTPPLSECVTWIVL |
| | KEPISVSPSQMAKFRSLLFSSEGETACCMVDNYRPPQPLKGRT |
| | VRASF |
| 352 | MQEITVLEFSNITKNEVTPTNPKPSTPVIDPTSYVDPNATVIGDV |
| | TIGKNVLIAANAVIRADEGAPIVIGDRSNVQDGVVLHALESVD |
| | DDGKILEDNVVEKGDEYYAVYIGKNVHLAHQAQVHGPARVG |
| | DDSFVGMKSLVFKSKVGSNCVIEPDAAAIGVTVPDNKYIPAGT |
| | VVTTQEEADKLPEVTPDYAYSDTNEAVVTVNVDLNEAYRNQ |
| | Q |
| 353 | MQEITVTKYENIRPSPVTSWNPEPKLPKIHPTAYIDPAAVVQGD |
| | VTIGENVMISALASIRSDEGYPIYIGNNSNVQDQVVLHALETVD |
| | ENGNVLEENVVTVGDKKYAVYIGDNVSLAHQAQVHGPAAVG |
| | NNTFIGMQSFVFRSVVGENCVLEPLAAAIGVTVPDGTYVPAGT |
| | VVTTQEEADKLPKVTPDHPFYNTNAAVVKVNVELAKGYLAL |
| | K |
| 354 | MQEITVLNYSNIEKNEVTSTNPKPTTPVIDPTSYIDPNATVTGD |
| | VTIGKNCYIGPFAVIRADEGAPIVIGDNSNVQDGVVLHALESVD |
| | DGGKIRKDNVVEVGDNNYAVYVGKNVSLAHQSQVHGPARV |
| | GDDSFVGMNATVFNSIVGSNCVIEPNAAAIGVTVPDGKYIPAG |
| | TVVTTQEEADKLPEITEDYPFYTAVEEVVKVNVALCKAYREK |
| | K |
| 355 | MLSRKPAGHTDIYEQFVCNWKNGGGKLHRSSATDPEGERQSPI |
| | DIQTSKVEVDQKLQPLTLTYDPSTSLEILNNGHSVQVTFKDKD |
| | NRSVLKGGPLTGPYRLKQFHFHWGKKDDVGSEHTVDGAKFA |
| | SELHLVHWNAKKYSSFAEAASKSDGLAVLGVFLQVGEHNAQ |
| | LQKITDILDSIKEKGKQTRFTNFDPLSLLPPCRDYWTYHGSLTV |
| | PPLLESVTWIILKQPISVSSQQLAKFRSLLCTSEGEKAVPMLSNH |
| | RPPQPLKGRQVRASF |
| 356 | MTMKLSVLGQLFLVICCLTVVINAMPNPQAQAPSGALVATAE |
| | SGHFSYDDPNKWKEHNSLCAGEHQSPINIDTRKSRTDKFPPFRF |
| | HNYAKGLPENLENNGHTVQLTIDNLIKDLPTISGGGLEGPYEFA |
| | QMHFHWGEDEFGSEHKINNKQYAGEVHIVHWNKKYGNFVNA |
| | TKHNDGLAVLGILIDLQDKENIAFSHIEQFDEIRDASKKNEKLP |
| | YSVPLKDLLPSNTASFFRYEGSLTDARCNEDVTGPFLKHQFTSP |
| | IIRQLNDDEGEPLSKNVRPTQEEHDRIVTYSGRAMQCGTCSTA |
| | TADRSSEGRSDSSESGESKEITKSKTRHYGY |
| 357 | MQEITVTRYENIRPSPVTPWNPEPRLPEIHPTAYIDPAAVVTGD |
| | VRIGANVMVSALASIRSDEGYPIVIGDNSNVQDQVVLHALETV |
| | DADGKVLEENVVEVGDERYAVYIGDNVSLAHQAQVHGPAAV |
| | GDNTFIGMQAFVFRSRVGKDCVLMPLAAAIGVEVPDGRYIPA |
| | GKVVTTQEEADKLPKVTPDHPFANTNAAVVAVNVALAKGYL |
| | ALA |
| 358 | MQEITVTTYNNIRPSPVTPWNPEPKLPKIHPTAYIDPAAVVQGD |
| | VTIGENVLVMANAVIRADEGYPIVIGNNSSVQDNVVLHALETV |
| | DEDGKRIEENVVKVGDKDYAVYVGDNVVLAHNAQVHGPAA |
| | VGDNTFVGMNALVFHSNVGKNCVLAPNAAAIGVTVPDGKYIP |
| | AGKVVTTQEEADKLPEVTPDHPFYDLNARVVKVNVALAKGY |
| | LALA |
| 359 | MAMLNKKRKEWKMRGRVFFALILAMCLSVAGYSLYEKEQNQ |
| | HDEERIEDVYYSYDEHGPDAANVCERGMMQSPVQITRKDALQ |
| | NQSPEIEIHYGEGRFEIIKKAHTAEAVSKSGQNYILIDHQQYKLE |
| | SFHFHLPSEHQVEGQSYEMELHFVHENKNGEQAVMAVFIQEG |
| | QANEMVKEIWSRLQDGFSKKDNVSIRLPEFIPKERRAFYYTGS |
| | LTTPPCTEGVKWIVFEMPVEFSEEQIGTFHRLFGNNSRQVQPLN |
| | GRKIYQLTVR |
| 360 | MKHCTLLAILLSGLLSAAVETDFSYADQGAWQTLPNSQCGGR |
| | RQSPVDLDLRNVTVDKILGQDLACTWQQNKAVIEGDLVNTGR |
| | TIELDVRSPHTCRGVPGSPSAHFRLAAVHIHYGSASDQGSEHTI |
| | NGRTSALEVHMVHFDTRFASLDKAREQPGGIMVAGLLFDEAD |
| | EAIANPELTKMAVISGTALRSTGGVLASRLNAAPLIEGTGLDKA |
| | RARFLTYAGSLTTPTCNEVVTWIVAAEPGLVGHQTMHLLRTV |
| | TGLGNKTISPNFRQVQPLNGRTITSSFQPACGLHGRC |
| 361 | MLKNPAGDWSYEQTVHFIRCTDFIPAVILPGGARQSPINIVTSQ |
| | AVYSPSLKPLELSYEACTSLSITNNGHSVQVEFNDSTDRTVIKG |
| | GPLEGPYRLKQFHFHWGARDSRGSEHTVDGARYPSELHLVHW |
| | NAKYASFGEAASQPDGLAVVGVFLKIGREKPGLQKVLDALDA |
| | IKTKGKQTRFTNFDPSTLLPGCLDYWTYDGSLTTPPLLESVTWI |
| | VLKEPISVSSGQMAKFRSLLFTSEGETACCMVDNYRPPQPLKG |
| | RQVRASF |
| 362 | MQEITVLEYSNIRKNEVTPWNPKPSTPVIDPTSYIDPNATVIGD |
| | VTIGANVLIGPNAVIRADEGRPIVIGDRSNVQDGVVLHALESVN |
| | DEGMEIGDNVVLEGNSYYAVYIGKNVVLAHQSQVHGPAAVG |
| | DDSFVGMQSLVFNSIVGSNCVIEPNAAAIGVTVPDGKYIPAGT |
| | VVTTQAEADKLPEVTPDHAFYTDVAKVVSVNVNLCRAYKEQ |
| | S |
| 363 | MIRKNPSGHLPVIAETAFIDQTAIICGKVIIYDNVFVGPYAVIRA |
| | DEVNEHGDMEAIVIKRDTNIQDGVVIHSKAGAAVTIGERSSIAH |
| | RSIIHGPCWVGDDVFIGFNSVVFNAKIGKGCVIRHNSVVDGLD |
| | LPEHFHVPPMTNIGADFDLSSISKVPPEYSAFSESVVSANHELV |
| | QGYRRIANEL |
| 364 | MRILVTACFYPNSEHDGQKWANIKPEWKTCGHGKMQSPIDLS |
| | SHRVSLVHDQTWNRDYKPAPAVIVNRGHDIMVSWKEDAGKV |
| | TIHQTDYKLVQCHWHSPSEHTVNGTRYDLELHMVHTSAQGKT |
| | AVIGVLYKLGRPNEFLAKLLDGIKSVGKEEKDLGIVDPRTIGFH |
| | TDKFYRYVGSLTTPPCTEGVIWTVVKKVNTVSMEQLAALREA |
| | V |
| 365 | MQEITVTRYENIRPSPVTSWNPEPKLPKIDPTAYVDPAAVVQG |
| | DVTIGKNVMVSANASIRSDEGYPIYIGDNSNVQDNVVLHALET |
| | VDADGKRIEKNIVTVGDKEYAVYVGDNVSLAHQAQVHGPAA |
| | VGDNTFIGMQSFVFNSTVGKNCYLAPLAAAIGVTIPDGTYIPAG |
| | KVVTTQEEAAKLPKITPDHPFANTNAAVVKVNVELAKGYLAL |
| | K |
| 366 | MSTPLVRWGYKEDNGAHQWCIFFPEACGKRQSPINIQTSKVV |
| | YDPGLRPLNLNYDPSTSLEILNNGHSVQVNFKETDDRSVLSGG |
| | PVTGTYRLRQFHFHWGAKDCRGSEHTVAGVKYPSELHLVHW |
| | NAVKYESFAEAALEENGLAVIGVFLKLGKHHDELQKLVDALP |
| | SIKHKDTLVEFGSFDPSCLMPTCPDYWTYSGSLTTPPLSESVTW |
| | IIKKQPVEVDHDQLEQFRSLLFTSE |
| 367 | MSSTFVVGRPCDTRLPRGSRKEPARVPHGFAAGIATVVSLALL |
| | CLAGGCAHAPVPREAGRSVAQSEADYYSDALAPWTYPEGPS |
| | WGAACAKQPPPQQSPIDLTRVTTAPWSASSVITQATFDGHDQN |
| | VVFQASPGPSVTMAPGVDGSGRAFVYTVAGFHFHYRNEHVIA |
| | GNPVYELHIKTVDQHGGVAVFAVLWTADDAAGEDPTLAAAY |
| | RSLSAPPDSVVAVDLGRALWRFGQQPFYSYVGSLTTPPCTTGI |
| | RWFVLQTPIRTSSASIGRLNAALIARGMPRDNVRTVRPVAQPQ |
| | PVVYLVTPK |
| 368 | MQEITVLEFSNITKNEVTPTNPKPTTPVIDPTSYVDPNATVTGD |
| | VTIGKNVLIGPNAVIRADEGRPIVIGDNSSVQDGVVLHALESVD |
| | DEGKIIEDNVVLYGNKYYAVYIGKNVVLAHQSQVHGPAAVGD |
| | DSFVGMNSLVFNSIVGSNCVIEPNAAAIGVTVPDGKYIPAGTV |
| | VTTQEEADKLPEVTEDYKFYTQVAKVVTVNVNLCEAYRNQA |
| 369 | MQEITVTTFTNIRPSPVTPWNPTPKLPKIHPTAYVDPAAVVQGD |
| | VTIGENVMISANASIRSDEGYPIYIGNNSNVQDNVVLHALETVD |
| | ENGKVIEENVVTVGDKKYAVYIGDNVSLAHQAQVHGPAAVG |
| | DNTFIGMQAFVFKSNIGKNCVLEPLAAAIGVTVPDNKYIPAGT |
| | VVTTQEEAAKLPEVTPDHPFANTNAAVVKVNNALAAGYLALS |
| 370 | MKLSNVFIAGCTYQEPWDHREWDYSKKGPATWGLINSAWSL |
| | CSIGKRQSPIDIELNQLLYDPFLPPLRLSSGGKKLGGTMYNTGR |
| | HVSFRPDKAQLVNISGGPLSYSHRLEEIRLHFGSEDSQGSEHLL |
| | NGEAFSGEVQLIHYNQELYSNFSEAARKPNGLLIISIFMKVADT |
| | SNPFLNRMLNRDTITRISYKNDAYFLMNLNIELLYPESFGFITY |
| | QGSMSTPPCYETATWILIDRPINITSLQMHSLRLLSQNLPSQIFLS |
| | MSDNSRPLQPLAHRALRGNR |
| 371 | MQEITVTRYENIRASPVTPWNPTPKLPKIHPTAYVHPLAEVVG |
| | DVTIGANVMVSANASIRSDEGYPIVIGDNSNVQDNVVLHALET |
| | VDANGNRIEENIVKVGDEEYAVYVGDNVSLAHQAQVHGPAA |
| | VGDNTFIGMQAFVFRSVVGKNCYLAPLAAAIGVTVPDNTYIPA |
| | GKVVTTQEEADKLPKMTPDHPFYNTNKAVVKVNVALAKGYL |
| | ALA |
| 372 | MRSPLAGWTIVFCQKDEYNHLDGILGGPGYWGLINPEWRMCS |
| | KGKMQSPIDIDPKVLLYDPNLSAVHLDKHKVSGTLENTGQSLV |
| | FRVDKGSRQHVNISGGPLAYRYQFHEIFLHYGLKDSMGSEHRI |
| | NGYSFPAEIQLYGFNSELYHNMSEAQHKSQGIVGVSLMVQIGE |
| | TPNPELRILTSQLERVRYRGQSAPIHHLSLRGLLPDTEHYMTYE |
| | GSTTHPGCWETTVWVILNKPIYITRQELYALRRLMQGSQSQPK |
| | APLGNNARPVQDLHGRTVRTNI |
| 373 | MLISRFATGHPVKENDCYQWFGPNGEKGPDKWGKINPKWKV |
| | CGEGKLQSPIDLLNQRVQILPNLGKLQKDYKPAPAVLKNRGH |
| | DIMVKWKGDAGKLNINGTYYKLVQCHWHTPSEHTINGTKFD |
| | MELHAVHKSSKGETAVIGIWYKIGRPDSFLSKLLKNIKSVGDK |
| | EIDLGVINPGDIKFGSRKYYRYMGSLTVPPCTEGVIWTIVKKVR |
| | TVSREQLRAL |
| 374 | MKRSLIFAVGTCPQEDWHYNYDEASGRGPSRWGLLKPEWRT |
| | CSVGKLQSPIDIGTVQVSSELGDLQRNYRSAPALLRNRTEDVA |
| | VIWLGNAGSITINGVVYRVVNCHWHSPSEHTFNGTRLPLEIHIV |
| | HRSSQNRIAVVGILYKYGLPDPFLSKLFHSIKSLGKEEKNLGIV |
| | NPESIGFQDKKYYRYIGSLTTPPCSEGVVWTVFKKVRTVSREQ |
| | LKALKDAVD |
| 375 | MQEITVTRFENIRPSPVTPWNPEPKLPKIHPTAYIDPAAVVQGD |
| | VTIGANVMVSANASIRSDEGYPIVIGDNSNVQDNVVLHALETV |
| | DENGNVLEENVVTVGDEKYAVYVGDNVSLAHQAQVHGPAIV |
| | GDNTFIGMQAFVFRSRVGKNCVLAPLAAAIGVTVPDGTYVPA |
| | GKVVTTQEEAAKLPKVTPDHPFANTNAAVVKVNVALAKGYL |
| | ALA |
| 376 | MQEITVTKYNNIRASPVTPWNPTPKLPNIHPTAYIDPAAVVQG |
| | DVTIGANVMVSANASIRSDEGYPIYIGDNSNVQDNVVLHALET |
| | VDENGNPIKENIVKVGDKDYAVYVGDNVSLAHQAQVHGPAA |
| | VGDNTFIGMQSFVFASEVGKNCYLAPLAAAIGVKVPDNTYIPA |
| | GKVVTTQEEAAKLPKITPDHPFANTNAAVVKVNVALAKGYLA |
| | QS |
| 377 | MQEITVLIYSNVTKNEVTTWNPKPKTPKIDPTSYVDPKATVIGD |
| | VTIGKNCMISPFASIRSDEGMPIVIGDNSNVQDGVVLHALETVD |
| | TNGKIIEDNVVIKGDKRYAVYVGKNVSLAHQSQVHGPARVGD |
| | DSFIGMQSFVFKSIVGSNCVIEPNAAAIGVTVPDNKYIPAGTVV |
| | TTQEEADKLPEITEDYKYYNTNDAVVYVNVKLCKAYRNKS |
| 378 | MQEITVTNFNNIQPSPVTPWNPEPKLPKIHPTAYIHPLAYVQGD |
| | VTIGENVLVMANAVIRADEGYPIVIGNNSSVQDNVVLHALETV |
| | DENGNRIEENIVKVGDEEYAVYIGDNVVLAHNAQVHGPAAVG |
| | DNTFVGMNALVFRSRVGKNCVLEPLAAAIGVTIPDGTYIPAGK |
| | VVTTQEEAAKLPKITPDHPFYNLVDRVVKVNVALAKGYLALS |
| 379 | MRKSLFACTVIGWNYEDQPHWSELDPAYAACATGKEQSPIDIR |
| | GARRADLPPLRFEYRSAPLKYVINNGYTIRVNYHDSPGSGNFLI |
| | VGDARYQLTQFHFHRPSEEYVHGKPYTMELHLMHQSSDGEV |
| | AGVAVLLKAGRANATIQRLWEHMPATEGQEQVLAGVTIDPAG |
| | LLPRETGYYVYMGSVTAPPCTEGVTWFVLKTPVEISAEQIAVF |
| | ARLYPHDVRPLQPL |
| 380 | MQEITVTRYENIRPSPVTPWNPEPRLPEIHPTAYIDPKAVVQGD |
| | VTIGENVLVMANAVIRADEGYPIVIGDNSSVQDNVVLHALETV |
| | DENGNRLKENVVTVGDKEYAVYIGKNVVIAHLAQVHGPAAV |
| | GDNTFVGMLALVFNSTIGKNCYLAPLAAAIGVTVPDGTYIPAG |
| | TVVTTQEEAAKLPKITPDHPFADLNARVVKVNIALAKGYLALA |
| 381 | MQEITVTVYENIRESPVTSWNPTPRRPEIHPTAYVDPAAVVVG |
| | DVRIGANVMVSANASIRSDEGYPIVIGDNSNVQDNVVLHALET |
| | VDAAGNRITENIVTVGDEEYAVYVGDNVSLAHQAQVHGPAA |
| | VGDNTFIGMQAFVFRSNVGKNCVLEPLAAAIGVTVPDGTYVP |
| | AGKVVTTQEEAAKLPKVTPDHPFANTNAAVVAVNVELAKGY |
| | LALA |
| 382 | MQEITVTRYENIRPSPVTPWNPEPKLPKIHPTAYVDPAAVVVG |
| | DVTIGANVMVSANASIRSDEGYPIYIGDNSNVQDNVVLHALET |
| | VDENGKVIEENVVTVGDKKYAVYIGKNVSLAHQAQVHGPAA |
| | VGDNTFIGMQAFVFRSVVGKDCVLEPLAAAIGVTVPDGTYIPA |
| | GKVVTTQEEAAKLPKITPDHPFANTNAAVVKVNVALAKGYLA |
| | LA |
| 383 | MQEITVFEFSNITKNEVTPTNPKPTTPVIDPTSYIDPNATVTGDV |
| | TIGKNVLIGPNAVIRADEGAPIVIGDNSSVQDGVVLHALESVDD |
| | EGEIIEDNVVLEGDEYYAVYIGKNVVLAHQAQVHGPAMVGDD |
| | SFVGMKALVFKSKVGSNCVIEPEAAAIGVTVPDGKYIPAGTVV |
| | TTQAEADKLPEVTDDYPFYTAVEEVVEVNVNLAEAYREQS |
| 384 | MQEITVMDFSNIVKNEVTPTNPKPTTPVIDPTSYIDPNATVIGD |
| | VTIGKNCYIGPFAVIRADEGAPIVIGDDSNVQDGVVLHALESVD |
| | AGGKIREDNVVTVGDRSYAVYVGKNVSLAHQSQVHGPARVG |
| | DDSFVGMNSLVFNSIVGDNCVIEPGAAAIGVTVPDGKYIPAGT |
| | VVTTQAEAAKLPEVTPDHAAYSANAAVVEVNVALSEAYRNL |
| | K |
| 385 | MVKRNILADFSPEGQTHWCYDCFIRPLPPVKWAKLFPKAKGN |
| | FQSPINIESRETRYDPSLKPLTLKYDPSTAKLISNSGHSFNVDFD |
| | DTEDKSVLRGGPLTGSYRLRQFHLHWGSADDHGSEHAVDGV |
| | KYAAELHVVHWNAVKFESFEEAALEENGLAVIGVFLKLGEHN |
| | PHLQKITDILYSIKFKDTLAEFTNFNPKCLLPTSLDYWTYSGSLT |
| | TPPLLESVTWIVLKEPISVSSEQMAKFRSLLFTSEGETACCMVD |
| | NYRPPQPLKGRQ |
| 386 | MLSKRIFGVATCQPENWHDYYKNANGEKQSPINIVTKETKYD |
| | SSLKPLTFKYDPSTAKEIVNVGHSFHVNFEDSENKSVLKGGPLT |
| | GTYRLKQFHFHWGSADDKGSEHTVDGVKYPSELHLVHWNAV |
| | KFESFAEAALEENGLAVIGVFLKLGEHHKELQKLTDTLPSIKHK |
| | DTLANFGSFDPSCLMPTCPDYWTYPGSLTTPPLSESVTWIVLK |
| | QPIEVSEEQLAAFRSLLFTSEGEK |
| 387 | MQEITVLEFSNVTKNEVTSWNPKPSTPVIDPTSYVDPNATVIGD |
| | VTIGKNCYIAASAVIRADEGKPIVIGDNSNVQDGVVLHALESV |
| | DDGGKIREDNVVIHGDKWYAVYIGKNVSLAHQSQVHGPAYV |
| | GDDSFVGMNSLVFKSIVGSNCVIEPNAAAIGVTVPDGKYIPAG |
| | TVVTTQAEADKLPEITPDYAFYTQVAAVVKVNVNLCRAYRNQ |
| | A |
| 388 | MQEITVLIYSNVTKNEVTSTNPKPVTPVIDPTSYVDPNATVTGD |
| | VTIGKNCLIGPSAVIRADEGAPIVIGDRSNVQDGVVLHALESVD |
| | DEGKIIEENVVVHGDKYYAVYIGKDVVLAHQAQVHGPARVG |
| | DHSFVGMKSLVFNSIVGSNCVIEPNAAAIGVTVPDGKYIPAGT |
| | VVTTQEEAAKLPEITPDHEKYTKIAEVVTVNVNLCRAYRNKA |
| 389 | MIKLSVAFCTGNQRPEWDYHNNGHGKEEWPEEYPSCGGQLQS |
| | PIDLHGDILQYDASLTPLQFQGYNVSATEQFTLTNNGHSVQLSL |
| | PSDMYLKGLPSRYTATQLHLHWGKKGDLEGSEHQINSEATAA |
| | ELHIVHYDSEKYSNISEAMNKPQGLAVLGILIEVGETENPAYDH |
| | ILSRLHEIRYKDQKTSVPGFNIRELLPEQLEEYYRYQGSLTTPPC |
| | YQSVLWTLFNRRAQISMGQLEKLQETLSSTESEPSEPLVQNYR |
| | VPQPLNQRTVFASF |
| 390 | MGHTWCNDEEGTRRGRSEGPTPAAAGVRVERMIREGGSRRR |
| | AATPHVRCGVLYAVRGVPMSARSWLTASALTVAAVTLIGCAQ |
| | AAPAETAPTERPVAEPAHWSYDGDSGPESWAGLDDAFQACEA |
| | GTDQSPIDLPAAVPAPSTSIELSAEEAEGDVFDSGHAVEIETDG |
| | QGETLTFADDDYSLQQLHAHVPSEHTVAGQPAAAELHLVHAD |
| | ADGNLLVLGVLVTEGAASDALTPFIEAASHLADDEEVTLDLAA |
| | VLPASLENYEYSGSLTTPPCTEDVQWVVMGTPISMSAEQIGTL |
| | AGAHNHNARPTQPLGDRTVVGGAGKVEITG |
| 391 | MARKNPSGHLPQVSETAFIDPTAIICGKVIIEDYVFIGPYAVIRA |
| | DELNAAGDMEPIVIGAHSNIQDGVVIHSKSGAAVTIGEFSSIAH |
| | RSIVHGPCWIGDRVFIGFNSVLFNCHIQSGCVVRYNAVVDGVT |
| | LPENTYIPSTERVGPDSDLSLYRQVDRGALQFSEEVAATNVEL |
| | VRGYQALRNEF |
| 392 | MQEITVTRYENIRESPVTPWNPTPKRPKIHPTAYIDPLAYVQGD |
| | VTIGENVMVSALASIRSDEGYPIYIGNNSNVQDNVVLHALETV |
| | DENGKEIEENIVTVGDEKYAVAIGDNVSLAHQAQVHGPAIVGD |
| | NTFIGMQAFVFRSKVGKNCVLEPLAAAIGVTVPDNTYIPAGTV |
| | VTTQEEAAKLPKVTPDHPFANTNAAVVKVNVALAKGYLALA |
| 393 | MNPITSFNPVQRYPKIDKTAFISPFSSVIGDVRIKDNVYVAPNVS |
| | IRADEGTPFYIGSNTNLQDGVILHGLLNKFVTVNDKKYSIYIGN |
| | QVSIAHDALIHGPCYIGDKVFVGFKAIVYNAIVGKGTVISYNAV |
| | VTNGVRIAPNRFVPPGANIDTQEKADALSRVPKDEEEFAREVQ |
| | RVNQEFPASYHLLFGENRCSCGLS |
| 394 | MQEITVLDFSNVTKNEVTPTNPKPKTPVIDPTSYIDPNATVIGD |
| | VTIGKNVMVWPSAVIRADEGKPIVIGDNSSVQDGVVLHALESV |
| | DDGGKVIEDNVVLEGNKRYAVYIGKNVTLAHQSQVHGPAYV |
| | GDDSFVGMSSFVFNSKVGSNCVIEPNAAAIGVTVPDGKYIPAG |
| | TVVTSQAEADKLPEITDDYPYSNAIAAVVKVNVQLCEAYKAQ |
| | A |
| 395 | MQEITVLRFSNIRKNEVTPENPEPETPVIDPTSYIDPNATVIGDV |
| | TIGANCYIGPFARIRADEGRPIVIGDRSNVQDGVVLHALESVDA |
| | EGEIIEDNVVIEGDELYAVYIGRDVSLAHQSQVHGPARVGDDS |
| | FVGMKSLVFKSDVGSNCVIEPFAAAIGVTIPDGKYIPAGTVVTS |
| | QAEAEKLPEITEDYPFYTTIEEVVKVNVNLAKAYREQK |
| 396 | MQEITVMEFSNVTKNEVTSTNPKPKTPVIDPTSYVDPEATVIGD |
| | VTIGKNCYIGPFARIRADEGAPIVIGDDSSVQDGVVLHALESVD |
| | ADGKIIEDNVVLHGDKLYAVHIGKNVSLAHQAQVHGPARVGD |
| | DSFVGMNSLVFNSVVGSNCVIEPNAAAIGVTVPDGKYIPAGTV |
| | VTTQEEADKLPEITPDYAKSTAIAAVVEVNVALCEAYREQA |
| 397 | MQEITVAEFSNITKNEVTSWNPKPKTPVIDPTSYVDPNATVIGD |
| | VTIGKNCYIAPFASIRADEGTPIVIGDDSNVQDGVVLHALESVD |
| | ADGKILEDNIVLHGDKRYAVYIGKNVSLAHQSQVHGPAHVGD |
| | DSFVGMMSLVFKSKVGNNCVIEPGAAAIGVTVPDGKYIPAGT |
| | VVTTQAEADKLPEITPDYAKSNQVAAVVKVNVALCEAYRKQS |
| 398 | MQTYDSSLRESLLPLPDKKESVRYWLILGGFVTAAAVAVFVIA |
| | ARSGSHADASVLNSALIAVAPHFEYAEANCDETKCEASEIVQL |
| | DTWSWAPTCITGRAQSPIDIVTKEVATAGLLDDAISLSIGSATL |
| | VPSNTGHGFQLTSTGGTPSAMFRGEKFNFKQTHWHTPSENTV |
| | DGEHAAMEGHFVFQLDDPLWVNTTLNLAVMAVFFELGDCNQ |
| | HLSAVWDTFPVDRLGTGSGTFSGEILASLLASVLGGGYYQFTG |
| | SLTTPPCTEGVAWNVMKKRTTVCQDQVDRLKHALSATANGV |
| | DISNRVVQPLHQRVVTQTSR |
| 399 | MSRLTGKIAVFCYQNPEHWDYDSPIAKGNRQSPIDIDLWSAKY |
| | DPGLKPLTFTGYDKKSLRTLLNNGHSVSVQFEDSEDKAVLSGG |
| | PLTGVYRLKQFHFHWGAADDKGSEHTVDGVKYPSELHLVHW |
| | NAVKFSSFAEAASKPDGLAVVGVFLKIGKEHVELNKLTDALY |
| | MVRFKGTKAQFSCFNPKCLLPASRHYWTYPGSLTTPPLSESVT |
| | WIVLREPISVSERQMEKFRSLLFTSEDDERIHMVNNFRPLQPLM |
| | NRTVRSSFR |
| 400 | MVPYPYPIVYQNPPVAEVTSVSYPKISRKAVIGTDSMIIGDITIA |
| | DDVYIGFKNLLRADSGHPYYVGPYTNIQDYVLMHVHPGREHV |
| | VVNNQKWGVFLEGMNSVLHHAAVHGPLFIGKNTFIGQHANIY |
| | DAVIGRDCVVMHGATVTNGVKIADNRFVAPGQSVWQQSEAD |
| | KLPPVPEKFKDLNRSIVDHYYRLGKSYGLNTPLAYSYSGG |
| 401 | MLALTLAILLLLNARAVLSSCAHGTYLLRRAIDDNKPIKLPNFG |
| | YGPFDGPTNWHSLSEDNILCGTGRRQSPIDIDDTISQVAAGFVS |
| | MDVPIQDVSFLNLRTTVEVILKGSTRINGREFVLEQFHFHTPSE |
| | HVLNGEIFVAEVHFVHSNKENPKELAVITLMVQVSADHSTRSL |
| | DRVIGEITRISTPGNKVAIPALNIGDITSLVNKQQLFTYTGSLTTP |
| | PCTEGVQFFILPQPIPMRATVFNALKSVTGHNARFLQNNNATR |
| | PNVLVAGCQVIAAEVWSNATQYSR |
| 402 | MLAATVPAGTALADEWGYAGDGAPVNWGALSPDFAVCSAG |
| | VQQSPVDLVPGIIADGVRPVLDFADVSGVEAERSAHGVTYHVP |
| | SGSAQLSLNGRSFDLLQFHFHAASEHWVEGQSYPLEVHFVTAS |
| | EGDLAVVGVLFERGEAHATVDTLWDAIGEPGDREEIDGPVSLA |
| | SLLPQDQAAFRYEGSLTTPPCSEIVSWTVFTTPLSVSDAQIDAF |
| | VETVGENARPPQPLNRRYVLLDN |
| 403 | TAYIDPQASVIGEVTIGANVMVSPMASIRSDEGMPIFVGDRSNV |
| | QDGVVLHALETINEEGEPIEDNIVEVDGKEYAVYIGNNVSLAH |
| | QSQVHGPAAVGDDTFIGMQAFVFKSKVGNNCVLEPRSAAIGV |
| | TIPDGRYIPAGMVVTSQAEADKLPEVTDDYAYSHTNEAVVYV |
| | NVHLAEGYKETS |
| 404 | TAFIAPNAEVIGDVTIEGNAMISPNASIRADEGMPIYLGKDVNL |
| | QDNVQLHALETVDEEGNLIEENLVEVNGKKYAVYLGENVSLG |
| | HQAQVHGPAYVGKDTFIGMNAVVFKSRIGNNCVLEPNATVIG |
| | VTIPDGRYVKAGTVIRTQADLPKLLDLKEVPEVKKKVDEYHN |
| | KLKELIKAKKAAA |
| 405 | TAYIHPSAQVIGEVEIGANVMVSPMASIRADEGMPIVLGDNAN |
| | VQDGVQLHALETVNEEGELIEENVVEVDGKKYAVYVGENVSL |
| | AHQAQVHGPAIVGKDTFIGMGAKVFKSTVGNGCVLEPGAEVI |
| | GVTIPDGRYVPAGTVVRTQEQIPSLREMTPDDPLLAVRDRVIA |
| | ENLKRAAELKARA |
| 406 | TAYIAEGAEVIGEVYIGENVMISPNASIRSDEGMPIYIGENANV |
| | QDGVELHALETKDEEGNLIEENVVEVNGKKYAVYVGENVSLA |
| | HQAQIHGPARVGKDTFIGMGAVVRGSTLGENVLLGEGVVIEN |
| | VTIEEGTLVEEGTVITKQEDVKKLRKLKPSDKMVKIKKEVLEK |
| | NKKLWEKLKEEE |
| 407 | TAYIHPSATVIGPVRIEEGVMISPNASIRADEGGPIYLGENVNVQ |
| | DGVTLHCLEVKREEGRVDESVFVEVDGEKYCVYLGEGVSLGH |
| | QATIHGPAKVGEDTFIGMGATVFRSTIGEGCVLEPGATVIGVTI |
| | PEGRYVPAGKTVTTQAEADALPLITDSYPYRETNENVVAVNL |
| | ALAEAARAAA |
| 408 | TAYIHPSAQVIGEVQIGANVMVSPMASIRSDEGMPIYIGDNAN |
| | VQDGVQLHALEARNEEGVEDESAWVEVNGKRYRVYVGENVS |
| | LAHQAQVHGPAAVGKDTFIGMGASVFKSRLGNGCVLEPGATV |
| | IGVTIPDGRYLPAGTVLRGRPIEEEIELREVTEELRARHQAVVE |
| | ANLARAAELKARA |
| 409 | TAYIHPSAEVIGEVEIGANVMVSPMASIRSDEGMPIYIGDNANV |
| | QDGVLLHALEALDEEGEEDEEAYVEVDGKRYRVYLGNNVSL |
| | AHQAQVHGPAKVGDDTFIGMGASVFKSILGNGCVLEPNATVI |
| | GVTIPDGRYIPAGAVLNVEDAEKTRELPEVTPELRAKRAAVLA |
| | ANAARYAELRAAA |
| 410 | TAYIHPSAQVIGDVQIGANVMVSPMASIRSDEGMPIYIGDNAN |
| | VQDGVQLHALEARDEEGVEDEEAYVEVNGKRYRVYLGENVS |
| | LAHQAQVHGPAAVGRDTFIGMGASVFKSRLGNGCVLEPQATV |
| | IGVTIPDGRYLPAGAVLEGETAEAVAALPEVTPEMREAVAAQQ |
| | AAAAAQYAAAKAAA |
| 411 | TAYIAPSAEVIGEVTIEDDCMISPNASIRADEGMPIYLGNGTNV |
| | QDGVTLHGLEVKREEGEEDESAYVEVNGKKYVVYLGDNVSL |
| | GHQAQIHGPAKVGDDTFIGMGATVFKSVIGNGCVLEPGATVIG |
| | VTIPDGRYVPAGATVTTQAEADALPLMTPDYALYHTNERVVA |
| | VNRALAAEARAAA |
| 412 | TAYIHPSASVIGDVEIGANVMVSPMASIRSDEGMPIHIGDNANV |
| | QDGVVLHGLEVKNEEGEEDESQYVEVNGKKYVVYLGKNVSL |
| | AHQAQIHGPAKVGDDTFIGMGAFVFKSTLGNNCVLEPGATVIG |
| | VTIPDGRYLPAGTTVTGKPLAEDVTVRPVTEEQRNKHKKVVE |
| | KNLKLAKLLKELS |
| 413 | TAYIHPSAQVIGPVQIGANVMVSPMASIRSDEGMPIYIGDNANV |
| | QDGVQLHALEARDEEGVEDEAAYVEVNGKRYRVYIGENVSL |
| | AHQAQVHGPASVGSDTFIGMGASVFKSRLGNGCVLEPQATVI |
| | GVTIPDGRYLPAGTVLLPGRLEDNTPLREVTPEQREAHKAVVT |
| | ENLARATELKAAL |
| 414 | TASIAPSATVIGDVEIADNVMISPNASIRSDEGMPIYLGANANIQ |
| | DNVTLHALETKDEEGNLIEENYVEVNGKKYAVYIGDGSTLPA |
| | GLTIKGNGYVEVLAGPGELLVVTEPYKVEITSPEPVLVLRRLSP |
| | EVRELLEVSPDSERLLAREADGGTALFAAFDARRAALKAANL |
| | AANAAAVAALTASIAPSATVIGDVEIADNVMISPNASIRSDEG |
| | MPIYLGANANIQDNVTLHALETKDEEGNLIEENYVEVNGKKY |
| | AVYIGDGSTLPAGLTIKGNGYVEVLAGPGELLVVTEPYKVEITS |
| | PEPVLVLRRLSPEVRELLEVSPDSERLLAREADGGTALFAAFDA |
| | RRAALKAANLAANAAAVAALTASIAPSATVIGDVEIADNVMIS |
| | PNASIRSDEGMPIYLGANANIQDNVTLHALETKDEEGNLIEENY |
| | VEVNGKKYAVYIGDGSTLPAGLTIKGNGYVEVLAGPGELLVV |
| | TEPYKVEITSPEPVLVLRRLSPEVRELLEVSPDSERLLAREADG |
| | GTALFAAFDARRAALKAANLAANAAAVAAL |
| 415 | TAYIHPSASVIGDVEIGENVMISPNASIRSDEGMPIYLGENVNV |
| | QDNVTLHGLEVYTEEGELIEENLVEVNGKRYVVYTGKNVSLG |
| | HQAQIHGPAKVGDDTFIGMNATVFKSVIGNNCVLEPNATVIGV |
| | TIPDGRYVPAGKTVTTQAEADALPVLTPDYALYHTNERVNAV |
| | NLKLAQEANAAATAYIHPSASVIGDVEIGENVMISPNASIRSDE |
| | GMPIYLGENVNVQDNVTLHGLEVYTEEGELIEENLVEVNGKR |
| | YVVYTGKNVSLGHQAQIHGPAKVGDDTFIGMNATVFKSVIGN |
| | NCVLEPNATVIGVTIPDGRYVPAGKTVTTQAEADALPVLTPDY |
| | ALYHTNERVNAVNLKLAQEANAAATAYIHPSASVIGDVEIGEN |
| | VMISPNASIRSDEGMPIYLGENVNVQDNVTLHGLEVYTEEGELI |
| | EENLVEVNGKRYVVYTGKNVSLGHQAQIHGPAKVGDDTFIGM |
| | NATVFKSVIGNNCVLEPNATVIGVTIPDGRYVPAGKTVTTQAE |
| | ADALPVLTPDYALYHTNERVNAVNLKLAQEANAAA |
| 416 | TAYIAPGAEVIGEVEIGANVMVSPMASIRSDEGMPIYLGDNTN |
| | VQDGVTLHGLEVEDEEGEEDESVYVEVNGKKYRVYIGNNVSL |
| | AHQAQVHGPAYVGDDTFIGMGATVFKSRIGNGCVLEPGATVI |
| | GVTIPDGRYVPAGKTVTTQAEADALPVLTPDYAMYHTNETVV |
| | AVNLALAAAAKAAATAYIAPGAEVIGEVEIGANVMVSPMASI |
| | RSDEGMPIYLGDNTNVQDGVTLHGLEVEDEEGEEDESVYVEV |
| | NGKKYRVYIGNNVSLAHQAQVHGPAYVGDDTFIGMGATVFK |
| | SRIGNGCVLEPGATVIGVTIPDGRYVPAGKTVTTQAEADALPV |
| | LTPDYAMYHTNETVVAVNLALAAAAKAAATAYIAPGAEVIGE |
| | VEIGANVMVSPMASIRSDEGMPIYLGDNTNVQDGVTLHGLEV |
| | EDEEGEEDESVYVEVNGKKYRVYIGNNVSLAHQAQVHGPAY |
| | VGDDTFIGMGATVFKSRIGNGCVLEPGATVIGVTIPDGRYVPA |
| | GKTVTTQAEADALPVLTPDYAMYHTNETVVAVNLALAAAAK |
| | AAA |
| 417 | TAYIAPTAEVIGDVIIGDNVMISPNASIRSDEGMPIYIGENVNVQ |
| | DGVTITADRTKDEAGNDIPENWVTVNGKKYAVYLGKNVVLA |
| | HNATVNGRTVLGENVLVQENATLTASTLGENVIVQENATLTG |
| | VTVAEGKVVEAGKTITTQAEADKLKDLTKDHPLYNKNKEVV |
| | AKNLAILEEKKKLETAYIAPTAEVIGDVIIGDNVMISPNASIRSD |
| | EGMPIYIGENVNVQDGVTITADRTKDEAGNDIPENWVTVNGK |
| | KYAVYLGKNVVLAHNATVNGRTVLGENVLVQENATLTASTL |
| | GENVIVQENATLTGVTVAEGKVVEAGKTITTQAEADKLKDLT |
| | KDHPLYNKNKEVVAKNLAILEEKKKLETAYIAPTAEVIGDVIIG |
| | DNVMISPNASIRSDEGMPIYIGENVNVQDGVTITADRTKDEAG |
| | NDIPENWVTVNGKKYAVYLGKNVVLAHNATVNGRTVLGENV |
| | LVQENATLTASTLGENVIVQENATLTGVTVAEGKVVEAGKTIT |
| | TQAEADKLKDLTKDHPLYNKNKEVVAKNLAILEEKKKLE |
| 418 | TAYIAPTATVIGDVEIADNVMISPNASIRSDEGMPIYIGENANLQ |
| | DNVVLHALETKDEEGNDIEENWVEVDGKKYAVYIGRRVSLGH |
| | QAQIHGPALVGDDTFIGMNAKVFKSRIGNRCVLEPNAQVIGVT |
| | IPDGRYVPAGKVVTTQEEADKLPLLTPDYAMYHTNERVNAVN |
| | LALAAEARALATAYIAPTATVIGDVEIADNVMISPNASIRSDEG |
| | MPIYIGENANLQDNVVLHALETKDEEGNDIEENWVEVDGKKY |
| | AVYIGRRVSLGHQAQIHGPALVGDDTFIGMNAKVFKSRIGNRC |
| | VLEPNAQVIGVTIPDGRYVPAGKVVTTQEEADKLPLLTPDYAM |
| | YHTNERVNAVNLALAAEARALATAYIAPTATVIGDVEIADNV |
| | MISPNASIRSDEGMPIYIGENANLQDNVVLHALETKDEEGNDIE |
| | ENWVEVDGKKYAVYIGRRVSLGHQAQIHGPALVGDDTFIGMN |
| | AKVFKSRIGNRCVLEPNAQVIGVTIPDGRYVPAGKVVTTQEEA |
| | DKLPLLTPDYAMYHTNERVNAVNLALAAEARALA |
| 419 | TAYIHPTAEVIGDVEIGDNVMISPNASIRADEGMPIVIEENVNV |
| | QDGVEITALRSDLPEEEVEKLDLQEVDGKKVRAYFGKGAVLA |
| | HGAKILVASTRLKVEPVPGVTVLKQDNAVLRNVLLTEMHGLIL |
| | EVNAETGSIVIRESSDPALESKAKTWKVTPEDKAKIAAVIAANA |
| | AARQEALAAATAYIHPTAEVIGDVEIGDNVMISPNASIRADEG |
| | MPIVIEENVNVQDGVEITALRSDLPEEEVEKLDLQEVDGKKVR |
| | AYFGKGAVLAHGAKILVASTRLKVEPVPGVTVLKQDNAVLRN |
| | VLLTEMHGLILEVNAETGSIVIRESSDPALESKAKTWKVTPEDK |
| | AKIAAVIAANAAARQEALAAATAYIHPTAEVIGDVEIGDNVMI |
| | SPNASIRADEGMPIVIEENVNVQDGVEITALRSDLPEEEVEKLD |
| | LQEVDGKKVRAYFGKGAVLAHGAKILVASTRLKVEPVPGVTV |
| | LKQDNAVLRNVLLTEMHGLILEVNAETGSIVIRESSDPALESKA |
| | KTWKVTPEDKAKIAAVIAANAAARQEALAAA |
| 420 | TAYIEPNAEVIGDVKIGENVMISPNASIRSDEGMPIVIKENVNV |
| | QDGVVINAKLKKNENGEVDESQLNTINGEKVQIYLEKNVQLA |
| | HNVTIEDSVVLKENVLLQENVVLKNSTLGEGVVLAENVVIENV |
| | TLPENTVVEAGTVIKNQEEVKTLKQLTADSPAIVQLQAVLAKN |
| | AALWEELKAAETAYIEPNAEVIGDVKIGENVMISPNASIRSDEG |
| | MPIVIKENVNVQDGVVINAKLKKNENGEVDESQLNTINGEKV |
| | QIYLEKNVQLAHNVTIEDSVVLKENVLLQENVVLKNSTLGEGV |
| | VLAENVVIENVTLPENTVVEAGTVIKNQEEVKTLKQLTADSPA |
| | IVQLQAVLAKNAALWEELKAAETAYIEPNAEVIGDVKIGENV |
| | MISPNASIRSDEGMPIVIKENVNVQDGVVINAKLKKNENGEVD |
| | ESQLNTINGEKVQIYLEKNVQLAHNVTIEDSVVLKENVLLQEN |
| | VVLKNSTLGEGVVLAENVVIENVTLPENTVVEAGTVIKNQEEV |
| | KTLKQLTADSPAIVQLQAVLAKNAALWEELKAAE |
| 421 | TAYIHPSAEVIGDVTIGDNVMISPNASIRADEGMPIYLGDNANV |
| | QDGVTLHGLETKDEEGNIIEENLVEVNGKKYAVYVGDNVSLA |
| | HQAQIHGPAIVGDDTFIGMGATVRRSILGDGVLLGEGVQIENA |
| | TLPAGLCLGPGRVIRTPEELVDDCTEEQRAELKKKHAEVVAKN |
| | LALHEELKAAATAYIHPSAEVIGDVTIGDNVMISPNASIRADEG |
| | MPIYLGDNANVQDGVTLHGLETKDEEGNIIEENLVEVNGKKY |
| | AVYVGDNVSLAHQAQIHGPAIVGDDTFIGMGATVRRSILGDG |
| | VLLGEGVQIENATLPAGLCLGPGRVIRTPEELVDDCTEEQRAEL |
| | KKKHAEVVAKNLALHEELKAAATAYIHPSAEVIGDVTIGDNV |
| | MISPNASIRADEGMPIYLGDNANVQDGVTLHGLETKDEEGNIIE |
| | ENLVEVNGKKYAVYVGDNVSLAHQAQIHGPAIVGDDTFIGMG |
| | ATVRRSILGDGVLLGEGVQIENATLPAGLCLGPGRVIRTPEELV |
| | DDCTEEQRAELKKKHAEVVAKNLALHEELKAAA |
| 422 | TAYIAPNAQVIGEVTIGENVMISPNASIRSDEGMPIYIGENANLQ |
| | DNVVLHGLEVYTEEGELIEENLVEVDGKKYVVYIGKNVSLGH |
| | QAQIHGPAKVGDDTFIGMNAKVFKSVIGNRCVLEPNATVIGVT |
| | IPDGRYVPAGKVVTTQAEADALPVLTPDYALYHTNELVNEVN |
| | LALAAEGRAAATAYIAPNAQVIGEVTIGENVMISPNASIRSDEG |
| | MPIYIGENANLQDNVVLHGLEVYTEEGELIEENLVEVDGKKYV |
| | VYIGKNVSLGHQAQIHGPAKVGDDTFIGMNAKVFKSVIGNRC |
| | VLEPNATVIGVTIPDGRYVPAGKVVTTQAEADALPVLTPDYAL |
| | YHTNELVNEVNLALAAEGRAAATAYIAPNAQVIGEVTIGENV |
| | MISPNASIRSDEGMPIYIGENANLQDNVVLHGLEVYTEEGELIE |
| | ENLVEVDGKKYVVYIGKNVSLGHQAQIHGPAKVGDDTFIGMN |
| | AKVFKSVIGNRCVLEPNATVIGVTIPDGRYVPAGKVVTTQAEA |
| | DALPVLTPDYALYHTNELVNEVNLALAAEGRAAA |
| 423 | TAYIHPSAEVIGDVEIADNVMISPNASIRADEGMPIYLGENTNV |
| | QDGVSLHALENSSEEGEEDESNWVEVNGKKYRVYIGNNVSLG |
| | HQAQVHGPAIVGDDTFIGMGAKVFKSTIGNGCVLEPGVTVIGV |
| | TIPDGRYLEAGTVLRSQADIEKAKPIKEDMPTYKKVKEHKEKL |
| | KEEREKLKKERTAYIHPSAEVIGDVEIADNVMISPNASIRADEG |
| | MPIYLGENTNVQDGVSLHALENSSEEGEEDESNWVEVNGKKY |
| | RVYIGNNVSLGHQAQVHGPAIVGDDTFIGMGAKVFKSTIGNG |
| | CVLEPGVTVIGVTIPDGRYLEAGTVLRSQADIEKAKPIKEDMPT |
| | YKKVKEHKEKLKEEREKLKKERTAYIHPSAEVIGDVEIADNVM |
| | ISPNASIRADEGMPIYLGENTNVQDGVSLHALENSSEEGEEDES |
| | NWVEVNGKKYRVYIGNNVSLGHQAQVHGPAIVGDDTFIGMG |
| | AKVFKSTIGNGCVLEPGVTVIGVTIPDGRYLEAGTVLRSQADIE |
| | KAKPIKEDMPTYKKVKEHKEKLKEEREKLKKER |
| 424 | TAIIAPGATVIGEVHIADGVMISPNASIRADEGMPIYLGEYTNLQ |
| | DNVVLHALETYDEEGNLIEENLVEVNGKKYAVYVGKNVSLG |
| | HQAQLHGPTIVGDDTFIGMNAKVIRSTLGEGVVLEENVVVEGQ |
| | TLEKGTYLEKGMKLLTPEDLKKAKKIKEEDPVKKKLEAHIKEQ |
| | KAQAKAAQAAATAIIAPGATVIGEVHIADGVMISPNASIRADE |
| | GMPIYLGEYTNLQDNVVLHALETYDEEGNLIEENLVEVNGKK |
| | YAVYVGKNVSLGHQAQLHGPTIVGDDTFIGMNAKVIRSTLGE |
| | GVVLEENVVVEGQTLEKGTYLEKGMKLLTPEDLKKAKKIKEE |
| | DPVKKKLEAHIKEQKAQAKAAQAAATAIIAPGATVIGEVHIAD |
| | GVMISPNASIRADEGMPIYLGEYTNLQDNVVLHALETYDEEGN |
| | LIEENLVEVNGKKYAVYVGKNVSLGHQAQLHGPTIVGDDTFIG |
| | MNAKVIRSTLGEGVVLEENVVVEGQTLEKGTYLEKGMKLLTP |
| | EDLKKAKKIKEEDPVKKKLEAHIKEQKAQAKAAQAAA |
| 425 | TAYIHPSAEVIGEVEIGANVMVSPMASIRSDEGMPIKIGDNVNV |
| | QDGVVLHGLETKNEEGEEIEENLVEVDGEKYVVYLGKNVSLA |
| | HQAQVHGPSIVGDDTFIGMGAKVEGSTLGDGVFLGEGATVTG |
| | LTIPAGAVVSPGTVLTTPAQLASLKPLTADDPLLKKKKDVVEN |
| | NLATAAALKALETAYIHPSAEVIGEVEIGANVMVSPMASIRSD |
| | EGMPIKIGDNVNVQDGVVLHGLETKNEEGEEIEENLVEVDGEK |
| | YVVYLGKNVSLAHQAQVHGPSIVGDDTFIGMGAKVEGSTLGD |
| | GVFLGEGATVTGLTIPAGAVVSPGTVLTTPAQLASLKPLTADD |
| | PLLKKKKDVVENNLATAAALKALETAYIHPSAEVIGEVEIGAN |
| | VMVSPMASIRSDEGMPIKIGDNVNVQDGVVLHGLETKNEEGE |
| | EIEENLVEVDGEKYVVYLGKNVSLAHQAQVHGPSIVGDDTFIG |
| | MGAKVEGSTLGDGVFLGEGATVTGLTIPAGAVVSPGTVLTTP |
| | AQLASLKPLTADDPLLKKKKDVVENNLATAAALKALE |
| 426 | TAYIHPSASVIGDVEIADNVMISPNASIRADEGMPIKIGPNANV |
| | QDGVTLHGLETYDEEGNLIEENYVEVNGERYVVYIGDNVSLG |
| | HQAQIHGPAKVGDDTFIGMKATVFKSVIGNNCVLEPGATVIGV |
| | TIPDGRYVPAGKVVTTQAEADALPVLTPDYALYHTNERVNAV |
| | NLKLAEKARLEATAYIHPSASVIGDVEIADNVMISPNASIRADE |
| | GMPIKIGPNANVQDGVTLHGLETYDEEGNLIEENYVEVNGERY |
| | VVYIGDNVSLGHQAQIHGPAKVGDDTFIGMKATVFKSVIGNN |
| | CVLEPGATVIGVTIPDGRYVPAGKVVTTQAEADALPVLTPDYA |
| | LYHTNERVNAVNLKLAEKARLEATAYIHPSASVIGDVEIADNV |
| | MISPNASIRADEGMPIKIGPNANVQDGVTLHGLETYDEEGNLIE |
| | ENYVEVNGERYVVYIGDNVSLGHQAQIHGPAKVGDDTFIGMK |
| | ATVFKSVIGNNCVLEPGATVIGVTIPDGRYVPAGKVVTTQAEA |
| | DALPVLTPDYALYHTNERVNAVNLKLAEKARLEA |
| 427 | TAYIAPSAEVIGDVEIGANVMVSPMASIRADEGMPIYIGDNAN |
| | VQDGVVLHALETYDEEGNLIEEAYVEVDGKKYAVYVGDNVS |
| | LAHQAQIHGPAKVGEDTFIGMGAKVVGSTLGKGVFLAEGVVV |
| | ENATLPEGTILEKGTVVTPSDKELPKAPEELRAKLAAEHKAVV |
| | AANIAAAAAAKAAATAYIAPSAEVIGDVEIGANVMVSPMASIR |
| | ADEGMPIYIGDNANVQDGVVLHALETYDEEGNLIEEAYVEVD |
| | GKKYAVYVGDNVSLAHQAQIHGPAKVGEDTFIGMGAKVVGS |
| | TLGKGVFLAEGVVVENATLPEGTILEKGTVVTPSDKELPKAPE |
| | ELRAKLAAEHKAVVAANIAAAAAAKAAATAYIAPSAEVIGDV |
| | EIGANVMVSPMASIRADEGMPIYIGDNANVQDGVVLHALETY |
| | DEEGNLIEEAYVEVDGKKYAVYVGDNVSLAHQAQIHGPAKV |
| | GEDTFIGMGAKVVGSTLGKGVFLAEGVVVENATLPEGTILEKG |
| | TVVTPSDKELPKAPEELRAKLAAEHKAVVAANIAAAAAAKAA |
| | A |
| 428 | TAYIAPGAEVIGDVEIGANVMVSPMASIRADEGMPIYVGDNAN |
| | VQDGVVLHGLETLDEEGNLIEENWVEVDGKKYVVYLGKNVS |
| | LAHQAQIHGPAKVGEDTFIGMQALVFKSTIGNGCVLEPGAAVI |
| | GVTVPDGRYVPAGAVVTSQAEADALPKMTPDYAYAHTNETV |
| | VAVNNALAAGYKAAATAYIAPGAEVIGDVEIGANVMVSPMA |
| | SIRADEGMPIYVGDNANVQDGVVLHGLETLDEEGNLIEENWV |
| | EVDGKKYVVYLGKNVSLAHQAQIHGPAKVGEDTFIGMQALV |
| | FKSTIGNGCVLEPGAAVIGVTVPDGRYVPAGAVVTSQAEADA |
| | LPKMTPDYAYAHTNETVVAVNNALAAGYKAAATAYIAPGAE |
| | VIGDVEIGANVMVSPMASIRADEGMPIYVGDNANVQDGVVLH |
| | GLETLDEEGNLIEENWVEVDGKKYVVYLGKNVSLAHQAQIHG |
| | PAKVGEDTFIGMQALVFKSTIGNGCVLEPGAAVIGVTVPDGRY |
| | VPAGAVVTSQAEADALPKMTPDYAYAHTNETVVAVNNALAA |
| | GYKAAA |
| 429 | TAYIHPSATVIGQVNIGANVMVSPMASIRADEGMPITLEDNVN |
| | VQDGVLIQNESLKNESGEIDYSKVHPKNKRIESIVLKKNVSLAH |
| | QATVYSNTELSEGVFLQEGVVVKNSVIEGRVVLQRGVTVENV |
| | YIGEEVVIAEGTVLKGDEDLKKTTLAPLTPEQVAQIQAVIAQNL |
| | AAAAAAKAAATAYIHPSATVIGQVNIGANVMVSPMASIRADE |
| | GMPITLEDNVNVQDGVLIQNESLKNESGEIDYSKVHPKNKRIES |
| | IVLKKNVSLAHQATVYSNTELSEGVFLQEGVVVKNSVIEGRVV |
| | LQRGVTVENVYIGEEVVIAEGTVLKGDEDLKKTTLAPLTPEQV |
| | AQIQAVIAQNLAAAAAAKAAATAYIHPSATVIGQVNIGANVM |
| | VSPMASIRADEGMPITLEDNVNVQDGVLIQNESLKNESGEIDYS |
| | KVHPKNKRIESIVLKKNVSLAHQATVYSNTELSEGVFLQEGVV |
| | VKNSVIEGRVVLQRGVTVENVYIGEEVVIAEGTVLKGDEDLKK |
| | TTLAPLTPEQVAQIQAVIAQNLAAAAAAKAAA |
| 430 | TAYIAPGAQVIGDVEIADNVMISPNASIRADEGMPIYIGENANL |
| | QDNVQLHGLEVYTEEGELIEENFVEVDGKKYVVYIGRRVSLA |
| | HQAQVHGPAKVGDDTFIGMNAKVFKSIVGNRCVLEPNATVIG |
| | VTIPDGRYVPAGKTVTTQAEADALPVLTPDYALYHTNELVNA |
| | VNLALAAEARAAATAYIAPGAQVIGDVEIADNVMISPNASIRA |
| | DEGMPIYIGENANLQDNVQLHGLEVYTEEGELIEENFVEVDGK |
| | KYVVYIGRRVSLAHQAQVHGPAKVGDDTFIGMNAKVFKSIVG |
| | NRCVLEPNATVIGVTIPDGRYVPAGKTVTTQAEADALPVLTPD |
| | YALYHTNELVNAVNLALAAEARAAATAYIAPGAQVIGDVEIA |
| | DNVMISPNASIRADEGMPIYIGENANLQDNVQLHGLEVYTEEG |
| | ELIEENFVEVDGKKYVVYIGRRVSLAHQAQVHGPAKVGDDTFI |
| | GMNAKVFKSIVGNRCVLEPNATVIGVTIPDGRYVPAGKTVTTQ |
| | AEADALPVLTPDYALYHTNELVNAVNLALAAEARAAA |
| 431 | TAYIHPTARVIGEVTIAAGVMISPGASIRADEGMPIVIGENANV |
| | QDGVSLHGLEVYDEEGNLIEENLVEVNGEKYVVYIGENVSLG |
| | HQAQIHGPALVGDDTFIGMGAKVTRSILGEGVILEEGAQLTNVI |
| | VPDGAYVKSGQVFVSTGEPVVLSELKQTPEQKEKLAAQLAAE |
| | RAARAAAQAAATAYIHPTARVIGEVTIAAGVMISPGASIRADE |
| | GMPIVIGENANVQDGVSLHGLEVYDEEGNLIEENLVEVNGEKY |
| | VVYIGENVSLGHQAQIHGPALVGDDTFIGMGAKVTRSILGEGV |
| | ILEEGAQLTNVIVPDGAYVKSGQVFVSTGEPVVLSELKQTPEQ |
| | KEKLAAQLAAERAARAAAQAAATAYIHPTARVIGEVTIAAGV |
| | MISPGASIRADEGMPIVIGENANVQDGVSLHGLEVYDEEGNLIE |
| | ENLVEVNGEKYVVYIGENVSLGHQAQIHGPALVGDDTFIGMG |
| | AKVTRSILGEGVILEEGAQLTNVIVPDGAYVKSGQVFVSTGEP |
| | VVLSELKQTPEQKEKLAAQLAAERAARAAAQAAA |
| 432 | TAYIHPTAEVIGNVKIGENVMISPNASIRSDEGMPIVIKENANV |
| | QDGVVIRADPTKDENGNDIEENWVTVNGEKYAVYLEKNVVL |
| | AHNAVVEGRTVLKEGVLVQENAVVRRSTLGEGVILQENAVLE |
| | GVTVADGKIVPAGATIRTQAEADTLATLTPDHPLYNLNKVVN |
| | AKNLALLKENLAAKTAYIHPTAEVIGNVKIGENVMISPNASIRS |
| | DEGMPIVIKENANVQDGVVIRADPTKDENGNDIEENWVTVNG |
| | EKYAVYLEKNVVLAHNAVVEGRTVLKEGVLVQENAVVRRST |
| | LGEGVILQENAVLEGVTVADGKIVPAGATIRTQAEADTLATLT |
| | PDHPLYNLNKVVNAKNLALLKENLAAKTAYIHPTAEVIGNVKI |
| | GENVMISPNASIRSDEGMPIVIKENANVQDGVVIRADPTKDEN |
| | GNDIEENWVTVNGEKYAVYLEKNVVLAHNAVVEGRTVLKEG |
| | VLVQENAVVRRSTLGEGVILQENAVLEGVTVADGKIVPAGATI |
| | RTQAEADTLATLTPDHPLYNLNKVVNAKNLALLKENLAAK |
| 433 | TAIIAPGATVIGEVEIGDNVMISPNASIRSDEGMPIVLGEGANLQ |
| | DNVELHALEVYDEEGNLIEENYVEVNGKKYAVYIGNNVSLGH |
| | QAQIHGPAIVGDDTFIGMNAEVFKSIIGNGCVLEPNARVIGVTIP |
| | DGRYVKAGTTITDQAEIPSLKQLKDSDPIKAKVEAHKAALKAE |
| | RERLLAERTAIIAPGATVIGEVEIGDNVMISPNASIRSDEGMPIV |
| | LGEGANLQDNVELHALEVYDEEGNLIEENYVEVNGKKYAVYI |
| | GNNVSLGHQAQIHGPAIVGDDTFIGMNAEVFKSIIGNGCVLEP |
| | NARVIGVTIPDGRYVKAGTTITDQAEIPSLKQLKDSDPIKAKVE |
| | AHKAALKAERERLLAERTAIIAPGATVIGEVEIGDNVMISPNASI |
| | RSDEGMPIVLGEGANLQDNVELHALEVYDEEGNLIEENYVEV |
| | NGKKYAVYIGNNVSLGHQAQIHGPAIVGDDTFIGMNAEVFKSI |
| | IGNGCVLEPNARVIGVTIPDGRYVKAGTTITDQAEIPSLKQLKD |
| | SDPIKAKVEAHKAALKAERERLLAER |
| 434 | QEITVDEFSNIRENPVTPWNPEPSAPVIDPTAYIDPQASVIGEVTI |
| | GANVMVSPMASIRSDEGMPIFVGDRSNVQDGVVLHALETINEE |
| | GEPIEDNIVEVDGKEYAVYIGNNVSLAHQSQVHGPAAVGDDT |
| | FIGMQAFVFKSKVGNNCVLEPRSAAIGVTIPDGRYIPAGMVVT |
| | SQAEADKLPEVTDDYAYSHTNEAVVYVNVHLAEGYKETS |
| 435 | GKVYVKKPVFIPARHIPSDKTIIEPEIDEEAVIEEGAIITGGVIIKG |
| | RVYIASGATIRSDEGVPIVIEENSSIQDGALVHADETVDEDGNPI |
| | EENIVEVNGKPYAVYIGENVVLEHNATVHGPAAVGKNSLIGEG |
| | ALVRNSVIGENCVLEEGASAENVTIPAGRYVPAGVTVTTQAAA |
| | AALPAVTPDHPLYKRNEELVKENIEKAEKLLAEA |
| 436 | GSVLVEPSDIQCSPPNKYHKEPRCPTIAKGAYIEKGALIEGDVII |
| | EENVYIESGAIIRSDEGTPIYIGKNSVIQDGALVHADETVDEDG |
| | NPIEENIVEVNGKPYAVYIGENVVLEHNAEVHGPAAVGKNSLI |
| | GEGALVRNSIIGENCVLEEGASAENVTIPAGRYVPAGKTVTTQ |
| | AEAAALPKMTPDHPLYKRNEELVKENLEKVKKANAAA |
| 437 | GVVLVEEEGIRPSPATPRYPEPRAPIIHPSAYVADGALITGEVIIE |
| | DNVLIAEGAVIRSDEGRPIYIGKNSSVQDGAVIHADETVDAEGK |
| | EIEENIVEVNGKKYAVYIGENVVIEHGATVHGPAKIGENSLIGR |
| | GALVENSVIGKNCVLEEGASAIGVTIPEGRYIPAGVTVTTQEEA |
| | DALPEVTPDHPDYNRVAELVAKNIALAKELNAAR |
| 438 | PAPVRGHEAVFEDSLHPVTGKKLVTTIAETAYIEEGATISGAVI |
| | LADNVYVESGATIRSDEGIPIYVGENSAIQDGAVLHADETVDA |
| | DGNPIPENIVEVNGEPYAVYIGENVVLEHGATVHGPAAVGKNS |
| | LIGKNAVVRNSVVGENCVLEEGASAENVTIPAGRYVPAGKKV |
| | TTQEEADALPEVTPDHPDYKRNEKLVAENNAKVKAYNAAR |
| 439 | GVVLTPVSDIRPSAPTPRYKESKAPTIHPSAYIAPGATIVGDVTI |
| | AANVYVEAGATIRSDEGVPIYVGANSAVQDGAVLHADETVDE |
| | NGNPIEENIVEVNGKKYAVYVGENVVLEHGATVHGPAAIGAN |
| | SLVGEGALVANSIVGANCVLEPGASAINVTIPAGRYVPAGVTV |
| | TTQAAADALPAVTPDHPLANRNAELVAKNVAKAKAAVAAR |
| 440 | GTVVVATSPIRPSEPTPWRKESRAPTLAPGAYVHPDATVEGAV |
| | ILEEGALVQGGATIRSDEGVPIYVGRNSVIQDGATLHADETVDE |
| | EGNPIPENIVEVDGKPYAVYVGENVVIQHGATIHGPAAVGENS |
| | LIGENALVENSVVGKNCVIQEGGAARNVTIPEGRYIPAGKTVT |
| | TQAEADALPKVTPDHPYYNKNAALVAENLARRAELLAAR |
| 441 | MVVLVEEAGLRPSPPTPRHREPRAPTLAEGAWVAPGATIEGEV |
| | HIAAGAYIADGATIRSDEGTPIYVGANSVIQDGALLHADETVDL |
| | DGKVIEENVVTVNGEPYAVYIGENVVIEHNATIHGPAAVGANS |
| | LIGENALVRHSIIGENCVLEEGASAINVTIPAGRYVPAGKTVTT |
| | QAEAAALPKVTPDHPNYNKNAALVAENLALNKALVAAA |
| 442 | MLLVEEEPLIRPSEPTPWRGTRREPTIAEGAYIEPGAVITGDTIIE |
| | AGAYIESGAVIRSDEGVPIYIGANSAIQDGAVLHADETVDENG |
| | NLIEENVVEVNGKKYAVYVGANVVIEHNAVIHGPAAVGANSL |
| | IGEGAVVRNSIVGANCVLEPGASVENVTVPAGRYVPAGVRVT |
| | TQAAADALPAVTPDHPLANRNAELVAKNVAKVKAANAAK |
| 443 | GLKKRKLTPAELRKPDPRYTGPRVSTIGETCLFAPGAVISPGVT |
| | LGENVYIESGAVIRSDEGRPIVVGDNSVIQDGAVLHADETVDA |
| | DGNPIPENIVTVNGQPYAVYVGSNVVIDHGATIHGPAAVGANT |
| | LIGEGATVRNSTVGSNCVLEPGASAIGVTIPAGRYVPAGKTVTT |
| | QAEADALPAVTPDHPYANRVAELVAKNLAKVKAANAAR |
| 444 | GVVVAPVSDIQCSPPTPRFPESRCPTLHPSAYIEEGATIIGEVTIG |
| | DNVLIEKGATIRSDEGVPIYIGENSSIQDGATLHADETVDEAGN |
| | PIEENIVTVNGKPYAVYIGENVVIEHGATVHGPAAIGRNSLIGE |
| | GATVRNSIVGENCVLEEGASAIGVTIPAGRYIPAGKVVTTQEEA |
| | AALPEVTPDHPNYKRVEELVAKNIALVKALLAAR |
| 445 | TAYIHPSATVIGDVEIADNAMISPNASIRADEGMPIKIEKNAVV |
| | QDNATIEAKPTKDADGNLIEENIVEVNGKKYAVYIGEGAVLQK |
| | NATLEGGTIIGKNVLVQENATLTNSTLGENVIVQENATLTGVTI |
| | AEGKIVPEGATITTQEEAEKLAPLTPDHPLYNKRAEIVAKALAE |
| | REAALAAA |
| 446 | TAYIHPSAKVIGDVEIGANVMVSPMASIRSDEGMPIKIEENANV |
| | QDGVKIEGKRVYSASGELIEENIVEVNGKKYVVYIGENVSLAH |
| | QATIIGGTVLKKNVFIQEGAYIENSVLGENVIVQKNATIIGVTIK |
| | EGKVVPEGAVITTQEEADKLPKLTPEHPLYNLNAQVVAANIAK |
| | AAALKAAE |
| 447 | TAYIHPTATVIGNVKIGENVMISPEASLRADEGMPIVLEENVNV |
| | QDGVVIRGDRVLDEAGNLIEENLVEVNGERYVVYLGKGVVLA |
| | HGAVVEGSTVLGEGVLVQEGAVLRRSTLGERVIVQEGAVLEG |
| | VTVAPGAVVPAGAVIRTQAEAATLAALTPDHPLYNENARVVA |
| | KNLALLEELKALE |
| 448 | TAYIHPSATVIGDVIIGKNAMISPNASIRSDEGMPIVLEENAIVQ |
| | DNATITAKPTKTASGELIEENVVEVNGKKYAVYLGENAILQKN |
| | ATIEGGTVVGKNVLVQENATLTGSTLGENVIVQENATITGVTIA |
| | EGAIVPEGATITTQEEAEKLEKLTPDHPLYNLNAELNAKALAL |
| | RDLLLALS |
| 449 | TAYIHPSATVIGDVEIGENAMISPNASIRSDEGMPIVLEKNVVV |
| | QDNATIEANPVLDENGELIEENVREVNGKKYAVYLEEGVVLQ |
| | KNATIKGGTVVKKNVLVQENATLTNSTLGENVIVQENATLTG |
| | VTVAEGKVVPEGATITTQAEAEKLAPLTPDHPLYNLGAEIRAK |
| | ALALRELKLALE |
| 450 | TAYIHPSAKVEGEVEIGANVMVSPMATINSTEGEPIFLGDRVNV |
| | QDGVTITCEPVYNADGELDESKLVEIDGKKYCVYVAENVSLA |
| | HQSTLTSGTVLKKNVFVQEGAVLKNSTLGENVVVRENAVIEG |
| | VTIEEGTVAEEGTVVLTEEDLAKLRPLTPEDPLLEIHKKVVEEN |
| | IAKAKALKAAA |
| 451 | TAYIHPSAEVIGDVEIGANVMISPNASIRADEGMPIVLGDNTNV |
| | QDGVSLHALETLDEEGNLIEENVVEVDGKKYAVYVGDNVSLA |
| | HQAQVHGPAIVGEDTFIGMGAVVRRSVLGKGVILREGATVEG |
| | VTIEEGTVVEENTVLTKQEEVKKLKKLTPEHPYYNLNKKVVE |
| | QNIKKVQAARAAA |
| 452 | TAVIHPSATVIGDVTIADNVMISPNASIRADEGMPIVLGENVNL |
| | QDNVSLHALETLDEEGNLIEENVVEVNGKKYAVYLGEGVSLA |
| | HQAQVHGPAIVGKDTFIGMNAKVSGSILGEGVILQDNATVEGA |
| | TIAEGKVVPEGAVITTQEEADKLAPLTPDHPYYELVKRTREEN |
| | LRLRDLLLELE |
| 453 | TAYIHPSAKVIGDVEIGENVMISPNASIRADEGMPIKLEKNVIV |
| | QDNAVIEADRIYDENGELIESAVVTVNGKKYAVYLGENVILQK |
| | NATVRGGTVLGKNVLVQENAVLTNSTLGENVIVQENATVTGA |
| | TLKEGTIVPEGATITTQEEADKLEKLTPDHPLYNLHAALLAAGL |
| | ALRDLLLALE |
| 454 | TAYIHPSAEVIGDVTIGANVMVSPMASIRADEGMPIVLGNNVN |
| | VQDGVVLHGLEVLNEEGELIEENVVEVDGKKYVVYVGEGVSL |
| | AHQATVVGATVLGKGVFVGEGVVLERSILGEGVIVGENAVIK |
| | GVTIAEGKVVKEGTTVTTQEEADKLEKLTPDHPYYELNARVV |
| | AENIAKARLLKLLE |
| 455 | TAYIAPGASVIGEVEIGDNVMISPNASLRSDEGMPIKLGDNVNV |
| | QDGVTLHGLETKDEEGNLIEENVVEVNGEKYVVYVGKNVVL |
| | AHNVTLTSRTVVEDNVYLEENVTLTRSTLGKYVYVEKGVVIE |
| | GVTIKEGMYAKEGTVIRTQEDVKSLEMIKDLAKHKAKVQAVI |
| | DANLRIHQEALAAATAYIAPGASVIGEVEIGDNVMISPNASLRS |
| | DEGMPIKLGDNVNVQDGVTLHGLETKDEEGNLIEENVVEVNG |
| | EKYVVYVGKNVVLAHNVTLTSRTVVEDNVYLEENVTLTRSTL |
| | GKYVYVEKGVVIEGVTIKEGMYAKEGTVIRTQEDVKSLEMIK |
| | DLAKHKAKVQAVIDANLRIHQEALAAATAYIAPGASVIGEVEI |
| | GDNVMISPNASLRSDEGMPIKLGDNVNVQDGVTLHGLETKDE |
| | EGNLIEENVVEVNGEKYVVYVGKNVVLAHNVTLTSRTVVEDN |
| | VYLEENVTLTRSTLGKYVYVEKGVVIEGVTIKEGMYAKEGTVI |
| | RTQEDVKSLEMIKDLAKHKAKVQAVIDANLRIHQEALAAA |
| 456 | TAYIHPTATVIGSVTIADGVMISPYASIRADEGMPIYIGEGANV |
| | QDGVQLHGLETRDEEGNLIEENVVEVNGKKYVVYIGKGVSLG |
| | HQAQVHGPAIVGDDTFIGMGAQVTGAILPEGLVLAEGVRVES |
| | VDLSGYRYLPPGTVIRTQADKERVREDESLAAEVEARRAALA |
| | AERAAAEAARAAATAYIHPTATVIGSVTIADGVMISPYASIRAD |
| | EGMPIYIGEGANVQDGVQLHGLETRDEEGNLIEENVVEVNGK |
| | KYVVYIGKGVSLGHQAQVHGPAIVGDDTFIGMGAQVTGAILP |
| | EGLVLAEGVRVESVDLSGYRYLPPGTVIRTQADKERVREDESL |
| | AAEVEARRAALAAERAAAEAARAAATAYIHPTATVIGSVTIAD |
| | GVMISPYASIRADEGMPIYIGEGANVQDGVQLHGLETRDEEGN |
| | LIEENVVEVNGKKYVVYIGKGVSLGHQAQVHGPAIVGDDTFIG |
| | MGAQVTGAILPEGLVLAEGVRVESVDLSGYRYLPPGTVIRTQA |
| | DKERVREDESLAAEVEARRAALAAERAAAEAARAAA |
| 457 | TAFIHPSATVIGDVTIGANVMISPMASIRSDEGMPIYVGANANL |
| | QDQVTLHALEVFDEEGNLIEENWVEVNGEKYAVYLGDNVSLA |
| | HQAQVHGPAIVGEDTFIGMQASVFKSTIGNGCVLEPGAAVIGV |
| | TIPDGRYVPAGKVVTSQAEADALPKMTPDYAYYHTNEKVVA |
| | VNRALAAGYRAAQTAFIHPSATVIGDVTIGANVMISPMASIRS |
| | DEGMPIYVGANANLQDQVTLHALEVFDEEGNLIEENWVEVNG |
| | EKYAVYLGDNVSLAHQAQVHGPAIVGEDTFIGMQASVFKSTI |
| | GNGCVLEPGAAVIGVTIPDGRYVPAGKVVTSQAEADALPKMT |
| | PDYAYYHTNEKVVAVNRALAAGYRAAQTAFIHPSATVIGDVT |
| | IGANVMISPMASIRSDEGMPIYVGANANLQDQVTLHALEVFDE |
| | EGNLIEENWVEVNGEKYAVYLGDNVSLAHQAQVHGPAIVGE |
| | DTFIGMQASVFKSTIGNGCVLEPGAAVIGVTIPDGRYVPAGKV |
| | VTSQAEADALPKMTPDYAYYHTNEKVVAVNRALAAGYRAA |
| | Q |
| 458 | TAYIHPSATVIGDVTIGANVMVSPMASIRSDEGMPIYLGDNVN |
| | VQDGVSLHGLETVDEEGNVIEENLVEVDGKKYVVYLGDNVSL |
| | AHQAVVEGGTVLGENVFLQEGVRVRRSTLGEGVILREGATVE |
| | GVTIAPGKVVAAGQTVTTQAAADALPTLTASDPLYSEHATVV |
| | AANIAAAAAAKAAATAYIHPSATVIGDVTIGANVMVSPMASIR |
| | SDEGMPIYLGDNVNVQDGVSLHGLETVDEEGNVIEENLVEVD |
| | GKKYVVYLGDNVSLAHQAVVEGGTVLGENVFLQEGVRVRRS |
| | TLGEGVILREGATVEGVTIAPGKVVAAGQTVTTQAAADALPTL |
| | TASDPLYSEHATVVAANIAAAAAAKAAATAYIHPSATVIGDVT |
| | IGANVMVSPMASIRSDEGMPIYLGDNVNVQDGVSLHGLETVD |
| | EEGNVIEENLVEVDGKKYVVYLGDNVSLAHQAVVEGGTVLG |
| | ENVFLQEGVRVRRSTLGEGVILREGATVEGVTIAPGKVVAAGQ |
| | TVTTQAAADALPTLTASDPLYSEHATVVAANIAAAAAAKAAA |
| 459 | TAYIAPSATVIGDVTIGANVMISPMASIRADEGMPIKVGDNAN |
| | VQDQVTLHALETKDEEGNDIEENWVEVNGEKYAVYVGANVS |
| | LAHQAQLHGPCIVGDDTFIGMQARVFKSSIGNGCVLEPQAAVI |
| | GVTIPDGRYVPAGAVVTSQAEADALPKLTDDYAYAHTNEKVV |
| | AVNLALAKGYLTTPTAYIAPSATVIGDVTIGANVMISPMASIRA |
| | DEGMPIKVGDNANVQDQVTLHALETKDEEGNDIEENWVEVN |
| | GEKYAVYVGANVSLAHQAQLHGPCIVGDDTFIGMQARVFKSS |
| | IGNGCVLEPQAAVIGVTIPDGRYVPAGAVVTSQAEADALPKLT |
| | DDYAYAHTNEKVVAVNLALAKGYLTTPTAYIAPSATVIGDVTI |
| | GANVMISPMASIRADEGMPIKVGDNANVQDQVTLHALETKDE |
| | EGNDIEENWVEVNGEKYAVYVGANVSLAHQAQLHGPCIVGD |
| | DTFIGMQARVFKSSIGNGCVLEPQAAVIGVTIPDGRYVPAGAV |
| | VTSQAEADALPKLTDDYAYAHTNEKVVAVNLALAKGYLTTP |
| 460 | TAYIHPSAVVIGQVEIGANVMVSPMASIRSDEGMPIKIEANANV |
| | QDGVLIQSLVSKENDKELLEKLKKLNNGEYYNIYLEEGVSLAH |
| | QATILNSCYLSSGCFLAEGVVLENSVLNDAVFLGRGVTVTNAE |
| | VLEPHVFEAGDVITEEKVEPVEIPEELRAAIAAQRAAVIAANLA |
| | AAAAAKAAATAYIHPSAVVIGQVEIGANVMVSPMASIRSDEG |
| | MPIKIEANANVQDGVLIQSLVSKENDKELLEKLKKLNNGEYYN |
| | IYLEEGVSLAHQATILNSCYLSSGCFLAEGVVLENSVLNDAVFL |
| | GRGVTVTNAEVLEPHVFEAGDVITEEKVEPVEIPEELRAAIAAQ |
| | RAAVIAANLAAAAAAKAAATAYIHPSAVVIGQVEIGANVMVS |
| | PMASIRSDEGMPIKIEANANVQDGVLIQSLVSKENDKELLEKL |
| | KKLNNGEYYNIYLEEGVSLAHQATILNSCYLSSGCFLAEGVVL |
| | ENSVLNDAVFLGRGVTVTNAEVLEPHVFEAGDVITEEKVEPVE |
| | IPEELRAAIAAQRAAVIAANLAAAAAAKAAA |
| 461 | TAYIHPQANVIGDVEIGANVMVSPMASIRSDEGMPIFVGENAN |
| | VQDQVTLHALETYDEEGNPIEENIVEVDGKKYAVYLGKNVSL |
| | AHQAQIHGPSIVGDDTFIGMQALVFKSVLGNNCVLEPQAAAIG |
| | VTIPDGRYIPAGKVVTSQAEADALPEVTPDYAYYHTNEQVVY |
| | VNTQLAEGYRAAATAYIHPQANVIGDVEIGANVMVSPMASIRS |
| | DEGMPIFVGENANVQDQVTLHALETYDEEGNPIEENIVEVDGK |
| | KYAVYLGKNVSLAHQAQIHGPSIVGDDTFIGMQALVFKSVLG |
| | NNCVLEPQAAAIGVTIPDGRYIPAGKVVTSQAEADALPEVTPD |
| | YAYYHTNEQVVYVNTQLAEGYRAAATAYIHPQANVIGDVEIG |
| | ANVMVSPMASIRSDEGMPIFVGENANVQDQVTLHALETYDEE |
| | GNPIEENIVEVDGKKYAVYLGKNVSLAHQAQIHGPSIVGDDTFI |
| | GMQALVFKSVLGNNCVLEPQAAAIGVTIPDGRYIPAGKVVTSQ |
| | AEADALPEVTPDYAYYHTNEQVVYVNTQLAEGYRAAA |
| 462 | TAYIHPSAEVIGSVEIGENVMISPNASIRSDEGMPIVIGDNANVQ |
| | DGVTLHGLETKTEEGELIEENYVEVDGKKYVVYIGENVSLAH |
| | QAQVHGPAKVGEDTFIGMGATVTQSILGEGVLLREGAQITGVT |
| | LAPGTVVDRGTVLTTQADVASLRKLEPSDPLLKENEEVRKKN |
| | LALWEELKKAETAYIHPSAEVIGSVEIGENVMISPNASIRSDEG |
| | MPIVIGDNANVQDGVTLHGLETKTEEGELIEENYVEVDGKKY |
| | VVYIGENVSLAHQAQVHGPAKVGEDTFIGMGATVTQSILGEG |
| | VLLREGAQITGVTLAPGTVVDRGTVLTTQADVASLRKLEPSDP |
| | LLKENEEVRKKNLALWEELKKAETAYIHPSAEVIGSVEIGENV |
| | MISPNASIRSDEGMPIVIGDNANVQDGVTLHGLETKTEEGELIE |
| | ENYVEVDGKKYVVYIGENVSLAHQAQVHGPAKVGEDTFIGM |
| | GATVTQSILGEGVLLREGAQITGVTLAPGTVVDRGTVLTTQAD |
| | VASLRKLEPSDPLLKENEEVRKKNLALWEELKKAE |
| 463 | TAVIAPNAQVIGEVHIGDNVMISPNASIRSDEGMPIYIGENANL |
| | QDNVQLHGLEVLDEEGNVIEEALVEVDGKKYVVYIGKNVSLG |
| | HQAQIHGPALVGDDTFIGMNAKVFKSRIGNGCVLEPNAQVIGV |
| | TIPDGRYVPAGKVVTTQAEADALPVLTPDYAMAHTNERVVAV |
| | NLALAAAARAAATAVIAPNAQVIGEVHIGDNVMISPNASIRSD |
| | EGMPIYIGENANLQDNVQLHGLEVLDEEGNVIEEALVEVDGK |
| | KYVVYIGKNVSLGHQAQIHGPALVGDDTFIGMNAKVFKSRIG |
| | NGCVLEPNAQVIGVTIPDGRYVPAGKVVTTQAEADALPVLTPD |
| | YAMAHTNERVVAVNLALAAAARAAATAVIAPNAQVIGEVHIG |
| | DNVMISPNASIRSDEGMPIYIGENANLQDNVQLHGLEVLDEEG |
| | NVIEEALVEVDGKKYVVYIGKNVSLGHQAQIHGPALVGDDTFI |
| | GMNAKVFKSRIGNGCVLEPNAQVIGVTIPDGRYVPAGKVVTT |
| | QAEADALPVLTPDYAMAHTNERVVAVNLALAAAARAAA |
| 464 | TAYIHPQATVIGDVTIGANVMVSPMASIRSDEGMPIFVGDNAN |
| | VQDGVTLHALETYDEEGNPIEENWVEVDGKKYAVYLGDNVS |
| | LAHQAQVHGPAAVGEDTFIGMQATVFKSKLGNNCVLEPGAA |
| | AIGVTIPDGRYIPAGKVVTSQAEADALPEVTPDYAYYHTNEDV |
| | VYVNIALAEGYKKLSTAYIHPQATVIGDVTIGANVMVSPMASI |
| | RSDEGMPIFVGDNANVQDGVTLHALETYDEEGNPIEENWVEV |
| | DGKKYAVYLGDNVSLAHQAQVHGPAAVGEDTFIGMQATVFK |
| | SKLGNNCVLEPGAAAIGVTIPDGRYIPAGKVVTSQAEADALPE |
| | VTPDYAYYHTNEDVVYVNIALAEGYKKLSTAYIHPQATVIGD |
| | VTIGANVMVSPMASIRSDEGMPIFVGDNANVQDGVTLHALET |
| | YDEEGNPIEENWVEVDGKKYAVYLGDNVSLAHQAQVHGPAA |
| | VGEDTFIGMQATVFKSKLGNNCVLEPGAAAIGVTIPDGRYIPA |
| | GKVVTSQAEADALPEVTPDYAYYHTNEDVVYVNIALAEGYK |
| | KLS |

The enzyme comprising silicase activity may be specific or substantially specific to a substrate in the silicate such that the enzyme acts specifically on that substrate and facilitates extracting the metal from the mineral material. In some cases, the enzyme having silicase activity may degrade, digest, and/or disintegrate the silicate. As a result, metals, such as in the form of metal ions, metal atoms, or metal precipitates, may be released from the mineral material (e.g., into a solution). In some cases, the method may comprise collecting the metal or the solution containing the metal. In some cases, the method may comprise separating the metal from the solution. In some cases, the metal may be water soluble. Alternatively or in addition, the metal may precipitate in the solution. The solution may be an aqueous solution comprising water and/or a buffer described anywhere herein.

In some embodiments, the enzyme having silicase activity has a pKd of at least about 5, at least about 6, at least about 7, at least about 8, at least about 9, at least about 10, at least about 11, or higher. In some embodiments, the enzyme having silicase activity has a Kcat value of at least about 2 mol per second (mol/s), at least about 10 mol/s, at least about 50 mol/s, at least about 100 mol/s, at least about 200 mol/s, at least about 300 mol/s, at least about 400 mol/s, at least about 500 mol/s, at least about 600 mol/s, at least about 700 mol/s, at least about 800 mol/s, at least about 900 mol/s, at least about 1000 mol/s, or higher.

In some cases, the specificity of the enzyme having silicate activity to the substrate may be higher than a wild-type version of the enzyme. For example, a wild-type enzyme may be engineered to improve its specificity for a substrate. In some cases, the enzyme provided herein may be engineered by directed evolution. In some cases, machine learning and artificial intelligence may be used for performing such enzyme engineering and directed evolution methods to design the enzymes of the present disclosure. The engineered enzyme may be synthesized or semi-synthesized. In some embodiments, the enzyme (e.g., the engineered enzyme) has the sequence of any one of SEQ ID NOS: 19-402, or an amino acid sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more sequence identity to any one of SEQ ID NOS: 19-402. In some embodiments, the enzyme (e.g., the engineered enzyme) has the sequence of any one of SEQ ID NOS: 403-464, or an amino acid sequence having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more sequence identity to any one of SEQ ID NOS: 403-464.

In some embodiments, the enzyme having silicase activity comprises a purification tag. The purification tag can comprise commonly used tags known in the art to aide in the purification of the enzyme having silicase activity from a host cell. The purification tag can comprise, for example, a GST tag, a His tag, a NEXT tag, a FLAG tag, or any other tags known in the art. In some cases, the purification tag is conjugated to the N-terminus of the enzyme having silicase activity. In some cases, the purification tag is conjugated to the C-terminus of the enzyme having silicase activity. In some cases, the purification tag is cleaved from the enzyme having silicase activity after purification. In some cases, the purification tag is not cleaved from the enzyme having silicase activity after purification. In some cases, the purification tag aides in enzyme stability. In some cases, the purification tag does not affect enzymatic efficacy. In some cases, the purification tag does not affect the enzyme having silicase activity polymerization.

In some cases, the enzyme having silicase activity may have a catalytic activity that is superior to the wild-type version of the same enzyme. For example, the enzymes provided herein may have a higher catalytic rate. This may decrease the time required to extract a given amount of metal from the mineral material, compared to when using a wild-type enzyme. In some cases, the enzymes provided herein may have reduced energy requirements, as compared to a wild-type enzyme. In some cases, catalytic activity/rate/efficiency may be characterized by using one or more metrics.

In some embodiments, the enzyme having silicase activity has a catalytic efficiency of at least about 80%, at least about 85%, at least about 90%, at least about 91%, at least about 92%, at least about 93%, at least about 94%, at least about 95%, at least about 98% at least about 99%, at least about 99.5% or higher. In some embodiments, the method has a maximum metal extraction rate of at least about 80%, at least about 85%, at least about 90%, at least about 91%, at least about 92%, at least about 93%, at least about 94%, at least about 95%, at least about 98% at least about 99%, at least about 99.5% or higher. In some embodiments, the enzyme having silicase activity has a catalytic efficiency of at least about 1.1, 1.2, 1.3, 1.4, 1.5, 1.6, 1.7, 1.8, 1.9, 2, 2.25, 2.5, 2.75, 3, 3.25, 3.5, 3.75, 4, 4.25, 4.5, 4.75, 5, 5.5, 6, 6.5, 7, 8, 9, 10 times or higher. In some embodiments, the method has a maximum metal extraction rate of at least about 1.1, 1.2, 1.3, 1.4, 1.5, 1.6, 1.7, 1.8, 1.9, 2, 2.25, 2.5, 2.75, 3, 3.25, 3.5, 3.75, 4, 4.25, 4.5, 4.75, 5, 5.5, 6, 6.5, 7, 8, 9, 10 times or higher.

In some embodiments, the enzyme having silicase activity has a pKd of at least about 5, at least about 6, at least about 7, at least about 8, at least about 9, at least about 10, at least about 11, or higher. In some embodiments, the enzyme having silicase activity has a Kcat value of at least about 2 mol per second (mol/s), at least about 10 mol/s, at least about 50 mol/s, at least about 100 mol/s, at least about 200 mol/s, at least about 300 mol/s, at least about 400 mol/s, at least about 500 mol/s, at least about 600 mol/s, at least about 700 mol/s, at least about 800 mol/s, at least about 900 mol/s, at least about 1000 mol/s, or higher.

The methods of the present disclosure, such as the enzymatic reactions provided herein, may be performed under a set of reaction conditions. In some cases, the reaction conditions may comprise a reaction temperature (e.g., a temperature under which the enzymatic reaction, or a portion thereof, is performed). In some cases, the reaction temperature is from about 20 to about 90 degrees Celsius (C). In some cases, the reaction temperature is from about 23 to about 90 degrees Celsius (C). In some cases, the reaction temperature is from about 23 to about 85 degrees Celsius (C). In some cases, the reaction temperature is from about 30 to about 90 degrees Celsius (C). In some cases, the reaction conditions comprise a temperature from about 30 to about 80 degrees Celsius (C). In some cases, the reaction conditions comprise a temperature from about 30 to about 70 degrees Celsius (C). In some cases, the reaction conditions comprise a temperature from about 30 to about 60 degrees Celsius (C). In some cases, the reaction conditions comprise a temperature from about 30 to about 50 degrees Celsius (C). In some cases, the reaction conditions comprise a temperature from about 45 to about 55 degrees Celsius (C). In some cases, the reaction conditions comprise a temperature from about 45 to about 50 degrees Celsius (C). In some cases, the reaction conditions comprises a temperature about 20 degrees Celsius (C). In some cases, the reaction conditions comprises a temperature about 23 degrees Celsius (C). In some cases, the reaction conditions comprises a temperature about 25 degrees Celsius (C). In some cases, the reaction conditions comprises a temperature about 30 degrees Celsius (C). In some cases, the reaction conditions comprises a temperature about 35 degrees Celsius (C). In some cases, the reaction conditions comprises a temperature about 40 degrees Celsius (C). In some cases, the reaction conditions comprises a temperature about 45 degrees Celsius (C). In some cases, the reaction conditions comprises a temperature about 50 degrees Celsius (C). In some cases, the reaction conditions comprises a temperature about 55 degrees Celsius (C). In some cases, the reaction conditions comprises a temperature about 60 degrees Celsius (C). In some cases, the reaction conditions comprises a temperature about 70 degrees Celsius (C). In some cases, the reaction conditions comprises a temperature about 75 degrees Celsius (C). In some cases, the reaction conditions comprises a temperature about 80 degrees Celsius (C). In some cases, the reaction conditions comprises a temperature about 85 degrees Celsius (C). In some cases, the methods of the present disclosure may be performed in near-ambient temperatures and/or pressures. In some cases, the temperature ranges of the methods of the present disclosure may be significantly lower than temperatures required in acid roasting. (e.g., 200 degrees Celsius (C) and above). This may reduce the energy requirements of the process performed using the methods of the present disclosure

In some cases, the enzymatic reaction may be performed at a pH from about 4 to about 11. In some cases, the enzymatic reaction may be performed at a pH from about 4 to about 10. In some cases, the enzymatic reaction may be performed at a pH from about 4 to about 9. In some cases, the enzymatic reaction may be performed at a pH from about 4 to about 8. In some cases, the enzymatic reaction may be performed at a pH from about 4 to about 7. In some cases, the enzymatic reaction may be performed at a pH from about 4 to about 6. In some cases, the enzymatic reaction may be performed at a pH from about 5 to about 11. In some cases, the enzymatic reaction may be performed at a pH from about 5 to about 10. In some cases, the enzymatic reaction may be performed at a pH from about 5 to about 9. In some cases, the enzymatic reaction may be performed at a pH from about 5 to about 8. In some cases, the enzymatic reaction may be performed at a pH from about 5 to about 7. In some cases, the enzymatic reaction may be performed at a pH from about 5 to about 6. In some cases, the enzymatic reaction may be performed at a pH from about 6 to about 11. In some cases, the enzymatic reaction may be performed at a pH from about 6 to about 10. In some cases, the enzymatic reaction may be performed at a pH from about 7 to about 11. In some cases, the enzymatic reaction may be performed at a pH from about 7 to about 10. In some cases, the enzymatic reaction may be performed at a pH from about 8 to about 11. In some cases, the enzymatic reaction may be performed at a pH from about 8 to about 10. In some cases, the enzymatic reaction may be performed at a pH from about 9 to about 11. In some cases, the enzymatic reaction may be performed at a pH from about 9 to about 10. In some cases, the enzymatic reaction may be performed at a pH of 4. In some cases, the enzymatic reaction may be performed at a pH of 5. In some cases, the enzymatic reaction may be performed at a pH of 6. In some cases, the enzymatic reaction may be performed at a pH of 7. In some cases, the enzymatic reaction may be performed at a pH of 8. In some cases, the enzymatic reaction may be performed at a pH of 9. In some cases, the enzymatic reaction may be performed at a pH of 10. In some cases, the enzymatic reaction may be performed at a pH of 11. In some cases, the methods of the present disclosure may be performed in pH ranges that are substantially neutral, or in other words, not highly/strongly acidic or highly/strongly basic. In some cases, the method/reaction of the present disclosure may not comprise using a strong acid, such as is used in acid leaching. Instead, the enzymes presented throughout the disclosure may perform enzymatic degradation of the mineral material in neutral pH conditions.

In some cases, the methods provided herein comprise crushing or grinding the rocks or ores to achieve a particulate size. In some cases, the ground ores comprise a size of about 50 μm to 1 mm. In some cases, the ground ores comprise a size from about 50 μm to 750 μm. In some cases, the ground ores comprise a size from about 50 μm to 500 μm. In some cases, the ground ores comprise a size from about 50 μm to 250 μm. In some cases, the ground ores comprise a size from about 50 μm to 150 μm. In some cases, the ground ores comprise a size from about 50 μm to 100 μm. In some cases, the ground ores comprise a size of about 50 μm. In some cases, the ground ores comprise a size of about 100 μm. In some cases, the ground ores comprise a size of about 150 μm. In some cases, the ground ores comprise a size of about 250 μm. In some cases, the ground ores comprise a size of about 500 μm. In some cases, the ground ores comprise a size of about 750 μm. In some cases, the ground ores comprise a size of about 1 mm.

In some cases, the methods provided herein comprise creating a slurry of crushed rock and liquid. In some cases, the rock to liquid ratio is from about 1-40% (w/v). In some cases, the rock to liquid ratio is from about 1-35% (w/v). In some cases, the rock to liquid ratio is from about 1-30% (w/v). In some cases, the rock to liquid ratio is from about 1-25% (w/v). In some cases, the rock to liquid ratio is from about 1-20% (w/v). In some cases, the rock to liquid ratio is from about 1-15% (w/v). In some cases, the rock to liquid ratio is from about 1-10% (w/v). In some cases, the rock to liquid ratio is from about 1-5% (w/v). In some cases, the rock to liquid ratio is from about 10-40% (w/v). In some cases, the rock to liquid ratio is from about 10-35% (w/v). In some cases, the rock to liquid ratio is from about 10-30% (w/v). In some cases, the rock to liquid ratio is from about 10-25% (w/v). In some cases, the rock to liquid ratio is from about 15-35% (w/v). In some cases, the rock to liquid ratio is from about 15-30% (w/v). In some cases, the rock to liquid ratio is from about 20-35% (w/v). In some cases, the rock to liquid ratio is from about 20-30% (w/v). In some cases, the rock to liquid ratio is from about 25-35% (w/v). In some cases, the rock to liquid ratio is from about 25-30% (w/v). In some cases, the rock to liquid ratio is about 1% (w/v). In some cases, the rock to liquid ratio is about 5% (w/v). In some cases, the rock to liquid ratio is about 10% (w/v). In some cases, the rock to liquid ratio is about 15% (w/v). In some cases, the rock to liquid ratio is about 20% (w/v). In some cases, the rock to liquid ratio is about 25% (w/v). In some cases, the rock to liquid ratio is about 30% (w/v). In some cases, the rock to liquid ratio is about 35% (w/v). In some cases, the rock to liquid ratio is about 40% (w/v).

In some cases, the methods provided herein comprise an enzymatic reaction that proceeds for a set period of time. In some cases, the method comprises the enzymatic reaction proceeding for about 1-72 hours. In some cases, the method comprises the enzymatic reaction proceeding for about 1-60 hours. In some cases, the method comprises the enzymatic reaction proceeding for about 1-48 hours. In some cases, the method comprises the enzymatic reaction proceeding for about 1-36 hours. In some cases, the method comprises the enzymatic reaction proceeding for about 1-24 hours. In some cases, the method comprises the enzymatic reaction proceeding for about 1-12 hours. In some cases, the method comprises the enzymatic reaction proceeding for about 1-6 hours. In some cases, the method comprises the enzymatic reaction proceeding for about 12-72 hours. In some cases, the method comprises the enzymatic reaction proceeding for about 12-60 hours. In some cases, the method comprises the enzymatic reaction proceeding for about 12-48 hours. In some cases, the method comprises the enzymatic reaction proceeding for about 12-36 hours. In some cases, the method comprises the enzymatic reaction proceeding for about 12-24 hours. In some cases, the method comprises the enzymatic reaction proceeding for about 24-72 hours. In some cases, the method comprises the enzymatic reaction proceeding for about 24-60 hours. In some cases, the method comprises the enzymatic reaction proceeding for about 24-48 hours. In some cases, the method comprises the enzymatic reaction proceeding for about 24-36 hours. In some cases, the method comprises the enzymatic reaction proceeding for about 36-72 hours. In some cases, the method comprises the enzymatic reaction proceeding for about 36-60 hours. In some cases, the method comprises the enzymatic reaction proceeding for about 36-48 hours. In some cases, the method comprises the enzymatic reaction proceeding for about 48-72 hours. In some cases, the method comprises the enzymatic reaction proceeding for about 48-60 hours. In some cases, the method comprises the enzymatic reaction proceeding for about 1 hour. In some cases, the method comprises the enzymatic reaction proceeding for about 6 hours. In some cases, the method comprises the enzymatic reaction proceeding for about 12 hours. In some cases, the method comprises the enzymatic reaction proceeding for about 24 hours. In some cases, the method comprises the enzymatic reaction proceeding for about 36 hours. In some cases, the method comprises the enzymatic reaction proceeding for about 48 hours. In some cases, the method comprises the enzymatic reaction proceeding for about 60 hours. In some cases, the method comprises the enzymatic reaction proceeding for about 72 hours.

In some cases, the methods provided herein comprise contacting the enzyme having silicase activity with a co-factor. The co-factor may help further increase the reaction rate when used in combination with the enzyme having silicase activity. In some cases, the co-factor is selected from the group consisting of: iron, zinc, copper, nickel, cobalt, and any combination thereof. Such co-factor may be used with any enzyme disclosed anywhere in the present disclosure. In some cases, the co-factor may be a non-natural co-factor, such as a co-factor that is typically not used by an enzyme as a co-factor in nature.

In some cases, the enzyme having silicase activity depolymerizes silicate mineral in the mineral material (e.g., ore/rock). In some cases, the enzyme having silicase activity cleaves one or more Si—O bonds in the mineral material to generate silicic acid (Si(OH)4). In some cases, the metal is lithium, aluminum, iron, nickel, cobalt, strontium, and/or a rare earth element. In some cases, the metal is lithium. In some cases, the metal is aluminum. In some cases, the metal is iron. In some cases, the metal is strontium. As a result of enzymatic degradation and/or disintegration of the mineral material, the metal may be released and extracted from the mineral, in some cases, in a solution, in some cases in the form of a metal ion or a metal atom. The solution may be an aqueous solution.

In some cases, the method comprises extracting the metal from the solution. In some cases, the method comprises purifying the metal from the solution, thereby generating a purified metal, a metal ion, a metal atom, a solid metal complex, a metal precipitate, or any combination thereof. In some cases, the purified metal has a purity of at least about 60%, at least about 70%, at least about 80%, at least about 90%, at least about 95%, at least about 99%, at least about 99.9%, at least about 99.99%, at least about 99.999%, at least about 99.9999%, or higher purity. In some cases, the purified metal is lithium, aluminum, iron, nickel, cobalt, strontium, a rare earth element, and/or uranium. In some cases, the purified metal is industry-grade, battery-grade, and/or pharmaceutical grade. In some cases, the purified metal is industry-grade lithium, battery-grade lithium, and/or pharmaceutical-grade lithium.

In some embodiments, the enzyme having silicase activity has a catalytic efficiency of at least about 80%, at least about 85%, at least about 90%, at least about 91%, at least about 92%, at least about 93%, at least about 94%, at least about 95%, at least about 98% at least about 99%, at least about 99.5% or higher. In some embodiments, the method has a maximum metal extraction rate of at least about 80%, at least about 85%, at least about 90%, at least about 91%, at least about 92%, at least about 93%, at least about 94%, at least about 95%, at least about 98% at least about 99%, at least about 99.5% or higher. In some embodiments, the enzyme having silicase activity has a catalytic efficiency of at least about 1.1, 1.2, 1.3, 1.4, 1.5, 1.6, 1.7, 1.8, 1.9, 2, 2.25, 2.5, 2.75, 3, 3.25, 3.5, 3.75, 4, 4.25, 4.5, 4.75, 5, 5.5, 6, 6.5, 7, 8, 9, 10 times or higher. In some embodiments, the method has a maximum metal extraction rate of at least about 1.1, 1.2, 1.3, 1.4, 1.5, 1.6, 1.7, 1.8, 1.9, 2, 2.25, 2.5, 2.75, 3, 3.25, 3.5, 3.75, 4, 4.25, 4.5, 4.75, 5, 5.5, 6, 6.5, 7, 8, 9, 10 times or higher.

In some aspects, the enzyme having silicase activity is recombinantly produced in a host cell or in a cell-free production system. In some cases, the host cell is a bacterial cell or yeast cell. In some cases, the bacterial cell is Escherichia coli. In some cases, the yeast cell is Pichia pastoris or Saccharomyces cerevisiae.

In some embodiments, the methods of the present disclosure comprise performing a reaction involving an enzyme and a mineral material, as described throughout the disclosure. The reaction may be performed inside any suitable container. In some cases, the reaction may be performed on a rock or ore. In some cases, the mineral material may be placed inside a container and added to a solution comprising the enzyme, water, a buffer, and/or potential other reagents for performing the reaction. In some cases, the reaction may be performed in one or more of a container, a dish, a beaker, a device, a tank, a reactor, and/or any combination thereof. The containers (e.g., one or more reactors and/or tanks) may be connected to one another to perform one or more reactions according to the embodiments of the present disclosure. The containers may also be reaction units and/or process units each of which may serve a function as part of the method and/or in combination with the method. For example, one or more tanks, reactors, and/or processing units, may be connected to each other with any configuration, such as in series, in parallel, or any combination thereof to perform the method steps such as contact the enzyme with the mineral, extracting the metal, separating the metal from the solution, purifying the metal, processing the metal, and converting the metal into an industry-grade, battery-grade, or pharmaceutical-grade metal. In some cases, the method further comprises grinding the mineral material (e.g., rock/ore) prior to performing the reaction (e.g., the enzymatic degradation). In some cases, the method further comprises using a filtration/chelating system, a precipitation system, a recycle system, or any combination thereof.

In an aspect, provided herein is a non-naturally occurring enzyme having silicase activity, the enzyme comprising at least one amino acid variation relative to a wild-type enzyme, and having increased ability to release metals from a mineral material (e.g., rock/ore) as compared to the wild-type enzyme. In some cases, the enzyme having silicase activity is used in a reaction/process of the present disclosure for metal extraction, as described anywhere in the present disclosure. In some cases, a wild-type carbonic anhydrase, gamma carbonic anhydrase, or alpha carbonic anhydrase may be used as a starting point for the enzyme engineering and performing the modifications to design and synthesize the non-naturally occurring enzyme having silicase activity. The non-naturally occurring enzyme having silicase activity may in some cases be a modified, engineered, and semi-synthetic enzyme (e.g., the enzyme having silicase activity for performing the methods and reactions of the present disclosure). In some cases, a wild-type carbonic anhydrase, gamma carbonic anhydrase, or alpha carbonic anhydrase may be used as a starting point for the enzyme engineering and performing the modifications to design and synthesize the non-naturally occurring enzyme (e.g., the enzyme having silicase activity for performing the methods and reactions of the present disclosure). As such, the resulting modified enzyme may have some similarities, for example with respect to characteristics and sequence, to a wild-type Carbonic Anhydrase, gamma Carbonic Anhydrase, or alpha Carbonic Anhydrase, and some differences, for example with respect to characteristics and sequence, to a wild-type carbonic anhydrase, gamma carbonic anhydrase, or alpha carbonic anhydrase.

In some cases the wild-type enzyme is selected from the group consisting of: Methanosarcina thermophila gamma carbonic anhydrase, Bacillus lichenmformis CG-B52 gamma carbonic anhydrase, Pelobacter carbinolicus gamma carbonic anhydrase, Syntrophus aciditrophicus gamma carbonic anhydrase, Methanosarcina barkeri gamma carbonic anhydrase, Methanosarcina mazei carbonic anhydrase, Bacillus halodurans alpha carbonic anhydrase, Alkalihalobacillus clausii (strain KSM-K16) (Bacillus clausii) alpha carbonic anhydrase, Methanosarcina acetivorans carbonate dehydratase, Kofleriaceae bacterium SLC26A/SulP transporter domain-containing protein, Thermodesulfitimonas autotrophica carbonic anhydrase/acetyltransferase-like protein (Isoleucine patch superfamily), Fischerella thermalis/Mastigocladus laminosus JSC-11 carboxysome assembly protein CcmM, Thermosynechococcus vestitus BP-1/(Thermosynechococcus elongatus BP-1) carboxysome assembly protein CcmM, Methanothrix thermoacetophila carbonate dehydratase, Thermosyntropha lipolytica carbonic anhydrase or acetyltransferase, isoleucine patch superfamily, Desulfofundulus thermobenzoicus transferase, Archaeoglobus veneficus carbonate dehydratase, Suberites domuncula carbonic anhydrase, and any combination thereof.

In some cases, the non-naturally occurring enzyme is derived from an organism selected from the group consisting of: Methanosarcina thermophila, Bacillus lichenmformis CG-B52, Pelobacter carbinolicus, Syntrophus aciditrophicus, Methanosarcina barkeri, Methanosarcina mazei, Bacillus halodurans, Alkalihalobacillus clausii (strain KSM-K16) (Bacillus clausii), Methanosarcina acetivorans, Kofleriaceae bacterium, Thermodesulfitimonas autotrophica, Fischerella thermalis/Mastigocladus laminosus JSC-11 carboxysome assembly protein CcmM, Thermosynechococcus vestitus BP-1/(Thermosynechococcus elongatus BP-1) carboxysome assembly protein CcmM, Methanothrix thermoacetophila, Thermosyntropha lipolytica, Desulfofundulus thermobenzoicus, Archaeoglobus veneficus, Suberites domuncula, and any combination thereof.

In some cases, the non-naturally occurring enzyme comprises an amino acid sequence having at least about 10%, at least about 20%, at least about 30%, at least about 40%, having at least about 50%, at least about 60%, at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or more sequence identity to an amino acid sequence of any one of SEQ ID NOS: 1-402. In some cases, the non-naturally occurring enzyme comprises an amino acid sequence having at least about 10%, at least about 20%, at least about 30%, at least about 40%, having at least about 50%, at least about 60%, at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or more sequence identity to an amino acid sequence of any one of SEQ ID NOS: 403-464. In some cases, the wild-type enzyme is a carbonic anhydrase. In some cases, the carbonic anhydrase is a gamma carbonic anhydrase or alpha carbonic anhydrase.

In some cases, the methods of the present disclosure are performed on a mineral material. In some cases, the mineral material comprises or is a rock, an ore, a deposit, a clay, a natural mineral material, a man-made mineral material, or any combination thereof. The method may be according to the embodiments described anywhere herein. According to the embodiments described anywhere in the present disclosure, in some cases, an enzyme having silicase activity acts on a mineral material. The enzyme and the method of the present disclosure may degrade, digest, or disintegrate the mineral material and extract a metal therefrom. The mineral material such as a rock, ore, natural mineral materials, and/or man-made mineral materials may by abundant sources of valuable metals such as lithium, aluminum, iron, nickel, cobalt, copper, strontium, rare earth elements, uranium, and other metals with vast industrial use and applications. The methods and enzymes of the present disclosure facilitate access to such sources. In some cases, the mineral material comprises a silicate. In some cases, the mineral material comprises an inosilicate, a phyllosilicate, an amorphous silicate, a tectosilicate or any combination thereof. In some cases, the inosilicate is selected from the group consisting of: spodumene, wollastonite, balangeroite, eveslogite, holmquistite, jadeite, shattuckite, augite, tremolite, and any combination thereof. In some cases, the phyllosilicate is selected from the group consisting of: lepidolite, hectorite, kaolinite, vermiculite, muscovite, montmorillonite, and any combination thereof. In some cases, amorphous silicate is selected from the group consisting of obsidian, coal fly ash, pumice, glass, and any combination thereof. In some cases, the tectosilicate comprises quartz, sand, or glass.

In some cases, the non-naturally occurring enzyme has an increased ability to depolymerize silicate mineral in the mineral material, rock, ore, or other kind of mineral material as compared to the wild-type enzyme, increased selectivity or specificity toward a mineral structure in the mineral material, or both. In some cases, the non-naturally occurring enzyme has increased ability to cleave one or more Si—O bonds in the mineral material to generate silicic acid (Si(OH)4) as compared to the wild-type enzyme. In some cases, the metal comprises lithium, aluminum, iron, nickel, cobalt, or a rare earth element. In some cases, the metal comprises lithium. In some cases, the metal comprises aluminum. In some cases, the metal comprises iron. In some cases, the metal comprises strontium. In some cases, the non-naturally occurring enzyme is recombinantly produced in a host cell or in a cell-free production system. In some cases, the host cell is a bacterial cell or yeast cell. In some cases, the bacterial cell is Escherichia coli. In some cases, the yeast cell is Pichia pastoris or Saccharomyces cerevisiae.

In an aspect, provided herein is a reaction mixture comprising a mineral material and a non-naturally occurring enzyme having silicase activity, wherein the non-naturally occurring enzyme comprises at least one amino acid variation relative to a wild-type enzyme, and has increased ability to release metal from the mineral material as compared to the wild-type enzyme. The mineral material may be according to any embodiment described anywhere herein. It may comprise silicates and/or Si—O bonds. In some cases, the mineral material comprises silicates. In some cases, the mineral material may comprise an inosilicate, a phyllosilicate, an amorphous silicate, or any combination thereof. In some cases, the inosilicate is selected from the group consisting of: spodumene, wollastonite, balangeroite, eveslogite, holmquistite, jadeite, shattuckite, augite, tremolite, and any combination thereof. In some cases, the phyllosilicate is selected from the group consisting of: lepidolite, hectorite, kaolinite, vermiculite, muscovite, montmorillonite, and any combination thereof. In some cases, the amorphous silicate is selected from the group consisting of obsidian, coal fly ash, pumice, glass, and any combination thereof. The mineral material may be a source of a metal.

The metal may be extracted from the mineral material (in some cases a rock or ore) by using the methods and enzymes described anywhere in the present disclosure. The enzyme having silicase activity may be any enzyme disclosed anywhere in the present disclosure. In some cases, the reaction mixture has a pH from about 4 to about 11. In some cases, the reaction mixture has a pH from about 4 to about 10. In some cases, the reaction mixture has a pH from about 4 to about 9. In some cases, the reaction mixture has a pH from about 4 to about 8. In some cases, the reaction mixture has a pH from about 4 to about 7. In some cases, the reaction mixture has a pH from about 4 to about 6. In some cases, the reaction mixture has a pH from about 5 to about 11. In some cases, the reaction mixture has a pH from about 5 to about 10. In some cases, the reaction mixture has a pH from about 5 to about 9. In some cases, the reaction mixture has a pH from about 5 to about 8. In some cases, the reaction mixture has a pH from about 5 to about 7. In some cases, the reaction mixture has a pH from about 5 to about 6. In some cases, the reaction mixture has a pH from about 6 to about 11. In some cases, the reaction mixture has a pH from about 6 to about 10. In some cases, the reaction mixture has a pH from about 7 to about 11. In some cases, the reaction mixture has a pH from about 7 to about 10. In some cases, the reaction mixture has a pH from about 8 to about 11. In some cases, the reaction mixture has a pH from about 8 to about 10. In some cases, the reaction mixture has a pH from about 9 to about 11. In some cases, the reaction mixture has a pH from about 9 to about 10. In some cases, the reaction mixture has a pH of 4. In some cases, the reaction mixture has a pH of 5. In some cases, the reaction mixture has a pH of 6. In some cases, the reaction mixture has a pH of 7. In some cases, the reaction mixture has a pH of 8. In some cases, the reaction mixture has a pH of 9. In some cases, the reaction mixture has a pH of 10. In some cases, the reaction mixture has a pH of 11. In some cases, the reaction mixture has a temperature from about 20 to about 90 degrees Celsius (C). In some cases, the reaction mixture has a temperature from about 23 to about 90 degrees Celsius (C). In some cases, the reaction mixture has a temperature from about 23 to about 85 degrees Celsius (C). In some cases, the reaction mixture has a temperature from about 30 to about 90, from about 30 to about 80, from about 30 to about 70, from about 30 to about 60, or from about 30 to about 50 degrees Celsius (C). In some cases, the reaction mixture has a temperature from about 45 to about 55 degrees Celsius (C). In some cases, the reaction mixture has a temperature from about 45 to about 50 degrees Celsius (C). In some cases, the reaction mixture has a temperature about 20 degrees Celsius (C). In some cases, the reaction mixture has a temperature about 23 degrees Celsius (C). In some cases, the reaction mixture has a temperature about 25 degrees Celsius (C). In some cases, the reaction mixture has a temperature about 30 degrees Celsius (C). In some cases, the reaction mixture has a temperature about 35 degrees Celsius (C). In some cases, the reaction mixture has a temperature about 40 degrees Celsius (C). In some cases, the reaction mixture has a temperature about 45 degrees Celsius (C). In some cases, the reaction mixture has a temperature about 50 degrees Celsius (C). In some cases, the reaction mixture has a temperature about 55 degrees Celsius (C). In some cases, the reaction mixture has a temperature about 60 degrees Celsius (C). In some cases, the reaction mixture has a temperature about 70 degrees Celsius (C). In some cases, the reaction mixture has a temperature about 75 degrees Celsius (C). In some cases, the reaction mixture has a temperature about 80 degrees Celsius (C). In some cases, the reaction mixture has a temperature about 85 degrees Celsius (C). In some cases, the reaction mixture further comprises a co-factor of the non-naturally occurring enzyme. In some cases, the co-factor is selected from the group consisting of iron, zinc, copper, nickel, and cobalt. In some cases, the co-factor is copper. In some cases, the co-factor is iron. In some cases, the reaction mixture further comprises a buffered saline solution. In some cases, the buffered solution comprises saline, glycine, iron ions, or any combination thereof. In some cases the buffered solution comprises TRIS, PBS, citrate, monosodium glutamate, or any combination thereof. In some cases, the reaction mixture further comprises an activator co-factor of the non-naturally occurring enzyme. In some cases, the activator co-factor is glycine. The reaction mixture may be used to perform any method described anywhere herein using any enzyme, any co-factor, and/or any reaction condition disclosed anywhere herein.

In some cases, the reaction mixture provided herein comprise crushing or grinding the rocks or ores to achieve a particulate size. In some cases, the ground ores comprise a size of about 50 μm to 1 mm. In some cases, the ground ores comprise a size from about 50 μm to 750 μm. In some cases, the ground ores comprise a size from about 50 μm to 500 μm. In some cases, the ground ores comprise a size from about 50 μm to 250 μm. In some cases, the ground ores comprise a size from about 50 μm to 150 μm. In some cases, the ground ores comprise a size from about 50 μm to 100 μm. In some cases, the ground ores comprise a size of about 50 μm. In some cases, the ground ores comprise a size of about 100 μm. In some cases, the ground ores comprise a size of about 150 μm. In some cases, the ground ores comprise a size of about 250 μm. In some cases, the ground ores comprise a size of about 500 μm. In some cases, the ground ores comprise a size of about 750 μm. In some cases, the ground ores comprise a size of about 1 mm.

In some cases, the reaction mixture provided herein comprise creating a slurry of crushed rock and liquid. In some cases, the rock to liquid ratio is from about 1-40% (w/v). In some cases, the rock to liquid ratio is from about 1-35% (w/v). In some cases, the rock to liquid ratio is from about 1-30% (w/v). In some cases, the rock to liquid ratio is from about 1-25% (w/v). In some cases, the rock to liquid ratio is from about 1-20% (w/v). In some cases, the rock to liquid ratio is from about 1-15% (w/v). In some cases, the rock to liquid ratio is from about 1-10% (w/v). In some cases, the rock to liquid ratio is from about 1-5% (w/v). In some cases, the rock to liquid ratio is from about 10-40% (w/v). In some cases, the rock to liquid ratio is from about 10-35% (w/v). In some cases, the rock to liquid ratio is from about 10-30% (w/v). In some cases, the rock to liquid ratio is from about 10-25% (w/v). In some cases, the rock to liquid ratio is from about 15-35% (w/v). In some cases, the rock to liquid ratio is from about 15-30% (w/v). In some cases, the rock to liquid ratio is from about 20-35% (w/v). In some cases, the rock to liquid ratio is from about 20-30% (w/v). In some cases, the rock to liquid ratio is from about 25-35% (w/v). In some cases, the rock to liquid ratio is from about 25-30% (w/v). In some cases, the rock to liquid ratio is about 1% (w/v). In some cases, the rock to liquid ratio is about 5% (w/v). In some cases, the rock to liquid ratio is about 10% (w/v). In some cases, the rock to liquid ratio is about 15% (w/v). In some cases, the rock to liquid ratio is about 20% (w/v). In some cases, the rock to liquid ratio is about 25% (w/v). In some cases, the rock to liquid ratio is about 30% (w/v). In some cases, the rock to liquid ratio is about 35% (w/v). In some cases, the rock to liquid ratio is about 40% (w/v).

In some cases, the reaction mixture provided herein comprise an enzymatic reaction that proceeds for a set period of time. In some cases, the reaction mixture comprises the enzymatic reaction proceeding for about 1-72 hours. In some cases, the reaction mixture comprises the enzymatic reaction proceeding for about 1-60 hours. In some cases, the reaction mixture comprises the enzymatic reaction proceeding for about 1-48 hours. In some cases, the reaction mixture comprises the enzymatic reaction proceeding for about 1-36 hours. In some cases, the reaction mixture comprises the enzymatic reaction proceeding for about 1-24 hours. In some cases, the reaction mixture comprises the enzymatic reaction proceeding for about 1-12 hours. In some cases, the reaction mixture comprises the enzymatic reaction proceeding for about 1-6 hours. In some cases, the reaction mixture comprises the enzymatic reaction proceeding for about 12-72 hours. In some cases, the reaction mixture comprises the enzymatic reaction proceeding for about 12-60 hours. In some cases, the reaction mixture comprises the enzymatic reaction proceeding for about 12-48 hours. In some cases, the reaction mixture comprises the enzymatic reaction proceeding for about 12-36 hours. In some cases, the reaction mixture comprises the enzymatic reaction proceeding for about 12-24 hours. In some cases, the reaction mixture comprises the enzymatic reaction proceeding for about 24-72 hours. In some cases, the reaction mixture comprises the enzymatic reaction proceeding for about 24-60 hours. In some cases, the reaction mixture comprises the enzymatic reaction proceeding for about 24-48 hours. In some cases, the reaction mixture comprises the enzymatic reaction proceeding for about 24-36 hours. In some cases, the reaction mixture comprises the enzymatic reaction proceeding for about 36-72 hours. In some cases, the reaction mixture comprises the enzymatic reaction proceeding for about 36-60 hours. In some cases, the reaction mixture comprises the enzymatic reaction proceeding for about 36-48 hours. In some cases, the reaction mixture comprises the enzymatic reaction proceeding for about 48-72 hours. In some cases, the reaction mixture comprises the enzymatic reaction proceeding for about 48-60 hours. In some cases, the reaction mixture comprises the enzymatic reaction proceeding for about 1 hour. In some cases, the reaction mixture comprises the enzymatic reaction proceeding for about 6 hours. In some cases, the reaction mixture comprises the enzymatic reaction proceeding for about 12 hours. In some cases, the reaction mixture comprises the enzymatic reaction proceeding for about 24 hours. In some cases, the reaction mixture comprises the enzymatic reaction proceeding for about 36 hours. In some cases, the reaction mixture comprises the enzymatic reaction proceeding for about 48 hours. In some cases, the reaction mixture comprises the enzymatic reaction proceeding for about 60 hours. In some cases, the reaction mixture comprises the enzymatic reaction proceeding for about 72 hours.

The reaction mixture comprises a non-naturally occurring enzyme described anywhere herein. As described throughout the present disclosure, in some cases, the non-naturally occurring enzyme may be an engineered and/or semi-synthetic enzyme having a modification compared to a wild-type enzyme. In some cases, the wild-type enzyme is selected from the group consisting of: Methanosarcina thermophila gamma carbonic anhydrase, Bacillus lichenmformis CG-B52 gamma carbonic anhydrase, Pelobacter carbinolicus gamma carbonic anhydrase, Syntrophus aciditrophicus gamma carbonic anhydrase, Methanosarcina barkeri gamma carbonic anhydrase, Methanosarcina mazei carbonic anhydrase, Bacillus halodurans alpha carbonic anhydrase, Alkalihalobacillus clausii (strain KSM-K16) (Bacillus clausii) alpha carbonic anhydrase, Methanosarcina acetivorans carbonate dehydratase, Kofleriaceae bacterium SLC26A/SulP transporter domain-containing protein, Thermodesulfitimonas autotrophica carbonic anhydrase/acetyltransferase-like protein (Isoleucine patch superfamily), Fischerella thermalis/Mastigocladus laminosus JSC-11 carboxysome assembly protein CcmM, Thermosynechococcus vestitus BP-1/(Thermosynechococcus elongatus BP-1) carboxysome assembly protein CcmM, Methanothrix thermoacetophila carbonate dehydratase, Thermosyntropha lipolytica carbonic anhydrase or acetyltransferase, isoleucine patch superfamily, Desulfofundulus thermobenzoicus transferase, Archaeoglobus veneficus carbonate dehydratase, Suberites domuncula carbonic anhydrase, and any combination thereof.

In some cases, the non-naturally occurring enzyme comprises an amino acid sequence having at least about 5%, at least about 10%, at least about 20%, at least about 30%, at least about 40%, at least about 50%, at least about 60%, at least about 70%, at least about 80% at least about 85%, at least about 90%, at least about 91%, at least about 92%, at least about 93%, at least about 94%, at least about 95%, at least about 96%, at least about 97%, at least about 98%, at least about 99%, or more sequence identity to an amino acid sequence of any one of SEQ ID NOS: 1-402. In some cases, the non-naturally occurring enzyme comprises an amino acid sequence having at least about 5%, at least about 10%, at least about 20%, at least about 30%, at least about 40%, at least about 50%, at least about 60%, at least about 70%, at least about 80% at least about 85%, at least about 90%, at least about 91%, at least about 92%, at least about 93%, at least about 94%, at least about 95%, at least about 96%, at least about 97%, at least about 98%, at least about 99%, or more sequence identity to an amino acid sequence of any one of SEQ ID NOS: 403-464. In some cases, the wild-type enzyme is a carbonic anhydrase. In some cases, the carbonic anhydrase is a gamma carbonic anhydrase or alpha carbonic anhydrase.

In some cases, the non-naturally occurring enzyme has increased ability to depolymerize silicate mineral in the mineral material as compared to the wild-type enzyme. In some cases, the non-naturally occurring enzyme has increased ability to cleave one or more Si—O bonds in the mineral material (e.g., rock/ore) to generate silicic acid (Si(OH)4) as compared to the wild-type enzyme. In some cases, the metal comprises lithium. In some cases, the metal comprises aluminum. In some cases, the metal comprises iron. In some cases, the metal comprises strontium. In some cases, the metal may comprise lithium, aluminum, nickel, iron, cobalt, copper, a rare earth element, uranium, strontium, another metal, or any combination thereof. In some cases, the non-naturally occurring enzyme is recombinantly produced in a host cell. In some cases, the host cell is a bacterial cell or yeast cell. In some cases, the bacterial cell is Escherichia coli. In some cases, the yeast cell is Pichia pastoris or Saccharomyces cerevisiae.

In an aspect, provided herein is a polynucleotide comprising a nucleotide sequence encoding the non-naturally occurring enzyme of any one of the preceding embodiments. In an aspect, provided herein is a vector comprising the polynucleotide comprising the nucleotide sequence encoding the non-naturally occurring enzyme. The non-naturally occurring enzyme may be according to any embodiment mentioned anywhere in the present disclosure. The polynucleotide and vector may be designed and engineered. The polynucleotide and the vector may get synthesized. The polynucleotide and/or vector may be used to generate and/or produce the non-naturally occurring enzyme of the present disclosure.

In an aspect, provided herein is a method of increasing silicase activity of an enzyme, the method comprising contacting or combining the enzyme with a non-natural co-factor. In some cases, the enzyme and the co-factor may be added to a solution. In some cases, the co-factor may be brought in proximity of the enzyme. In some cases, the enzyme and the co-factor may be part of the same system, the same reaction mixture, or the same kit for performing the methods of the present disclosure. In some cases, the non-natural co-factor increases silicase activity of the enzyme as compared to the enzyme in the presence of a natural co-factor. In some cases, the non-natural co-factor may be copper. In some cases, natural co-factor is zinc. In some cases, natural co-factor is iron. In some cases, the method is performed in the absence of the natural co-factor. In some cases, the non-natural co-factor does not act as a co-factor for the enzyme having silicase activity in nature. In some cases, the method further comprises contacting the enzyme and the non-natural co-factor with the mineral material under reaction conditions such that a metal contained within the mineral material is solubilized and released from the mineral material such as rock/ore.

As an example, a method of the present disclosure comprises using an enzyme having silicase activity (wild-type or engineered/modified/semi-synthetic) to degrade a mineral material such as a rock/ore comprising metal-bearing silicates so as to extract the metal from the mineral material. Zinc may act as a natural co-factor for the enzyme comprising silicase activity in nature. For example, a natural silicate rock may get degraded by a wild-type silicase enzyme such as a gamma carbonic anhydrase in nature, zinc may act as a co-factor for the wild-type enzyme, catalyze the digestion/degradation reaction, and speed it up and/or increase its efficiency. Alternatively, in some cases, the method of the present disclosure may comprise bringing an enzyme having silicase activity to a mineral material such as a rock/ore comprising metal-bearing silicates, and further provide a co-factor other than zinc (the natural co-factor) to increase the catalytic effects of the enzyme having silicase activity on the mineral material. The co-factor other than zinc may be a non-natural co-factor. The non-natural co-factor is in some cases, copper, iron, nickel, cobalt, or glycine. In some cases, the non-natural co-factor may work better than the natural co-factor. For example, copper, iron, nickel, cobalt, or glycine may work better than zinc in increasing the catalytic efficiency of the enzyme and increasing the reaction rate. The enzyme used in the method of the present disclosure may comprise using a wild-type enzyme or a modified enzyme described anywhere in the present disclosure.

In some cases, the amount of metal extracted, solubilized/precipitated in the extraction solution, and/or released from the mineral material (e.g., ore/rock) is greater than an amount of metal extracted from the mineral material when the enzyme is contacted with the natural co-factor (e.g., in unit time when other conditions are equal). In some cases, the amount of metal extracted from the mineral material is greater than an amount of metal extracted from the mineral material when the enzyme is contacted with the natural co-factor. In some cases, the mineral material comprises silicate. In some cases, the mineral material comprises an inosilicate, a phyllosilicate, an amorphous silicate, a tectosilicate, or any combination thereof. In some cases, the inosilicate is selected from the group consisting of: spodumene, wollastonite, balangeroite, eveslogite, holmquistite, jadeite, shattuckite, augite, tremolite, and any combination thereof. In some cases, the phyllosilicate is selected from the group consisting of: lepidolite, hectorite, kaolinite, vermiculite, muscovite, montmorillonite, and any combination thereof. In some cases, the amorphous silicate is selected from the group consisting of obsidian, coal fly ash, pumice, glass, and any combination thereof. In some cases, the enzyme having silicase activity is a carbonic anhydrase. In some cases, the carbonic anhydrase is a gamma carbonic anhydrase or an alpha carbonic anhydrase. In some cases, the enzyme is a wild-type enzyme. In some cases, the enzyme is a modified or engineered enzyme.

In some cases, the enzyme having silicase activity is derived from an organism selected from the group consisting of: Methanosarcina thermophila, Bacillus lichenmformis CG-B52, Pelobacter carbinolicus, Syntrophus aciditrophicus, Methanosarcina barkeri, Methanosarcina mazei, Bacillus halodurans, Alkalihalobacillus clausii (strain KSM-K16) (Bacillus clausii), Methanosarcina acetivorans, Kofleriaceae bacterium, Thermodesulfitimonas autotrophica, Fischerella thermalis/Mastigocladus laminosus JSC-11 carboxysome assembly protein CcmM, Thermosynechococcus vestitus BP-1/(Thermosynechococcus elongatus BP-1) carboxysome assembly protein CcmM, Methanothrix thermoacetophila, Thermosyntropha lipolytica, Desulfofundulus thermobenzoicus, Archaeoglobus veneficus, Suberites domuncula, and any combination thereof.

In some cases, the enzyme having silicase activity comprises an amino acid sequence having at least about 5%, at least about 10%, at least about 20%, at least about 30%, at least about 40%, having at least about 50%, at least about 60%, at least about 70%, tale at least about 80%, at least about 85%, at least about 90%, at least about 91%, at least about 92%, at least about 93%, at least about 94%, at least about 95%, at least about 96%, at least about 97%, at least about 98%, at least about 99%, at least about 99.5%, at least about 99.9%, or more sequence identity to an amino acid sequence of any one of SEQ ID NOS: 1-402. In some cases, the enzyme having silicase activity comprises an amino acid sequence having at least about 5%, at least about 10%, at least about 20%, at least about 30%, at least about 40%, having at least about 50%, at least about 60%, at least about 70%, tale at least about 80%, at least about 85%, at least about 90%, at least about 91%, at least about 92%, at least about 93%, at least about 94%, at least about 95%, at least about 96%, at least about 97%, at least about 98%, at least about 99%, at least about 99.5%, at least about 99.9%, or more sequence identity to an amino acid sequence of any one of SEQ ID NOS: 403-464. In some cases, the enzyme having silicase activity is an engineered enzyme. In some cases, the enzyme having silicase activity is an enzyme having at least one amino acid variation as compared to a wild-type enzyme.

In some embodiments, the enzyme having silicase activity has a catalytic efficiency of at least about 80%, at least about 85%, at least about 90%, at least about 91%, at least about 92%, at least about 93%, at least about 94%, at least about 95%, at least about 98% at least about 99%, at least about 99.5% or higher. In some embodiments, the method has a maximum metal extraction rate of at least about 80%, at least about 85%, at least about 90%, at least about 91%, at least about 92%, at least about 93%, at least about 94%, at least about 95%, at least about 98% at least about 99%, at least about 99.5% or higher. In some embodiments, the enzyme having silicase activity has a catalytic efficiency of at least about 1.1, 1.2, 1.3, 1.4, 1.5, 1.6, 1.7, 1.8, 1.9, 2, 2.25, 2.5, 2.75, 3, 3.25, 3.5, 3.75, 4, 4.25, 4.5, 4.75, 5, 5.5, 6, 6.5, 7, 8, 9, 10 times or higher. In some embodiments, the method has a maximum metal extraction rate of at least about 1.1, 1.2, 1.3, 1.4, 1.5, 1.6, 1.7, 1.8, 1.9, 2, 2.25, 2.5, 2.75, 3, 3.25, 3.5, 3.75, 4, 4.25, 4.5, 4.75, 5, 5.5, 6, 6.5, 7, 8, 9, 10 times or higher. In some embodiments, the enzyme having silicase activity has a pKd of at least about 5, at least about 6, at least about 7, at least about 8, at least about 9, at least about 10, at least about 11, or higher. In some embodiments, the enzyme having silicase activity has a Kcat value of at least about 2 mol per second (mol/s), at least about 10 mol/s, at least about 50 mol/s, at least about 100 mol/s, at least about 200 mol/s, at least about 300 mol/s, at least about 400 mol/s, at least about 500 mol/s, at least about 600 mol/s, at least about 700 mol/s, at least about 800 mol/s, at least about 900 mol/s, at least about 1000 mol/s, or higher.

In some cases, the reaction conditions comprise a temperature from about 20 to about 90 degrees Celsius (C). In some cases, the reaction conditions comprise a temperature from about 23 to about 90 degrees Celsius (C). In some cases, the reaction conditions comprise a temperature from about 23 to about 85 degrees Celsius (C). In some cases, the reaction conditions comprise a temperature from about 30 to about 90, from about 30 to about 80, from about 30 to about 70, from about 30 to about 60, or from about 30 to about 50 degrees Celsius (C). In some cases, the reaction conditions comprise a temperature from about 45 to about 55 degrees Celsius (C). In some cases, the reaction mixture has a temperature from about 45 to about 50 degrees Celsius (C). In some cases, the reaction conditions comprise a temperature about 20 degrees Celsius (C). In some cases, the reaction conditions comprise a temperature about 23 degrees Celsius (C). In some cases, the reaction conditions comprise a temperature about 25 degrees Celsius (C). In some cases, the reaction conditions comprise a temperature about 30 degrees Celsius (C). In some cases, the reaction conditions comprise a temperature about 35 degrees Celsius (C). In some cases, the reaction conditions comprise a temperature about 40 degrees Celsius (C). In some cases, the reaction conditions comprise a temperature about 45 degrees Celsius (C). In some cases, the reaction conditions comprise a temperature about 50 degrees Celsius (C). In some cases, the reaction conditions comprise a temperature about 55 degrees Celsius (C). In some cases, the reaction conditions comprise a temperature about 60 degrees Celsius (C). In some cases, the reaction conditions comprise a temperature about 70 degrees Celsius (C). In some cases, the reaction conditions comprise a temperature about 75 degrees Celsius (C). In some cases, the reaction conditions comprise a temperature about 80 degrees Celsius (C). In some cases, the reaction conditions comprise a temperature about 85 degrees Celsius (C). In some cases, the reaction conditions comprise a pH from about 4 to about 11. In some cases, the reaction conditions comprise a pH from about 4 to about 10. In some cases, the reaction conditions comprise a pH from about 4 to about 9. In some cases, the reaction conditions comprise a pH from about 4 to about 8. In some cases, the reaction conditions comprise a pH from about 4 to about 7. In some cases, the reaction conditions comprise a pH from about 4 to about 6. In some cases, the reaction conditions comprise a pH from about 5 to about 11. In some cases, the reaction conditions comprise a pH from about 5 to about 10. In some cases, the reaction conditions comprise a pH from about 5 to about 9. In some cases, the reaction conditions comprise a pH from about 5 to about 8. In some cases, the reaction conditions comprise a pH from about 5 to about 7. In some cases, the reaction conditions comprise a pH from about 5 to about 6. In some cases, the reaction conditions comprise a pH from about 6 to about 11. In some cases, the reaction conditions comprise a pH from about 6 to about 10. In some cases, the reaction conditions comprise a pH from about 7 to about 11. In some cases, the reaction conditions comprise a pH from about 7 to about 10. In some cases, the reaction conditions comprise a pH from about 8 to about 11. In some cases, the reaction conditions comprise a pH from about 8 to about 10. In some cases, the reaction conditions comprise a pH from about 9 to about 11. In some cases, the reaction conditions comprise a pH from about 9 to about 10. In some cases, the reaction conditions comprise a pH of 4. In some cases, the reaction conditions comprise a pH of 5. In some cases, the reaction conditions comprise a pH of 6. In some cases, the reaction conditions comprise a pH of 7. In some cases, the reaction conditions comprise a pH of 8. In some cases, the reaction conditions comprise a pH of 9. In some cases, the reaction conditions comprise a pH of 10. In some cases, the reaction conditions comprise a pH of 11. In some cases, the reaction conditions comprise contacting the enzyme having silicase activity with a co-factor. In some cases, the co-factor is selected from the group consisting of: Iron, zinc, copper, nickel, and cobalt.

In some cases, the reaction conditions provided herein comprise crushing or grinding the rocks or ores to achieve a particulate size. In some cases, the ground ores comprise a size of about 50 μm to 1 mm. In some cases, the ground ores comprise a size from about 50 μm to 750 μm. In some cases, the ground ores comprise a size from about 50 μm to 500 μm. In some cases, the ground ores comprise a size from about 50 μm to 250 μm. In some cases, the ground ores comprise a size from about 50 μm to 150 μm. In some cases, the ground ores comprise a size from about 50 μm to 100 μm. In some cases, the ground ores comprise a size of about 50 μm. In some cases, the ground ores comprise a size of about 100 μm. In some cases, the ground ores comprise a size of about 150 μm. In some cases, the ground ores comprise a size of about 250 μm. In some cases, the ground ores comprise a size of about 500 μm. In some cases, the ground ores comprise a size of about 750 μm. In some cases, the ground ores comprise a size of about 1 mm.

In some cases, the reaction conditions provided herein comprise creating a slurry of crushed rock and liquid. In some cases, the rock to liquid ratio is from about 1-40% (w/v). In some cases, the rock to liquid ratio is from about 1-35% (w/v). In some cases, the rock to liquid ratio is from about 1-30% (w/v). In some cases, the rock to liquid ratio is from about 1-25% (w/v). In some cases, the rock to liquid ratio is from about 1-20% (w/v). In some cases, the rock to liquid ratio is from about 1-15% (w/v). In some cases, the rock to liquid ratio is from about 1-10% (w/v). In some cases, the rock to liquid ratio is from about 1-5% (w/v). In some cases, the rock to liquid ratio is from about 10-40% (w/v). In some cases, the rock to liquid ratio is from about 10-35% (w/v). In some cases, the rock to liquid ratio is from about 10-30% (w/v). In some cases, the rock to liquid ratio is from about 10-25% (w/v). In some cases, the rock to liquid ratio is from about 15-35% (w/v). In some cases, the rock to liquid ratio is from about 15-30% (w/v). In some cases, the rock to liquid ratio is from about 20-35% (w/v). In some cases, the rock to liquid ratio is from about 20-30% (w/v). In some cases, the rock to liquid ratio is from about 25-35% (w/v). In some cases, the rock to liquid ratio is from about 25-30% (w/v). In some cases, the rock to liquid ratio is about 1% (w/v). In some cases, the rock to liquid ratio is about 5% (w/v). In some cases, the rock to liquid ratio is about 10% (w/v). In some cases, the rock to liquid ratio is about 15% (w/v). In some cases, the rock to liquid ratio is about 20% (w/v). In some cases, the rock to liquid ratio is about 25% (w/v). In some cases, the rock to liquid ratio is about 30% (w/v). In some cases, the rock to liquid ratio is about 35% (w/v). In some cases, the rock to liquid ratio is about 40% (w/v).

In some cases, the reaction conditions provided herein comprise an enzymatic reaction that proceeds for a set period of time. In some cases, the reaction conditions comprise the enzymatic reaction proceeding for about 1-72 hours. In some cases, the reaction conditions comprise the enzymatic reaction proceeding for about 1-60 hours. In some cases, the reaction conditions comprise the enzymatic reaction proceeding for about 1-48 hours. In some cases, the reaction conditions comprise the enzymatic reaction proceeding for about 1-36 hours. In some cases, the reaction conditions comprise the enzymatic reaction proceeding for about 1-24 hours. In some cases, the reaction conditions comprise the enzymatic reaction proceeding for about 1-12 hours. In some cases, the reaction conditions comprise the enzymatic reaction proceeding for about 1-6 hours. In some cases, the reaction conditions comprise the enzymatic reaction proceeding for about 12-72 hours. In some cases, the reaction conditions comprise the enzymatic reaction proceeding for about 12-60 hours. In some cases, the reaction conditions comprise the enzymatic reaction proceeding for about 12-48 hours. In some cases, the reaction conditions comprise the enzymatic reaction proceeding for about 12-36 hours. In some cases, the reaction conditions comprise the enzymatic reaction proceeding for about 12-24 hours. In some cases, the reaction conditions comprise the enzymatic reaction proceeding for about 24-72 hours. In some cases, the reaction conditions comprise the enzymatic reaction proceeding for about 24-60 hours. In some cases, the reaction conditions comprise the enzymatic reaction proceeding for about 24-48 hours. In some cases, the reaction conditions comprise the enzymatic reaction proceeding for about 24-36 hours. In some cases, the reaction conditions comprise the enzymatic reaction proceeding for about 36-72 hours. In some cases, the reaction conditions comprise the enzymatic reaction proceeding for about 36-60 hours. In some cases, the reaction conditions comprise the enzymatic reaction proceeding for about 36-48 hours. In some cases, the reaction conditions comprise the enzymatic reaction proceeding for about 48-72 hours. In some cases, the reaction conditions comprise the enzymatic reaction proceeding for about 48-60 hours. In some cases, the reaction conditions comprise the enzymatic reaction proceeding for about 1 hour. In some cases, the reaction conditions comprise the enzymatic reaction proceeding for about 6 hours. In some cases, the reaction conditions comprise the enzymatic reaction proceeding for about 12 hours. In some cases, the reaction conditions comprise the enzymatic reaction proceeding for about 24 hours. In some cases, the reaction conditions comprise the enzymatic reaction proceeding for about 36 hours. In some cases, the reaction conditions comprise the enzymatic reaction proceeding for about 48 hours. In some cases, the reaction conditions comprise the enzymatic reaction proceeding for about 60 hours. In some cases, the reaction conditions comprise the enzymatic reaction proceeding for about 72 hours.

In some cases, the enzyme having silicase activity depolymerizes silicate mineral in the mineral material (e.g., ore/rock). In some cases, the enzyme having silicase activity cleaves one or more Si—O bonds in the mineral material to generate silicic acid (Si(OH)4). In some cases, the metal (e.g., metal ion) extracted from the mineral material is lithium, aluminum, iron, nickel, cobalt, uranium, strontium, a rare earth element, or any combination thereof. In some cases, the metal is lithium. In some cases, the metal ion is aluminum. In some cases, the metal ion is iron. In some cases, the metal ion is strontium.

In some cases, the metal is released into a solution. In some cases, the metal is extracted and released in form of metal ion. In some cases, the metal is extracted and released in form of a metal atom. In some cases, the metal is solubilized in a solution comprising water and/or buffer. In some cases the buffer comprises TRIS, PBS, citrate, monosodium glutamate, or any combination thereof. In some cases, the metal precipitates in the solution. In some cases, the metal is released and/or extracted in form of a metal complex. In some cases, the method further comprises extracting, and/or separating the metal from the solution. Any proper separation technique may be used. In some cases, an electromagnetic force may be used to separate metal ions from the solution. In some cases, a solid-liquid separation technique may be used to separate a metal precipitate from the solution. Any combination of separation and processing methods may be used. The method may comprise collecting the metal from the mineral material (e.g., source rock/ore) and from the system or solution used to perform the extraction according to the embodiments of the present disclosure and provide the metal for use in its intended application. In some cases, the metal may be processed as an industry-grade metal, battery-grade metal, or pharmaceutical-grade metal. An example of this may comprise industry-grade metal, battery-grade metal, or pharmaceutical-grade lithium.

In some cases, the method further comprises comprising purifying the metal from the solution, thereby generating a purified metal, a metal ion, a metal atom, a solid metal complex, a metal precipitate, or any combination thereof. In some cases, the purified metal ion a purity of at least about 50%, at least about 60%, at least about 70%, at least about 80%, at least about 90%, at least about 95%, at least about 99%, at least about 99.9%, at least about 99.99%, at least about 99.999%, at least about 99.9999% or greater.

In some cases, the method is performed in situ or ex-situ. In some embodiments, the enzyme having silicase activity has a catalytic efficiency of at least about 80%, at least about 85%, at least about 90%, at least about 91%, at least about 92%, at least about 93%, at least about 94%, at least about 95%, at least about 98% at least about 99%, at least about 99.5% or higher. In some embodiments, the method has a maximum metal extraction rate of at least about 80%, at least about 85%, at least about 90%, at least about 91%, at least about 92%, at least about 93%, at least about 94%, at least about 95%, at least about 98% at least about 99%, at least about 99.5% or higher. In some embodiments, the enzyme having silicase activity has a catalytic efficiency of at least about 1.1, 1.2, 1.3, 1.4, 1.5, 1.6, 1.7, 1.8, 1.9, 2, 2.25, 2.5, 2.75, 3, 3.25, 3.5, 3.75, 4, 4.25, 4.5, 4.75, 5, 5.5, 6, 6.5, 7, 8, 9, 10 times or higher. In some embodiments, the method has a maximum metal extraction rate of at least about 1.1, 1.2, 1.3, 1.4, 1.5, 1.6, 1.7, 1.8, 1.9, 2, 2.25, 2.5, 2.75, 3, 3.25, 3.5, 3.75, 4, 4.25, 4.5, 4.75, 5, 5.5, 6, 6.5, 7, 8, 9, 10 times or higher.

In some cases, the enzyme having silicase activity is recombinantly produced in a host cell or in a cell-free production system. In some cases, the host cell is a bacterial cell or a yeast cell. In some cases, the bacterial cell is Escherichia coli or the yeast cell is Pichia pastoris or Saccharomyces cerevisiae.

In an aspect, provided herein is a reaction mixture comprising an enzyme having silicase activity, and a non-natural co-factor. In some cases, the non-natural co-factor is bound to the enzyme having silicase activity. In some cases, the non-natural co-factor increases a function of the enzyme having silicase activity as compared to a reaction mixture comprising the enzyme having silicase activity and a natural co-factor. In some cases, the non-natural co-factor is copper, iron, nickel, cobalt, or glycine. In some cases, the natural co-factor is zinc. In some cases, the natural co-factor is iron. In some cases, the reaction mixture does not contain the natural co-factor. In some cases, the non-natural co-factor does not act as a co-factor for the enzyme having silicase activity in nature. In some cases, the reaction mixture further comprises the mineral material comprising silicate. In some cases, the reaction mixture has reaction conditions such that a metal contained within the mineral material is extracted, released, or solubilized, or precipitated into a solution from the mineral material. For example, a solution may be provided in proximity of a mineral material comprising a metal-bearing silicate. An enzyme having silicase activity and a non-natural co-factor according to the embodiments disclosed anywhere herein may be present in the solution. The enzyme may facilitate breaking Si—O bonds in the mineral material, thereby digesting and/or degrading the mineral material (e.g., rock/ore) and releasing the metal encased in the mineral material in form of metal ion, metal atom, metal solubilized in solution, metal precipitated in solution, or any combination thereof. The non-natural co-factor combined with or bound to the enzyme may further enhance the catalytic efficiency of the enzyme in releasing the metal from the mineral material in any of the mentioned forms.

In some cases, the enzyme having silicase activity has increased ability to release metal in any form mentioned anywhere herein, from mineral materials (e.g., ore/rock) in the presence of the non-natural co-factor as compared to the enzyme having silicase activity in the presence of the natural co-factor. In some cases, the mineral material may comprise silicates. In some cases, the mineral material may comprise a metal bearing silicate. In some cases, the mineral material comprises an inosilicate, a phyllosilicate, an amorphous silicate, or any combination thereof. In some cases, the inosilicate is selected from the group consisting of: spodumene, wollastonite, balangeroite, eveslogite, holmquistite, jadeite, shattuckite, augite, tremolite, and any combination thereof. In some cases, the phyllosilicate is selected from the group consisting of: lepidolite, hectorite, kaolinite, vermiculite, muscovite, montmorillonite, and any combination thereof. In some cases, the amorphous silicate is selected from the group consisting of obsidian, coal fly ash, pumice, glass, and any combination thereof.

In some cases, the enzyme having silicase activity has a sequence identity of at least about 5%, at least about 10%, at least about 20%, at least about 30%, at least about 40%, at least about 50% or more identity with a carbonic anhydrase. In some cases, the enzyme having silicase activity has a sequence identity of at least about 5%, at least about 10%, at least about 20%, at least about 30%, at least about 40%, at least about 50% or more identity with an alpha carbonic anhydrase or a gamma carbonic anhydrase. In some cases, the enzyme having silicase activity is derived from an organism selected from the group consisting of: Methanosarcina thermophila, Bacillus lichenmformis CG-B52, Pelobacter carbinolicus, Syntrophus aciditrophicus, Methanosarcina barkeri, Methanosarcina mazei, Bacillus halodurans, Alkalihalobacillus clausii (strain KSM-K16) (Bacillus clausii), Methanosarcina acetivorans, Kofleriaceae bacterium SLC26A/SulP, Thermodesulfitimonas autotrophica, Fischerella thermalis/Mastigocladus laminosus, Thermosynechococcus vestitus BP-1/(Thermosynechococcus elongatus BP-1) carboxysome assembly protein CcmM, Methanothrix thermoacetophila, Thermosyntropha lipolytica, isoleucine patch superfamily, Desulfofundulus thermobenzoicus, Archaeoglobus veneficus, Suberites domuncula, and any combination thereof.

In some cases, the non-naturally occurring enzyme comprises an amino acid sequence having at least about 5%, at least about 10%, at least about 20%, at least about 30%, at least about 40%, at least about 50%, at least about 60%, at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, at least about 99.5%, at least about 99.9%, or more sequence identity to an amino acid sequence of any one of SEQ ID NOS: 1-402. In some cases, the non-naturally occurring enzyme comprises an amino acid sequence having at least about 5%, at least about 10%, at least about 20%, at least about 30%, at least about 40%, at least about 50%, at least about 60%, at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, at least about 99.5%, at least about 99.9%, or more sequence identity to an amino acid sequence of any one of SEQ ID NOS: 403-464. In some cases, the enzyme having silicase activity is an engineered enzyme. In some cases, the enzyme having silicase activity is an enzyme having at least one amino acid variation as compared to a wild-type enzyme.

In some embodiments, the enzyme having silicase activity has a catalytic efficiency of at least about 80%, at least about 85%, at least about 90%, at least about 91%, at least about 92%, at least about 93%, at least about 94%, at least about 95%, at least about 98% at least about 99%, at least about 99.5% or higher. In some embodiments, the method has a maximum metal extraction rate of at least about 80%, at least about 85%, at least about 90%, at least about 91%, at least about 92%, at least about 93%, at least about 94%, at least about 95%, at least about 98% at least about 99%, at least about 99.5% or higher. In some embodiments, the enzyme having silicase activity has a catalytic efficiency of at least about 1.1, 1.2, 1.3, 1.4, 1.5, 1.6, 1.7, 1.8, 1.9, 2, 2.25, 2.5, 2.75, 3, 3.25, 3.5, 3.75, 4, 4.25, 4.5, 4.75, 5, 5.5, 6, 6.5, 7, 8, 9, 10 times or higher. In some embodiments, the method has a maximum metal extraction rate of at least about 1.1, 1.2, 1.3, 1.4, 1.5, 1.6, 1.7, 1.8, 1.9, 2, 2.25, 2.5, 2.75, 3, 3.25, 3.5, 3.75, 4, 4.25, 4.5, 4.75, 5, 5.5, 6, 6.5, 7, 8, 9, 10 times or higher.

In some embodiments, the enzyme having silicase activity has a pKd of at least about 5, at least about 6, at least about 7, at least about 8, at least about 9, at least about 10, at least about 11, or higher. In some embodiments, the enzyme having silicase activity has a Kcat value of at least about 2 mol per second (mol/s), at least about 10 mol/s, at least about 50 mol/s, at least about 100 mol/s, at least about 200 mol/s, at least about 300 mol/s, at least about 400 mol/s, at least about 500 mol/s, at least about 600 mol/s, at least about 700 mol/s, at least about 800 mol/s, at least about 900 mol/s, at least about 1000 mol/s, or higher.

In some cases, the reaction mixture has a pH from about 4 to about 11. In some cases, the reaction mixture has a pH from about 4 to about 10. In some cases, the reaction mixture has a pH from about 4 to about 9. In some cases, the reaction mixture has a pH from about 4 to about 8. In some cases, the reaction mixture has a pH from about 4 to about 7. In some cases, the reaction mixture has a pH from about 4 to about 6. In some cases, the reaction mixture has a pH from about 5 to about 11. In some cases, the reaction mixture has a pH from about 5 to about 10. In some cases, the reaction mixture has a pH from about 5 to about 9. In some cases, the reaction mixture has a pH from about 5 to about 8. In some cases, the reaction mixture has a pH from about 5 to about 7. In some cases, the reaction mixture has a pH from about 5 to about 6. In some cases, the reaction mixture has a pH from about 6 to about 11. In some cases, the reaction mixture has a pH from about 6 to about 10. In some cases, the reaction mixture has a pH from about 7 to about 11. In some cases, the reaction mixture has a pH from about 7 to about 10. In some cases, the reaction mixture has a pH from about 8 to about 11. In some cases, the reaction mixture has a pH from about 8 to about 10. In some cases, the reaction mixture has a pH from about 9 to about 11. In some cases, the reaction mixture has a pH from about 9 to about 10. In some cases, the reaction mixture has a pH of 4. In some cases, the reaction mixture has a pH of 5. In some cases, the reaction mixture has a pH of 6. In some cases, the reaction mixture has a pH of 7. In some cases, the reaction mixture has a pH of 8. In some cases, the reaction mixture has a pH of 9. In some cases, the reaction mixture has a pH of 10. In some cases, the reaction mixture has a pH of 11. In some cases, the reaction mixture has a temperature from about 20 to about 90 degrees Celsius (C). In some cases, the reaction mixture has a temperature from about 23 to about 90 degrees Celsius (C). In some cases, the reaction mixture has a temperature from about 23 to about 85 degrees Celsius (C). In some cases, the reaction mixture has a temperature from about 30 to about 90, from about 30 to about 80, from about 30 to about 70, from about 30 to about 60, or from about 30 to about 50 degrees Celsius (C). In some cases, the reaction mixture has a temperature from about 45 to about 55 degrees Celsius (C). In some cases, the reaction mixture has a temperature from about 45 to about 50 degrees Celsius (C). In some cases, the reaction mixture has a temperature about 20 degrees Celsius (C). In some cases, the reaction mixture has a temperature about 23 degrees Celsius (C). In some cases, the reaction mixture has a temperature about 25 degrees Celsius (C). In some cases, the reaction mixture has a temperature about 30 degrees Celsius (C). In some cases, the reaction mixture has a temperature about 35 degrees Celsius (C). In some cases, the reaction mixture has a temperature about 40 degrees Celsius (C). In some cases, the reaction mixture has a temperature about 45 degrees Celsius (C). In some cases, the reaction mixture has a temperature about 50 degrees Celsius (C). In some cases, the reaction mixture has a temperature about 55 degrees Celsius (C). In some cases, the reaction mixture has a temperature about 60 degrees Celsius (C). In some cases, the reaction mixture has a temperature about 70 degrees Celsius (C). In some cases, the reaction mixture has a temperature about 75 degrees Celsius (C). In some cases, the reaction mixture has a temperature about 80 degrees Celsius (C). In some cases, the reaction mixture has a temperature about 85 degrees Celsius (C). In some cases, the buffered solution comprises saline, glycine, iron ions, or any combination thereof. In some cases the buffered solution comprises TRIS, PBS, citrate, monosodium glutamate, or any combination thereof. In some cases, the reaction mixture further comprises an activator co-factor of the non-naturally occurring enzyme. In some cases, the activator co-factor comprises glycine, iron ion, or both. In some cases, the metal is lithium, aluminum, iron, nickel, cobalt, strontium, or a rare earth element. In some cases, the metal is lithium. In some cases, the metal is aluminum. In some cases, the metal is iron. In some cases, the metal is strontium. In some cases, the enzyme having silicase activity is recombinantly produced in a host cell or in a cell-free production system. In some cases, the host cell is a bacterial cell or yeast cell. In some cases, the bacterial cell is Escherichia coli, or the yeast cell is Pichia pastoris or Saccharomyces cerevisiae.

In some cases, the reaction mixture provided herein comprise crushing or grinding the rocks or ores to achieve a particulate size. In some cases, the ground ores comprise a size of about 50 μm to 1 mm. In some cases, the ground ores comprise a size from about 50 μm to 750 μm. In some cases, the ground ores comprise a size from about 50 μm to 500 μm. In some cases, the ground ores comprise a size from about 50 μm to 250 μm. In some cases, the ground ores comprise a size from about 50 μm to 150 μm. In some cases, the ground ores comprise a size from about 50 μm to 100 μm. In some cases, the ground ores comprise a size of about 50 μm. In some cases, the ground ores comprise a size of about 100 μm. In some cases, the ground ores comprise a size of about 150 μm. In some cases, the ground ores comprise a size of about 250 μm. In some cases, the ground ores comprise a size of about 500 μm. In some cases, the ground ores comprise a size of about 750 μm. In some cases, the ground ores comprise a size of about 1 mm.

In some cases, the reaction mixture provided herein comprise creating a slurry of crushed rock and liquid. In some cases, the rock to liquid ratio is from about 1-40% (w/v). In some cases, the rock to liquid ratio is from about 1-35% (w/v). In some cases, the rock to liquid ratio is from about 1-30% (w/v). In some cases, the rock to liquid ratio is from about 1-25% (w/v). In some cases, the rock to liquid ratio is from about 1-20% (w/v). In some cases, the rock to liquid ratio is from about 1-15% (w/v). In some cases, the rock to liquid ratio is from about 1-10% (w/v). In some cases, the rock to liquid ratio is from about 1-5% (w/v). In some cases, the rock to liquid ratio is from about 10-40% (w/v). In some cases, the rock to liquid ratio is from about 10-35% (w/v). In some cases, the rock to liquid ratio is from about 10-30% (w/v). In some cases, the rock to liquid ratio is from about 10-25% (w/v). In some cases, the rock to liquid ratio is from about 15-35% (w/v). In some cases, the rock to liquid ratio is from about 15-30% (w/v). In some cases, the rock to liquid ratio is from about 20-35% (w/v). In some cases, the rock to liquid ratio is from about 20-30% (w/v). In some cases, the rock to liquid ratio is from about 25-35% (w/v). In some cases, the rock to liquid ratio is from about 25-30% (w/v). In some cases, the rock to liquid ratio is about 1% (w/v). In some cases, the rock to liquid ratio is about 5% (w/v). In some cases, the rock to liquid ratio is about 10% (w/v). In some cases, the rock to liquid ratio is about 15% (w/v). In some cases, the rock to liquid ratio is about 20% (w/v). In some cases, the rock to liquid ratio is about 25% (w/v). In some cases, the rock to liquid ratio is about 30% (w/v). In some cases, the rock to liquid ratio is about 35% (w/v). In some cases, the rock to liquid ratio is about 40% (w/v).

In some cases, the reaction mixture provided herein comprise an enzymatic reaction that proceeds for a set period of time. In some cases, the reaction mixture comprises the enzymatic reaction proceeding for about 1-72 hours. In some cases, the reaction mixture comprises the enzymatic reaction proceeding for about 1-60 hours. In some cases, the reaction mixture comprises the enzymatic reaction proceeding for about 1-48 hours. In some cases, the reaction mixture comprises the enzymatic reaction proceeding for about 1-36 hours. In some cases, the reaction mixture comprises the enzymatic reaction proceeding for about 1-24 hours. In some cases, the reaction mixture comprises the enzymatic reaction proceeding for about 1-12 hours. In some cases, the reaction mixture comprises the enzymatic reaction proceeding for about 1-6 hours. In some cases, the reaction mixture comprises the enzymatic reaction proceeding for about 12-72 hours. In some cases, the reaction mixture comprises the enzymatic reaction proceeding for about 12-60 hours. In some cases, the reaction mixture comprises the enzymatic reaction proceeding for about 12-48 hours. In some cases, the reaction mixture comprises the enzymatic reaction proceeding for about 12-36 hours. In some cases, the reaction mixture comprises the enzymatic reaction proceeding for about 12-24 hours. In some cases, the reaction mixture comprises the enzymatic reaction proceeding for about 24-72 hours. In some cases, the reaction mixture comprises the enzymatic reaction proceeding for about 24-60 hours. In some cases, the reaction mixture comprises the enzymatic reaction proceeding for about 24-48 hours. In some cases, the reaction mixture comprises the enzymatic reaction proceeding for about 24-36 hours. In some cases, the reaction mixture comprises the enzymatic reaction proceeding for about 36-72 hours. In some cases, the reaction mixture comprises the enzymatic reaction proceeding for about 36-60 hours. In some cases, the reaction mixture comprises the enzymatic reaction proceeding for about 36-48 hours. In some cases, the reaction mixture comprises the enzymatic reaction proceeding for about 48-72 hours. In some cases, the reaction mixture comprises the enzymatic reaction proceeding for about 48-60 hours. In some cases, the reaction mixture comprises the enzymatic reaction proceeding for about 1 hour. In some cases, the reaction mixture comprises the enzymatic reaction proceeding for about 6 hours. In some cases, the reaction mixture comprises the enzymatic reaction proceeding for about 12 hours. In some cases, the reaction mixture comprises the enzymatic reaction proceeding for about 24 hours. In some cases, the reaction mixture comprises the enzymatic reaction proceeding for about 36 hours. In some cases, the reaction mixture comprises the enzymatic reaction proceeding for about 48 hours. In some cases, the reaction mixture comprises the enzymatic reaction proceeding for about 60 hours. In some cases, the reaction mixture comprises the enzymatic reaction proceeding for about 72 hours.

In some cases, provided herein are engineered enzymes. In some cases, the method of enzyme engineering comprises performing directed molecular evolution on an enzyme sequence and generating an evolved enzyme sequence, wherein the evolved enzyme sequence has a higher specificity to a substrate in a mineral material (e.g., a silicate rock), a higher catalytic rate for acting on the mineral material, or both, compared to the first enzyme sequence. The first enzyme sequence may be a wild-type enzyme. In some cases, the wild-type enzyme is a carbonic anhydrase, a gamma carbonic anhydrase, or an alpha carbonic anhydrase.

The evolved enzyme, the wild-type enzyme, or both may cleave Si—O bonds in the substrate and extract a metal from the mineral material, in some cases a silicate rock. In some examples, directed molecular evolution is performed using a Machine Learning (ML) or Artificial Intelligence (AI) Algorithm. In some cases, performing directed molecular evolution comprises deoxyribonucleic acid (DNA) shuffling. In some cases, the ML or AI algorithm comprises one or more of structural sequence generation, sequence ranking, and sequence fine-tuning. In some cases, the ML or AI algorithm comprises a transformer model system. In some cases, the ML or AI algorithm comprises using natural language processing (NLP). In some cases, the evolved enzyme sequence is the sequence of the synthetic enzyme used in the methods of any of the embodiments of the present disclosure.

### EXAMPLES

### Example 1. Methods for Enzymatic Degradation Assay

### Materials

Buffer recipe: 50 micro-Molar (μM) amino acid activator, 250 micro-Molar (μM) co-factor, and 0.15 Molar (M) Sodium Chloride (NaCl) diluted in distilled water
Sample comprising mineral material and enzyme in buffer (positive sample): 0.175 grams (g) of mineral material (e.g., ore or rock or other mineral), 7 milliliters (mL) of buffer, 350 microliters (μL) of purified enzyme (0.51 mg/mL enzyme). The enzyme having silicase activity according to the embodiments and sequences described anywhere in the present disclosure.
Negative control sample: 0.175 g of mineral material, 7350 μL of buffer.

### Procedure

Mineral samples were crushed and sifted to a grain size of 150 μM. they were subsequently washed with distilled water and ethanol. Samples were then centrifuged, and the pellets dried for 12 hours at 100° C. 2.5% (weight/volume (w/v)) solutions were made for both the positive sample and the negative control. Samples were resuspended in 96-well flat bottom plates and placed in an OT-2 heater shaker set to 300 rounds per minute (rpm) and a temperature from about 50 to about 60° C., overnight with periodic samples taken at regular time intervals. Samples were degraded and assayed in triplicate. The molybdenum blue photometry method in solution was used to determine the concentration of colloidal silica and soluble silica in the samples as well as for the standard curve. The assays were conducted with an automated procedure on an OT-2 Opentrons and absorbance taken on a Byuonoy Absorbance 96.

Positive reaction rates minus the negative baseline rates were calculated and normalized based on an established silica concentration reference curve.

### Results

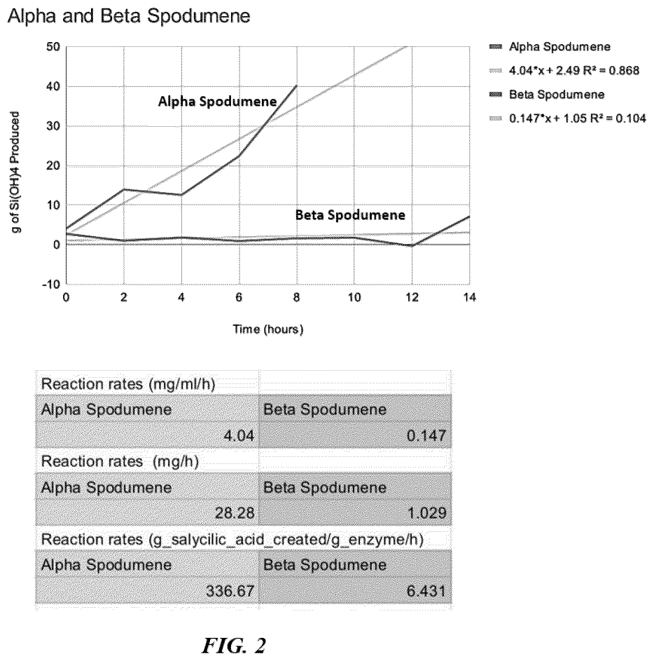

The method was performed on a variety of mineral materials, and the results are present in FIGS. 2-7.

FIG. 2 presents the rate of production of Si(OH)₄ corresponding to reaction rates of degrading silicate minerals(Alpha Spodumene and Beta Spodumene) using an enzyme having silicase activity according to the embodiments of the present disclosure.

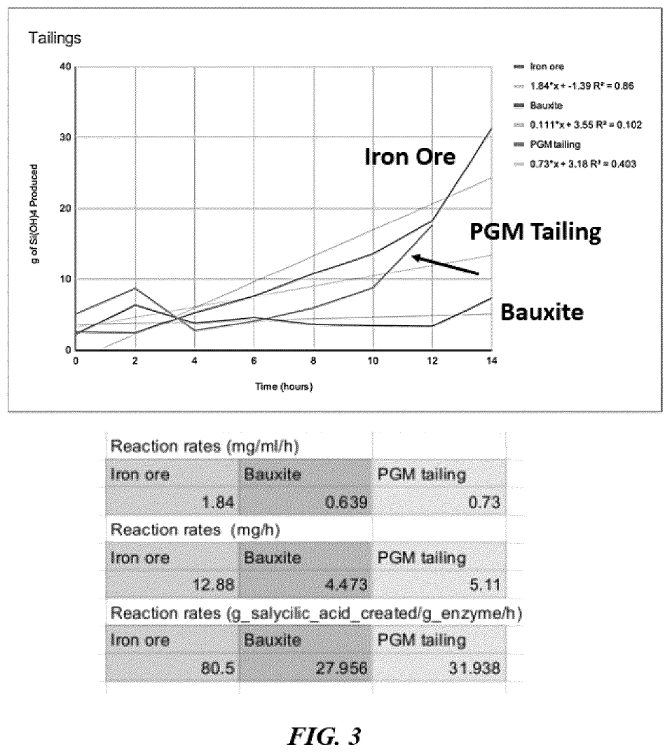

FIG. 3 presents the rate of production of Si(OH)₄ corresponding to reaction rates of degrading silicate minerals (iron ore, platinum group metal (PGM) tailing, and Bauxite) using an enzyme having silicase activity according to the embodiments of the present disclosure.

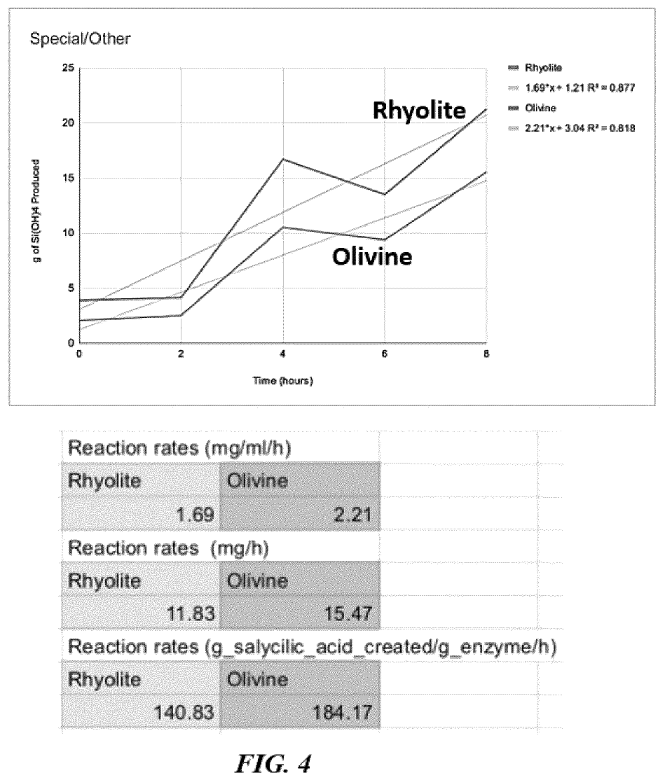

FIG. 4 presents the rate of production of Si(OH)₄ corresponding to reaction rates of degrading silicate minerals (Rhyolite and Olivine) using an enzyme having silicase activity according to the embodiments of the present disclosure.

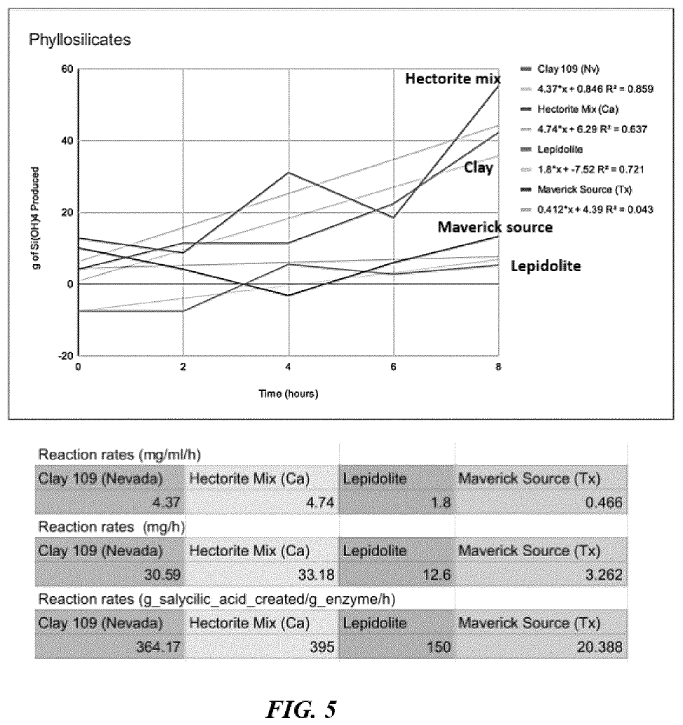

FIG. 5 presents the rate of production of Si(OH)₄ corresponding to reaction rates of degrading silicate minerals (Hectorite mix, Clay, a silicate named Maverick source, and a Lepidolite) using an enzyme having silicase activity according to the embodiments of the present disclosure.

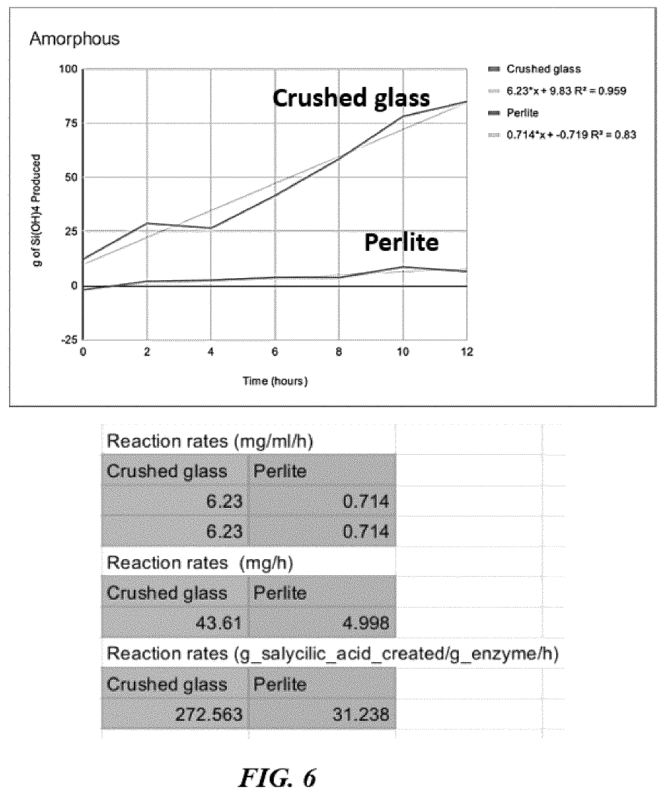

FIG. 6 presents the rate of production of Si(OH)₄ corresponding to reaction rates of degrading silicate minerals (crushed glass and Perlite) using an enzyme having silicase activity according to the embodiments of the present disclosure.

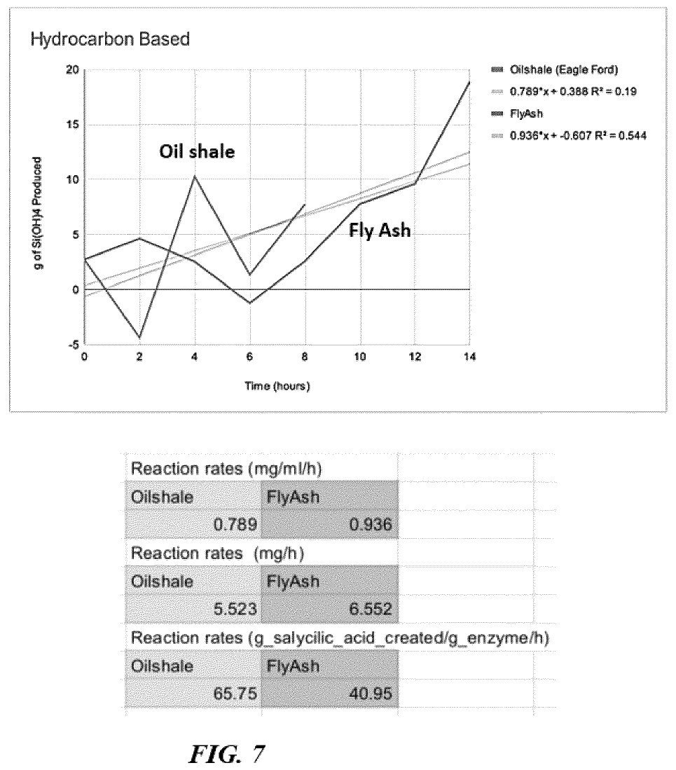

FIG. 7 presents the rate of production of Si(OH)4 corresponding to reaction rates of degrading silicate minerals (Oil Shale and Fly Ash) using an enzyme having silicase activity according to the embodiments of the present disclosure.

### Example 2. Inosilicate Degradation with Enzymes Having Silicase Activity

The enzyme Gamma Carbonic Anhydrase from M. thermophila degrades silicate mineral material which allows for the extraction of metals such as lithium and aluminum, for example. Enzymes having silicase activity were designed from gamma carbonic anhydrase to improve the efficiency of the degradation reaction of silicate mineral material, specifically inosilicates, such as alpha spodumene, augite, and tremolite.

The 17 amino acid residue signal sequence as well as part of the disordered region (about residues 18 to 64, starting from the N-terminus) of wildtype Gamma Carbonic Anhydrase from M. thermophila were truncated. The wildtype truncation is as shown in SEQ ID NO: 403. From there, further mutations were generated to optimize for enzymatic efficacy, as shown in SEQ ID NOs: 404-433. Table 3 shows the percentage identity, as calculated by comparing sequence information using the advanced BLAST computer program, of the enzymes having silicase activity compared to wildtype gamma carbonic anhydrase from M. thermophila.

TABLE 3
Percentage Identity of Enzymes Having Silicase
Activity to Wildtype Gamma Carbonic Anhydrase

| | % Identity to Gamma |
|---|---|
| | Carbonic Anhydrase from |
| | M. thermophila (SEQ ID |
| SEQ ID NO: | NO: 1) |
| 403 | 100 |
| 404 | 65.0 |
| 405 | 67.8 |
| 406 | 54.7 |
| 407 | 61.1 |
| 408 | 65.4 |
| 409 | 73.6 |
| 410 | 71.5 |
| 411 | 67.6 |
| 412 | 66.5 |
| 413 | 64.3 |
| 414 | 58.0 |
| 415 | 69.5 |
| 416 | 72.8 |
| 417 | 46.1 |
| 418 | 70.5 |
| 419 | 51.7 |
| 420 | 38.3 |
| 421 | 60.1 |
| 422 | 71.6 |
| 423 | 67.1 |
| 424 | 50.0 |
| 425 | 58.9 |
| 426 | 68.9 |
| 427 | 58.2 |
| 428 | 74.3 |
| 429 | 42.9 |
| 430 | 69.3 |
| 431 | 58.7 |
| 432 | 46.3 |
| 433 | 67.5 |

Enzymes having silicase activity of SEQ ID NOs: 403-433 were generated comprising a GST tag and expressed in E. coli BL21 using a pGEX-6P1 GST codon-optimized vector. E. coli cultures were grown at 37° C. with shaking and induced. After induction, the cells were harvested by centrifugation and lysed. The lysate was supplemented with ferrous gluconate to provide a source of Fe. The lysate was heated to 55° C. to aide in protein folding and stability. Enzymes were generated comprising an N-terminal GST tag. Other common purification tags, such as NEXT or 6-HIS, or no tags may be utilized in purification and experimental use of the enzymes. The enzymes were purified using a GST affinity column. The GST tag was found to not affect enzymic activity under reaction conditions.

The degradation reactions were tested for the various enzymes having silicase activity with alpha spodumene, augite, or tremolite. Degradation reactions were conducted in plastic containers to avoid silicate dissolution from glass. A range of reaction conditions were tested. The reaction was operational in a buffer comprising TRIS, PBS, 0.1 M citrate, or 0.9% monosodium glutamate in a pH range of 4-11, the rock to liquid rock ratio was tested between 1-40% (w/v), the minerals were crushed with a small grinder to ground ore sizes between 50 μm to 1 mm, the reaction was shaken with a range of 1-220 RPM, at a temperature between 23-85° C., the enzymes having silicase activity of SEQ ID NOs: 403-433 with GST, NEXT, 6-His or no N-terminal tag were tested, Zn, Fe, Cu, or Co metal cofactors were included, and the reaction ran for a time range of 1-48 hours. The range of reaction conditions tested resulted in degradation of silicate material. Data not shown.

Optimal reaction conditions were developed and used to compare the enzymes having silicase activity of SEQ ID NOs: 403-433. The enzymes were tested with alpha spodumene, augite, and tremolite. Optimized reaction conditions were found to be the following: the minerals were crushed with a small grinder and sifted to achieve a particulate size between 50 and 150 μm. The reaction proceeded in a 0.1 M TRIS buffer at pH 10 in a rock to liquid rock ration of 30% (w/v) at a temperature of 51° C. and shaking at 220 RPM for 48 hours. Reactions were completed in triplicate. After the reaction proceeded for 48 hours, the suspensions were centrifuged at 14,000 RPM for 15 minutes. The supernatant was filtered using a 45 μm, 13 mm diameter syringe filter.

The elemental composition of the supernatant was determined using a Laser Induced Breakdown Spectroscopy (LIBS) lithium brine analyzer and X-ray fluorescence analyzer. The results of the extracted metals were measured as PPM in solution as shown in Table 4. Degradation results were normalized to compared to a truncated wildtype Gamma Carbonic Anhydrase from M. thermophila, SEQ ID NO: 403.

The hydrolytic activity of the enzymes having silicase activity on the crystalline structure was also assessed using a silica degradation assay. The assay measured the amount of free silica (Si(OH)₄, orthosilicic acid) using a molybdenum blue assay. The reaction proceeded as described above. 100 μL of the supernatant was deposited into a 96-well plate. 10 μL 1:1 sulfuric acid solution was added to the sample and mixed with the sample. 20 μL of 5% ammonium molybdate solution was added to the acidified sample and allowed to rest. Next, 20 μL of 0.5% ascorbic acid reducing reagent was added to the mixture and allowed the color to develop for 10 minutes. The molybdenum blue assay reaction mixture was transferred to a microcuvette and the absorbance was measured at 810 nm. The amount of free orthosilicic acid was calculated against a calibrated standard curve The results of the degradation of enzymes having silicase activity of SEQ ID NOs: 404-433 were normalized to activity of SEQ ID NO: 403 as shown in Table 5. The results show superior extraction of Fe, Li and Al as well as enzyme degradation activity in the enzymes having silicase activity of SEQ ID NOs: 404-433 as compared to the designed truncated wildtype enzyme having silicase activity of SEQ ID NO: 403.

TABLE 4
Metal Extraction Reaction Results

| | |
|---|---|
| | Degradation activity |
| | compared to truncated |
| SEQ ID NO: | wildtype enzyme |

Degradation of alpha spodumene

| | |
|---|---|
| 403 | 1 |
| 404 | 2.3 |
| 405 | 4.6 |
| 406 | 1.6 |
| 407 | 3.5 |
| 408 | 1.7 |
| 409 | 1.5 |
| 410 | 2.1 |
| 411 | 2 |
| 412 | 1.5 |
| 413 | 1.1 |

Degradation of augite

| | |
|---|---|
| 403 | 1 |
| 414 | 3.9 |
| 415 | 1.4 |
| 416 | 5 |
| 417 | 1.5 |
| 418 | 3.4 |
| 419 | 1.3 |
| 420 | 6.9 |
| 421 | 2.1 |
| 422 | 1.5 |
| 423 | 3.2 |

Degradation of tremolite

| | |
|---|---|
| 403 | 1 |
| 424 | 2.1 |
| 425 | 1.2 |
| 426 | 1.2 |
| 427 | 1.8 |
| 428 | 1.4 |
| 429 | 4.2 |
| 430 | 1.2 |
| 431 | 3.2 |
| 432 | 2.5 |
| 433 | 2 |

TABLE 5
Degradation Activity of Enzymes Having Silicase Activity

| | | | |
|---|---|---|---|
| SEQ ID NO: | Fe (PPM) | Li (PPM) | Al (PPM) |

Metal extraction of alpha spodumene

| | | | |
|---|---|---|---|
| 403 | 29177 | 2500 | 29744 |
| 404 | 33654 | 3128 | 35912 |
| 405 | 58434 | 4970 | 71002 |
| 406 | 31598 | 2774 | 33592 |
| 407 | 51460 | 4350 | 62887 |
| 408 | 32134 | 2802 | 34201 |
| 409 | 31025 | 2659 | 32378 |
| 410 | 33380 | 3020 | 35312 |
| 411 | 33014 | 2982 | 34789 |
| 412 | 30896 | 2640 | 32157 |
| 413 | 29756 | 2536 | 30298 |

Metal extraction of augite

| | | | |
|---|---|---|---|
| 403 | 21072 | 2877 | 31841 |
| 414 | 41234 | 5604 | 62197 |
| 415 | 23918 | 3265 | 36029 |
| 416 | 51043 | 6820 | 77102 |
| 417 | 24368 | 3321 | 36901 |
| 418 | 39692 | 5382 | 59748 |
| 419 | 22996 | 3104 | 34219 |
| 420 | 68129 | 9072 | 102432 |
| 421 | 30112 | 4098 | 45412 |
| 422 | 24560 | 3350 | 37129 |
| 423 | 37850 | 5137 | 57092 |

Metal extraction of tremolite

| | | | |
|---|---|---|---|
| 403 | 27594 | 29727 | 31841 |
| 424 | 31267 | 33564 | 39753 |
| 425 | 28345 | 30218 | 32053 |
| 426 | 28409 | 30301 | 32198 |
| 427 | 30567 | 32674 | 31252 |
| 428 | 29123 | 31089 | 34506 |
| 429 | 35981 | 38374 | 45098 |
| 430 | 28390 | 30296 | 39587 |
| 431 | 34012 | 36102 | 39584 |
| 432 | 32878 | 34906 | 38128 |
| 433 | 31856 | 33870 | 37656 |

### Example 3. Inosilicate Degradation with Host Cells Expressing Enzymes Having Silicase Activity

The enzyme Gamma Carbonic Anhydrase from M. thermophila degrades silicate mineral material which allows for the extraction of metals such as lithium and aluminum, for example. Enzymes having silicase activity are designed to improve the efficiency of the degradation reaction of silicate mineral material, specifically inosilicates, such as alpha spodumene, augite, and tremolite.

Enzymes having silicase activity are generated comprising a tag and expressed in a bacteria host cell, such as E. coli, using a host cell appropriate vector. Host cell cultures are grown and induced. After induction, the cells are harvested.

The degradation reactions are tested using the host cells expressing the enzymes having silicase activity with inosilicate materials, such as alpha spodumene, augite, and tremolite. Degradation reactions are conducted in plastic containers to avoid silicate dissolution from glass. A range of reaction conditions are tested. The reaction is tested in a buffer comprising TRIS, PBS, 0.1 M citrate, or 0.9% monosodium glutamate in a pH range of 4-11, the rock to liquid rock ratio is tested between 1-40% (w/v), the minerals are crushed with a small grinder to ground ore sizes between 50 μm to 1 mm, the reaction is shaken with a range of 1-220 RPM, at a temperature between 23-85° C., Zn, Fe, Cu, or Co metal cofactors are included, and the reaction is run for a time range of 1-48 hours.

The elemental composition of the supernatant is determined using a Laser Induced Breakdown Spectroscopy (LIBS) lithium brine analyzer and X-ray fluorescence analyzer. The results of the extracted metals are measured as PPM in solution.

The hydrolytic activity of the enzymes on the crystalline structure is also assessed using a silica degradation assay. The assay measures the amount of free silica (Si(OH)4, orthosilicic acid) using a molybdenum blue assay. Supernatant is deposited into a 96-well plate. 1:1 sulfuric acid solution is added to the sample and mixed with the sample. 5% ammonium molybdate solution is added to the acidified sample and allowed to rest. 0.5% ascorbic acid reducing reagent is added to the mixture and allowed the color to develop for 10 minutes. The molybdenum blue assay reaction mixture is transferred to a microcuvette and the absorbance is measured at 810 nm. The amount of free orthosilicic acid is calculated against a calibrated standard curve.

### Example 4. Phyllosilicate Degradation with Enzymes Having Silicase Activity

The enzyme gamma carbonic anhydrase from M. thermophila degrades silicate mineral material which allows for the extraction of metals such as lithium and aluminum, for example. Enzymes having silicase activity were designed from Gamma Carbonic Anhydrase to improve the efficiency of the degradation reaction of silicate mineral material, specifically phyllosilicates, such as lepidolite, montmorillonite, and muscovite.

The 17 amino acid residue signal sequence or the 17 amino acid residue signal sequence and part of the disordered region (about residues 18 to 64, starting from the N-terminus) of wildtype Gamma Carbonic Anhydrase from M. thermophila were truncated (SEQ ID NO: 403 and SEQ ID NO: 434 respectively). From there, further mutations were generated to optimize for enzymatic efficacy, as shown in SEQ ID NOs: 435-464. Table 6 shows the percentage identity, as calculated by comparing sequence information using the advanced BLAST computer program, of the enzymes having silicase activity compared to wildtype gamma carbonic anhydrase from M. thermophila.

TABLE 6
Percentage Identity of Enzymes Having Silicase
Activity to Wildtype Gamma Carbonic Anhydrase

| | % Identity to Gamma |
|---|---|
| | Carbonic Anhydrase from |
| | M. thermophila (SEQ ID |
| SEQ ID NO: | NO: 1) |
| 403 | 100 |
| 434 | 100 |
| 435 | 52.2 |
| 436 | 51.3 |
| 437 | 54.2 |
| 438 | 52.6 |
| 439 | 55.1 |
| 440 | 54.4 |
| 441 | 48.8 |
| 442 | 49.3 |
| 443 | 53.3 |
| 444 | 54.2 |
| 445 | 42.8 |
| 446 | 50.6 |
| 447 | 43.3 |
| 448 | 43.6 |
| 449 | 42.8 |
| 450 | 43.9 |
| 451 | 59.0 |
| 452 | 59.0 |
| 453 | 41.1 |
| 454 | 53.9 |
| 455 | 45.5 |
| 456 | 56.5 |
| 457 | 73.3 |
| 458 | 55.3 |
| 459 | 74.9 |
| 460 | 41.1 |
| 461 | 81.7 |
| 462 | 51.2 |
| 463 | 66.7 |
| 464 | 84.2 |

Enzymes having silicase activity of SEQ ID NOs: 403 and 434-464 were generated comprising a GST tag and expressed in E. coli BL21 using a pGEX-6P1 GST codon-optimized vector. E. coli cultures were grown at 37° C. with shaking and induced. After induction, the cells were harvested by centrifugation and lysed. The lysate was supplemented with ferrous gluconate to provide a source of Fe. The lysate was heated to 55° C. to aide in protein folding and stability. Enzymes were generated comprising an N-terminal GST tag. Other common purification tags, such as NEXT or 6-HIS, or no tags may be utilized in purification and experimental use of the enzymes. The enzymes were purified using a GST affinity column. The GST tag was found to not affect enzymic activity under reaction conditions.

The degradation reactions were tested for the various enzymes having silicase activity with lepidolite, montmorillonite, and muscovite. Degradation reactions were conducted in plastic containers to avoid silicate dissolution from glass. A range of reaction conditions were tested. The reaction was operational in a buffer comprising TRIS, PBS, 0.1 M citrate, or 0.9% monosodium glutamate in a pH range of 4-11, the rock to liquid rock ratio was tested between 1-40% (w/v), the minerals were crushed with a small grinder to ground ore sizes between 50 μm to 1 mm, the reaction was shaken with a range of 1-220 RPM, at a temperature between 23-85° C., the enzymes having silicase activity of SEQ ID NOs: 403 and 434-464 with GST, NEXT, 6-His or no N-terminal tag were tested, Zn, Fe, Cu, or Co metal cofactors were included, and the reaction ran for a time range of 1-48 hours. The range of reaction conditions tested resulted in degradation of silicate material. Data not shown.

Optimal reaction conditions were developed and used to compare the enzymes having silicase activity of SEQ ID NOs: 403 and 434-464. The enzymes were tested with lepidolite, montmorillonite, and muscovite. Optimized reaction conditions were found to be the following: the minerals were crushed with a small grinder and sifted to achieve a particulate size between 50 and 150 μm. The reaction proceeded in a 0.1 M TRIS buffer at pH 10 in a rock to liquid rock ration of 30% (w/v) at a temperature of 51° C. and shaking at 220 RPM for 48 hours. Reactions were completed in triplicate. After the reaction proceeded for 48 hours, the suspensions were centrifuged at 14,000 RPM for 15 minutes. The supernatant was filtered using a 45 μm, 13 mm diameter syringe filter.

The elemental composition of the supernatant was determined using a Laser Induced Breakdown Spectroscopy (LIBS) lithium brine analyzer and X-ray fluorescence analyzer. The results of the extracted metals were measured as PPM in solution as shown in Table 7. Degradation results were normalized to compared to a truncated wildtype Gamma Carbonic Anhydrase from M. thermophila (either of SEQ ID NOs: 403 or 434).

The hydrolytic activity of the enzymes having silicase activity on the crystalline structure was also assessed using a silica degradation assay. The assay measured the amount of free silica (Si(OH)₄, orthosilicic acid) using a molybdenum blue assay. The reaction proceeded as described above. 100 μL of the supernatant was deposited into a 96-well plate. 10 μL 1:1 sulfuric acid solution was added to the sample and mixed with the sample. 20 μL of 5% ammonium molybdate solution was added to the acidified sample and allowed to rest. Next, 20 μL of 0.5% ascorbic acid reducing reagent was added to the mixture and allowed the color to develop for 10 minutes. The molybdenum blue assay reaction mixture was transferred to a microcuvette and the absorbance was measured at 810 nm. The amount of free orthosilicic acid was calculated against a calibrated standard curve. The results of the degradation of enzymes were normalized to activity of a truncated wildtype Gamma Carbonic Anhydrase from M. thermophila as shown in Table 8. The results of the degradation using enzymes having silicase activity of SEQ ID NOs: 435-464 were normalized to activity of SEQ ID NOs: 403 or 434 as shown in Table 5. The results show superior extraction of Fe, Li and Al as well as enzyme degradation activity in the enzymes having silicase activity of SEQ ID NOs: 435-464 as compared to the designed truncated wildtype enzyme having silicase activity of SEQ ID NO: 403 and SEQ ID NO: 434.

TABLE 7
Metal Extraction Reaction Results

| | |
|---|---|
| | Degradation activity |
| | compared to SEQ ID NO: |
| SEQ ID NO: | 403 (truncated wildtype) |

Degradation of lepidolite

| | |
|---|---|
| 434 | 1 |
| 435 | 2 |
| 436 | 2.2 |
| 437 | 1.9 |
| 438 | 1.5 |
| 439 | 1.7 |
| 440 | 1.5 |
| 441 | 1.6 |
| 442 | 1.7 |
| 443 | 1.7 |
| 444 | 1.6 |

Degradation of montmorillonite

| 434 | 1 |
|---|---|
| 445 | 3.6 |
| 446 | 2.4 |
| 447 | 1.2 |
| 448 | 1.1 |
| 449 | 2 |
| 450 | 1.5 |
| 451 | 2.2 |
| 452 | 2.4 |
| 453 | 1.3 |
| 454 | 1.2 |
| | Degradation activity |
| | compared to SEQ ID NO: |
| SEQ ID NO: | 424 (truncated wildtype) |

Degradation of muscovite

| | |
|---|---|
| 403 | 1 |
| 455 | 1.7 |
| 456 | 2.8 |
| 457 | 2 |
| 458 | 1.5 |
| 459 | 1 |
| 460 | 3 |
| 461 | 1.7 |
| 462 | 1.4 |
| 463 | 4 |
| 464 | 2.4 |

TABLE 8
Degradation Activity of Enzymes Having Silicase Activity

| | | | | |
|---|---|---|---|---|
| SEQ ID NO: | Fe (PPM) | Li (PPM) | Al (PPM) | Sr (PPM) |

Metal extraction of lepidolite

| | | | | |
|---|---|---|---|---|
| 434 | 21287 | 3514 | 24903 | N.D. |
| 435 | 24021 | 3598 | 25968 | N.D. |
| 436 | 24341 | 3678 | 26245 | N.D. |
| 437 | 23123 | 3627 | 25873 | N.D. |
| 438 | 21786 | 3552 | 25514 | N.D. |
| 439 | 22259 | 3642 | 26031 | N.D. |
| 440 | 21892 | 3557 | 25487 | N.D. |
| 441 | 22018 | 3589 | 25716 | N.D. |
| 442 | 22230 | 3638 | 25955 | N.D. |
| 443 | 22242 | 3643 | 26028 | N.D. |
| 444 | 22011 | 3594 | 25732 | N.D. |

Metal extraction of montmorillonite

| | | | | |
|---|---|---|---|---|
| 434 | 21061 | 450 | 25105 | 2194 |
| 445 | 41584 | 789 | 55613 | 4216 |
| 446 | 32235 | 654 | 46147 | 3798 |
| 447 | 23018 | 478 | 26695 | 2389 |
| 448 | 23240 | 461 | 26821 | 2453 |
| 449 | 34012 | 652 | 37334 | 3728 |
| 450 | 24287 | 634 | 27568 | 3560 |
| 451 | 34763 | 658 | 32849 | 3851 |
| 452 | 35047 | 662 | 38312 | 3917 |
| 453 | 23391 | 592 | 26743 | 2329 |
| 454 | 23674 | 594 | 27058 | 2290 |

Metal extraction of muscovite

| | | | | |
|---|---|---|---|---|
| 403 | 20364 | 29624 | 24959 | N.D. |
| 455 | 21547 | 31789 | 28125 | N.D. |
| 456 | 25432 | 34812 | 38912 | N.D. |
| 457 | 23489 | 32950 | 34968 | N.D. |
| 458 | 22015 | 30970 | 27605 | N.D. |
| 459 | 20456 | 29730 | 24964 | N.D. |
| 460 | 26710 | 35890 | 40025 | N.D. |
| 461 | 21570 | 31810 | 28060 | N.D. |
| 462 | 21030 | 30200 | 25302 | N.D. |
| 463 | 28020 | 37250 | 46390 | N.D. |
| 464 | 24080 | 33420 | 32705 | N.D. |

N.D. = no data

### Example 5. Phyllosilicate Degradation with Host Cells Expressing Enzymes Having Silicase Activity

The enzyme Gamma Carbonic Anhydrase from M. thermophila degrades mineral material which allows for the extraction of metals such as lithium and aluminum, for example. Enzymes having silicase activity are designed to improve the efficiency of the degradation reaction of silicate mineral material, specifically phyllosilicates, such as lepidolite, montmorillonite, and muscovite.

Enzymes having silicase activity are generated comprising a tag and expressed in a bacteria host cell, such as E. coli, using a host cell appropriate vector. Host cell cultures are grown and induced. After induction, the cells are harvested.

The degradation reactions are tested using the host cells expressing the enzymes with phyllosilicate materials, such as lepidolite, montmorillonite, and muscovite. Degradation reactions are conducted in plastic containers to avoid silicate dissolution from glass. A range of reaction conditions are tested. The reaction is tested in a buffer comprising TRIS, PBS, 0.1 M citrate, or 0.9% monosodium glutamate in a pH range of 4-11, the rock to liquid rock ratio is tested between 1-40% (w/v), the minerals are crushed with a small grinder to ground ore sizes between 50 μm to 1 mm, the reaction is shaken with a range of 1-220 RPM, at a temperature between 23-85° C., Zn, Fe, Cu, or Co metal cofactors are included, and the reaction is run for a time range of 1-48 hours.

The elemental composition of the supernatant is determined using a Laser Induced Breakdown Spectroscopy (LIBS) lithium brine analyzer and X-ray fluorescence analyzer.

The results of the extracted metals are measured as PPM in solution.

The hydrolytic activity of the enzymes on the crystalline structure is also assessed using a silica degradation assay. The assay measures the amount of free silica (Si(OH)₄, orthosilicic acid) using a molybdenum blue assay. Supernatant is deposited into a 96-well plate. 1:1 sulfuric acid solution is added to the sample and mixed with the sample. 5% ammonium molybdate solution is added to the acidified sample and allowed to rest. 0.5% ascorbic acid reducing reagent is added to the mixture and allowed the color to develop for 10 minutes. The molybdenum blue assay reaction mixture is transferred to a microcuvette and the absorbance is measured at 810 nm. The amount of free orthosilicic acid is calculated against a calibrated standard curve.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of extracting a metal from a mineral material, the method comprising:
(a) contacting the mineral material with an enzyme having silicase activity under reaction conditions such that the metal contained within the mineral material is solubilized and released; and
(b) collecting the released metal,
thereby extracting the metal from the mineral material.

2. The method of claim 1, wherein the mineral material comprises an ore, a rock, a natural mineral material, a man-made mineral material, or any combination thereof.

3. The method of claim 1, wherein the mineral material comprises a silicate.

4. The method of claim 1, wherein the mineral material comprises an inosilicate, a phyllosilicate, an amorphous silicate, a tectosilicate, or any combination thereof.

5. The method of claim 1, wherein the enzyme having silicase activity has a sequence identity of at least about 30%, at least about 40%, about 50%, at least about 60%, at least about 70%, at least about 80%, at least about 90%, or more with an amino acid sequence of a gamma carbonic anhydrase.

6. The method of claim 1, wherein the enzyme having silicase activity has a sequence identity of at least about 30%, at least about 40%, at least about 50%, at least about 60%, at least about 70%, at least about 80%, at least about 90% or more, with an amino acid sequence of an enzyme selected from the group consisting of: Methanosarcina thermophila gamma carbonic anhydrase, Bacillus licheniformis CG-B52 gamma carbonic anhydrase, Pelobacter carbinolicus gamma carbonic anhydrase, Syntrophus aciditrophicus gamma carbonic anhydrase, Methanosarcina barkeri gamma carbonic anhydrase, Methanosarcina mazei carbonic anhydrase, Bacillus halodurans alpha carbonic anhydrase, Alkalihalobacillus clausii (strain KSM-K16) (Bacillus clausii) alpha carbonic anhydrase, Methanosarcina acetivorans carbonate dehydratase, Kofleriaceae bacterium SLC26A/SulP transporter domain-containing protein, Thermodesulfitimonas autotrophica carbonic anhydrase/acetyltransferase-like protein (Isoleucine patch superfamily), Fischerella thermalis/Mastigocladus laminosus JSC-11 carboxysome assembly protein CcmM, Thermosynechococcus vestitus BP-1/(Thermosynechococcus elongatus BP-1) carboxysome assembly protein CcmM, Methanothrix thermoacetophila carbonate dehydratase, Thermosyntropha lipolytica carbonic anhydrase or acetyltransferase, isoleucine patch superfamily, Desulfofundulus thermobenzoicus transferase, Archaeoglobus veneficus carbonate dehydratase, Suberites domuncula carbonic anhydrase.

7. The method of claim 1, wherein the enzyme having silicase activity has an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, or at least 99.9%, or more sequence identity to an amino acid sequence of any one of SEQ ID NOS: 1-402.

8. The method of claim 1, wherein the reaction conditions comprise a temperature from about 23 to about 85 degrees Celsius (C).

9. The method of claim 1, wherein the reaction conditions comprise a pH from about 4 to about 11.

10. The method of claim 1, wherein the reaction conditions comprise contacting the enzyme having silicase activity with a co-factor.

11. The method of claim 10, wherein the co-factor is selected from the group consisting of: iron, zinc, copper, nickel, and cobalt.

12. The method of claim 1, wherein the metal is selected from the group consisting of: lithium, aluminum, iron, nickel, cobalt, strontium, and a rare earth element.

13. The method of claim 1, wherein the metal is released into a solution.

14. The method of claim 13, further comprising extracting the metal from the solution.

15. The method of claim 13, further comprising purifying the metal from the solution, thereby generating a purified metal.

16. The method of claim 15, wherein the purified metal has a purity of at least about 80%.

17. The method of claim 1, wherein the method is performed in situ or ex situ.

18. The method of claim 1, wherein the reaction conditions comprise a rock to liquid ratio from about 1-40% (w/v).

19. The method of claim 1, wherein the reaction conditions comprise a buffer.

20. The method of claim 19, wherein the buffer is selected from the group consisting of: TRIS, PBS, citrate, monosodium glutamate, and any combination thereof.


SEQUENCE LISTING (464 sequences)

SEQ ID NO: 1 - protein, 247 aa, synthetic construct
  source 1..247: note = Carbonic Anhydrase P40881
MMFNKQIFTILILSLSLALAGSGCISEGAEDNVAQEITVDEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKETS

SEQ ID NO: 2 - protein, 236 aa, synthetic construct
  source 1..236: note = Carbonic Anhydrase T5H8M4
MKLSSKLILGLTVSSLAGKFLEKLLIQDNVSPNITASFNQEADIPDIDASSYIHHFASVIGSVVIGRNVFIGPFSSIRGDVGLKIFISHDCNIQDGVVLHGLKNYEYNSPVTEHSVFKDRESYSIYIGEKVSLAPQCQIYGPVRIDKNVFVGMQSLVFDAYIQEDTVIEPGAKIIGVTIPPKRFVSAGRVISNQEDANRLPEITDSYPYHDLNSKMTSVNLELAKGYKKEERQWKL

SEQ ID NO: 3 - protein, 205 aa, synthetic construct
  source 1..205: note = Carbonic Anhydrase Q3A3H4
MIEKNVVTDFCSEASEPVIDASTYVHPLAAVIGNVILGKNIMVSPTAVVRGDEGQPLHVGDDSNIQDGVVIHALETEMNGKPVAKNLYQVDGRSYGAYVGCRVSLAHQVQIHGPAVVLDDTFVGMKSLVFKSFVGKGCVIEPGSIVMGVTVADGRYVPAGSVIRTQEDADALPEIGADYPFRAMNPGVVHVNTALAKGYMVKQGN

SEQ ID NO: 4 - protein, 204 aa, synthetic construct
  source 1..204: note = Carbonic Anhydrase
MIGKNVLTDFSARASEPVIGSFTFVHPLAAVIGNVILGDNIMVSPGASIRGDEGQPLYVGSDSNVQDGVVIHALETELDGKPVEKNLVEVDGKKYAVYVGNRVSLAHQVQVHGPAVIRDDTFVGMKSLVFKSYVGSNCVIEPGVLLMGVTVADGRYVPAGSVVKTQEQADALPVITDDYPMKEMNKGVLHVNKALARGYLAAGS

SEQ ID NO: 5 - protein, 248 aa, synthetic construct
  source 1..248: note = Carbonic Anhydrase Q2LUP7
MRFNKQTFTILILSLSLALLGSGCISEGEGAEGNVTQGITESEFSNIRENPVTPWNPVPVAPVIDPTAFIDPQASVIGNVTIGASVMVSPMASIRSDEGMPIFVGDRSNVQDGVVLHALETIDEEGEPVENNIVEVGGKKYAVYIGENVSLAHQAQVHGPASVGNDTFIGMQAFVFKSKIGNNCVLEPTSAAIGVTVPDGRYIPAGMVVTSQAEADNLSEITDDYAYKHTNEAVVYVNVHLAEGYNKA

SEQ ID NO: 6 - protein, 243 aa, synthetic construct
  source 1..243: note = Carbonic Anhydrase Q467M8
MALLLSLAITLAGSGCVSQGEGAEEGENIEAEEVEANVEESNIRANPVTPWNPEPTEPVIDPTAYIHPQASVIGDVTIGASVMVSPMASVRSDEGMPIFVGDECNIQDGVILHALETVNEEGEPVEENQVEVDGKKYAVYIGERVSLAHQAQVHGPSLVGNDTFIGMQTFVFKAKIGNNCVLEPTSAAIGVTVPDGRYIPAGTVVTSQDEADKLPEVTDDYAYKHTNEAVVYVNTNLAEGYNA

SEQ ID NO: 7 - protein, 275 aa, synthetic construct
  source 1..275: note = Carbonic Anhydrase Q8PSJ1
MKKYLWGKTCLVVSLSVMVTACSSAPSTEPVDEPSETHEETSGGAHEVHWSYTGDTGPEHWAELDSEYGACAQGEEQSPINLDKTEAIDTDTEIHVHYEPSSFTIKNNGHTIQAETTSDKNTIEIDGKEYTLVQFHFHIPSEHEMEGKNLDMELHFVHKNENDELAVLGVLMKAGEENEELAQLWSKLPAEETEENISLDESIDLNVLLPESKEGFHYNGSLTTPPCSEGVKWTVLSEPITVSQEQIDAFAEIFPDNHRPVQPWNDRDVYDVITE

SEQ ID NO: 8 - protein, 263 aa, synthetic construct
  source 1..263: note = Carbonic Anhydrase A0A0N9WRG3
MKRSHLFTSITLASVVTLATAPAASAASFLSPLQALKASWSYEGETGPEFWGDLDEAFAACSNGKEQSPINLFYDREQTSKWNWAFSYSEAAFSVENNGHTIQANVENEDAGGLEINGEAYQLIQFHFHTPSEHTIEETSFPMELHLVHANHAGDLAVLGVLMEMGNDHEGIEAVWEVMPEEEGTAAYSISLDPNLFLPESVTAYQYDGSLTTPPCSEGVKWTVLNDTISISETQLDAFRDIYPQNYRPVQELGDREIGFHYH

SEQ ID NO: 9 - protein, 247 aa, synthetic construct
  source 1..247: note = Carbonate dehydratase Q5WD44
MKINRIFLALLFSLALTLAGSGCVSQGEGAEDGESADTEVESEVSNIRANPVTPWNPEPTEPVIDSTAYIHPQAAVIGDVTIGASVMVSPMASVRSDEGTPIFVGDETNIQDGVVLHALETVNEEGEPVESNLVEVDGEKYAVYVGERVSLAHQSQIHGPAYVGNDTFIGMQALVFKANVGDNCVLEPKSGAIGVTIPDGRYIPAGTVVTSQAEADELPEVTDDYGYKHTNEAVVYVNVNLAAGYNA

SEQ ID NO: 10 - protein, 666 aa, synthetic construct
  source 1..666: note = Kofleriaceae bacterium Carbonic Anhydrase Q8TMW3
MRTNRVRTAGASKWSGVSDIRTTLRERWSEIAAQGLSYHDVLAGLTVATVAIPLNVALAISAGLPPSAGLLAGAVGGLFAAAFGGSNFQVSGPAAALNVMVFGVVAKFGLGGAAAAALVCGIVGIALGVSGLGKYSNLMPKLVLAGFTTGVGLKLLDQQIPILLGSDLALWHMLSNFWAMEWLREVEWFSVVCGLLVAWITVGLAHLKSFPSALLGIVLATLIAYELDWNVARVGEVDLSDLALALPSIADGTSWFALIAVALPLAVLSSVESLISAKAVDAMANGKSGYSANTELFGQGVGSIASALVGGMPLAGVVVRSSVNQQSGARTRLAAMCHAVFLGIVAYFFGGLLGVIPVAALAGLLVVIATRLMKLSYFFSALRENKLHALAFLAAAIGTLLGYLISGLALGCALVYIAHKLAHRPVKDAPVLRPSPTIRAVISQAGERAQDHTPSIDEQAKWSRHVRTRPKIHPTAYVHPTASVIGWVELGREVNIAADTSVRADEGAPFYVGDRSNVQDGVVIHALKDKWVMVDGRRWAVWIGSDVSLAHQALVHGPSMIGSRSFIGFKAIVHDSVVGEGCFIGLGAVVVGVEIPAGKRVPNGWIVDSPEKVRELPDVEHAHAHFNEDVVQVNRGLVVAYSRHVPTEELPQRTPSDSPLFHLKPL

SEQ ID NO: 11 - protein, 251 aa, synthetic construct
  source 1..251: note = Thermodesulfitimonas autotrophica Carbonic Anhydrase A0A7Y6PMB4
MRLPKMLTVVAVGATLCFTAGCASTQTTATKEPAKPANIRPNVVTTFNPTTETPVIAKDAYIDPLASVIGNVEIGSKVYVAPFASVRGDEGQPIYVGEGSNVQDGVVLHALETEDNGKPVEKNLVEYGGKKYAVYIGKHVSLAHQAQVHGPALVDDGTFVGMQALVFKAQVGKNCVIEPGAKLLNGVKVPDGRYVPAGTVVTTQAQADKLPVITDAYPLKNLNKGVLHVNEQLAEGYLKAQEGATGETKSH

SEQ ID NO: 12 - protein, 547 aa, synthetic construct
  source 1..547: note = Fischerella thermalis/Mastigocladus laminosus JSC-11 Carbonic Anhydrase A0A3N5BJ34
MAVRSIAEAAPPTPWSRNLAEPTIHPSAFLHSFSNIIGDVRIGANVIIAPGTSVRADEGTPFYIGENTNLQDGVVVHGLEKGRVIGDDRQEYSVWIGKNNCITHMALIHGPCYIGDDCFIGFRSTVFNARVGAGCIVMMHALIQDVEIPPGKYVPSGAIITNQQQADRLPDVQADDKEFAHHVVGINQALRAGYLCAADSKCIRAIRDELNNSYTSIEVDVLERSDEVSSNSLGAETVEQVRYLLQQGYHIGTEHVDQRRFRTGSWTSCKPIEARSLGEAIAALEACLRDHSGEYVRLFGIDPKGKRRVLENIIQRPDGVVQASSSLKAPAYSSNNGSYNGNGSSRLSSETIDQIRQLLAGGYKIGTEHVDERRFRTGSWQSCKPIESSSPGDVVAALEDCMDNHQGEYVRLIGIDPKAKRRVLESIIQRPNGPVSTPSSKSTATTTSYAASGTTATATSSKLSSEAIEQLQQLLAGGFKISAEHVDGRRFRTGSWASCGQIQANSIREAIAALEGYMNEYQGEYVRLIGIDPKVKRRVLELIVQRP

SEQ ID NO: 13 - protein, 183 aa, synthetic construct
  source 1..183: note = Thermosynechococcus vestitus BP-1/(Thermosynechococcus elongatus BP-1 Carbonic Anhydrase G6FUV4
MLRKNPRTSWNSQESMPSVATTAYVDETAVVIGDVRIGERVYVGPCASIRADEATPIVISEECNVQDGAIFHGLKGSSIKLGKKVSVAHGAVVHGPMTIGDESFIGFNAVVHASTVGERCFIGHRALVMGVKLKDGSFVPHGSVIDTQDKADALGPVPDSLKGFNAEVVEVNCEFAKGYRSLR

SEQ ID NO: 14 - protein, 185 aa, synthetic construct
  source 1..185: note = Methanothrix thermoacetophila Carbonic Anhydrase Q8DKB5
MSENLRLNPQGDKPVIDPSSYVDPTAVIIGPVTIGKNCYIGPHTVIRADEVDEKTGKVAPVIIGDNVNLQDGVIIHALAGTSVEVGSNTSLAHGCVVHGPCKIEAGCFIGFRAVVFKTVIGSGSMVKHGAIVEGVNIPSGKLVPTGEIITSEDHLVKLKEVGQAEKEFMQEVVHVNMELAHGYKK

SEQ ID NO: 15 - protein, 185 aa, synthetic construct; identical to SEQ ID NO: 14
  source 1..185: note = Thermosyntropha lipolytica Carbonic Anhydrase A0B700

SEQ ID NO: 16 - protein, 185 aa, synthetic construct; identical to SEQ ID NO: 14
  source 1..185: note = Desulfofundulus thermobenzoicus Carbonic Anhydrase A0A1M5PQH8

SEQ ID NO: 17 - protein, 192 aa, synthetic construct
  source 1..192: note = Archaeoglobus veneficus Carbonic Anhydrase A0A6N7IXF4
MLQKSPAVSWKPAGYPRISSLAFVHPTAVLIGEVVIHDGAIIFPLAIIRADEGFPIIVGENTNIQDGVIIHCLKGGRVEIGRRVSLAHGAVIHGPCVIGDETFVGFRAMVINSRIGRGCFIDHGALIEGVEIPDGKYIPGLTRVSSQEQVSRLAGITEQQKDFAAEVLAVNGELKEAMQVIITSRDDAYPGQ

SEQ ID NO: 18 - protein, 256 aa, synthetic construct
  source 1..256: note = Carbonic Anhydrase F2KNT3
MRWAIILTTVLFAALLLGCAAEKGAIEPLETPEEKASNIHANPITEWNDEQTMPDIDPTAFVHPYATVIGDVHIGKYVCISPHASVRGDEGMPIYVGDYSNIQDCVVIHALETRDAEGNPIEKNLVVGDDGKKYAVYIADHVSLAHQSQVHGPAYVGSGTFIGMQALVFKAKVGKNCVIEPGAKVIGVTIPDGRYVPAGMAVTNQSVADNLPEITEDYPFKHTNEAVVHVNIELAKGYNAMFGGESTEGTEGEGGH

SEQ ID NO: 19 - protein, 214 aa, synthetic construct
  source 1..214: note = Library of modified or engineered enzymes
MQEITVTRFENIRPSPVTPWNPEPRYPEIHPTAYIDPAAVVQGDVKIGANVLVMANAVIRADEGYPIYIGDNSSVQDNVVLHALETRDADGRDLEENIVRVGDERYAVYVGDNVVLAHNAQVHGPAAVGDNTFVGMNALVFRSRVGADCVLAPLAAAIGVTVPDGRYVPAGTVVTTQAAAAALPAVTPDHPFAGLNARVVAVNVALAKGYLALS

SEQ ID NO: 20 - protein, 259 aa, synthetic construct
  source 1..259: note = Library of modified or engineered enzymes
MSKIYLAFVCGPEQWHRDFPTANGLRQSPIDIIPSKAVYDPKLRPLELKYDPSTCLHILNNGHSFQVEFDDSQDKSVLKGGPLDGIYRLIQFHFHWGSVDGQGSEHTVDKKKYAAELHLVHWNTKYGDFGKAVQQPDGLAVLGIFLKVGRHKPELQKLVDALSSIKHKDTLVDFGNFDPSCLMPTCPDYWTYSGSLTTPPLSESVTWIIKKQPVEVDHDQLEQFRSLLFTSEGEKEKRMVDNFRPLQPLMNRTVRSSFR

SEQ ID NO: 21 - protein, 270 aa, synthetic construct
  source 1..270: note = Library of modified or engineered enzymes
MKRSLVATIFGYCPEWNDHQSEWGYGETNGPKTWGKHFPEANGLLQSPIDIKTEETQHDPNLRPLTLKYDPSTAKEILNNGHSFQVTFVDDTDSSTLTDGPITGTYRLKQFHFHWGSSDDKGSEHTVDGAKYPAELHLVHWNTKYASFGEAASKPDGLAVVGVFLKIGKEHPGLKKLTDALYMVRFKGTKAQFTNFNPKCLLPTSLDYWTYPGSLTTPPLSECVTWIVLKEPISVSSAQMEKFRNLLFTSEGEKACCMVDNYRPPQPLKG

SEQ ID NO: 22 - protein, 214 aa, synthetic construct
  source 1..214: note = Library of modified or engineered enzymes
MQEITVTNYNNIRPSPVTSWNPTPKLPKIHPTAYIDPAAVVQGDVTIGENVMVSANASIRSDEGYPIYIGDNSNVQDNVVLHALETVDADGNVLEENVVTVGDKKYAVYIGKNVSLAHQAQVHGPAAVGDNTFIGMQAFVFNSVVGKDCVLMPLAAAIGVTIPDGKYIPAGTVVTTQEEADKLPEVTPDHPFANTNKAVVAVNVELAKGYLALA

SEQ ID NO: 23 - protein, 310 aa, synthetic construct
  source 1..310: note = Library of modified or engineered enzymes
MRFFECSCSPFPSQLSSFLTHLLILYTLSSSVEASSRNNYQWSYDSDVFGGPDFWGLVEKDWWMCRKGRLQSPIDIQPDRLLFDASVKPVRLDKLPVLSEFVNTGQMVRIRIGYSTKKPSVNITNGPLYGYRYRVQRIDFHMGRGKENGSEHTINGRRFPMEVQLVAFNTDLYPNFTAASKSPHGIAILSVLVDFGAQTNQELTKLTIATASISYKDQRVQMADFEPWRLLPFTRDIITYEGSLTSPGCHETVTWIILNQPIFITREHFEEWSHLYHTMEGAEKVPVAPNYRKIQETNNRLVRTNIQHKV

SEQ ID NO: 24 - protein, 214 aa, synthetic construct
  source 1..214: note = Library of modified or engineered enzymes
MQEITVLEFSNVTKNEVTPWNPKPVTPVIDPTAYIDPTATVIGDVTIGANCYIAASAVIRADEGKPIVIGDRSNVQDGVVLHALESVDDGGKVREDNVVIHGDNWYAVYIGENVSLAHQSQVHGPAYVGDDSFVGMKSLVFKSIVGSNCVIEPEAAAIGVTIPDGKYIPAGTVVTTQAEADKLPEVTPDYAFYTQVAAVVTVNVNLCRAYRNLS

SEQ ID NO: 25 - protein, 214 aa, synthetic construct
  source 1..214: note = Library of modified or engineered enzymes
MQEITVAEYSNITKNEVTPWNPKPSTPVIDPTSYVDPNATVIGDVTIGKNCYIAASAVIRADEGKPIVIGDRSNVQDGVVLHALESVDDGGMIIGDNVVVEGDKYYAVYIGNNVKLAHQSQVHGPAMVGDDSFVGMQSFVFNSIVGSNCVIEPEAAAIGVTVPDNKYIPAGTVVTTQAEADKLPEVTPDDAAFTKNAAVVNVNVGLAKAYREKA

SEQ ID NO: 26 - protein, 214 aa, synthetic construct
  source 1..214: note = Library of modified or engineered enzymes
MQEITVTNYNNIRPSPVTSWNPEPKLPEIHPTAYIDPAAVVQGDVTIGENVLVMANAVIRADEGYPIVIGDNSAVQDNVVLHALETVDENGNRIEENIVKVGDEEYAVYIGKNVVLAHNAQVHGPAIVGDNTFVGMNALVFRSRVGKNCVLEHNAAAIGVTVPDGKYIPAGTVVTTQEEADKLPEVTPDHPHYKLNERVVKVNVELAKGYLALK

SEQ ID NO: 27 - protein, 214 aa, synthetic construct
  source 1..214: note = Library of modified or engineered enzymes
MQEITVTRYENIQPSPVTPWNPTPKRPQIHPTAYVHPLAYVQGDVTIGANVMISPNASIRSDEGYPIKIGDNSNVQDNVVLHALETVDADGKRIEENIVKVGDEEYAVYIGDNVSLAHQAQVHGPAAVGDNTFIGMQAFVFRSIVGKNCVLEPLAAAIGVTIPDGTYIPAGTVVTTQEEADKLPKVTPDHPFAKTNAAVVAVNVALAKGYLALA

SEQ ID NO: 28 - protein, 186 aa, synthetic construct
  source 1..186: note = Library of modified or engineered enzymes
MLRKNPSGHIPQVAETAFIDPTAIICGKVIIEDYVFIGPYAVIRADEVNEQGDMEAIVIKRDTNIQDGVVIHSKAGAAVTIGERSSIAHRSIIHGPCWVGDDVFIGFNSVVFNAKIGKGCVIRHNSVVDGLDLPENFHVPPMTNIGPGFDLESISKVPPEYSAFSESVVSANHELVQGYRRIANEL

SEQ ID NO: 29 - protein, 281 aa, synthetic construct
  source 1..281: note = Library of modified or engineered enzymes
MGRSCLTLSRYQAKVSANFLKNRVMASWGYKTDNGPSQWHIGYPVAKTGTRQSPVNIVPSTVTRDDLLKALKYEYTPSMIKMINTGSSWRMDFSPEGSNLSGGPLGDDYKVLQMHAHWGDKAGRGSEHTMDGKMFDAELHIVHYNSKYGEPAIALDKPDGLAVLGMFIKTGWRSHPEFDKLCDNLKLIEMKGESLQLQEYLNPANCLPNNKTFVTYPGSLTTPPLFESVTWIVFLEPIEMSSKQLDSMRALKIGDTADCGCMVNNYRPPCALGNRKIRVKV

SEQ ID NO: 30 - protein, 282 aa, synthetic construct
  source 1..282: note = Library of modified or engineered enzymes
MSLVPIERETARRGRPPVAPRALGALLALASAVAATPAIAWQSGIAVPDPNAMPQWRYTGERGPEHWSELDPSYGACAHTDTQSPVALTESMAVAVACEPLRFRYRSGPLYVTNDGRALRLGYDRGSHLLVEGLSYELVELRFHAPAEHVINGSRADAELQLIHANNRGDIAVVAVALMPGPRANSMLQRLLKHAPRLSGESFYGRNVGVNPLFLLPGRKDYFAYRGSVTRPPCTEGVRWYVLRTPLEVADADLQRLVGFMEPNARPLQPLGGRRVTKACGP

SEQ ID NO: 31 - protein, 214 aa, synthetic construct
  source 1..214: note = Library of modified or engineered enzymes
MQEITVTRYENIRPSPVTPWNPEPKLPKIHPTAYIDPAAVVQGDVTIGANVMVSANASIRSDEGYPIYIGDNSNVQDNVVLHALETVDENGNVIEENVVTVGDKKYAVAIGDNVSLAHQAQVHGPAIVGDNTFIGMQAFVFRSRVGKNCVLAPLAAAIGVTVPDGTYIPAGKVVTTQEEAAKLPKVTPDHPFANTNAAVVKVNVALAKGYLALA

SEQ ID NO: 32 - protein, 214 aa, synthetic construct
  source 1..214: note = Library of modified or engineered enzymes
MQEITVHHYSNVTKNEVTPTNPKPVTPVIDPTSYVDPNATVIGDVTIGKNVLIAANAVIRADEGAPIVIGDRSTVQDGVVLHALESVDDAGKVREDNVVIEGDEEYAVYIGKDVSLAHQSQVHGPARVGDHSFVGMKSLVFKSIVGSNCVLEPEAAAIGVTVPDGKYIPAGTVVTTQEEAAKLPEITPDYPLYHANQVVVNVNVLLCQAYKALS

SEQ ID NO: 33 - protein, 269 aa, synthetic construct
  source 1..269: note = Library of modified or engineered enzymes
MAKTSFFPVVLSFIFILSYTMCINANATGKHEVDDEEPFSYLLGTAEGPYKWGTLKPDWEICNTGLFQSPINFRNKTVKVTKHIPHFTPNYKIASATIMNRGHDIKLQWEGDAGSITLNGTVYKLIQCHWHTPSEHKVDGQSLAMEAHLIHQSVNGKLIAVIGILFNIGPPDPFLNELIHHAKKVDHKGKKVGLVDPNKLGVKAEPFYRYIGSLTIPPCTEGIVWNVLHQPRTVSMDQMMALRNAVNDGFQANARPAQGLRRRPVYLVM

SEQ ID NO: 34 - protein, 214 aa, synthetic construct
  source 1..214: note = Library of modified or engineered enzymes
MQEITVTTYNNIRPSPVTSWNPEPRLPKIHPTAYIDPAAVVTGDVTIGANVLVMANAVIRADEGYPIVIGDNSSVQDNVVLHALETVDADGNVLEENVVLVGDERYAVYVGDNVVIAHNAQVHGPAIVGDNTFVGMNALVFRSRVGANCVLAPLAAAIGVTIPDGTYIPAGKVVTTQEEAAKLPRVTPDHPFADLVARVVKVNVELAKGYLALS

SEQ ID NO: 35 - protein, 214 aa, synthetic construct
  source 1..214: note = Library of modified or engineered enzymes
MQEITVTDYSNITKNEVTSTNPKPTTPVIDPTSYVDPNATVTGDVTIGKNVMISDSASIRSDEGRPIVIGDRSNVQDGVVLHALESVDDDGEILEDNVVEVGDENYAVYVGKNVSLAHQSQVHGPAAVGDDSFIGMQAFVFKSKVGSNCVIEPDAAAIGVTVPDGKYIPAGTVVTTQEEAAKLPEITPDYEYSDTVEAVVEVNVALREAYKEKS

SEQ ID NO: 36 - protein, 343 aa, synthetic construct
  source 1..343: note = Library of modified or engineered enzymes
MLAFVALVSLIFLGVQAQHGADWTYSEGMLDETHWPEEYPDCGGQRQSPIDLQRRKVRFNPDLQPLELTGYGDSQGSPFLMTNNGHTVQITLPPTMQLTAPDGAVYKATQMHYHWGGASYELSGSEHTIDGIRRVIEMHLVHYNAKYESYDVAKDKPDGLAVMAAFVEIEEYAENTHYSSLISHLANIRYPGQTTYLTDFDILDMLPGDMYHYYTYNGSLTTPPCTQNVRWFVMSDSVKISKAQVIKLENSVMNHQNQTLHNGYRKTQPLHSRVVEANFPYFPNTMPGEGSGLRAKDPAREFGSRRHCYAWRGWQPAAAAALEGHGEPRRRWRPLEEASTPPP

SEQ ID NO: 37 - protein, 214 aa, synthetic construct
  source 1..214: note = Library of modified or engineered enzymes
MQEITVTRYNNIRPSPVTPWNPEPKLPEIHPTAYIDPAAVVQGDVTIGANVMVSANASIRSDEGYPIYIGDNSNVQDNVVLHALETVDADGKRIEENVVKVGDKDYAVYVGDNVSLAHQAQVHGPAAVGDNTFIGMQSFVFRSIVGKNCVLEPLAAAIGVTVPDNTYIPAGKVVTTQEEADKLPKVTPDHPFANTNAAVVKVNVALAKGYLALA

SEQ ID NO: 38 - protein, 291 aa, synthetic construct
  source 1..291: note = Library of modified or engineered enzymes
MKNRRIKPEIMKTKFLFAILTLFFFSGCQFFDKNKSTEIESKPSSHEKWSYTGESGPEHWAELEDQAVCDGQHQSPVNISDIDIKPGKLIQESLDLSYQEVTTIKSITNNGHTIQYNFDANSNLVSLHDKQYKLKQFHFHSPSEHTINGTHSPLEIHLVHHSEATNSYIVIAILVQQGEPDDAFDFLEKYLPINVGETKEINSKYYFGSTFPEMYGKDTLNIYTYEGSLTTPPCTESVLWVVIKDPAYASSSQIVMLQKLMPKDNYREVQSLNGRLIYNEIIEDDISVLNH

SEQ ID NO: 39 - protein, 230 aa, synthetic construct
  source 1..230: note = Library of modified or engineered enzymes
MTKLSFAVIGPENWHRYCDQAQGDQQSPINIQTRDVKHDPTLRPLTLRYDPSTAREIVNNGHSFNVEFEDSTDRSVLRGGPLTDRYRLTQFHFHWGSSDDHGSEHTVDGVKYAAELHLVHWNTKYGDFGEAASKPDGLAVVGVFLKVGRHNPRLQKILDALHAIKTKGKRASFTNFDPSVLLPGCLDYWTYSGSLTTPPLSESVTWIVLREPISVSPSQMAKFRSLLFTS

SEQ ID NO: 40 - protein, 214 aa, synthetic construct
  source 1..214: note = Library of modified or engineered enzymes
MQEITVTTYNNIQPSPVTPWNPEPKLPKIHPTAYIHPKAVVQGDVTIGKNVMVSANASIRSDEGYPIVIGDNSNVQDNVVLHALETVDENGNEIEENIVTVGDKKYAVYIGKNVSLAHQAQVHGPAIVGDNTFIGMQAFVFNSNVGSNCYLAPLAAAIGVTVPDGTYIPAGKVVTTQEEAAKLPKITPDHPFYNTNAAVVKVNVALAKGYLALS

SEQ ID NO: 41 - protein, 214 aa, synthetic construct
  source 1..214: note = Library of modified or engineered enzymes
MQEITVDEFSNITKNEVTPFNPKPTIPVIDPTAYVDPNATVIGDVTIGKNCYIAPFASIRADEGKPIVIGDNSNVQDGVVLHALESIDDGGKLIEENVVVEGEKRYAVYIGKNVSLAHQSQVHGPARVGDDSFVGMNSLVFNSKVGSNCVIEPFAAAIGVTVPDGKYVPAGTVVTTQEEADKLPEITDDYAFAGTNEAVVKVNVKLCKAYREKA

SEQ ID NO: 42 - protein, 214 aa, synthetic construct
  source 1..214: note = Library of modified or engineered enzymes
MQEITVLEYSNVTKNEVTSQNPKPVTPVIDPTSYVDPNATVVGDVTIGENCLVWPTAVIRADEGRPIVIGNESSVQDGVVLHALESVDDGGELVEDNVVVVGDKNYAVYVGKNVSLAHQAQVHGPARVGDDSFVGMKSLVFKSDVGSNCVIEPFAAAIGVTVPDGKYIPAGTVVTTQEEAAQLPEVTPDHAEYTTQATVVTVNVELNEAYRNQR

SEQ ID NO: 43 - protein, 261 aa, synthetic construct
  source 1..261: note = Library of modified or engineered enzymes
MTEKLWGYDSHNGPARWFQICVPAQGKRQSPIDIQPDKAVLDSTLKPLELKYDPSTARRIVNVGHSFHVEFEDSTDKSVLQGGPLTGSYRLRQFHFHWGKKDDVGSEHVLDGVKYSAELHVVHWNADKYSSFVEAAHEPDGLVVLGVFLQIGDQHPGLQRLTDALYAVRFKGTKAQFACFNPKCLLPTSRHYWTYPGSLTTPPLSESVTWIVLREPISVSERQMEKFRSLLFTSEDDERIHMVNNFRPLQPLMNRTVRSSF

SEQ ID NO: 44 - protein, 302 aa, synthetic construct
  source 1..302: note = Library of modified or engineered enzymes
MYHNALFLTPITVFYVAAHKFGYDAEDGPSTWRGVCQTGKRQSPVDIRAFEIEIAPLDPLQFLNYDLTGHIHLANNGHTVVGSGFERWGEKRPYISGGGLNGTYQLSQFHFHWSQQNDTGSEHTIASLHYPGELHLVHIKKEPSPDEVNTIAVVAAFIKLDDHAGSLHNLKPYVHNIRMPNTELVVPGFSVSSLLPEHRENFYRYEGSLTTPGCDEVVVWTLMADPIAVTPSQMGAFHQVHFASGKTGHNWRPTQPLNGRKILFRPSITLRTFKSGGAMLKPVFQPFISIWLYGIYHIISVF

SEQ ID NO: 45 - protein, 214 aa, synthetic construct
  source 1..214: note = Library of modified or engineered enzymes
MQEITVTKYNNIRPSPVTPWNPEPKLPEIHPTAYIDPAAVVRGDVKIGENVLVMANAVIRADEGYPIYIGNNSSVQDNVVLHALETVDENGNRIEENIVLVGDKEYAVYIGDNVVIAHNAQVHGPAAVGDNTFIGMNSLVFRSRVGSNCVLAPLAAAIGVTVPDGTYIPAGKVVTTQEEAAKLPKITPDHPFANLNDRVVKVNVALAKGYLAQA

SEQ ID NO: 46 - protein, 353 aa, synthetic construct
  source 1..353: note = Library of modified or engineered enzymes
MKMFPLDCLILPCCYFFFISTPHFANADVHIADWDHDHHHTHPDNWEGMCKEGQRQSPIDIITNETTKEKWGQPFIFHGYERKLSMNVKNNRHSMVVEFDNDKKYEDIWIRGGGLGESKFRFAQLHFHWGSTNDQGSEHTIDGKASPMEMHIVHWNLDVGKDVKEATEKDAYNSLEVLGVLFKLGKFNKDYDAIFNAARKVEKENTNATLEKDVRLRDLLPEDTNAFYRYVGSLTTPPCNQIVMWTIFKDPIEISQEQLDIMRKGSYRLEGENDVRYIANNYRSTQTLYERDVLDIDTHIVHLACNSKGSTRYHFEEGSEGFVHNTGNSLNSPIVTCMLFYLSIFVISMRLLH

SEQ ID NO: 47 - protein, 214 aa, synthetic construct
  source 1..214: note = Library of modified or engineered enzymes
MQEITVTRYENIRPSPVTPWNPEPRRPVIHPTAYVDPLAYVQGDVTIGANVMVSANASIRSDEGYPIVIGDNSNVQDNVVLHALETRDADGRVLEENVVVVGDERYAVYVGDNVSLAHQAQVHGPAAVGDNTFIGMQAFVFRSRVGKNCVLEPLAAAIGVTVPDGTYVPAGKVVTTQEEAAKLPKVTPDHPFATTNAAVVAVNVALAKGYLALA

SEQ ID NO: 48 - protein, 214 aa, synthetic construct
  source 1..214: note = Library of modified or engineered enzymes
MQEITVLEFSNITKNEVTSFNPEPVTPVIDPTAYIDPNATVIGDVTIGANVLIWPTAVIRADEGKPIVIGDRSNVQDGVVLHALESVDDGGKVREDNVVIEGDEEYAVYIGKNVTLAHQSQVHGPARVGDDSFVGMKSLVFNSDVGENCVIEPFAAAIGVTVPDGKYIPAGTVVTTQAEAATLPEVTPDYAFYTQVAAVVSVNVGLCQAYKNEA

SEQ ID NO: 49 - protein, 214 aa, synthetic construct
  source 1..214: note = Library of modified or engineered enzymes
MQEITVDEFSNVTKNEVTPWNPKPTTPVIDPTSYIDPEATVIGDVTIGKNCYIAPFAVIRADEGSPIVIGDDSTIQDGVVLHALESVDDGGKLIEDNVVLEGDQYYAVYIGRNVVLAHQSQVHGPAWVGDDSFVGMKSLVFKSTVGSNCVLEPNAAAIGVTVPDGKYIPAGQVVTTQAEADNLPEVTADDAYYTKVAAVVKVNVALCEAYREQS

SEQ ID NO: 50 - protein, 214 aa, synthetic construct
  source 1..214: note = Library of modified or engineered enzymes
MQEITVTKYNNIRPSPVTPWNPEPKLPEIHPTAYIDPAAVVQGDVTIGANVMVSANASIRSDEGYPIKIGDNSNVQDNVVLHALETVDADGKELTENVVTVGDEKYAVYVGDNVSLAHQAQVHGPAAVGDNTFIGMQAFVFRSTVGKNCVLAPLAAAIGVTVPDGRYIPAGLVVTTQEEADKLPKVTPDHPFYNTNAAVVAVNVALAKGYLAQA

SEQ ID NO: 51 - protein, 256 aa, synthetic construct
  source 1..256: note = Library of modified or engineered enzymes
MSRPVALTIFGYEDKNQWHCCYPSAQGNRQSPINIDIKKTVYDPKLKPLELSYDPATAKGILNNGHSFNVEFEDSQDKSVLKGGPLTGTYRLIQFHFHWGATDDKGSEHTVDGVKYPSELHLVHWNAVKYSSFAEAASKPDGLAVLGVFLKVGDHNAALQKLTDALYMVRFKGTKAQFTGFNPKCLLPASLDYWTYSGSLTTPPLLESVTWIVLKEPISVSSEQMAKFRSLLFTSEGEAECCMVDNYRPPQPLKGR

SEQ ID NO: 52 - protein, 214 aa, synthetic construct
  source 1..214: note = Library of modified or engineered enzymes
MQEITVTTYTNIRKSPVTSWNPTPKYPKIHPTAYIDPAAVVQGDVTIGENVMVSANASIRSDEGYPIYIGDNSNVQDNVVLHALETVDANGNVIEENVVTVGDKKYAVYVGNNVSLAHQAQVHGPAAVGDNTFIGMQAFVFNSRVGKNCVLEPLAAAIGVTVPDGTYIPAGEVVTTQEAADKLPKVTPDHPFANTNAAVVKVNIELAKGYLAQA

SEQ ID NO: 53 - protein, 214 aa, synthetic construct
  source 1..214: note = Library of modified or engineered enzymes
MQEITVTVYTNIQPSPVTSWNPTPKLPKIDETAYVHPQAVVQGDVTIGKNVMISANASIRSDEGYPIVIGDNSNVQDNVVLHALETVDADGKEIEENVVTVGDKKYAVYIGDNVSLAHQAQVHGPAAVGDNTFIGMQAFVFRSNVGKDCVLEPLAAAIGVTVPDGTYIPAGKVVTTQEEAAKLPKVTPDHPFYKTNAAVVKVNVELAKGYLALA

SEQ ID NO: 54 - protein, 186 aa, synthetic construct
  source 1..186: note = Library of modified or engineered enzymes
MLRKNPSGHIAVIDQTAYIDETAIICGKVIIEANVFVGPYAVIRADEVNEQGDMEPIVIKRDTNIQDGVVIHSKAGAAVTIGERSSIAHRSIIHGPCWVGDDVFIGFNSVVFNAKIGKGCVIRHNSVVDGLDLPENFHVPPMTNIGPDFDLNSISKVPPEYSAFSESVVSANHELVQGYRRIANEL

SEQ ID NO: 55 - protein, 214 aa, synthetic construct
  source 1..214: note = Library of modified or engineered enzymes
MQEITVLEFSNITKNEVTPWNPKPSTPVIDPTSYVDPNATVIGDVTIGKNCYIAASAVIRADEGKPIVIGDRSNVQDGVVLHALESVNDGGKIREDNVVLEGDKYYAVYIGKNVVLAHQSQVHGPAAVGDDSFVGMKSLVFKSIVGSNCVIEPEAAAIGVTVPDGKYIPAGTVVTTQAEADKLPEITPDYAFYTQVAAVVKVNVDLCEAYRNKA

SEQ ID NO: 56 - protein, 214 aa, synthetic construct
  source 1..214: note = Library of modified or engineered enzymes
MQEITVTTYTNIRPSPVTPWNPEPKLPEIHPTAYVDPAAVVQGDVTIGKNVMVSANASIRSDEGYPIKIGDNSNVQDNVVLHALETVDEDGNVIEENVVTVGDEKYAVYVGDNVSLAHQAQVHGPAAVGDNTFIGMQAFVFRSNVGKNCYLAPLAAAIGVTIPDGTYIPAGTVVTTQEEAAKLPKMTPDHPGYNTNAAVVKVNIALAKGYLALS

SEQ ID NO: 57 - protein, 214 aa, synthetic construct
  source 1..214: note = Library of modified or engineered enzymes
MQEITVDNFSNITKNEVTPTNPKPSTPVIDPTSYVDPNATVTGDVTIGKNCLIAANAKIRADEGKPIVIGDRSSVQDGVVLHALESVNDDGKVLEDNVVLEGDEYYTVYIGKNVVLAHQAQVHGPAAVGDDSFVGMKALVFKSKVGKNCVIEPGAAAIGVTVPDGKYIPAGTVVTTQEEADKLPEITPDYPLSDANEAVVKVNVGLCEAYRNKS

SEQ ID NO: 58 - protein, 214 aa, synthetic construct
  source 1..214: note = Library of modified or engineered enzymes
MQEITVTKYENIRPSPVTSWNPTPKLPKIHPTAYIDPLAYVQGDVTIGENVMVSANASIRSDEGYPIYIGNNSNVQDNVVLHALETVDKNGKVLEENVVTVGDKKYAVYVGDNVSLAHQAQVHGPAAVGDNTFIGMQAFVFRSTVGKNCYLAPLAAAIGVTVPDGTYIPAGKVVTTQEEAAKLPKMTPDHPGYKTNEAVVEVNVELAKGYLALA

SEQ ID NO: 59 - protein, 293 aa, synthetic construct
  source 1..293: note = Library of modified or engineered enzymes
MASKLLRRNLLFTIQKRAVKSSVTRNSIPWLRKSAPSSNWGYNGSELDPEDWPKEYQCGNCQSPIDIDLSKVTYSSELSPLEYSYPDNFKYMVNDGKNIRIHWRGETALSGGPLKGTYELVQLHFHWGSAEGKGAEHLVNGESVEGEAHLVHWNPKYGSIREALKHQDGIAVVGVFLKEADDGAESPLSSILNRFPTLSKFNEKYIFENDVFNVGNLIPKNSDFICYDGGLTTPPLTECVQWIVLLKPLVVTKREMDIFRSLEGSFGNNFTDNFRPCQPVGDRVVSSSFEPEK

SEQ ID NO: 60 - protein, 266 aa, synthetic construct
  source 1..266: note = Library of modified or engineered enzymes
MKITALSVFCWGPNYEDHRQKWSNLYPIAKGNRQSPINIVPGSAVYDSSLKPLKLKYDPSTCLEIWNNGHSFQVTFEDTDDKSVLSGGPLTDKYKLKQFHFHWGKTDDHGSEHTVDGVKYAAELHLVHWNAKYGSFGEAADKPDGLAVVGIFLKIGREKGEFKLILDALDSIKTKGKQTTFTNFDPSCLFPSCPDYWTYSGSLTTPPLSESVTWIILKQPIEVDHDQLEKFRTLLFTSEGEKEKRMVDNFRPLQPLMNRTVRSSFR

SEQ ID NO: 61 - protein, 214 aa, synthetic construct
  source 1..214: note = Library of modified or engineered enzymes
MQEITVTVYNNIRPSPVTPWNPEPKLPKIHPTAYVHPLADVTGDVTIGANVMVSAHASIRSDEGYPIYIGDNSNVQDNVVLHALETVDAAGKEIEKNIVTVGDKKYAVYIGDNVSLAHQAQVHGPAAVGNNTFIGMQAFVFNSVVGENCVLEPLAAAIGVTVPDGTYIPAGKVVTTQEEAAKLPKVTPDHPFYNTNKAVVAVNVALAKGYLALA

SEQ ID NO: 62 - protein, 214 aa, synthetic construct
  source 1..214: note = Library of modified or engineered enzymes
MQEITVMEYSNVVKNEVTSTNPKPTTPKIDPTSYVDPNATVIGDVEIGKNVLIAPFAVIRADEGSPIVIGDNSNVQDGVVLHALESVDDGGKINEDNVVVKGDKYYAVYIGKNVHLAHQAQVHGPAYVGDDSFVGMKALVFKAKVGNNCVIEPNAAAIGVTVPDGKYVPAGTVVTTQEEADKLPEITEDYPFSTANEVVVKVNVNLAKAYRNLA

SEQ ID NO: 63 - protein, 226 aa, synthetic construct
  source 1..226: note = Library of modified or engineered enzymes
MAAHGAHWGYSGEAGPENWAKLTPEYGACTGKNQSPINLTGFIEAELKPIKIAYKAGAKEIVNNGHTVQVNYQPGSFITIDGQQFELKQFHFHAPSENTIEGKSFPLEAHFVHANSKGELAVVAVMYEEGKENPLIAKAWQQMPEKAGEKNELKSTISAESLLPKDKDYYRFSGSLTTPPCSEGVRWIVLKNYSTVSKEQVEQFLHTMHHANNRPVQPVNARKVLK

SEQ ID NO: 64 - protein, 226 aa, synthetic construct; identical to SEQ ID NO: 63
  source 1..226: note = Library of modified or engineered enzymes

SEQ ID NO: 65 - protein, 263 aa, synthetic construct
  source 1..263: note = Library of modified or engineered enzymes
MKSTLIAGFVCEQNPDHWYRQYPVAKGHHQSPIDIISHTAKYDPSLKPLSISYDPSTSLEILNNGHSFQVTFEDSNDKSVLKGGPLDGVYRLKQFHFHWGKKHSVGSEHTVNGKSFPSELHLVHWNAVKYESFGEAALEENGLAVVGVFLELGEHNAELQKITDALYMVRFKGTKTTFSCFNPKCLLPSSLDYWTYSGSLTTPPLSESVTWIVLREPISISPSQLAKFRSLLFTSEGEKAVCMVDNFRPLQPLMNRSVRSSFR

SEQ ID NO: 66 - protein, 214 aa, synthetic construct
  source 1..214: note = Library of modified or engineered enzymes
MQEITVTRYENIRPSPVTPWNPEPKLPEIHPTAYIDPKAVVQGDVRIGANVMVSAHASIRSDEGYPIVIGDNSNVQDNVVLHALETVNENGEVIEENVVEVGDERYAVYVGDNVSLAHQAQVHGPAAVGDNSFIGMQAFVFRSRVGKNCVLAPLAAAIGVEVPDGTYIPAGKVVTTQEEAAKLPKVTPDHPFANTNAAVVKVNVALAKGYLALS

SEQ ID NO: 67 - protein, 224 aa, synthetic construct
  source 1..224: note = Library of modified or engineered enzymes
MSQIWSYTGDTGPEFWPELCEEFYTAAQFPLQSPIALSYEETQALEEALKFTYVEQNIYVQKVNETMHFVPVDAASFVEFAQNRYYLTDIHFHMPSEHVINKQQAPLEFHLVHKDEGGNPLVCAVLFDLVENEDKKCNKDKLILEADKDKEQLLNPEIFLPENITYFHYEGSLTTPPTQGPVQWFVFDQIGVMSRSFIEDFKTSLLPNNRPLQNKNQRPIFYKK

SEQ ID NO: 68 - protein, 262 aa, synthetic construct
  source 1..262: note = Library of modified or engineered enzymes
MKGLTPSIAVFCYRQENWDHIFPIAAGNRQSPINIDTRKAKYDSSLKPLNLKYDPSTSLEILNNGHSFQVNFEDTDNKSVLKGGPLTGSYRLRQFHFHWGASDDKGSEHTVDGVKYASELHVVHWNAVKYSSFAEAASKPDGLAVVGVFLKVGQHNPQLQKITDALSSIKHKDTQALFSNFDPSSLLPSCPDYWTYSGSLTTPPLSESVTWIVLKQPINVSPAQLAQFRSLLFTSEGEKACCMVDNYRPLQPLKGRQVRASF

SEQ ID NO: 69 - protein, 262 aa, synthetic construct
  source 1..262: note = Library of modified or engineered enzymes
MSARLVTWGYKEDNGPHQWCIFFPEANGECQSPIDIITSETKHDPSLKPLSLSYNPATSKEIINVGHSFHVNFEDNDNRSVLKGGPLTDSYRLTQFHFHWGKKNDRGSEHTIDKKKYSSELHLVHWNTKYGDFGKAVQQPDGLAVLGIFLKVGKHNPSLQKVLDTLNSIKTKGKQTTFTNFDPSTLLPGCLDYWTYSGSLTTPPLLESVTWIILKEPISVSSEQMAKFRSLLFTSEGEKACCMVDNYRPLQPLMNRTVRSSF

SEQ ID NO: 70 - protein, 214 aa, synthetic construct
  source 1..214: note = Library of modified or engineered enzymes
MQEITVSAFSNIRKNEVTPWNPEPSTPVIDPTAYVDPQATVIGDVTIGANVLVSASASIRADEGRPIVVGDRSNVQDGVVLHALESVDDGGEVIEDNVVLEGGELYAVYVGENVSLAHQSQVHGPALVGDDSFVGMKSLVFKSKVGSNCVLEPGAAAIGVTIPDGKYIPAGTVVTSQAEADNLPEVTPDYAYYTTNEAVVKVNVALAEAYRNLS

SEQ ID NO: 71 - protein, 278 aa, synthetic construct
  source 1..278: note = Library of modified or engineered enzymes
MRLSAIFVTGWCPEKQDHNYYQWGYGKHNGPEHWKDHFPAANGLQQSPIDIQISKVQHDPALKPLSLSYDPATARRILNNGHSFNVEFDDSQDKAVLKGGPLTGSYRLIQFHFHWGSADGQGSEHTVDKKKYAAELHLVHWNAVKYESFAEAAKQENGLAVLGVFLKVGEHNAQLQKLLDALSAIKHKGKQTAFTNFDPSCLLPACPDYWTYSGSLTTPPLSESVTWIVLKEPISVSSEQMAKFRSLLFTSEGETACCMVDNYRPPQPLKGRKVRASF

SEQ ID NO: 72 - protein, 214 aa, synthetic construct
  source 1..214: note = Library of modified or engineered enzymes
MQEITVTRFENIRPSPVTSWNPEPKRPVIDPTAYIDPAAVVQGDVTIGKNVMISANASIRSDEGYPIYIGDNSNVQDNVVLHALETVDADGKRLEENVVKVGDKEYAVAIGDNVSLAHQAQVHGPAIVGDNSFIGMQAFVFRSRVGKNCVLAPLAAAIGVEVPDGKYIPAGKVVTTQEEADKLPEVTPDHPYATTNAAVVKVNVELAKGYLALS

SEQ ID NO: 73 - protein, 268 aa, synthetic construct
  source 1..268: note = Library of modified or engineered enzymes
MARTVLGIFCSPNKEWQYDHAKNGPEVWKEYFPIADGDQQSPIEIKTKEVKHDSSLKPLSISYNPATAKEILNVGHSFHVNFEDNDNRSVLKGGPLSDSYRLSQFHFHWGSSDDHGSEHTVDGVKYASELHLVHWNAKYGKFGEASKKPDGLAVVGIFLKVGSAKPGLQKVVDALGSIKTKGKQASFTNFDPSVLLPGCLDYWTYDGSLTTPPLLESVTWIVLKEPISVSPSQMAKFRSLLFSSEGEAACCMVDNYRPPQPLKGRQVK

SEQ ID NO: 74 - protein, 268 aa, synthetic construct
  source 1..268: note = Library of modified or engineered enzymes
MPLPNARERRRDWRAVVTAAAVFGIVVPIGTGLHAEDWGYSGTHGPRFWAKTPGWEACAGTAATERQSPIDIDEVVADKELTRLQADLKETPVAVVNNGHTIEEEYRLGSSLTLAGVRYDLKQFHFHTPSEHTVRGAHAAMEMHVVFKDAGSDKLVVIGVLFEVGKANAFLSALMADGLPGKRGEEVDAHSRPVNVAQALTDTSQYYTYPGSLTTPPCSENVTWFVLKGRPEMSAEQLAAFHRVLGDNARPVQKLNHRVAHETVSGAR

SEQ ID NO: 75 - protein, 204 aa, synthetic construct
  source 1..204: note = Library of modified or engineered enzymes
MTSLQYNNIRPNLAGDYPQIDPTALIDPSAQIIGNVKIDRDVFVGPLTVIRADQRGPNGKVSPIQIDREANIQDGVIIHTDPGASVIIGSKTTVAHGAIIHGPCTIGQECFIAIRASLYKVTLEDHVWLGIGAIAKLVTLHSFTRVPAGAVIRDSPEVLPLRLITDKERKYMEEVWAANSLLRTDYLELRDKVESIRSTAKKKG

SEQ ID NO: 76 - protein, 214 aa, synthetic construct
  source 1..214: note = Library of modified or engineered enzymes
MQEITVTRYENIRPSPVTPWNPEPKRPKIHPTAYIDPAAVVTGDVTIGENVLVMANAVIRADEGYPIVIGDNSSVQDNVVLHALETVDENGNRIEENVVRVGDEDYAVYVGKNVVLAHNAQVHGPAAVGDNTFVGMNALVFRSRVGKNCVLAPNAAAIGVTVPDGTYVPAGLVVTTQEEAAKLPKVTPDHPFANLNARVVKVNVALAKGYLALA

SEQ ID NO: 77 - protein, 214 aa, synthetic construct
  source 1..214: note = Library of modified or engineered enzymes
MQEITVTKFENIRPSPVTPWNPTPKRPEIHPTAYVDPLAYVQGDVTIGANVMISANASIRSDEGYPIVIGDNSNVQDNVVLHALETVDANGNEIKENIVTVGDEKYAVYVGDNVSLAHQAQVHGPAAVGDNTFIGMQAFVFRSVVGKNCVLEPLAAAIGVTIPDGTYIPAGKVVTTQEEADKLPKMTPDHPFYNTNKAVVAVNIALAKGYLALS

SEQ ID NO: 78 - protein, 214 aa, synthetic construct
  source 1..214: note = Library of modified or engineered enzymes
MQEITVHHHHHITKNEVTPTNPKPTTPVIDPTSYIDPNATVIGDVTIGKNVLIAPFASIRADEGSPIVIGDRSNVQDGVVLHALETVDDGGKVIEDNVVLEGDKYYAVYIGRNVTLAHQAQVHGPAAVGDDSFVGMKALVFKAKVGKNCVIEPDAAAIGVTVPDGKYIPAGTVVTTQEEADKLPEITPDYAFYTQNETVVKVNVALCEAYREKA

SEQ ID NO: 79 - protein, 244 aa, synthetic construct
  source 1..244: note = MLSLSALLAATAFSASASAPHWEYSGEAGPAHWASLTPEFGACTGKNQSPV NLTGFVDAKLKPIKFAYQAGGKSIVNNGHTVQVNYQPGSSITLDGVTFELKQFHFHAP SENQIDGQSYPLEAHLVHADKEGNLAVVALMFKQGEANPELAKLWQAMPEKANQSQPL KASIRADQLLPENRDYYRFSGSLTTPPCSEGVRWIVMKQPITASAAQIEEFEHVMHHP NNRPVQPLNGKTIVTGLSS
MLSLSALLAATAFSASASAPHWEYSGEAGPAHWASLTPEFGACTGKNQSPVNLTGFVDAKLKPIKFAYQAGGKSIVNNGHTVQVNYQPGSSITLDGVTFELKQFHFHAPSENQIDGQSYPLEAHLVHADKEGNLAVVALMFKQGEANPELAKLWQAMPEKANQSQPLKASIRADQLLPENRDYYRFSGSLTTPPCSEGVRWIVMKQPITASAAQIEEFEHVMHHPNNRPVQPLNGKTIVTGLSS

SEQ ID NO: 80 - protein, 214 aa, synthetic construct
  source 1..214: note = Library of modified or engineered enzymes
MQEITVTVFSNVEKNEVTSQNPRPTTPVIDPTSYIDPNATVIGDVTIGKNCYIAASASIRGDEGRPIHIGDRSNVQDGVVLHALESVDTDGEVLEDNVVLEGDEDYAVYIGKNVSLAHQSQVHGPARVGDDSFIGMKSLVFKSIVGSNCVLEPDAAAIGVTVPDGKYIPAGTVVTTQAEADKLPEVTPDYAYYTKNAAVVAVNVALCEAYRNQS

SEQ ID NO: 81 - protein, 259 aa, synthetic construct
  source 1..259: note = Library of modified or engineered enzymes
MLKIVSAFTGYCPENWHRQFDKAAGSQQSPIDIQTKDIQHDPCLQPLKLSYDPSTCLEIWNNGHSFLVQFEDSGDKSVIEGGPLEGVYRLKQFHFHWGAKDSEGSEHTVDGVKFPCELHLVHWNAKYGSFAEAASKPDGLAVVGVFLKIGKEHAEFQKLLDALDAIKTKGKQTTFTNFDPSCLLPACRDYWTYDGSLTTPPLLESVTWIVLKEPISVSPGQMAKFRSLLFTSEGEAACCMVDNYRPPQPLKGRHVRASF

SEQ ID NO: 82 - protein, 214 aa, synthetic construct
  source 1..214: note = Library of modified or engineered enzymes
MQEITVLSFSNVQKNKVTPTNPKPTTPVIDPTAYIDPDATVIGDVTIGKNCFIGAFAVIRADEGKPIVIGDRSNVQDGVVLHALESVDDEGKVIEDNVVVKGDKEYAVYIGKNVSLAHQSQVHGPARVGDDSFVGMNATVFNSIVGSNCVIEPFAAAIGVVVPDNTYIPAGTVVTSQEEADKLPEVTPDYAYYTQVAAVVKVNVKLCEAYREKA

SEQ ID NO: 83 - protein, 264 aa, synthetic construct
  source 1..264: note = Library of modified or engineered enzymes
MKITFLSAVCGWNYQDPERWHDDFPIAKGERQSPIDIDLSKVQRDPSLKPLSFKYDPSTSRRILNNGHSFNVEFEDSEDKSVLKGGPLTGSYRLKQFHFHWGATDDKGSEHTVDGVKYASELHLVHWNAKYGDFGEAASKPDGLAVVGVFLKIGRHHEEFQKLLDALPAIKHKDTLVDFGSFDPSCLMPTCPDYWTYSGSLTTPPLLESVTWIVLKQPIEVDHDQLEQFRTLLFTSEGEKEKRMVDNFRPLQPLMNRTVRSSFR

SEQ ID NO: 84 - protein, 214 aa, synthetic construct
  source 1..214: note = Library of modified or engineered enzymes
MQEITVDEFSNVTKNEVTPWNPKPSTPVIDPTSYIDPDATVIGDVTIGANVLIGPNAVIRADEGKPIVIGDNSNVQDGVVLHALESVDDAGKVIEDNVVKVGNNSYAVYVGKNVVLAHNAQVHGPAAVGDDSFVGMNAFVFNSIVGSNCVIEPNAAAIGVTVPDGKYIPAGTVVTTQEEADKLPEITEDYEYYTKVAEVVEVNVALCEAYKEKA

SEQ ID NO: 85 - protein, 214 aa, synthetic construct
  source 1..214: note = Library of modified or engineered enzymes
MQEITVLLFSNVTKNEVTTTNPKPTTPVIDPTSYVDPNATVTGDVTIGANVMISANASIRSDEGRPIVIGDRSNVQDGVVLHALESVDDDGKIIEENVVIHGDEDYAVFIGKDVSLAHQAQVHGPAYVGDDSFIGMQSFVFKSKVGSNCVIEPEAAAIGVTVPDGKYIPAGTVVTTQAEAEKLPDVTPDHAQYTTQAAVVTVNVQLTKAYRNLK

SEQ ID NO: 86 - protein, 214 aa, synthetic construct
  source 1..214: note = Library of modified or engineered enzymes
MQEITVLKFSNVTKNEVTVTNPKPTTPVIDPTSYVDPKATVTGDVTIGKNVLIAANATIRADEGKPIVVGDRSTVQDGVVLHALESVDDTGKVIEENVVIKGNEDYAVYIGNNVSLAHQAQVHGPAHVGDDSFVGMKALVFKSKVGKNCVIEPDAAAIGVTVPDGKYIPAGTVVTTQEEAAKLPEITPDYPFYTTNAEVVSVNVKLCEAYKGEA

SEQ ID NO: 87 - protein, 282 aa, synthetic construct
  source 1..282: note = Library of modified or engineered enzymes
MIVRILVVTLLVLSGFPALSTSGTLQDKKAASECSDQPFSYDHGASGQQSWCGRCNESGALPLPQAPINIPKIAESAQPAIVFNGYNENTSLVIYPHNPYNLKVDYKSSSNPVATIDIGSSANSRFKLLEFHFHRPSEEAIDNRRFPMVLHLVHLREVEGCEPGKPGCVAAVAILIKEGTPSQQTTDLLNALFSHFPPPDKPKDVEINLEGLLPPDHVNAGYWSYGGSLTTPPCTENITFYLLKPMLTFSAAQIAEFERRYPTPNARDIQPLHDRHRVVNRH

SEQ ID NO: 88 - protein, 186 aa, synthetic construct
  source 1..186: note = Library of modified or engineered enzymes
MIKSNPRGDLPQVHETAFVDPTAILCGYVIVEENVFIGPYAVIRADETDADGRIAPIVIGAHSNIQDGVVIHSKSGAWVTIGQRTSIAHRAIVHGPCTVGDGVFIGFNSVLFNCTIDDGCVVRYNAVVDGCHLPPGFYVRSTERIGPETDLAALPQVTADASDFSEDVARTNNALVLGYKHIQNEF

SEQ ID NO: 89 - protein, 214 aa, synthetic construct
  source 1..214: note = Library of modified or engineered enzymes
MQEITVFEFSNVEKNEVTSTNPKPTTPKIDPTSYVDPNATVIGDVTIGENCMISATASIRSDEGRPIVIGDRSNVQDGVVLHALESVDDQGMVREDNVVLEGDEYYAVYVGDRVSLAHQSQVHGPAKVGDDSFIGMQSFVFKSTVGSNCVIEPGAAAIGVTVPDGKYIPAGTVVTTQEEADKLPEITDDYPFYSTQAAVVEVNVGLCEAYRGKA

SEQ ID NO: 90 - protein, 214 aa, synthetic construct
  source 1..214: note = Library of modified or engineered enzymes
MQEITVFDFSNVRKNKVTGTNPKPVTPVIDPTSYVDPNATVIGDVKIGKNCLIGASAVIRADEGHPIVIGDRSNVQDGVVLHALESVNDDGKILEENVVIEGDEYYAVYVGKNVVLAHQSQVHGPAAVGDDSFVGMKSLVFRSIVGSNCVIEPNAAAIGVTVPDNKYIPAGTVVTTQEEADNLPEITPDYPYYDTVEQVVKVNVNLCEAYREKE

SEQ ID NO: 91 - protein, 214 aa, synthetic construct
  source 1..214: note = Library of modified or engineered enzymes
MQEITVTRYENIRPSPVTPWNPEPRRPEIHPSAYIDPAAVVQGDVTIGANVYVAANAVIRADEGYPIVIGDNSSVQDNVVLHALETVDADGRRIEENIVEVGGERYAVYVGANVVLAHNAQVHGPAIVGDNTFVGMNALVFRSRVGANCVLMHLAAAIGVTVPDGTYVPAGKVVTTQEEAAKLPKVTPDHPFYKLNARVVAVNVALAKGYLALS

SEQ ID NO: 92 - protein, 214 aa, synthetic construct
  source 1..214: note = Library of modified or engineered enzymes
MQEITVTKFENIQPSPVTPWNPEPKKPEIDPTAYIHPAAVVQGDVKIGKNVMISALASIRSDEGYPIVIGDNSNVQDQVVLHALETVDENGNVIEENVVTVGDEKYAVYIGKNVSLAHQAQVHGPAIVGDNTFIGMQAFVFRSKVGENCVLEPLAAAIGVTIPNNTYIPAGKVVTTQEEAAKLPKITPDHPFANTNAAVVKVNVALAKGYLALS

SEQ ID NO: 93 - protein, 233 aa, synthetic construct
  source 1..233: note = Library of modified or engineered enzymes
MKSILAVFTGCYQPNDEHWRYEDENGPEKWAEIEKNSDCGGKHQSPINIIHKETDSVHGPLDLQINYEPSTLITEVRNNGHSIQFDFEKGDSINYKNETYYLKQIHFHEPSEHKINGIIYPIEMHLVHMNKSGKITVLGILGEEGEESQLFEFFESFLPLKNGETKDIHQKIDLSSLFLEDKHYYSYDGSLTTPPCSENVNWIVFKEPIVLSVEEVIKLRNNMPLNNYRNEQP

SEQ ID NO: 94 - protein, 214 aa, synthetic construct
  source 1..214: note = Library of modified or engineered enzymes
MQEITVSLFSNVTKNEVTSWNPKPTTPVIDPTSFIDPNATVTGDVTIGKNCLIGPNAVIRADEGSPIVIGDRSNVQDGVVLHALESVNDEGKIIEENVVLYGSKLYAVYIGKNVSLAHQSQVHGPARVGDDSFVGMNSLVFNSIVGSNCVIEPNAAAIGVTVPDGKYIPAGTVVTSQAEADKLPEITPDHAYYTQNFAVVNVNVNLCRAYRNKS

SEQ ID NO: 95 - protein, 230 aa, synthetic construct
  source 1..230: note = Library of modified or engineered enzymes
MASSAFAAEGAHWGYTGHGGPAHWGDLSADYATCKLGKHQSPIDIRGAKEADLPAIQFDYKASPLKILNNGHTVQVNYAPGSGIVVDGKPYELVQFHFHKPSEEKIDGKAYPMVAHLVHRDAAGHLAVVAVLIKEGKENPLIKTLWPHLPAEEGPEQAVAGATINAADLLPADRGYYAFDGSLTTPPCSEGVRWHVLKQPITMSKAQIDAFQKLYKPNARPLQPLNGRIM

SEQ ID NO: 96 - protein, 214 aa, synthetic construct
  source 1..214: note = Library of modified or engineered enzymes
MQEITVTRYENIRASPVTPWNPEPKLPKIHPTAYIDPAAVVQGDVTIGENVLVMANAVIRADEGYPIVIGNNSSVQDNVVLHALETVDENGNEIEENVVTVGDKKYAVYVGDNVVLAHNAQVHGPAAVGDNTFVGMNALVFRSRVGKNCVLAPNAAAIGVTVPDGKYIPAGKVVTTQEEADKLPEITPDHPFANLNARVVKVNIALAKGYLAQA

SEQ ID NO: 97 - protein, 214 aa, synthetic construct
  source 1..214: note = Library of modified or engineered enzymes
MQEITVLIFSNITKNEVTSTNPKPKTPKIDPTSYIDPNAKVIGDVTIGKNVLIAAFAVIRADEGKPIVIGDRSNVQDGVVLHALESINDDGKIIEDNVVIEGNNHYAVYVGNNVSLAHQSQVHGPAHVGNDSFVGMKSLVFKSDVGDNCVIEPEAAAIGVTVPDGKYIPAGTVVTTQEEAAKLPEITEDYPFYTKVAEVVKVNVDLCLAYRNKQ

SEQ ID NO: 98 - protein, 214 aa, synthetic construct
  source 1..214: note = Library of modified or engineered enzymes
MQEITVHHFSNVRKNEVTPTNPKPTTPVIDPTSYIDPNATVIGDVTIGKNCYVAHSAVIRADEGHPIVIGDRSNVQDGVVLHALESVDDGGEIREDNVVEVGDESYAVYVGKNVVLAHQSQVHGPAAVGDDSFVGMKSLVFQSTVGSNCVIEPEAAAIGVTVPDGKYIPAGTVVTSQAEAAKLPEVTPDHADYTTQAAVVTVNVALCEAYKAQA

SEQ ID NO: 99 - protein, 287 aa, synthetic construct
  source 1..287: note = Library of modified or engineered enzymes
MGSYKTLTDIGKMMLKTLLLASTVSAWTYSDQTAWGGECKTSKSQSPINIVTSSAVCKNSKDDPIKADSFVAEKLGGKHAMTLNNVTSSGTHSATWTFKTMPENSQLKCAQHHCHFDVAEHSMDGEKHFGECHVVCMQAKYADLGKALESKATDALAVFGFLLAKGTATTADHAVTKQMIDAKKNYAEGKEYEMEIPATTQLADGYYRYNGGLTTPGCNEAVTWTVFKNVQYVSVAQYNEIMTWKDGNLRGNDRKVQPMNGRSLTFYKSSASKMMASLAIIGVMFMF

SEQ ID NO: 100 - protein, 258 aa, synthetic construct
  source 1..258: note = Library of modified or engineered enzymes
MRFNRFVTTLLAACLMPLMTQAAPWGYTGETGPAQWGKISKEYATCQTGINQSPVDIQTATTSKLGLPALNTQYIDNPTRFRSINYTLRATMSSYSSNFIEIEGRLYYLKHFDFHAPSEHTLNGKTYPLELQLVHKNQHGDIAIVAVMFDVGEPNQAIQNLWESFPTMVDNSMPIFSDVDINQLLPDNKAYWLYSGSLTTPPCTEGVTWVVLKKPVALSAEQLDNFHYIVGPANNRPPQPLNARTITDSHSGNTEILY

SEQ ID NO: 101 - protein, 214 aa, synthetic construct
  source 1..214: note = Library of modified or engineered enzymes
MQEITVTRFENIQPSPVTPWNPEPKLPEIHPTAYIHPAAVVQGDVTIGENVMVSANASIRSDEGYPIVIGDNSNVQDNVVLHALETVGEDGEVLEENVVVVGDERYAVYVGKNVSLAHQAQVHGPAAVGDNTFIGMQAFVFRSRVGKDCVLEPLAAAIGVTIPDGTYIPAGLVVTTQEEAAKLPKVTPDHPFANTNAAVVKVNVALAKGYLAQA

SEQ ID NO: 102 - protein, 214 aa, synthetic construct
  source 1..214: note = Library of modified or engineered enzymes
MQEITVLEFSNVTKNEVTPFNPKPETPVIDPTSYIDPNATVIGDVTIGKNCMISANASIRSDEGKPIVIGDRSNVQDGVVLHALESVDDGGMVLGDNVVVHGNSWFAVYVGKNVSLAHQAQVHGPAAVGDDSFIGMQSFVFNSIVGSNCVIEPNAAAIGVTVPDNKYIPAGTVVTSQEEADKLPEVTEDDKYFTTQEAVVKVNVNLAEAYRGLA

SEQ ID NO: 103 - protein, 266 aa, synthetic construct
  source 1..266: note = Library of modified or engineered enzymes
MLKRISAFTVCGWNYEDQHPEKWASLFPLCAGKRQSPINIVTSEVVYDPKLKPLKLSYEPATCREIVNNGHSFNVEFDDSQDKSVLKGGPLSGIYRLKQFHFHWGAADDKGSEHTVDGAKYSAELHLVHWNAKYGDFAEAASKPDGLAVVGVFLKVGKANPELQKLLDALGSIKTKGKQTRFTNFDPSTLLPSSLDYWTYDGSLTTPPLLESVTWIVLKEPISVSPAQMEQFRSLLFTSEGETACCMVDNYRPPQPLKGRQVRASF

SEQ ID NO: 104 - protein, 186 aa, synthetic construct
  source 1..186: note = Library of modified or engineered enzymes
MIKTNPRGDLPQVHESAFVDPTAILCGWVIVEEYVFIGPYAVIRADELNADGDMEPIVIGAHSNIQDGVVIHSKSGAAVTIGRHTSIAHRAIVHGPCRVGDGVFIGFNSVLFNCTIDDGCVVRYNAVVDGCHLPPGFYVRSTERIGPETDLAALPQVTADASDFSEDVARTNNALVLGYKHIQNEF

SEQ ID NO: 105 - protein, 214 aa, synthetic construct
  source 1..214: note = Library of modified or engineered enzymes
MQEITVTVFENIRPSPVTPWNPEPRLPEIHPTAYIDPAAVVQGDVTIGENVMISANASIRSDEGYPIYIGNNSNVQDNVVLHALETVDENGKRIEENIVTVGDKEYAVYIGDNVSLAHQAQVHGPAAVGDNTFIGMQAFVFASRVGKNCVLAPLAAAIGVTVPDGTYVPAGKVVTTQEEADKLPKMTPDHPFYKTNDAVVKVNVALAKGYLAQA

SEQ ID NO: 106 - protein, 266 aa, synthetic construct
  source 1..266: note = Library of modified or engineered enzymes
MKAISLVFTCGYWRENDPHQWHLTFPAAKGERQSPIDIQPAKAKYDPGLKPLKLSYDPATARRILNNGHSFNVEFEDSQDKAVLKGGPLTGSYRLLQFHFHWGSTDDHGSEHTVDGVKYASELHLVHWNAVKFSSFAEAASKADGLAVIGVFLKVGEPHAEMEKLLNALHAIKTKGKEAPFTNFDPSCLLPTCLDYWTYSGSLTTPPLLECVTWIVLKQPISVSSEQMAKFRSLLFTSEGEKEKRMVDNFRPLQPLMNRTVRSSFR

SEQ ID NO: 107 - protein, 260 aa, synthetic construct
  source 1..260: note = Library of modified or engineered enzymes
MTRRAVLNRRGALAALALLAVAGCAGSDPTAAAPHWDYDHEGPDHWADLGKQYATCRNGHAQSPIDLPDAGEAHPTDDIDIVYRRIRTATLTNNGHAIQVGVPADSGNRIVVDGTSFTLTQYHFHLPSEHTVAGAETAMELHLVHTDAHGRLAVLAVLLRAQEAPAPLSAILAAAPDRVGATRTLSNIDPRAFLPDNRAQFRYEGSLTTPPCTEGVAWIVLREPSPVAVADVDRYRRLFPHSNRPTQPRNDRPVILAGTN

SEQ ID NO: 108 - protein, 239 aa, synthetic construct
  source 1..239: note = Library of modified or engineered enzymes
MKIISWLFIFLLGACATDWSYSGRGSPQNWAEISESNKFCKIGYNQSPIDINLSMNKDFILNDLKFDYKISEIEKVNEKYYQKINFYSKSFVLRGKKKYWLKYIEFRHPSEHFLDSSPHSLEMQIYHKSEDEQWLATSYFLEIPAMNNNENLYFNNLIDFLKSKKIEDKFDLSKIIDETSLSFFYEGSFTTPPCTEGVKWYIMKNPIFISKEQMNTIIKSTIFVKSNARGIQKFNPEKF

SEQ ID NO: 109 - protein, 288 aa, synthetic construct
  source 1..288: note = Library of modified or engineered enzymes
MQPSAFHKLLLLLPLAYHRTPNVGDDKDEHWNYETNGKNWGGICASGERQSPISLSVQKSYIISIPRIVFGNYDIKLRGPLTITNNGHTAHMDIPETTNGKKPFITEGMLNGRYVAESLHFHWGSPGSRGSEHAINKQRYDVEMHIVHRNAKYKDMSEAVGKKDGLAVIGVMLKIVKNPKLMFLGLHNVLGAVSRITKTKAKTYVPGSFSLGQVLGIVNPRSYFTYRGSLTTPFCQEAVTWTVFTQVLPVSYTLVSKLWRLRDSEGHRLINNFRDIQPTNRRAVFYRP

SEQ ID NO: 110 - protein, 214 aa, synthetic construct
  source 1..214: note = Library of modified or engineered enzymes
MQEITVTKYENIQASPVTPWNPEPKLPEIHPTAYIHPAAVVQGDVTIGANVLVMANAVIRADEGYPIVIGDNSSVQDNVVLHALETVDADGKVIEENVVTVGDKKYAVYVGDNVVLAHNAQVHGPAAVGDNTFVGMNALVFNSVVGKNCVLAPLAAAIGVTVPDGKYIPAGKVVTTQEEAAKLPEVTPDHPFYNLVDRVVAVNVALAAGYLAQA

SEQ ID NO: 111 - protein, 214 aa, synthetic construct
  source 1..214: note = Library of modified or engineered enzymes
MQEITVTRFENIQPSPVTPWNPEPKLPEIDETAYVHPAAVVQGDVKIGKNVLIMANAVIRADEGYPIVIGDNSSVQDNVVLHALETVDENGNVIEENVVTVGDEKYAVYVGDNVVIAHNAQVHGPAAVGDNTFVGMNALVFRSTVGKNCYLAPNAAAIGVTVPDGTYVPAGTVVTTQEEAAKLPKITPDHPFANLNKRVVKVNVALAKGYLALS

SEQ ID NO: 112 - protein, 283 aa, synthetic construct
  source 1..283: note = Library of modified or engineered enzymes
MSYSRVSTYLLALSVLCFSVFVVVPTGVCQAINPPEKVQPGQHVHNGQHHMLHMMLGEEKCGPTYTYEEGVKGPSHWPEVCTTGKMQAPIDIQSTQKLPINNLKFNYQPADLDILNDCNQYRVLVKFPDNYWLMVGKKPYNLAEIHFREPGETAVNGKRPKMSIEFLHFSPEGVFLVIEVPVVAGKENPTMQAILQNVPAPGKEEKVAGVKINPTDLLPIDRRSFYRYPGSLTTPDCTEVVTWYVMKTPIEMSEAQIAEYSKHYHDTARPLQPVNGRPVVEDQ

SEQ ID NO: 113 - protein, 252 aa, synthetic construct
  source 1..252: note = Library of modified or engineered enzymes
MTCTGKSTDYWNYDNPSEWGTHFPAANGLCQSPIDIDSHKTIRHVYPKFQFSKKYHSSELFKLINQTYQVTATLADRTYGQNDNDLWFTGGGLEGTFYFVNFHLHWGRDDRHGSEHEIDGHQFPAEGHVVFQNRQTKQAAVFAFLFTVADRFHKENKEWCKYADAASQLTNDEDSIQCLFNLHDLMNVNDRLFYRYTGSLTTPPCTEGIVWTIFSQKIAIKQESLQKLRKNILTKVYRPVQPLNDRIVYKNH

SEQ ID NO: 114 - protein, 234 aa, synthetic construct
  source 1..234: note = Library of modified or engineered enzymes
MKLSNQIRFYGVATCPEWHDYYPIADGDRQSPINIISSQARYDPSLRPLELKYDPSTSLEILNNGHSFQVTFADDSDSSTLKDGPISGVYRLKQFHFHWGAADDKGSEHTVDGVKYPAELHLVHWNAVKYSSFAEAASKENGLAVIGVFLKIGQHNANLQKIVDALNAIKTKGKQTTFTNFDPSTLLPGCLDYWTYDGSLTTPPLLESVTWIVCKEPISVSSEQMAKFRSLLFS

SEQ ID NO: 115 - protein, 282 aa, synthetic construct
  source 1..282: note = Library of modified or engineered enzymes
MRIMTRGALTGVLWMLSVVGLQAAEPGSIPWGYEGDLGPNHWGSLGSEFALCEKGMSQSPIDLVQTHKLALTDIQFSYRDAPFHVINTGHTLEELEPLSETVKSRYPKHGQTVLHFQKDSTIVFDDDLYLLEQFHFHSPSEHTLHEKHYPMELHLVHHNERHEAAVVAVFMKEGKHNPFFETFLDHAPKTVGEFVEDRERVINPVNLLPKNHTYYRYFGSYTTPPCHEGVIWAVMHDPIEVSREQVQRFRSLVGHDNARPTQPLHKRFVLESNDVRAPGKLK

SEQ ID NO: 116 - protein, 232 aa, synthetic construct
  source 1..232: note = Library of modified or engineered enzymes
MSLKEWGYDAHNGPQTWCRVFIAAEGKRQSPIDIQTKEVESDLTLKPLKLNYEPASSLRILNNGHSFQVEFDDSTDKSVLTGGPLTGTYRLRQFHFHWGSCDDHGSEHTVDGVKYASELHLVHWNAKYESFAEAAKQPDGLAVVGVFLKIGKENPKLQRVLDALNAIKTKGKQTTFTNFDPSTLLPPCLDYWTYHGSLTVPPLLESVTWIILKEPISVSPSQMSKFRSLLFT

SEQ ID NO: 117 - protein, 214 aa, synthetic construct
  source 1..214: note = Library of modified or engineered enzymes
MQEITVLNYSNIVKNEVTSTNPKPEVPVIDPTSYVDPNATVIGDVTIGKNCYIAAFARIRADEGKPIVIGDRSNVQDGVVLHALESIDDTGEVNKDNVVIEGNELYAVYIGDNVSLAHQSQVHGPARVGDDSFVGMKSLVFKSDVGDNCVIEPEAAAIGVTVPDNKYIPAGTVVTSQEEAAKLPEVTPDYAYYTTQEAVVEVNVALTEAYKGKM

SEQ ID NO: 118 - protein, 214 aa, synthetic construct
  source 1..214: note = Library of modified or engineered enzymes
MQEITVMDFSNITKNEITSWNPEPSTPKIDPTSYIDPNATVIGDVTIGKNCYIGPFAVIRADEGAPIVIGDESNVQDGVVLHALESVDAGGKIREDNVVLHGDKLYAVYIGKNVSLAHQAQVHGPAYVGDDSFVGMNSLVFKSKVGSNCVIEPFAAAIGVTVPDGKYIPAGTVVTTQAEADKLPEVTPDHAEYTKNAAVVNVNVALCEGYKSLA

SEQ ID NO: 119 - protein, 265 aa, synthetic construct
  source 1..265: note = Library of modified or engineered enzymes
MRRKRVSRFNAPQRLPYMHKLAIAAALFLAAAIPSFAADDCPVPWGYTVDNGPATWGRYSAICASGLSQSPVKINNLLPSPATNLPTLSFQGGPSRFRVKNNQHDLEVYPVNQWTLQPFGARLTKFHFHVPAEHLDGNTRHDAEAHFVYELGNRIFAIAVWIDQVNQGGNAALQKIAAVQRPGLCLMSPLSLPAATLNILDFLPDRNNYAAYHGSLTTPPCTENVTFFIMRTPITATATQINALTLVAPAPPGNARPVQQTKWRR

SEQ ID NO: 120 - protein, 214 aa, synthetic construct
  source 1..214: note = Library of modified or engineered enzymes
MQEITVTNYNNIRPSPVTPWNPEPKLPEIHPTAYIDPKAVVQGDVTIGKNVLVMANAVIRADEGYPIVIGDNSSVQDNVVLHALETVDENGNVLEENVVEVGDKRYAVYIGDNVVLAHNAQVHGPAAVGDNTFVGMNALVFRSRIGKNCVLAPLAAAIGVEVPDGTYIPAGTVVTTQEEAAKLPKVTPDHPFANLNERVVKVNVALAKGYLALA

SEQ ID NO: 121 - protein, 214 aa, synthetic construct
  source 1..214: note = Library of modified or engineered enzymes
MQEITVLLFSNIRKNEVTPTNPKPTTPVIDPTSYIDPNATVIGDVTIGANCFVGPFAVIRADEGAPIVIGDRSNVQDGVVLHALESVDDGGKVREDNVVVHGDEWYAVYIGRNVSLAHQSQVHGPAHVGDDSFVGMKSLVFKSKVGSNCVIEPGAAALGVTVPDGKYIPAGTVVTTQAEADTLPEVTPDYAFYTTQAAVVSVNVNLCEAYRAQA

SEQ ID NO: 122 - protein, 291 aa, synthetic construct
  source 1..291: note = Library of modified or engineered enzymes
MKRSLAFVTIGCQHNPEDWYPFIEGDEFGYSDSLQREWVMCKSGRMQSPIDISPENLLFDPNLRSLQIDKHKVSATLENLGQLPLLTINDSKIRPDSINISGGPASPYKYRLHHIIIHFGRSIDEEKGSEHTIDHIRFPAELQLLAYNTDLYSNFSEAMTQPRGLLAISIIVDIGKITNTELRKLTVASQSITYKGQKTILKRFNAYGLLPETEDYITYEGSLTFPGCYETVTWVIMNNPIYITKEDLHIWNDLQQTEFKQPNPVFMFPNYRPLKPLNGRLLRTNINIKYK

SEQ ID NO: 123 - protein, 214 aa, synthetic construct
  source 1..214: note = Library of modified or engineered enzymes
MQEITVLEFSNIKKNEVTSYNPKPKTPVIDPTSYIDPNATVIGDVTIGKNCYIGPFAVIRADEGAPIHIGDNSNVQDGVVLHALESVDDGGKVREDNVVLYGDKYYAVYIGKNVSLAHQSQVHGPARVGDDSFVGMNSLVFNSIVGNNCVIEPNAAAIGVTVPDNKFIPAGTVVTSQAEADKLPEITPDHAFYTDIAKVVSVNVKLCKAYLEKQ

SEQ ID NO: 124 - protein, 214 aa, synthetic construct
  source 1..214: note = Library of modified or engineered enzymes
MQEITVLTYSNVTKNEVTSTNPKPTTPVIDPSSYVDPNATVTGDVTIGKNCLIGANAVIRADEGAPIVIGDNSSVQDGVVLHALESVDDGGKVIEDNVVLHGDNWYAVYVGKNVVLAHNAQVHGPAYVGDDSFVGMKSLVFKAIVGSNCVIEPDAAAIGVTVPDGKYIPAGTVVTTQEEADKLPEITPDDAKYTKVAEVIAVNVALCKAHREKA

SEQ ID NO: 125 - protein, 260 aa, synthetic construct
  source 1..260: note = Library of modified or engineered enzymes
MRKISFLVAGCTPENWHDYQPVAGGERQSPINIITKEAKYDPSLKPLSFTYDPSTSLEILNNGHSFQVTFADNSDSSTLTGGPLTDKYRLTQFHFHWGSTDDHGSEHTVDGVKYASELHLVHWNADKYSSFAEAASKPDGLAVLGVFLKVGEHNPSLQKLTDALYSVRFKGTKAQFTNFNPKCLLPSSLDYWTYSGSLTTPPLLESVTWIVLKEPISVSSEQMEKFRSLLFTSEGETACCMVDNYRPLQPLKGRKVRASF

SEQ ID NO: 126 - protein, 325 aa, synthetic construct
  source 1..325: note = Library of modified or engineered enzymes
MVFSIATFGLLLLLGFCLGDDFGYDGNHGPSHWGEEYHTCIGKHQSPINIEEHNVKNVSLPPLKLIGIDDPYQSFVTNNGHTVMLKINESKVIMLSGGPLGNKVYVFEQLHFHWGQNDFEGSEDLINNHSFPMEMHAVFYKEDYKSMNEALNHSDGLAILAYLYEVSPNPNVMYEPIVEVLPDIETVGSEKVLREPLMLRKLFISDITTMQDYFTYNGSLTTPPCLEVAIWIDFKDHLRLSHEQIAAFRNLRSTEGDKLTHNFRPVQSLEDRIVLHNIPREQNIPRNIPPKTYHRFDEHSGQHNVEMPLSIIALAVLFAVILFAI

SEQ ID NO: 127 - protein, 210 aa, synthetic construct
  source 1..210: note = Library of modified or engineered enzymes
MTLFSKIRENVYGHAPQCDWCYEGEGAPESWGRLRPEFATCAVGRRQSPIDIRDGIAVDLEPIRFDYRPTSFRIVDTGNTIQVNVAPGNTIEVMGRRYELVQFHFHRPSEERIDGRQFDMVAHLVHKDGEGRLAVVAVLLERGDDQPLVRTVWNNLPLEKGDEVAARTPIDLNALLPEDRRYYTYMGSLTTPPCSEGVLWMVMKQPVQLS

SEQ ID NO: 128 - protein, 214 aa, synthetic construct
  source 1..214: note = Library of modified or engineered enzymes
MQEITVTTYNNIRPSPVTPWNPEPKLPKIHPTAYVDPAAVVQGDVTIGKNVMISALASIRSDEGYPIYIGDNSNVQDNVVLHALETVDENGNVIEENVVTVGDKKYAVYVGDNVSLAHQAQVHGPAAVGDNTFIGMQAFVFRSNVGKDCVLEPLAAAIGVTVPDGTYVPAGKVVTTQEEADKLPKVTPDHPFYKTNEAVVKVNVALAKGYLALS

SEQ ID NO: 129 - protein, 214 aa, synthetic construct
  source 1..214: note = Library of modified or engineered enzymes
MQEITVLEFSNITKNEVTPWNPKPKTPVIDPTSYIDPDATVIGDVTIGANCYIGASAVIRADEGKPIVIGDDSNVQDGVVLHALESINDEGKVIEDNVVIHGNKRYAVYVGKNVSLAHQSQVHGPAAVGDDSFVGMQSLVFNSKVGSNCVIEPNAAAIGVTIPDGRYIPAGTVVTSQAEADKLPEITPDYAKSNAVAAVVNVNVGLCEAYREEA

SEQ ID NO: 130 - protein, 214 aa, synthetic construct
  source 1..214: note = Library of modified or engineered enzymes
MQEITVGEFSNVTKNEVTTTNPKPETPVIDPSSYVDPSSTVIGDVTIGKNCYIAANAVIRADEGAPIVIGDNSNVQDGVVLHALESVNDGGKLREDNVVLEGDEYYAVYVGKNVHLAHQAQVHGPAAVGDDSFVGMKSLVFNSIVGSNCVIEPNAAAVGVVVPDGKFIPAGTVVTTQEEADNLPDITPDHAAYTTQAAVVKVNVALCEAYKAEA

SEQ ID NO: 131 - protein, 214 aa, synthetic construct
  source 1..214: note = Library of modified or engineered enzymes
MQEITVTKFENIRPSPVTPWNPTPKLPKIHPTAYIDPAAVVQGDVTIGENVMVSANASIRSDEGYPIYIGNNSNVQDNVVLHALETVDENGKRIEENIVRVGDKDYAVYIGDNVSLAHQAQVHGPAAVGDNTFIGMQAFVFRSRVGKNCVLEPLAAALGVEVPDGTYIPAGEVVTTQEAAAKLPKITPDHPFANTNAAVVKVNVALAKGYLALS

SEQ ID NO: 132 - protein, 277 aa, synthetic construct
  source 1..277: note = Library of modified or engineered enzymes
MRARAVTGGIHGRIRSTLGAALLAPLFLAAGCGGEGGGGTGEESGLAETHLAAWSHAGADGPDEWASLDPAYATCGTGERQSPIDIVGAKRRPFPPVELDYAPVRATLIDNGHAIEAELEDSGSSARIGGDEFTLEQFHFHMPAEEVVGGKSFAASIHLVHLDEDGGAAVVGLLVEPGPENPVIERLAEEVPEETDEPVEVEGELDLAGLVPDGDAFRYEGSLTTPPCTEGITWTVFEDPVTMSPEQLEAFAGAYDANARPVQARNGREISVGPGLG

SEQ ID NO: 133 - protein, 214 aa, synthetic construct
  source 1..214: note = Library of modified or engineered enzymes
MQEITVTVFENIRPSPVTPWNPTPRRPEIHPTAYVDPLATVVGDVRIGANVMVSANASIRSDEGYPIVIGDNSNVQDNVVLHALETVDAAGRRLEENVVEVGDELYAVYVGANVSLAHQAQVHGPAAVGDNTFIGMQAFVFRSRVGANCVLEPLAAAIGVTIPDGTYIPAGKVVTTQEEAAKLPKITPDHPFANTNAAVVAVNVALAAGYRALA

SEQ ID NO: 134 - protein, 214 aa, synthetic construct
  source 1..214: note = Library of modified or engineered enzymes
MQEITVLTFSNVTKNDVTATNPKPVTPVIDPTSYVDPNATVTGDVTIGKNCLIEANATIRADEGHPIVIGDRSSVQDGVVLHALESVDDGGELIEDNVVLEGDEEYAVYIGKNVHLAHQSQVHGPAKVGDDSFVGMKSTVFKSIVGSNCVIEPDAAAIGVTVPDGKYIPAGTVVTTQEEADKLPEITPDHAKYTANAAVVTVNVALCEAYRNEA

SEQ ID NO: 135 - protein, 214 aa, synthetic construct
  source 1..214: note = Library of modified or engineered enzymes
MQEITVLEFSNITKNEVTPWNPKPKTPEIDPTSYIDPQATVIGDVTIGKNCYIGPFAVIRADEGAPIVIGDDSNVQDGVVLHALESINEKGEIIEDNVVIKGNKRYAVYIGKDVSLAHQSQVHGPARVGDHSFVGMNSLVFNSIVGDNCVIEPNAAAIGVTVPDGKYIPAGTVVTSQAEADKLPEITPDHAYYTKNAAVVNVNVALCRAYKSKE

SEQ ID NO: 136 - protein, 230 aa, synthetic construct
  source 1..230: note = Library of modified or engineered enzymes
MSTIPWRLGAVFCNYQKHEDAVEEKEFSYDEGSERGPSRWGEIRPEWRTCGNGEMQSPIDLLNQRVEIVSKLGKLKRDYKPSNATLKNRGHDISLEWKGGAGSIEINGTEYVLQQCHWHSPSEHTINGRRFDMELHMVHESRDGKVAVVGIVYKLGRPDSFLSSLMDHLEAISDTKDRERAVGVIDPRHIKFGSRKYYRYMGSLTVPPCTENVIWTIVKRVRTVSREQLK

SEQ ID NO: 137 - protein, 243 aa, synthetic construct
  source 1..243: note = Library of modified or engineered enzymes
MKYGVLVLILSFIQFTYAQNKKDWGYKDSGAPQYWANINPLYLGCTEGNQQSPINIITKNVNKGAAHFELKYSVAKGVNLILSHNTFKMVYPQGNFLEMNGNRYQLKEIYFKTPGENAIDSLRGMLEAQLLHEDSKGNKVILAVFFIEGRSNPIIDMLVKNLPTQPDKANFIANVDVHQLLPSDLASYQFDGSLTMPPCSQGVRWIVLKQTMTITQSQVDSMRDITGVNSRPTQEIFNRLIVK

SEQ ID NO: 138 - protein, 214 aa, synthetic construct
  source 1..214: note = Library of modified or engineered enzymes
MQEITVLIFSNIRKNEVTPTNPKPVIPVIDPTSYVDPNATVIGDVTIGKNCYIAHSAVIRADEGKPIVIGDRSNVQDGVVLHALESVNDGGKIREDNVVIEGDEEYAVYIGKDVSLAHQSQVHGPARVGDHSFVGMKSLVFNSIVGSNCVIEPDAAAIGVTVPDNKYIPAGTVVTSQEEADKLPEITPDHAKYTAIAAVVNVNVALCQAYKEKS

SEQ ID NO: 139 - protein, 285 aa, synthetic construct
  source 1..285: note = Library of modified or engineered enzymes
MKRTFLIAVSLCPGLIQYNHWDEWWTYEGISGPAYWGLINPAWSLCNKGRRQSPVDIDPEKLLFDPNLKSLHLDKHKVSGTLENTGQSLVFRVDKDTKHHVNISGGPLAYKYQFQEIYFHWGVHDGLGSEHTINHQSFPAELQLYGFNSELYSNMSEAQEKPHGVVGISLLVQIGKTPNPELKILTSQLENIRYKGQSAPIKNFSLRGLLPNTEHYVTYEGSTTHPGCWETTVWVVLNKPVYITKQELYALRRLMQGSKEHPKAPLGNNARPTQDLHHRTVRTNI

SEQ ID NO: 140 - protein, 214 aa, synthetic construct
  source 1..214: note = Library of modified or engineered enzymes
MQEITVTRYENIRESPVTPWNPTPRRPEIHPTAYVDPAAVVVGDVTIGENVMISANASIRSDEGYPIYIGDNSNVQDNVVLHALETVDAAGKRITENIVTVGDKEYAVYIGKNVSLAHQAQVHGPAAVGDNTFIGMQAFVFRSRVGKNCVLEPLAAAIGVTVPDGTYIPAGKVVTTQEEAAKLPKITPDHPFAKTNAAVVAVNVALAAGYRALA

SEQ ID NO: 141 - protein, 268 aa, synthetic construct
  source 1..268: note = Library of modified or engineered enzymes
MKLTSIAVFCGYPEQNRDHWGYQDHNGPEMWKEKFPSAGGKKQSPIDIQTAETTFDPKLKPLELKYDPSTAKEILNNGHSFQVTFVDDTDSSTLKDGPISGIYRLKQFHFHWGASDDHGSEHTVDGVKYAAELHLVHWNAKYGKFGEAASQPDGLAVVGIFLKIGRHHEEFQKLLDALDSIKTKGKQTTFTNFDPSTLLPGCLDYWTYFGSLTTPPLLESVIWIVLKEPISVSSEQLAKFRSLLFTSEGEKEKRMVDNFRPLQPLMNR

SEQ ID NO: 142 - protein, 214 aa, synthetic construct
  source 1..214: note = Library of modified or engineered enzymes
MQEITVAEFSNVTKNEVTPYNPKPVTPVIDPTAYVDPEATVIGDVTIGKDCMISANASIRSDEGHPIVIGDRSNVQDGVVLHALESVNDGGEVIEDNVVVEGNELYAVYVGKNVSLAHQAQVHGPAAVGDDSFIGMQSFVFNSIVGSNCVIEPEAAAIGVIVPDNKYIPAGTVVTTQEEADKLPEITPDYAYYTTVAAVVNVNVALCKAYRRLM

SEQ ID NO: 143 - protein, 278 aa, synthetic construct
  source 1..278: note = Library of modified or engineered enzymes
MPAPAPKAAPKAGHGAKKAAPAPKAAPKAAPKAAPRAKVVKAAPAPPPPEPAHAHWSYEGEGAPARWGQLKPEWKQCAVGTRQSPIDIRDGIKVDLDPIQFDYKASGFSVIDNGHTVQVNLAPGNFITVLGRRYELVQFHFHKPSEERINGKPYDMVAHLVHKDAEGRLAVVAVLLRPGEANPLIEKVWTYMPLDAGDRVRMPTELIDLNQLLPADRAYFTYMGSLTTPPCSEGVLWMVMKQPVPVSADQIAIFARLYPMNARPLQAVSGKIIKETLM

SEQ ID NO: 144 - protein, 264 aa, synthetic construct
  source 1..264: note = Library of modified or engineered enzymes
MNRKKLNSLIAAVIVFFATSAFSESPHWDHAEQSTWWAIEDTTQTYPPKRFPFAVCGVGQHQSPIDLAAAEVIDTIQINPLEILYDVDHAPVFFNSGHGIQVNTSIEYSGKLKVGEELFPLIQFHFHAPGEHVIGDTKFPAELHYVHIQADGKIAVLAVAINIGDENSAFQTILENVPSVSGGKNENSGLQFDPAALLPPLDHPIKYYTVAGSLTTPPCSEGVQWYFLPTAITISEAQLNQLRSLYADNNRLPQDVNGRSLLTQ

SEQ ID NO: 145 - protein, 214 aa, synthetic construct
  source 1..214: note = Library of modified or engineered enzymes
MQEITVTRYENIRESPVTPWNPTPRRPKIHPTAYVDPAAVVVGDVTIGANVMVSANASIRSDEGYPIYIGDNSNVQDNVVLHALETVDADGKTLEENVVTVGDKKYAVYVGDNVSLAHQAQVHGPAAVGNNTFIGMQAFVFRSTVGENCVLEPLAAAIGVTVPNGKYIPAGKVVTTQEEADKLPEVTPDHPFANTNAAVVKVNVALAAGYRALA

SEQ ID NO: 146 - protein, 214 aa, synthetic construct
  source 1..214: note = Library of modified or engineered enzymes
MQEITVDEFSNITKNEVTGTNPEPSTPVIDPTAYVDPNATVIGDVTIGANVLVAANAVIRADEGRPIVVGDRSSVQDGVVLHALESVDDEGEVREDNVVLVGDENYAVYVGKNVSLAHQSQVHGPAAVGDDSFVGMKANVFRSTVGSNCVIEPDAAAIGVTVPDGKYIPAGTVVTTQAEADKLPDVTPDHAKSNDVAAVVAVNVALCEAYREQS

SEQ ID NO: 147 - protein, 214 aa, synthetic construct
  source 1..214: note = Library of modified or engineered enzymes
MQEITVLLFSNVTKNEVTPINPKPTTPVIDPTSYIDPNATVTGDVTIGKNCMISANASIRSDEGKPIVIGDRSNVQDGVVLHALESVDDGGMIIGDNVVVEGDEYYAVYVGDNVSLAHQAQVHGPAYVGDDSFIGMQSFVFKSIVGSNCVIEPEAAAIGVTVPDGKYIPAGTVVTTQEEADKLPEITEDDADYTTNVAVVNVNVALCKAYREKA

SEQ ID NO: 148 - protein, 214 aa, synthetic construct
  source 1..214: note = Library of modified or engineered enzymes
MQEITVTRFENIRPSPVTPWNPEPKRPVIHPTAYVHPAAVVEGDVTIGENVMVSANASIRSDEGYPIYIGDNSNVQDNVVLHALETVDENGKVLEENVVTVGDKKYAVYVGKNVSLAHQAQVHGPAAVGDNTFIGMQAFVFRSTVGKDCVLEPLAAAIGVTVPDGTYIPAGKVVTTQEEAAKLPKITPDHPFANTNAAVVKVNVALAKGYLALA

SEQ ID NO: 149 - protein, 214 aa, synthetic construct
  source 1..214: note = Library of modified or engineered enzymes
MQEITVAVFSNVTKNEVTGTNPKPRTPVIDPTSYVDPNATVIGDVTIGANCYIAHSAVIRADEGRPIHVGDNSNVQDGVVLHALESVDADGERLEDNVVIEGDKRYAVYIGKNVSLAHQAQVHGPARVGDDSFVGMKSLVFNSVVGSNCVIEPNAAAIGVTVPDGKYIPAGTVVTTQAEADKLPEVTPDHAAYTEIAKVVTVNVNLCRAYREQA

SEQ ID NO: 150 - protein, 274 aa, synthetic construct
  source 1..274: note = Library of modified or engineered enzymes
MYPIIHIKEGKYKMNYFFLFTILSSLTLSACSNSKIVQEVHPNKSIVSAARNEDWSYTGKTGPNYWSSINKKYALCSTGKQQSPVNIDQAIKKSLPLGINYHNDLFKIERSQYTVKFIPVNHSNSINLNGTNYTLLQFHFHTPSEHTLNGKQSDLEIHFINENSNKSIITIGVLVDRGRLNKEFQKILNANPMDEDLEGKVVKINLQSFIPYTSKKFSYTGSFTTPPCTEGIKWIIFNKPIQFSEEQIHSYQNYFEPNSRPVQPLNGRDLFESW

SEQ ID NO: 151 - protein, 214 aa, synthetic construct
  source 1..214: note = Library of modified or engineered enzymes
MQEITVFVFSNVEKNEVTSTNPKPTTPVIDPTSYVDPNATVTGDVTIGANVMISASASIRSDEGKPIVIGDRSNVQDGVVLHALESVDDEGEVIEDNVVIHGDKNYAVYVGKNVSLAHQAQVHGPARVGDDSFIGMQSFVFKSDVGSNCVIEPFAAAIGVTVPDGKYIPAGTVVTTQEEADKLPEVTDDYAYSDTNEAVVKVNVNLCEAYKGKA

SEQ ID NO: 152 - protein, 177 aa, synthetic construct
  source 1..177: note = Library of modified or engineered enzymes
MNPTGHMPVVSETAFIDPTAIICGKVIIEDNVFIGPYAVIRADEVNEQGDMEAIVIKRDTNIQDGVVIHSKAGAAVTIGERSSIAHRSIIHGPCQVCDDVFIGFNSVVFNAVIGKGCVIRHNSVVDGLDLPENFHVPPMTNIGADFDLNSISKVPPEYSSFSESVVSANHTLVKGYK

SEQ ID NO: 153 - protein, 281 aa, synthetic construct
  source 1..281: note = Library of modified or engineered enzymes
MLSRKPGAVTFCIWNHQEYDVKMASWGYTKENGPATWYKDFPVANGPRQSPINIDPGSAKYDPGLKALTLKYDPSTSLEILNNGHSFQVTFADDSDSSTLTDGPISGVYRLKQFHFHWGASDDKGSEHTVDGVKYAAELHLVHWNAVKYSSFGEAASKEKGLAVLGVFLKVGEHNANLQKVLDALDSIKTKGKQAPFTNFDPSTLLPASLDYWTYHGSLTTPPLLESVTWIVLKEPISVSPAQMAKFRSLLFSSEGEKEKRMVDNFRPLQPLMNRTVRSSF

SEQ ID NO: 154 - protein, 249 aa, synthetic construct
  source 1..249: note = Library of modified or engineered enzymes
MKKGLVLICLSLSLLGAFGGEHWGYSKGVGPRYWGKLSRDYEICKSGKTQSPINIQHYYHSPDKEDLSFEYENTKPLSIAYSHYTLVAQFNEPGNAVIFRDHEYSLVNLHFHIPMEFAIHGKKQPLSMHLVHRDKEGDLLVVGIGFSIGKKNPFFTPILNAYKYHTEPKLLALKTLLPDTIHYYHFNGSLTTPPCSEGVTWFIIEETLSISKEQFDEMQQIMHHQSNQRPLQKDYNRVIVKSSAIVREH

SEQ ID NO: 155 - protein, 214 aa, synthetic construct
  source 1..214: note = Library of modified or engineered enzymes
MQEITVDEFSNVTKNEVTATNPKPTTPVIDPTSYVDPEATVTGDVTIGKNCLIAANAKIRADEGKPIVIGDRSSVQDGVVLHALESVDDGGKVIEDNVVLEGNELYAVYVGENVVLAHNSQVHGPARVGDDSFVGMKSLVFKSDVGSNCVIEPFAAAIGVTVPDGKYIPAGTVVTTQEEAAKLPEVTEDYPFYTKQAEVVKVNVGLCEAYRNKA

SEQ ID NO: 156 - protein, 237 aa, synthetic construct
  source 1..237: note = Library of modified or engineered enzymes
MIGVKPKSYPAQNKKKKWSYDGDNGPQNWGDLSADYLSCEVGLNQSPVDFSKSFSSPDLHSIRFNYGISAGRVYLARGSITFKVIPGNVAYFKGKQWVLERVVLRTPSEHSIEGHSFDGELQLYHSYKGESFLWVSVLLEAGSALKPFRQIVDKAKGISGASKLMGVDLRLLIPRRKNYYFYPGSDTIPPCKEGRSWVVLRQPVGASIKYIEHLESQVGKGARPTQPLYARVPLRYD

SEQ ID NO: 157 - protein, 343 aa, synthetic construct
  source 1..343: note = Library of modified or engineered enzymes
MKGLCVMAIVALIGLQTATGYTRQDLSCGGRMDYSKPVCHNIPKHAHKKCDVYNQWSHHLFGTSQKCWGKLNPACRGMRQSPININEHKVEPNHNYGDLCITGPHHLKVHIHNTGHDLQAKLDESSSRATLVTGGPLGNKKYRVLQFHFHFASHPGGKGSEHSINCHFSDIEMHIVLQNVAYGSFDVAKDHRDGLSVIAVMLSEDVRPNTVSNAMRNNPSWSQYYINTLIYYASLRKHCDLHEVPGNTRFSLFHLLPSDYARNYYAYGGSLTTPPCSESVSWIIMRTRFHINRYHLNLLKDVSLLSRYHRGFEPMSQNKRNLQLLNNRKVYYPAGHGRCPSRG

SEQ ID NO: 158 - protein, 268 aa, synthetic construct
  source 1..268: note = Library of modified or engineered enzymes
MRLKSNIFVAGTCQPEDYHWGYEDHNGPATWAKHFPAAKGEKQSPIDIQLSNVKNVSFPPLVFNYKDSTLKEIINVGHSVQVNLEDSDNRSVLKGGPLSGPYRLKQFHFHWGKTNDVGSEHTIDGKSFPSELHLVHWNAKKYASFGEAASKPDGLAVVGVFLEIGDEHPEMNRLTDALYMVRFKGTKAQFSCFNPKCLLPASRHYWTYPGSLTTPPLSENVTWIVLREPISISERQMEKFRSLLFTSEGEKEKRMVDNFRPLQPLMNR

SEQ ID NO: 159 - protein, 348 aa, synthetic construct
  source 1..348: note = Library of modified or engineered enzymes
MKFLTPSIFFTSLRVASAATGVKFYYNDQSQWPAVPATPEGTNVCDGQQQSPINIDTGDFSCQADAQGYSFYTGDCTLGDYEFTMNDHGLKASVEKSNCEKPKMIIPGTGKVYEVLQFHIHTGCENKFNNTGCDAELHLVHIAKTDIALPAATTSADLPDLAVLGLMMYGVDEKHASVDALIDSWSEVSCSNQKCMTVSDELKSQKFSPYSLIPSDTSIYNFQGSLTTPPCWEVVTWNVAEKPIKMSFKQVLAITNLINKYSGYRAEDGSCVADSTVADDAGITSRDVQSLNGRQIVKNCEPLTVMSDFPAAAQEQSPMTASSESSAQFLNIKNIFGVVSVLASIVLF

SEQ ID NO: 160 - protein, 214 aa, synthetic construct
  source 1..214: note = Library of modified or engineered enzymes
MQEITVTRYENIRPSPVTPWNPEPKLPKIHPTAYVDPAAVVQGDVTIGENVMVSANASIRSDEGYPIYIGDNSNVQDNVVLHALETVDEDGKVLEENVVTVGDKKYAVYVGKNVSLAHQAQVHGPAAVGDNTFIGMQAFVFRSVVGKDCVLEPLAAAIGVTVPDGTYIPAGKVVTTQEEAAKLPKITPDHPFANTNAAVVKVNVALAKGYLALA

SEQ ID NO: 161 - protein, 316 aa, synthetic construct
  source 1..316: note = Library of modified or engineered enzymes
MIVLIIPLLFIVQSQTTTTTNTTTTTATTISYASQGSDWTSGVCSSSTSQSPINLEVSSGTCDNSMVLDIQFKKDAMQIVMERVQYTIQSKAAVSNLYATDINGNLYGYTATSFMFHSPSEHTIEGTRYDLEMQIVHDLKSEFSATITKAIVSILFEVSSTDQPFFTTYDFALVASASTNTTTTNTTNSTNTTTAASVTSTIASINFNDLLGSQLDANPAYYTYVGSLTIPDCDENVNWYILDSILPITQTQLDAFNTYFLSNSTFASGNGNNRAIQSTNDRTIKKGGVACEEQFVYFFSFFILYIFINYFIFKLL

SEQ ID NO: 162 - protein, 214 aa, synthetic construct
  source 1..214: note = Library of modified or engineered enzymes
MQEITVLEFSNITKNEVTATNPKPTTPVIDPTSYVDPQATVIGDVTIGKNCYIAASAVIRADEGKPIYIGDRSNVQDGVVLHALESVNDGGKIREDNVVVHGDEYYAVYIGKNVVLAHQAQVHGPAAVGDDSFVGMKSLVFKSIVGSNCVIEPDAAAIGVTVPDNKYIPAGTVVTTQEEADNLPEITPDYAYYNDVAAVVKVNVDLCKAYREKA

SEQ ID NO: 163 - protein, 214 aa, synthetic construct
  source 1..214: note = Library of modified or engineered enzymes
MQEITVLEFSNVTKNEVTSTNPKPVTPVIDPTSYVDPNATVIGDVTIGKNCYIAASAVIRADEGKPIVIGDRSNVQDGVVLHALESVDDGGKVREDNVVLEGDEYYAVYIGKNVVLAHQAQVHGPAAVGDDSFVGMKSLVFNSIVGSNCVIEPEAAAIGVTVPDGKYIPAGTVVTTQAEADKLPEITPDYAKSNAIAAVVKVNVALCEAYRNQS

SEQ ID NO: 164 - protein, 186 aa, synthetic construct
  source 1..186: note = Library of modified or engineered enzymes
MLKTNPRGDWPQVHASAFIEPTAILCGYVIVEENVFIGPYAVIRADETDADGRIAPIVIGAHSNIQDGVVIHSKSGASVTIGRHTSIAHRAIVHGPCKVGDGVFIGFNSVLFNCTIDDGCVVRYNAVVDGCHLPPGFYVRSTERIGPETDLAALPQVTADASDFSEDVARTNNALVLGYKHIQNEF

SEQ ID NO: 165 - protein, 240 aa, synthetic construct
  source 1..240: note = Library of modified or engineered enzymes
MKRNFLAISTVCGYDPEQWHMDYPIANGNRQSPINIITKDAKYDPNLKPLTLSYDPATAKEIVNVGHSFNVEFEDTDNKSVLKGGPLTGSYRLTQFHFHWGSVDGQGSEHTVDNVKYASELHLVHWNSVKFSSFAEAALKDNGLAVLGIFLKVGEHNPHLQKITDILNSIKTKGKQTTFTNFDPSCLLPASLDYWTYHGSLTVPPLLESVTWIVLKEPISVSSEQLAKFRSLLFTSEGET

SEQ ID NO: 166 - protein, 214 aa, synthetic construct
  source 1..214: note = Library of modified or engineered enzymes
MQEITVTRYENIRPSPVTPWNPTPKLPKIDPTAYVDPLAYVQGDVTIGKNVMISAHASIRSDEGYPIVIGDNSNVQDNVVLHALETVDANGNPLEANIVKVGDKDYAVYIGDNVSLAHQAQVHGPAAVGDNTFIGMQAFVFNSRVGKDCYLAPLAAAIGVTVPDGTYIPAGKVVTTQEEAAKLPKMTPDHPFYNTNAAVVKVNVALAKGYLALA

SEQ ID NO: 167 - protein, 214 aa, synthetic construct
  source 1..214: note = Library of modified or engineered enzymes
MQEITVLVFSNIRKNEVTPENPEPVTPVIDPTSYIDPKATVIGDVTIGKNCYIGASAVIRGDEGYPIVVGDESNVQDGVVLHALESVDEQGKVIEDNVVRKGDKLYAVYVGKNVVLAHQSQVHGPAMVGDDSFVGMNSLVFKSRVGSNCVIEPYAAAIGVTVPDGKYIPAGTVVTTQAEADKLPEVTDDYKFYTQVAAVVTVNVKLCEAYREQM

SEQ ID NO: 168 - protein, 310 aa, synthetic construct
  source 1..310: note = Library of modified or engineered enzymes
MGSSLILLPRVQTSRNGMKLFGILLSFGSVLGALAGNSWNYAGHGEYWPTSHAKSSTGAESYWDCDGIRQSPIDINSSMVQDVYFWNQLNLANYAASYAGKFKNNGHTLQFDLDDAETSGATLPTFSSPFMCTGCSYELQQFHFHWGSTAYQGSEHTKEGIAFPMELHLVHKKTSYSSVTASLSYNDGLAVIGIMFQLADTSDAGLTEIINAAVAIKNAADQHTHKEAKSIDMSTFLYQTGRSYYYYKGSLTTPPCTETVDWHLMEGAIRITEADLEKLRDLTYTDDAPLVDNYRLPMPLNNRIIKRVFN

SEQ ID NO: 169 - protein, 214 aa, synthetic construct
  source 1..214: note = Library of modified or engineered enzymes
MQEITVTNYNNIQPSPVTSWNPTPKLPDIHPTAYIHPKAVVQGDVTIGKDVMISANASIRSDEGYPIVIGDNSNVQDNVVLHALETVDANGNRIEENIVTVGDEEYAVYIGKDVSLAHQAQVHGPAAVGDNTFIGMQSFVFRSRVGKDCVLEPLAAAIGVTVPDGTYVPAGKVVTTQEEAAKLPKITPDHPFANTNAAVVKVNVELAKGYLALA

SEQ ID NO: 170 - protein, 340 aa, synthetic construct
  source 1..340: note = Library of modified or engineered enzymes
MGLLDSKVWFVTQVLAAVPLVYSGPYPRTTSSYYDPNGFFQFADKTHHRFYNFYQHVTEAARAEKLISNNEVWPSDKSISQMASGSFSYREEDDYGPSNWGALNATCEGMYQSPINLIANRSVIVQQKRALELKGSRNVPMAMVVENEGGAAAFFPEFRTNEQPRLRGGPLRGEYLFYQFHYHLGSEHTFDKKRYSAEMHLVFYNELYGSFKAARDQANGVAVIALTFDVLKSRRINSLNKWTRSLAEVVEAESEYSIPRQELFSVSDVLGDMEWPYFAYEGSLTTPPCSETVQWIVASERQLLTRSELKTMRMLKGRGGDWVQTARPTQALNFRRVFIY

SEQ ID NO: 171 - protein, 214 aa, synthetic construct
  source 1..214: note = Library of modified or engineered enzymes
MQEITVLEFSNVTKNEVTPWNPKPKTPVIDPTSYIDPQATVIGDVTIGKNCYIAASAVIRADEGKPIVIGDRSNVQDGVVLHALESVDDGGEIREENVVIEGDEEYAVYIGKNVSLAHQSQVHGPARVGDDSFVGMKSLVFNSDVGSNCVIEPFAAAIGVTVPDGKYIPAGTVVTTQEEAAKLPEVTEDYPFYTAIQEVVKVNVKLCEAYREQK

SEQ ID NO: 172 - protein, 214 aa, synthetic construct
  source 1..214: note = Library of modified or engineered enzymes
MQEITVFEFSNVRKNEVTAWNPKPSVPVIDPTAYIDPNATVIGDVTIGKDCYIAASAVIRADEGSPIFIGDRSNVQDGVVLHALESVNPDGMYREENVVLKGNNLYAVYVGRNVSLAHQAQVHGPAAVGDDSFVGMNSLVFNSKVGSNCVIEPNAAAIGVTVPDGKYIPAGTVVTTQEEADKLPEITPDYAFYTQVAAVVQVNVELCRAYRGKA

SEQ ID NO: 173 - protein, 214 aa, synthetic construct
  source 1..214: note = Library of modified or engineered enzymes
MQEITVTRYENIRPSPVTPWNPTPRLPKIHPTAYVDPLAYVQGDVTIGDNVMISPHASIRSDEGYPIVIGNNSNVQDNVVLHALETVDADGNEIEENIVTVGDEKYAVYIGDNVSLAHQAQVHGPAAVGDNTFIGMQAFVFKSRVGKNCVLEPLAAAIGVTVPDGTYIPAGKVVTTQEEADKLPKVTPDHPFYNTNAAVVKVNVALAKGYLALK

SEQ ID NO: 174 - protein, 214 aa, synthetic construct
  source 1..214: note = Library of modified or engineered enzymes
MQEITVTRYENIQPSPVTPWNPEPKLPEIDPTAYIHPAAVVQGDVTIGKNVMVSALASIRSDEGYPIVIGDNSNVQDNVVLHALETVDADGKRLTENIVTVGDEEYAVYVGDNVSLAHQAQVHGPAAVGDNTFIGMQAFVFRSTVGKNCVLEPLAAAIGVTVPDGKYVPAGKVVTTQEEAAKLPEVTPDHPFANTNAAVVKVNVALAKGYLALA

SEQ ID NO: 175 - protein, 214 aa, synthetic construct
  source 1..214: note = Library of modified or engineered enzymes
MQEITVTVFENIRESPVTPWNPTPRRPKIHPTAYVDPQAVVQGDVTIGANVMISANASIRSDEGYPIVIGDNSNVQDNVVLHALETVDENGKTLEENVVTVGDKKYAVYIGDNVSLAHQAQVHGPAAVGDNTFIGMQAFVFRSTVGKNCVLEPLAAAIGVTIPDGKYIPAGTVVTTQEEADKLPEVTPDHPFANTNAAVVKVNVALAKGYLAQA

SEQ ID NO: 176 - protein, 346 aa, synthetic construct
  source 1..346: note = Library of modified or engineered enzymes
MGSCQVHAGGSRHFSFVLTFGRAYNGVVMLVPTSYLLLLLVTLLTPTFCADWSYKLGDQSGPDHWEYECKKEYQSPVNIPKGETTSTVFPALSFWNYELQPATATIENNGHTVKLATEPHRPKETPLLSGGGLLHSYKFAQIHFHWGAEDFKGSEHLVGDTQYPMEMHLVHYKAVHDTIKDALAEGAYDSLAVIGIFFEVSEQRNPALDLLMPYLAKIKAAHSEAAATPFPISSFLWGGDMSSFYRYNGSLTTPTCNEIVQWSVMKVPVPVTVDQLEVFRQLMTKDYEPLVDNFRPPQALGGRDVLDVMTVEMLRKGSHSGCEILAGPAALLASLILILCCRTWDL

SEQ ID NO: 177 - protein, 214 aa, synthetic construct
  source 1..214: note = Library of modified or engineered enzymes
MQEITVTVYNNIRPSPVTPWNPEPRLPKIHPSAYIDPAAVVQGDVTIGENVMVSPNASIRSDEGYPIYIGNNSNVQDNVVLHALETVDENGKEIEENIVTVGDKKYAVAVGDNVSLAHQAQVHGPAIVGDNTFIGMQAFVFRSKVGKNCVLEPLAAAIGVTVPDGTYIPAGTVVTTQEEAAKLPKVTPDHPFANTNAAVVKVNVALAKGYLALK

SEQ ID NO: 178 - protein, 264 aa, synthetic construct
  source 1..264: note = Library of modified or engineered enzymes
MSTLVKAIRENPGCDWQYHFNPKISLIGRGQHQSPIDIHTKDALFDPSLKPLSVSYDPATARLVNNGHTIQVEFEDSTDKSVVEGGPLEGPYRLKQFHFHWGKKDGVGSEHTVDGKSFPSELHLVHWNAEKYASFGEAAAAPDGLAVLGVFLQVGEHHPSMNRLTDALYMVRFKGTKAQFSCFNPKCLLPASRHYWTYPGSLTTPPLSESVTWIVLREPISVSERQMEKFRSLLFTSEGEKEKRMVDNFRPLQPLMNRTVRSSF

SEQ ID NO: 179 - protein, 195 aa, synthetic construct
  source 1..195: note = Library of modified or engineered enzymes
MSEKGPAYWGEIKEEWAACSNGTMQSPIDLLNERVEVVPGLGELKRNYKPSNATLKNRGHDIALEWNGEAGSILINGTPYFLKQCHWHSPSEHSINGRRYDMELHLVHQSPENKIAVIGILYEIGPPDTFLSSLMDHIKAVTDTTEAERSVGVINPREIKRGSRKYYRYIGSLTVPPCTESVIWTVLAEIKKKIN

SEQ ID NO: 180 - protein, 290 aa, synthetic construct
  source 1..290: note = Library of modified or engineered enzymes
MLMRSFLIPTIVLSLILVSSNFAAEQDGADFDYNERGPEHWSQLDAKYKLCKDGERQSPINFITSIDLAIKLPNVNFIKQNTPNVKFPTISMKKEGHATKFLPQQLIGSSFDSVRYIFNQVHFHTPSEHRFDGIHTDLEAHFVFEDSVTKKYSVIGVLYEVDCAVGSSSFFDSIIKLYNQDPNAKDNVPVDINSEVFSHIKEVYKYFGSLTTPNCTEDVTWWVVNKPLLISTSQLVKLRKHIGFNSRPTQPRNGRKESNRLILFHVKRFLLNYQVTVFAVFGLVGGITIE

SEQ ID NO: 181 - protein, 263 aa, synthetic construct
  source 1..263: note = Library of modified or engineered enzymes
MKLYTVIRGNPACSFHEDQWFAPSVQPGGHQSPINIVTSQTKYDPNLKPLTISYDPATSLEILNNGHSFQVTFDDTQDKSVLRGGPLDGVYRLVQFHFHWGSSDEQGSEHTVDKKKYAAELHLVHWNAVKYETFAEAAQEPDGLAVLGIFLKVGEHNAELQKITDILDSIKHKGKQTRFTNFDPICLLPPCPDYWTYPGSLTTPPLSESVTWIVLKQPIEVSPSQLAKFRSLLFTSEGETACCMVDNYRPLQPLMNRTVRSSF

SEQ ID NO: 182 - protein, 214 aa, synthetic construct
  source 1..214: note = Library of modified or engineered enzymes
MQEITVSFFSNVSKNEVTSTNPKPVTPVIDPTSYVDPKATVIGDVHIGKNCYIAASAVIRADEGAPIYIGDRSNVQDGVVLHALESVADGGKVLEDNVVLEGDENYAVYVGKDVTLAHQAQVHGPAAVGDHSFVGMKALVFNAKVGKNCVIEPEAAAIGVTVPDNKYIPAGTVVTTQEEADKLPEITPDHENYTKVAEVVAVNVKLCEAYKSKA

SEQ ID NO: 183 - protein, 272 aa, synthetic construct
  source 1..272: note = Library of modified or engineered enzymes
MLSVPVSIAIATRAPDAVDASAPGEWGYADSSNGPARWSDILDADGKASYPACGCAACQQSPIDLVRTAAKGNVRVGSLADRLVAPAKPVTLAVSQKHGTPNYVATDQNNDAAVVAPDGVRYTFNSLHFHTPAENTVDGVANAMEMHMVHLSEAGDIAVLGVLFRHADADLPANAEVTKLLRKIDADGGKTKVAVDLGGLYDGGAGFWEWTGSLTTPPCSGNVRWLLQKEVRGVDARQAEAFKKHVGGFPGNARPTQPLNGRAVLSFDPTGV

SEQ ID NO: 184 - protein, 214 aa, synthetic construct
  source 1..214: note = Library of modified or engineered enzymes
MQEITVFEFSNITKNEVTSTNPKPVTPVIDPTSYIDPNATVIGDVTIGKNVMIWPTAVIRADEGKPIVIGDNSNVQDGVVLHALESVNDGGKIREDNVVIEGNELYAVYIGKNVTLAHQSQVHGPARVGDDSFVGMKSLVFKSDVGSNCVIEGNAAAIGVTVPDGKYIPPGTVVTTQAEAEKLPEITEDYPFSDANQAVVEVNVKLCKAYRGLQ

SEQ ID NO: 185 - protein, 227 aa, synthetic construct
  source 1..227: note = Library of modified or engineered enzymes
MLAAGAHWEYSGEAGPANWAKLTPEFGACSGKNQSPINLTGFIEAELEPLAFAYQASATQVLNNGHTVQVNYAEGSTLTLDGQTFTLKQFHFHSPSENRIEGKSFPLEAHFVHASEQGALAVVALMFQEGAANPELEKAWRVMPAHADQPVALPRPLDVQALLPKDHAYYRFNGSLTTPPCSEGVRWLVLKQPVEASKAQIEKFQKIMGYPNNRPVQPVNARTVLSS

SEQ ID NO: 186 - protein, 214 aa, synthetic construct
  source 1..214: note = Library of modified or engineered enzymes
MQEITVLEYSNVTKNEVTSTNPKPTTPVIDPTSYVDPNATVTGDVTIGKNVLIGPNAVIRADEGAPIVIGDNSSVQDGVVLHALESVDDDGEVIEDNVVLYGNKDYAVYVGKNVVLAHQAQVHGPAAVGDDSFVGMKALVFKSIVGSNCVIEPDAAAIGVTVPDGKYIPAGTVVTSQEEAANLPEITPDHEDYTTQEAVVKVNVGLCEAYRNQA

SEQ ID NO: 187 - protein, 302 aa, synthetic construct
  source 1..302: note = Library of modified or engineered enzymes
MMSVATALLLLSAVGTLAADWRYPTPGPDGSVGSPENWGGSCDHGRRQSPIDIAYAASVRGSYPEFIFDSYDSLPDSAYIVNNGHTVQINLDSSASSSVYGGGFRSKYVLEQLHFHWSSEHTIEDRRYALEMHLVHRQSRYASVEQASSHKAGIAVLAVLFHVDEHPNEAIQLILNSTSPIKAKVDDRQPLRGSLHLNDLLPKDRTVYFRYEGSLTTPVCAESVVWTVFPESLPISLGQVQDFMTIHDADNRTLVVNYRPVQPLNTRVLVLVSDTEVEASGARRIASGMFAAVLLSLAISLF

SEQ ID NO: 188 - protein, 242 aa, synthetic construct
  source 1..242: note = Library of modified or engineered enzymes
MKILVTFASCGYEPRNDHWQEPWSYEGISGPDHWGELNPEYSLCSTGKEQSPIDIDHTIKAQLPALKFDYKSEPLKYVINNGYTIRVNYHDAPGSGNFLIVDDTRYQLTQFHFHRPSEEYIHGKPYDMELHLMHQSSDGKVAGVTVFIKTGRANSTTQKIWEHMPKTEGQQEVAGVEINPADMLPHDTGYYVYMGSVTAPPCTEGVNWFVLKTPVEISADQIEAFAKLYPHDVRPLQPLNGR

SEQ ID NO: 189 - protein, 255 aa, synthetic construct
  source 1..255: note = Library of modified or engineered enzymes
MKNSLFATIVGYCPEWRDHQPVAPLGQRQSPIDIVPADAQYDSSLKPLKLQYDPSTCLDILNNGHSFQVTFVDDTDSSTLTDGPISGVYRLKQFHFHWGASDDKGSEHTVNGAKYAAELHLVHWNAVKYASFAEAALEPNGLAVLGVFLKVGEHNPALQKLTDILPSIKHKDTQASFGKFDPSCLMPTCPDYWTYAGSLTTPPLSESVTWIVLKEPIEVSEEQLGKFRSLLFTSEGEKEKRMVDNFRPLQPLMNR

SEQ ID NO: 190 - protein, 214 aa, synthetic construct
  source 1..214: note = Library of modified or engineered enzymes
MQEITVLTYSNVTKNEVTATNPKPTTPVIDPTSYVDPNATVTGDVTIGKNVLIAANAKIRADEGKPIVIGDRSTVQDGVVLHALESVDDDGKVIEENVVLEGDEYYAVYVGKNVVLAHNAQVHGPAAVGDDSFVGMNSLVFNSVVGSNCVIEPNAAAIGVTVPDGKYIPAGTVVTTQEEADKLPEVTEDYEFYTQIAEVVKVNVNLCEAYRKQA

SEQ ID NO: 191 - protein, 277 aa, synthetic construct
  source 1..277: note = Library of modified or engineered enzymes
MKFIATTTALVAACALAISTVSAVSAVSEAGVKGAPWGYKPDDTTQASPVQWADHYPDCNGTHQSPIDLVTADVKQQTKAQNTLRFRGDCASFNLTQSAEGYKAEVVGGSCQVRGNKARYDLLQFHVHAPSEHTLNGEPLDGEVHFVHSNKDGSALLVVGLFMEIDPSGNTDPWLETLIDGIDDVSPTKEVMLDLTSYSALVKKTVRGGSLFNYPGSLTTPGCSEIVDWWVVEKPMKISAKDLTRIRENQGEIDLNYKSESARPVQPLNDRIVKSFQ

SEQ ID NO: 192 - protein, 279 aa, synthetic construct
  source 1..279: note = Library of modified or engineered enzymes
MINSRPFLIFAVYDHGTAICGPADWKNVSAHCVENTQSPINIKTDKIFMHMFPYFDGFHFIVDNVVGSVSGVLVNNGHAPTLVIDQFETPAILTGGPWANKVYRLNQIHFHFGCDASKGSEHTVDGRVYSGEIHFVTYNTKYFDFHAAADKPDGLSVVAVFLLDNGDKSNWKQLTDEMKKIIKADSFTKVPMYYINLYKMVPELRALFRAPFYTYKGSLTTPPCYQSVKWVVLQNPVSTSRELMTAMRSLKNHEGHSLCNNFRPTQPLNGRILAKHLKY

SEQ ID NO: 193 - protein, 251 aa, synthetic construct
  source 1..251: note = Library of modified or engineered enzymes
MSLKRIFGVATPEDQHNYCWCYEEENGPSEWKEHFPIANGPRQSPIDIKTSETKYDSSLKPLSVSYDPSTAREILNVGHSFHVTFEDSENKSVLKDGPITGVYRLKQFHFHWGAADDKGSEHTVDGAKYAAELHLVHWNAVKYKSFEEAALEENGLAVIGVFLKLGKHHEELQKLVDTLPAIKHKDALVTFGSFDPSCLMPTCPDYWTYPGSLTTPPLSESVTWIIKKQPVEVDHDQLEQFRTLLFTSEGE

SEQ ID NO: 194 - protein, 214 aa, synthetic construct
  source 1..214: note = Library of modified or engineered enzymes
MQEITVTRYENIQPSPVTSWNPEPKLPEIHPTAYIHPAAVVQGDVTIGKNVLVMANAVIRADEGYPIYIGDNSSVQDNVVLHALETVDKNGNTIEENVVTVGDEKYAVYVGDNVVLAHNAQVHGPAAVGDNTFVGMNALVFRSRVGKNCVLEPLAAAIGVTVPDNKYVPAGTVVTTQEEADKLPEITPDHPFANLNKRVVEVNVELAKGYLALS

SEQ ID NO: 195 - protein, 287 aa, synthetic construct
  source 1..287: note = Library of modified or engineered enzymes
MKLIRSAVTGFCPEWNDHYQYDGISGPAYWGLINPDWTLCNSGKRQSPIDIDPNKLLFDPNLKSLHIDKHKVSGVLENTGQSLVFRVDKDTKQHVNISGGPLAYKYQFHEIFIHYGLEDSNGSEHSVDGYSFPAEIQLYGYNSDLYSNMSEAQEKSQGLVGISLLVQIGDMSNPELRVLTTALEKVKYKGQTTRIRKFNVRGLLPDTQHYMTYEGSTTHPGCWETTVWIILNKPIYITKQELYALRRLMQGSKEHPKAPLGNNARPTQPLHHRTVRTNIDFKHKKDG

SEQ ID NO: 196 - protein, 292 aa, synthetic construct
  source 1..292: note = Library of modified or engineered enzymes
MSQDEQKWSYAQDYLWKSPSCTGSKQSPINIDTSQIQRCGVLCDLKLYLKSEKPSVEFTSQNDVILSFVNSQSSITFNNRYFNLRSIRVHVPSLHTIDNSKTDMEVVCLFDSGNNNETSSNDSLQNVAKGVQLCFMMNQSNNEYGNIEQFFNQFIHKIPTVQDELPIEVNVSSSWSPELLIPNKQNFYYYEGSLAYPPCSEMYINIVYEEIGNIGVSNFRILKKYIRNNTRALKPKNNRVVYYSVDETNSASIQSNSVDKISDDRFLQCERRNNVVKTKKQVIASETIPEDN

SEQ ID NO: 197 - protein, 305 aa, synthetic construct
  source 1..305: note = Library of modified or engineered enzymes
MWLFSFLFYVAVHKNSGKNLHIKVLRAPKMIALLSHTQGAQPSLPDRTYMPIAKKLPYTKHHRLAAVILLIGMFVYHSALSEEQPWHFTTPAKADDCSQQSPEGAPCGCGELQSPINIKHSLRAHLPELVTRYSPGPATVKHIGHTLEVRTEMKGHLTLGAKSYDFVQLHFHLPGVDLIKGRSYPLVAHLVHRSSTGEVAVVAIVFKRGQENANLAQLLAVMPRHKGDAFVLGKFDIAQLLPQQRKYYAYKESMSAQPGIEGINWHILKTPMEVSDAQLHAFQLILPAHRRPAHPARNRSVRVGG

SEQ ID NO: 198 - protein, 214 aa, synthetic construct
  source 1..214: note = Library of modified or engineered enzymes
MQEITVLTFSNITKNEVTSTNPKPKTPIIDPTSYVHPLATVIGDVTIGKNCMISASASIRSDEGRPIYIGDNSNVQDGVVLHALESVDDGGKIIEENVVLEGNEYYAVYIGKNVSLAHQSQVHGPARVGDDSFIGMQSFVFNSIVGSNCVIEPNAAAIGVTVPDNKYIPAGTVVTTQEEADNLPEITPKHAAFTTQEAVVKVNVNLCRAYRNLA

SEQ ID NO: 199 - protein, 271 aa, synthetic construct
  source 1..271: note = Library of modified or engineered enzymes
MTTATDHIDYGYGPTNGPHTWCITCRTAAGTHQSPINIITHNCHFDPTLTPFKVFVSHHGHQILSRKQHNFQVSFKTDRPTYVEGGPLKNKYNLLQLHFHWGCYDEWGSEHHIDGHSYAGELHLVFMNEKYANINQAFNDPEGLCVIGIFLKPSVEGCSAMAPMMAAMKSSKPGCETSVKGEIDINGLIPNNSRYFTYEGSLTTPPCVECVRWIVCAKPLRLSKDQLAALRSMHCCETCYTNENFRPPVPVGDRVVVCSFPQSIRPQKCDT

SEQ ID NO: 200 - protein, 214 aa, synthetic construct
  source 1..214: note = Library of modified or engineered enzymes
MQEITVTTFNNIQPSPVTPWNPEPRLPEIHPTAYVHPAAVVQGDVTIGANVLVMANAVIRADEGYPIYIGDNSSVQDNVVLHALETVDADGRRIEENVVTVGDKEYAVYIGDNVVLAHNAQVHGPAAVGDNTFVGMNALVFNSRIGANCVLEPNAAAIGVTVPDGRYIPAGTVVTTQAAAAALPAVTPDHPFATLNARVVAVNNALAAGYLALA

SEQ ID NO: 201 - protein, 247 aa, synthetic construct
  source 1..247: note = Library of modified or engineered enzymes
MVAILGVLMSSCEEEEVTVERHSPHWDYESTMWQNIGYTDCGGIVQTPINIETANTIKSADLSDVTFNYNAFDIKIVDNGHTVQVNRDATKTNNMVIDGVTYDFLQFHYHTHSEHEIDGATDEMEIHLVHQDPITKNLAVVSVMLNANGTTPNDFIESYLENFPSTEENEVATTTSIDLDDLLPSNHNYYTYTGSLTTPPCSQGLKWIVLKDKVDVSVEQMHKFEETHGVNARPIQPLNGRLVLEKI

SEQ ID NO: 202 - protein, 214 aa, synthetic construct
  source 1..214: note = Library of modified or engineered enzymes
MQEITVLDFSNVTKNEVTSWNPKPTVPVIDPTSYVDPNATVIGDVTIGANCYIGPSASIRADEGRPIVIGDRSNVQDGVVLHALESVDDGGKIREDNVVVHGDEYYAVYIGKNVVLAHQAQVHGPAAVGDDSFVGMKSLVFKSDVGSNCVIEPFAAAIGVTVPDGKYIPAGTVVTTQAEAAKLPEITPDHANYTQQEAVVKVNVKLCRGYRRLQ

SEQ ID NO: 203 - protein, 214 aa, synthetic construct
  source 1..214: note = Library of modified or engineered enzymes
MQEITVLIYSNVEKNEVTSTNPKPKTPVIDPTSYVDPQATVIGDVTIGKNCYIAASAVIRADEGKPIVIGDNSNIQDGVVLHALESVDDGGKVLEDNVVIKGNKLYAVYIGKNVALAHQSQVHGPARVGDDSFVGMNSLVFNSIVGSNCVIEPFAAAIGVTVPDNKYIPAGTVVTTQAEADQLPEVTDDHPFYTEVAAVVKVNVALCQAHKGLS

SEQ ID NO: 204 - protein, 266 aa, synthetic construct
  source 1..266: note = Library of modified or engineered enzymes
MLTPARSIFCVGWKEQDHNYSLSPTLRPLVDGDRQSPINIVPGNAVYDPRLKPLTLSYDPATSLEILNNGHSFQVTFDDSQDKSVLKGGPLDGVYRLKQFHFHWGASDDHGSEHTVDGVKYPSELHLVHWNAKYGDFGEAASKPDGLAVVGVFLKIGHEKPHMQKVLDALDAIKTKGKQTTFTNFDPSTLLPGCLDYWTYDGSLTTPPLLESVTWIVLKEPISVSPAQMAKFRSLLFTSEGETACCMVDNYRPPQPLKGRQVRASF

SEQ ID NO: 205 - protein, 257 aa, synthetic construct
  source 1..257: note = Library of modified or engineered enzymes
MSLKNIFTAVCGPEQWHDYFRKANGNFQSPINIDTKETKYDSSLKPLTLSYDPATAKEILNNGHSFQVTFDDTDNKSVLKGGPLTGSYRLRQFHFHWGATDEKGSEHTVDGVKYASELHLVHWNAVKYASFAEAASKPDGLAVLGVFLKIGKHHEELQKITDTLNSIKTKGKQTTFTNFDPSCLLPSCLDYWTYFGSLTTPPLYESVTWIVCKQPISVSSEQLAQFRSLLSNAEGEKACCMVDNYRPPQPLKGRKVR

SEQ ID NO: 206 - protein, 214 aa, synthetic construct
  source 1..214: note = Library of modified or engineered enzymes
MQEITVTNYNNIRQSPVTSWNPTPKLPKIHPTAYIDPAAVVTGDVTIGKNVMVSANASIRSDEGYPIYIGDNSNVQDNVVLHALETVDANGKEIEENIVVVGDKKYAVYIGDNVSLAHQAQVHGPAAVGDNTFIGMQAFVFRSVVGKNCVLAPLAAAIGVTVPDNTYIPAGKVVTTQEEAAKLPKITPDHPFANTNKAVVAVNVELAKGYLALS

SEQ ID NO: 207 - protein, 267 aa, synthetic construct
  source 1..267: note = Library of modified or engineered enzymes
MRSIKLLCLPALLATTIANAGASLDQWDYSSHGERYWRSHFPACQGMQQSPINISTKRALKHPKAFPSLKPGPVLDFSPPAAPVHIENNGHSIQFEYRGNYSLLHRGESYQLKQFHFHHASETTIDGKHSPLEVHFVHKSQQGHTLVIAVLLDSGRAENILISSFKAADSSPQNGVNKSSFNPKKLLPKEKDFYYFEGSLTTPPCTEGVHWAVMKHKGLVSEQDVRYFAKFDYPANFRHTQPINGRSTYYFSDIDRSEDDIKNSSGR

SEQ ID NO: 208 - protein, 214 aa, synthetic construct
  source 1..214: note = Library of modified or engineered enzymes
MQEITVSNFSNVTKNEVTPYNPKPVTPVIDPTSYIDPNATVIGDVTIGANCMVSANASIRSDEGKPIVIGDRSNVQDGVVLHALESVNDEGKILEENVVTVGDRNYAVYVGKNVSLAHQSQVHGPAAVGDDSFIGMQSFVFRSKVGSNCVIEPQAAAIGVTIPDGKYIPAGTVVTTQAEADKLPEITPDYAQSNTQAAVVTVNVKLCEAYRNKQ

SEQ ID NO: 209 - protein, 214 aa, synthetic construct
  source 1..214: note = Library of modified or engineered enzymes
MQEITVTNYTNIQPSPVTPWNPEPKLPEIHPTAYIHPAAVVQGDVKIGENVLVMANAVIRADEGYPIYIGNNSSVQDNVVLHALETVDENGNRIEENIVKVGDKEYAVYIGDNVVIAHNAQVHGPAAVGDNTFVGMNSLVFRSRIGKNCVLEPLAAAIGVEVPDGKYIPAGTVVTTQEEAAKLPEVTPDHPFANLNERVVKVNIALAKGYLALA

SEQ ID NO: 210 - protein, 277 aa, synthetic construct
  source 1..277: note = Library of modified or engineered enzymes
MRKTLAVSIFCGWNYDPEHQRWDYDDQENGPHRWPKLYPECGGNAQSPIDIKTKETKYDPNLKPLTLVGYDKNGLEFSMTNNGHTVQISLPSSMYLKDSDGTVYIAKQMHFHWGGDSSEISGSEHTIDGMRYLIEIHVVHYNSKYKSYDVAQDAPDGLAVLAAFVEVKDYAENTYYSNFISHLENIKYPGQSTVLRGLDIQDMLPKNLHHYYSYLGSLTTPPCTENVHWFVLADSVKLSKTQVWKLENSLLDHQNKTIHNDYRKTQPLNHRVVEANF

SEQ ID NO: 211 - protein, 214 aa, synthetic construct
  source 1..214: note = Library of modified or engineered enzymes
MQEITVMEFSNVTKNAVTPTNPKPTTPVIDPTSYVDPEATVIGDVTIGENCMISAFASIRSDEGKPIHIGNRSNVQDGVVLHALESVNPTGMVNEENVVVAGDELYAVYVGKNVSLAHQAQVHGPAMVGDDSFIGMQSFVFKSIVGSNCVIEPNAAAIGVTVPDNKYIPAGTVVTTQAEADKLPDITPDYAYYTTVAAVVSVNVNLCKAYREQA

SEQ ID NO: 212 - protein, 254 aa, synthetic construct
  source 1..254: note = Library of modified or engineered enzymes
MKILTFASVCYGPENWHRDFQAAKGKRQSPIDIVPASAKYDSSLKPLTFTYEAGTSRCIVNNGHSFNVEFDDSQDKSVLSGGPLTDKYRLTQFHFHWGKTDDEGSEHTVDGHSYPAELHLVHWNADKFASFGEAASKPDGLAVVGVFLKVGDEHPGLKKVTDALYSVKFKGTKAEFKNFNPKCLLPASLDYWTYDGSLTTPPLSECVTWIVLKEPISVSSGQMGKFRSLLFTSEGETECCMVDNYRPPQPLKGR

SEQ ID NO: 213 - protein, 277 aa, synthetic construct
  source 1..277: note = Library of modified or engineered enzymes
MQRKLPSVAIFCTGYENHDWGYEDHNGPEHWHELFPIANGDNQSPIELHTKEVKYDSSLQPWSASYDPGSAKTILNNGKTCRVVFDDTYDRSMLRGGPLTGPYRLRQFHLHWGSSDDHGSEHTVDGVKYAAELHLVHWNAVKFESFEAAALEENGLAVIGVFLKIGRHNPELQKLVDVLPAIKHKDTLVEFGSFDPSCLMPTCPDYWTYPGSLTTPPLSESVTWIILKQPIEVDHDQLEQFRTLLFTSEGEKEKRMVDNFRPLQPLMNRTVRSSFRH

SEQ ID NO: 214 - protein, 191 aa, synthetic construct
  source 1..191: note = Library of modified or engineered enzymes
MLNSPSAANDERPEVEDGAWIHPSAALVGNVSIGSRAYVGPQASIRADEPGPDGSVAPVVIESEANVQDGAVLHALGGTSVVVRSRTSVAHGAVVHGPCQVGPGCFIGFNTVVYDAELGEQVVVMHGAVVENVEIPDGLIVPSRAAVCCQEDVRALDEASESALAFADEVSRTNVHLAEVKNSEQQTGYYE

SEQ ID NO: 215 - protein, 214 aa, synthetic construct
  source 1..214: note = Library of modified or engineered enzymes
MQEITVLEFSNVRKNEVTPTNPKPTTPVIDPTSYVDPNATVIGDVTIGANVLIWPTAVIRADEGRPIVIGDRSNVQDGVVLHALESVDDGGEIREDNVVRVGDENYAVYVGKNVSLAHQSQVHGPAAVGDDSFVGMKSLVFKSKVGSNCVIEPDAAAIGVTVPDGKYIPAGTVVTTQEEAAKLPEVTPDYAYYTTVEEVVTVNVALCEAYREEA

SEQ ID NO: 216 - protein, 214 aa, synthetic construct
  source 1..214: note = Library of modified or engineered enzymes
MQEITVTRYENIRESPVTPWNPEPRRPEIHPTAYIDPKAVVQGDVTIGANVLVMANAVIRADEGYPIVIGDNSSVQDNVVLHALETVDENGNPIKENIVKVGDKDYAVYIGDNVVIAHNAQVHGPAAVGDNTFIGMNALVFRSVVGKNCVLEPLAAAIGVTVPDGRYIPAGTVVTTQEEADKLPKVTPDHPFANLNARVVKVNVALAKGYLAQA

SEQ ID NO: 217 - protein, 214 aa, synthetic construct
  source 1..214: note = Library of modified or engineered enzymes
MQEITVTVYNNIQPSPVTPWNPEPKLPKIDPTAYIHPKAVVIGDVTIGKNVMISANASIRSDEGYPIYIGDNSNVQDNVVLHALETVDENGNRIEENIVVVGDKEYAVYVGDNVSLAHQAQVHGPAAVGDNTFIGMQAFVFRSVVGKNCYLAPLAAAIGVTVPDGKYIPAGKVVTTQEEAAKLPEMTPDHPFYKTNEAVVKVNIALAKGYLALK

SEQ ID NO: 218 - protein, 214 aa, synthetic construct
  source 1..214: note = Library of modified or engineered enzymes
MQEITVTRFENIQPSPVTPWNPEPRRPEIHPTAYIHPLAYVQGDVTIGENVLVAAHAVIRADEGYPIVVGNNSSIQDNVVLHALETVDENGNRIEENIVTVGDEEYAVYVGDNVVIAHNAQVHGPAAVGDNTFVGMNSLVFRSRVGKNCVLEPLAAAIGVTVPDGTYVPAGTVVTTQEEAAKLPKITPDHPFANLNARVVKVNVALAKGYLALS

SEQ ID NO: 219 - protein, 281 aa, synthetic construct
  source 1..281: note = Library of modified or engineered enzymes
MKIGTVLSIFLGMAHAAVDDHSSPWNYNTWGSDWGSLTAIAGNECGNRNQSPIDLPSSVDSSQIYASKSDNFNKMYTDQTNAKIYWDGHTSKITIVNPGEDLQKFSSSFAKDYLQGPERFSGVQFHFHHGSEHTIDGERHDLEMHTVHVPDEGAKGGIKYAAMGIMFSVDKHTANAEEWEVKIIDDFFENLQWSETTTDPIVDLVSYGKVMMMVDTDNRWVYKGSVTTPPCATLVYWNVVRKIYPLKQKYLDQFKNQLKRGSLTGNYREIQAYDDHDLHII

SEQ ID NO: 220 - protein, 324 aa, synthetic construct
  source 1..324: note = Library of modified or engineered enzymes
MITFLVSFLAALVCEFVHSDNLPVAWCYNNPACNFPNWPNIAPQYCNGSSQSPIDIVTAQVQGNPNLTQFILTGFDANTTFTSITNSGTSVVVSLDEDIMSVQGGDLPGLYVSVQFHLHWGSSSSLPGSEHTVDGKQYAMELHIVNLHSTYDGNVSAALAANDSSALAVLGFFIEGTDEADKTNSWDIFTSFLSNIPNSGNTYTDIMDQITMNSLLEGVNKTKYYRYQGSLTTPPCNEDVIWTVFKEPIKVNNNLINRFCTKVFAKTAKASDLNVNNFRGVQPLNGRVVTSQVEQTGSSAAPSLVPTSISSLSLILLLTSLSCL

SEQ ID NO: 221 - protein, 214 aa, synthetic construct
  source 1..214: note = Library of modified or engineered enzymes
MQEITVYDYSNVTKNEVTSTNPKPTTPVIDPTSYVDPKATVTGDVTIGKNVLIGPFAVIRADEGAPIVIGDRSNVQDGVVLHALESVDDGGKVREDNVVRHGDELYAVYVGRNVSLAHQAQVHGPARVGDDSFVGMKSLVFRAKVGKNCVIEPGAAAIGVTVPDGKYIPAGTVVTTQAEAAKLPEITPDHPNYSKVDEVVAVNVGLCEAYRERA

SEQ ID NO: 222 - protein, 208 aa, synthetic construct
  source 1..208: note = Library of modified or engineered enzymes
MRSALVIPFYKGHQEDWNTCKNGGMQSPIDLLHERVEVVSHLGRLQKSYKPSNATLKNRGHDMMLRWGDAGGYLEINGTEYVLQQCHWHSPSEHTINGRRFDMELHMVHQSRDNKIAVIGIMYKIGRPDSFLSKLMDHISAIADTTEEEKAVGVIDPRNIKIGSRKYYRYIGSLTVPPCTQNVVWTIVRKVRTVTREQVRLLRVAVHD

SEQ ID NO: 223 - protein, 291 aa, synthetic construct
  source 1..291: note = Library of modified or engineered enzymes
MRPPPQRQGKTHREGKEMTTAAWKAIFAMVLASVLLVDADDAHVKFGYSGSIGPEKWASLSPGYQMCSKGERQSPVNIDKSKLAYNPGLAALERNYVPANATLVNKGYQIALLFDKNVGTLVVDGKNYSLKSVHWHSPSEHTINGKRFAVELHMVHMSDNGRIAVVAILYQIGRRDPFVVQIERKLKELAEEACKGDEEAYVPVGVVHTRSLKRHSSKYFRYSGSLTTPPCTENVIWSILGKVREMAEEQLAALQAPLSQENRNNARPTQPLNYRAVQLYHESRKHDEYSR

SEQ ID NO: 224 - protein, 214 aa, synthetic construct
  source 1..214: note = Library of modified or engineered enzymes
MQEITVLEFSNVTKNEVTPTNPKPTTPVIDPTSYVDPNATVTGDVTIGKNVMISDSASIRSDEGKPIVIGDRSNVQDGVVLHALESVDDDGEVIEDNVVIYGDENYAVYVGENVSLAHQAQVHGPAAVGDDSFIGMQAFVFKSTVGSNCVIEPEAAAIGVTVPDGKYIPAGTVVTTQEEAAKLPEVTPDYPFYTTQAAVVTVNVALCEAYRAER

SEQ ID NO: 225 - protein, 283 aa, synthetic construct
  source 1..283: note = Library of modified or engineered enzymes
MKKPKLFNLLGTFAATFAYEYNPGNSDYRPENWAQMDSPNNIQCDWANQSPIDLQTQFVLIQSRANSLSITNLRTMPENVVLTNVGHGAEISFEFANNDNMVVTGGPLNDQFIVAQAQWHWGTADCAGSEHMLNSQRYSAEVHIVTYNSKYASLEDAADKYDGLAVLGFLYEVDEAANSDFPQSVQTSLGGITFGCDSTTVSPFPLIDLFRTEFFDYIAYSGSLTTPPCYQTVQWMVSTKPLKIWSSDLDALRSINDVNGSPLLRNFRPCQNSYSRALNGYYL

SEQ ID NO: 226 - protein, 214 aa, synthetic construct
  source 1..214: note = Library of modified or engineered enzymes
MQEITVLDFSNITKNEVTSWNPKPTTPVIDPTSYVDPNATVIGDVTIGENCLIGASAVIRADEGHPIVIGDRSNVQDGVVLHALESVDDEGEYIEDNVVVKGDEEYAVYIGKNVSLAHQSQVHGPARVGDDSFVGMKSLVFKSDVGSNCVIEPFAAAIGVTVPDGKYIPAGTVVTSQEEAAKLPEVTDDYPFSTANEAVVKVNVALCEAYREQK

SEQ ID NO: 227 - protein, 214 aa, synthetic construct
  source 1..214: note = Library of modified or engineered enzymes
MQEITVTRYENIRPSPVTPWNPEPKLPKIHPTAYVDPAAVVQGDVTIGANVLIMANAVIRADEGYPIVIGDNSSVQDNVVLHALETRDENGNLLEENVVKVGDELYAVYVGDNVVLAHNAQVHGPAAVGDNTFVGMNALVFRSVVGKNCVLEPLAAAIGVTVPDGTYIPAGKVVTTQEEADKLPKITPDHPHYNLNERVVKVNVALAKGYLAQA

SEQ ID NO: 228 - protein, 214 aa, synthetic construct
  source 1..214: note = Library of modified or engineered enzymes
MQEITVTVFNNIRPSPVTPWNPEPKLPKIDPTAYIDPAAVVQGDVTIGKNVMVSANASIRSDEGYPIYIGDNSNVQDNVVLHALETVDENGKVLEENVVTVGDKKYAVYIGDNVSLAHQAQVHGPAAVGDNTFIGMQAFVFKSVVGKDCVLEPLAAAIGVTVPDGTYIPAGKVVTTQEEAAKLPKITPDHPFANTNAAVVKVNVALAKGYLALK

SEQ ID NO: 229 - protein, 229 aa, synthetic construct
  source 1..229: note = Library of modified or engineered enzymes
MKNSWRITAVLFGCYHQPEDYFSYDGISGPAYWGEINPEWSLCNQGKMQSPIDLLNERVEVVSKLERIKKNYKPSNATLKNRGHDMMLKWESGAGSIHINGTEYVLKQCHWHSPSEHTINGRRYDMELHMVHQSADNKTAVIGVTYKLGRPDSFLSSIMKHIKAISDTTEAEKAVGVIDPRHIKFGSRKYYRYMGSLTVPPCTEGVVWTIVKKVRTVSREQLRLLREAV

SEQ ID NO: 230 - protein, 320 aa, synthetic construct
  source 1..320: note = Library of modified or engineered enzymes
MELKVLSAHLFSWILVGPLFVVHIKAAEWSYADTSKWPKDYPSCSGYYQSPIDLTYKDSVYAPQLGQITITNLSKVEQTTYKVTNNGHTVEVSFNEKQWKISLGSDEDPYYPIQMHFHWGGPTREGSEHLIGDLRHAMETHIVCYNGRLYKSKEEATSSPNGLAVVGILHEEDKLAQTEQTEFGKMGEFETALASITTTKESKNIAAFDLAGLLGQVDTTQYFRYQGSLTTPPCTQNVMWTVFTTFVPVTPAQLELLRGLRTSSSTPLQDNYRPVQPLNDPHSPLPRTVYRTISAANRFTHSWWSFVMLSFLACCSHGIL

SEQ ID NO: 231 - protein, 214 aa, synthetic construct
  source 1..214: note = Library of modified or engineered enzymes
MQEITVTEFENIQPSPVTSWNPTPKKPVIHPTAYVHPAAVVQGDVTIGKNVMISPLASIRSDEGYPIYIGDNSNVQDNVVLHALETVDANGNTIEENVVTVGDKKYAVYIGDNVSLAHQAQVHGPAAVGDNTFIGMQAFVFRSIVGKNCVLEPLAAAIGVTIPDGTYIPAGTVVTTQEEADKLPKITPDHPFANTNKAVVKVNVELAKGYLALS

SEQ ID NO: 232 - protein, 256 aa, synthetic construct
  source 1..256: note = Library of modified or engineered enzymes
MTLSLFAAAAFADVQECPPRYSYCGYSGPEQWKNIVFKDKRNECNGTTQSPINLGTPTPTSGPTIHVEYVGNVAGNATIRNTGHDIEVTPMRGNNKIKVGSRVYTLLQLHFHVPNEHHVPRIGKAVAEMHILHQLDGGTDYAVIGVMLTIGTPTDSALAPVFENLPKEACAPPKPLEINFKKLLPEELTGYYTYVGSLTTPPCTEKEKTVTWYVLDAPREIPASDLLKLGALGKNARPIQTNPLTVTYVSPTPTPK

SEQ ID NO: 233 - protein, 214 aa, synthetic construct
  source 1..214: note = Library of modified or engineered enzymes
MQEITVAEFSNVVKNEVTPTNPKPTTPEIDPTSYVDPNATVEGDVTIGANVLIHAFAVIRADEGRPIVIGDRSNVQDGVVLHALESVDDGGEIREDNVVLHGDDLYAVYVGKNVHLAHQAQVHGPARVGDDSFVGMKSLVFKSDVGSNCVIEPFSAAIGVTIPDGKYIPAGTVVTTQEEADKLPEVTEDYAFSGQNEAVVTVNVDLNEAYRQQR

SEQ ID NO: 234 - protein, 214 aa, synthetic construct
  source 1..214: note = Library of modified or engineered enzymes
MQEITVTNFNNIRPSPVTPWNPEPKLPKIDPTAYVDPLATVTGDVTIGKNVMISANASIRSDEGYPIYIGDNSNVQDNVVLHALETVDENGNRIEENVVTVGDKEYAVYVGDNVSLAHQAQVHGPAAVGDNTFIGMQAFVFRSNVGKNCVLEPLAAAIGVTVPDNKYIPAGKVVTTQEEAAKLPEVTPDHPFYKTNAAVVKVNVALAKGYLALS

SEQ ID NO: 235 - protein, 275 aa, synthetic construct
  source 1..275: note = Library of modified or engineered enzymes
MLATFSRPNQGHVEIDYCKWTKYVSISGSSTWKDQFPIANGNRQSPIDIKTSETKYDSSLKPLSVSYDPSTSLEILNNGHSFQVTFADDSDSSTLKDGPITGVYRLKQFHFHWGASDDHGSEHTVDGVKYPAELHLVHWNTKYGDFGEAASKPDGLAVVGVFLKIGREKPEFQLVLDALESIKTKGKQASFTNFDPSTLLPGCLDYWTYDGSLTTPPLLESVTWIVLKEPISVSPAQMAKFRSLLFTSEGETACCMVDNYRPPQPLKGRQVRASF

SEQ ID NO: 236 - protein, 186 aa, synthetic construct
  source 1..186: note = Library of modified or engineered enzymes
MIKTNPRGDLPQVHESAFVDPTAILCGLVIVEEYVFIGPYAVIRADETDAAGRIAPIVIGAHSNIQDGVVIHSKSGASVWIGQRTSIAHRAIVHGPCRVGDGVFIGFNSVLFNCTIDDGCVVRYNAVVDGCHLPPGFYVRSTERIGPETDLAALPQVTADASDFSEDVARTNNALVLGYKHIQNEF

SEQ ID NO: 237 - protein, 214 aa, synthetic construct
  source 1..214: note = Library of modified or engineered enzymes
MQEITVTNFNNIAESPVTPWNPEPKKPKIHPTAYIHPLAYVQGDVTIGENVMVSANASIRSDEGYPIYIGNNSNVQDNVVLHALETVDENGNEIEENIVKVGDKKYAVYVGDNVSLAHQAQVHGPAAVGDNTFIGMQAFVFRSNVGKNCVLEPLAAAIGVTIPDNTYIPAGKVVTTQEEAAKLPKITPDHPFYKTNEAVVKVNVALAKGYLALA

SEQ ID NO: 238 - protein, 231 aa, synthetic construct
  source 1..231: note = Library of modified or engineered enzymes
MAAWAGGGPHWSYEGAGGPANWARLTPEFGACAGRNQSPIDLTGFIEAELPPLAFAYRAGGRSIVDNGHTVQVTYAPGSVLEVGGRRFELQQFHFHTPSEERINGRSYPLVAHLVHRDAAGHLAVVAVLFKQGAENPALAPLWAAMPGKAGETRALKAPLDAGALLPARRDYFTYMGSLTTPPCSEGVRWMVLRQPLEVSAAQVARFREVMGENARPVQPLNGRTVLHRVM

SEQ ID NO: 239 - protein, 214 aa, synthetic construct
  source 1..214: note = Library of modified or engineered enzymes
MQEITVTVFENIRPSPVTPWNPEPKLPKIHPTAYIDPAAVVQGDVTIGKNVMVSANASIRSDEGYPIYIGDNSNVQDNVVLHALETVDENGNVIEENVVTVGDKKYAVYVGDNVSLAHQAQVHGPAAVGDNTFIGMQAFVFRSTVGKDCVLEPLAAAIGVTVPDGTYVPAGKVVTTQEEAAKLPKMTPDHPFYKTNEAVVKVNVALAKGYLALS

SEQ ID NO: 240 - protein, 214 aa, synthetic construct
  source 1..214: note = Library of modified or engineered enzymes
MQEITVTRYENIRESPVTPWNPTPKRPEIHPTAYVDPLAYVQGDVTIGANVMVSAHASIRSDEGYPIVIGDNSNVQDNVVLHALETVDENGNEITENIVTVGDKKYAVYVGDNVSLAHQAQVHGPAAVGDNTFIGMQAFVFNSVVGKNCFLAPLAAAIGVTVPDGTYIPAGKVVTTQEEAAKLPKITPDHPFANTNAAVVKVNVALAKGYLALS

SEQ ID NO: 241 - protein, 214 aa, synthetic construct
  source 1..214: note = Library of modified or engineered enzymes
MQEITVTLFNNIRPSPVTPWNPEPKLPEIHPTAYIDPAAVVQGDVTIGKNVLIMANAVIRADEGYPIKIGDNSSVQDNVVLHALETVDENGNVIEENVVVVGDERYAVYVGDNVVLAHQAQVHGPAAVGDNTFVGMQALVFRSRVGKNCVLAHQAAAIGVEVPDGRYIPAGLVVTTQEEAARLPEVTPDHPFANLVDRVVKVNVALAKGYLALK

SEQ ID NO: 242 - protein, 300 aa, synthetic construct
  source 1..300: note = Library of modified or engineered enzymes
MVPERCRRTPTLLLFVSLAMAVAVSGACADDDKATAMRTAMPKPVSRRASTASSAETVVTPSVKEKRNTTPSPHHGTQDSWSYDNVAAWPATCAGNQQSPMPLRHTPADAHGRGSIRTLLTAATTLRLRAVRDGSSIALLCNGYCGVVKVHGVTHMIKNMHWHTPSEHTIDGRRLDAELHMVAFAGGKIAVLSSLFKVANKNVLVDRTIRAMSGMRSMSATRKEVKDYFFSGAVKVSAAVYKGSLTTPPCTEGLSWVVNAKVSTMSKKQLSKIRELLGGHDNARPLQAPKGRVVEWMDVP

SEQ ID NO: 243 - protein, 266 aa, synthetic construct
  source 1..266: note = Library of modified or engineered enzymes
MKQNLFAITVSCYWREPGDHLVDKSAPSHWNKLYPIAQGNRQSPINIITSQAVYSPSLKPLELSYDAATSLSITNNGHSVQVDFNDSDDRTVLKGGPLTGPYRLKQFHLHWGKKDAVGSEHTVDGVKYASELHLVHWNAKYGKFGEAVKQPDGLAVLGIFLKVGREKGEFQIFLDALDKVKTKGKEAPFTKFDPSCLFPACRDYWTYHGSFTTPPCEECIVWLLLKEPMTVSSDQMAKLRSLYSSAENEPPVPLVSNWRPPQPIKG

SEQ ID NO: 244 - protein, 253 aa, synthetic construct
  source 1..253: note = Library of modified or engineered enzymes
MIKKISLVLSIAALVLTGCNYSEGGKPKANVSAGYKKNWNYGTNNGPTHWEEFSSTCGKGIHQSPVNIIPGKTLKMNHAYDLSMHDDITGLAKVIDNGHSIKVTPEHGGHIKLHGEIFDLLQYHFHGKSEHTIDGKRFDMVAHMVHQNPKTKQLAVVAVFFEEGAKNKVLEKIINHVGSTVQLDAQDFVPLQTEHYYHYIGSLTTPPCSENVQWYLLKQPQEASEEQIKHFRKFYVDNERPVQELHDRFIEVN

SEQ ID NO: 245 - protein, 199 aa, synthetic construct
  source 1..199: note = Library of modified or engineered enzymes
MKITFLAVSCQHNEPDYWGRIKEDWKICKTGKMQSPIDLSNQRVKIISHLGDLKMNYKPSNATLKNRGHDIELEWKGGAGSIEINGTEYVLQQCHWHSPSEHTINGRRYDLELHMVHESRDGKIAVIGILYKIGRPDSFLSKLMKNIKSISDTKDEERAVGVIDPRHIKIGSRKYYRYIGSLTTPPCSQNVIWTIVKKV

SEQ ID NO: 246 - protein, 214 aa, synthetic construct
  source 1..214: note = Library of modified or engineered enzymes
MQEITVLEFSNVTKNEVTSWNPKPTTPVIDPTSYVDPNATVIGDVTIGKNCLIAASAVIRADEGAPIVIGDRSNVQDGVVLHALESVNDGGKIREENVILHGDEEYAVYVGKDVSLAHQAQVHGPARVGDDSFVGMKSLVFKSDVGSNCVIEPFAAAIGVTVPDGKYIPAGTVVTTQAEAEKLPEVTEDYPFYTTQEEVVKVNVNLCEAYREQA

SEQ ID NO: 247 - protein, 253 aa, synthetic construct
  source 1..253: note = Library of modified or engineered enzymes
MSTPLVKWGYDEQNGAHIWCRFFPAANGKRQSPIDIDINTVKHDPSLKPLSVSYDPSTAKEILNVGHSFHVNFEDSDNRSVLKGGPLTGSYRLRQFHLHWGSADDHGSEHTVDGVKYAAELHLVHWNPKYNTFAEALKQPDGIAVVGVFLKIGREKGEFQILLDALDKIKTKGKEAPFTKFDPSCLFPACRDYWTYHGSLTVPPLLESVTWIILKQPISVSSEQLAKFRSLLCTSEGETAVFMLRNHRPPQPL

SEQ ID NO: 248 - protein, 214 aa, synthetic construct
  source 1..214: note = Library of modified or engineered enzymes
MQEITVLEFSNVRKNEVTPWNPKPSTPVIDPTAYIDPQATVIGDVTIGANVLIGPMAVIRADEGAPIVIGDRSNVQDGVVLHALESINEEGEVREDNVVEVGDENYAVYIGKNVSLAHQSQVHGPARVGDDSFVGMKSLVFKSDVGSNCVLEPGAAAIGVTVPDGKYIPAGTVVTTQAEAAKLPEVTADYPFYTAQEAVVEVNVALCQAYNEQS

SEQ ID NO: 249 - protein, 214 aa, synthetic construct
  source 1..214: note = Library of modified or engineered enzymes
MQEITVYEFSNVTKNEVTPTNPKPSVPVIDPTSYIDPNATVIGDVTIGKNVLIAAFAVIRADEGRPIVVGDRSTVQDGVVLHALESVDDDGKVIEDNVVIHGNKDYAVYIGKNVVLAHQSQVHGPARVGDDSFVGMNSLVFNSVVGNNCVIEPNAAAIGVTVPDGKYIPAGTVVTTQEEADKLPEVTPDYAFYTKNEVVVNVNVDLCKAYKEKA

SEQ ID NO: 250 - protein, 214 aa, synthetic construct
  source 1..214: note = Library of modified or engineered enzymes
MQEITVHHFSNVTKNEVTSWNPKPSTPVIDPTSYVDPNATVIGDVTIGENVLIGANAVIRADEGAPIVIGDRSNVQDGVVLHALESVDDGGEEIEDNVVIEGDEEYAVYIGKDVSLAHQAQVHGPAAVGDDSFVGMKSTVFNSTVGENCVIEPDAAAIGVTVPDGKYIPAGTVVTTQEEAAKLPEVTPDHPSHDEIEAVVEVNVALNEAHREQA

SEQ ID NO: 251 - protein, 317 aa, synthetic construct
  source 1..317: note = Library of modified or engineered enzymes
MCDLNCIMTKMDNSDYMIVVCCVLLSIFLLFEIVEWIFKVFTWTDNDVCLPPTFSFGYAHKNGPHTWKDLYPESAGSNQSPINITTRYAIVVQPSEPLRWINYNSVPLSTTLSNDGHTVILRGFWDQSSWPQLQGGPLSDKYDFFNILFHWGPSNQEGSEHTLDYIRYPMELQVIHMKHGLKSPKDAIILGARDGIVIVSFFLQINAMDNPYLDHIVSNLWKISNPSHYKTNIPPFPLEWIFAPFDRDYYTYSGSLSQPPCNEVVTWIIQKEPIVISALQVEKFREICSVDGPLLLNCRPVQPLNERDVYFYEESKL

SEQ ID NO: 252 - protein, 214 aa, synthetic construct
  source 1..214: note = Library of modified or engineered enzymes
MQEITVTVYNNIQPSPVTSWNPTPKLPEIHPTAYVHPAAVVIGDVKIGENVMISPHASIRSDEGMPIYIGDNSNVQDGVVLHALETVDANGNTIEENVVTVGDKKYAVYVGKNVSLAHQSQVHGPAAVGDNTFIGMQSFVFKSVVGKNCVLEPLAAAIGVTVPDGTYVPAGKVVTTQEEAAKLPKVTPDHPYANTNAAVVYVNVELAKGYLALA

SEQ ID NO: 253 - protein, 214 aa, synthetic construct
  source 1..214: note = Library of modified or engineered enzymes
MQEITVLVYSNVQKNEVTSQNPKPVVPVIDPTSYVDPKATVIGDVTIGKNCMISASASIRSDEGHPIVIGDRSNVQDGVVLHALESVNDGGMILEENVVLAGGEDYAVYVGKNVSLAHQSQVHGPAKVGDDSFIGMQSFVFNSIVGSNCVIEPNAAAIGVTVPDNKYIPAGTVVTTQEEADKLPEITPDHAYYTTVAAVVNVNVGLCRAYKNEA

SEQ ID NO: 254 - protein, 339 aa, synthetic construct
  source 1..339: note = Library of modified or engineered enzymes
MTWAVPLVLLPLVLASALGAVVEVPETCGAEAGACVDEESAMVQVKTQPSQRAPSAATASGDVDYQGFQLGDWPEIAPLCAGGSTTGFQAPINIAVEGADYEKMPQASWPKFYAKEGGCDEAHFVEKGTAWQVDFMNPKINLDCKNLEMEWKGKVYALVQFHFHTLSEDTVDFQPTAMQMHMVHLAADGSFAVVGVLIKTDGFFKNGFLEGIFETGFESDRMVTLLAKHRFNPYAGVLSKHGEFWHYEGSFTTPPCTEGVDFLIAQSPVVTSKSYVTSYMEYLKGNGKGNSYGQNHRPIQPLNGREITTGRFLEVCPKKPAPDCGKLDPKKVQFCEESA

SEQ ID NO: 255 - protein, 253 aa, synthetic construct
  source 1..253: note = Library of modified or engineered enzymes
MLSGPQTWYKRFAINCEDVHQSPINIVTKKTIPDPNLKPLELTYDATTTRTIVNNGHSVQVDFEDSSNRTVITGGPLTGPYRLKQFHFHWGASDDKGSEHTVDGVKYASELHLVHWNAEKYSSFVEAAHEPDGLVVLGVFLKIGEHNPNLQKLTDALYSVRFKGTKAQFTNFNPKCLLPPSLDYWTYPGSLTTPPLLESVTWIVLKEPISVSPSQLAKFRSLLFTSEGETACCMVDNYRPLQPLMNRKVRASF

SEQ ID NO: 256 - protein, 214 aa, synthetic construct
  source 1..214: note = Library of modified or engineered enzymes
MQEITVTRFENIAPSPVTPWNPEPKLPKIHPTAYVHPLAYVQGDVTIGENVLIAPLASIRADEGYPIYIGDNSSVQDNVVLHALETVDENGNVLEENVVTVGDKKYAVYIGKNVVIAHLAQVHGPAAVGDNTFVGMLALVFKSNVGKNCVLEPLAAAIGVTIPDGKYIPAGKVVTTQEEAAKLPEVTPDHPFYKLNERVVKVNVALAKGYLAQA

SEQ ID NO: 257 - protein, 286 aa, synthetic construct
  source 1..286: note = Library of modified or engineered enzymes
MLGMKNTENHSAVLVQGHPAPANGGQIKVINNQEEGPSTWAESYPDYCNGSSQSPIDIDDWEVSPNPCDLSFVNYDLPFTGYWKNNGHALQFTLDDGSGAVVSGPCLGNSTYQLLQVHFHWGSAKGQGSEHTIEGKQHDLEMHMVHTNTAYETDEAANYKDGYLVVGVLFDEAKQNKIRGFERTFRNFVKKSSKLQDSDEGTLTAMFDVSDILRKSGVARSHFQYSGSLTTPSCNEVVTWILATKILKEKRSELNALRSLQTHDDEALVDNFRPTQELNGRKIMMF

SEQ ID NO: 258 - protein, 214 aa, synthetic construct
  source 1..214: note = Library of modified or engineered enzymes
MQEITVAEYSNVTKNEVTPTNPKPTTPVIDPSSYVDPNATVTGDVTIGKNCLIGASAVIRADEGAPIVIGDNSSVQDGVVLHALESVDDDGEVIEDNVVLYGNEDYAVYVGKNVVLAHQAQVHGPAAVGDDSFVGMKALVFKSIVGSNCVIEPDAAAIGVTVPDNKYIPAGTVVTTQEEAAKLPEVTPDHEYYTEVAEVVKVNVALCEAHLAKA

SEQ ID NO: 259 - protein, 214 aa, synthetic construct
  source 1..214: note = Library of modified or engineered enzymes
MQEITVTRFENIQPSPVTPWNPTPKLPEIHPTAYIHPAAVVQGDVTIGANVLVMANAVIRADEGYPIVIGDNSSVQDNVVLHALETVDANGNVIEENVVVVGDERYAVYVGDNVVLAHLAQVHGPAAVGDNTFVGMLSLVFRSRVGKNCFLAPLAAAIGVTVPDGKYIPAGKVVTTQEEADKLPEITPDHPGANLNARVVAVNVALAAGYLAQA

SEQ ID NO: 260 - protein, 214 aa, synthetic construct
  source 1..214: note = Library of modified or engineered enzymes
MQEITVTEFSNITKNEVTATNPEPVTPVIDPTSYVDPNATVTGDVTIGKNCLIAANAVIRADEGKPIVIGDRSSVQDGVVLHALESVDDEGMPIEENVVLEGDKYYAVYIGENVTLAHQSQVHGPARVGDDSFVGMKSLVFKSDVGSNCVIEPFAAAIGVTVPDGKYIPAGTVVTTQAEADKLPEVTDDYPFSTAQEAVVEVNVNLCEAYRNKA

SEQ ID NO: 261 - protein, 214 aa, synthetic construct
  source 1..214: note = Library of modified or engineered enzymes
MQEITVLEFSNVRKNEVTPTNPKPTTPVIDPTSYVDPNATVIGDVTIGENCYIAPFASIRADEGSPIVIGNNSNVQDGVVLHALESVNDGGKLIEDNVVLEGNEYYAVYIGNNVKLAHQSQVHGPAYVGDDSFVGMKSLVFKSKVGSNCVIEPEAAAIGVTVPDGKYIPAGTVVTTQAEADKLPEVTPDYAKSNAQEAVVKVNVALCEAYKKLS

SEQ ID NO: 262 - protein, 214 aa, synthetic construct
  source 1..214: note = Library of modified or engineered enzymes
MQEITVTKYENIRPSPVTPWNPEPKLPEIHPTAYVDPAAVVQGDVTIGANVMVSAHASIRSDEGYPIYIGDNSNVQDQVVLHALETVDEAGNVIEENVVTVGDKKYAVYVGDNVSLAHQAQVHGPAAVGDNTFIGMQAFVFKSVVGKNCVLEPLAAAIGVTVPDGKYIPAGTVVTTQEEAAKLPEVTPDHPFYNTNAAVVKVNVALAKGYLALS

SEQ ID NO: 263 - protein, 333 aa, synthetic construct
  source 1..333: note = Library of modified or engineered enzymes
MCQLENAIEDIFELKEDIVQCQWTVPLVTITIVNEGTGEIEAQPNKIELQKVQNLCTIVKTEWMYQGADNQNDKWPQNCPSCDASLEGNERQSPIDLNPQMTNMVTKTLPKLTFTPNPNGDTLGKFENKVNTIQFTANDLSQNKMHGGPLSGEYSFWQMHCHWGKTNYEPGTTEPTKVEQHGSEHWIDGKQYDAECHWVHFNNKYATVGDAIASGDADALSVIGVMLEIDETNGQDEVEWIGTVKDAASALVTPDDGPAEDAPFNVYGFLDQLGDQSQCFGYYNYLGGLTTPGCNQLVSFIIIDTPIRINMAQVKNVKYNKIESFCSIYVRQY

SEQ ID NO: 264 - protein, 214 aa, synthetic construct
  source 1..214: note = Library of modified or engineered enzymes
MQEITVLRFSNVTKNAVTATNPKPTTPVIDPTAYVDPNATVIGDVTIGKNCYIAAFARIRADEGRPIVIGDNSNVQDGVVLHALESIDDAGKVIEDNVVVEGNKLYAVYVGRNVSLAHQSQVHGPARVGDDSFVGMKSLVFKSDVGSNCVIEPFAAAIGVTVPDGKYIPAGTVVTTQAEADALPEVTPDYAFYTQVAAVVTVNVALCEKYKAQA

SEQ ID NO: 265 - protein, 262 aa, synthetic construct
  source 1..262: note = Library of modified or engineered enzymes
MKLINFVGTACSPYEQWRDHYPIADGNRQSPIDIVPGSASYDSGLKPLTLKYDPSTSLEILNNGHSFQVTFVDDSDSSTLKGGPISGVYRLKQFHFHWGSSDDHGSEHVVDGVKYAAELHVVHWNAAKYSSFVEAAHEPDGLAVLGVFLKVGEHNSQLQKITDILNSIKEKGKQTRFTNFDPICLLPPCPDYWTYPGSLTVPPLLESVTWIVLKQPISVSSQQLAAFRNLLFTSEGEKACCMVNNYRPLQPLMNRTVRSSFR

SEQ ID NO: 266 - protein, 214 aa, synthetic construct
  source 1..214: note = Library of modified or engineered enzymes
MQEITVLVYSNVTKNERTSYNPKPTVPVIDPTSYVDPNATVIGDVKIGKNCYVAAFAVIRADEGKPIVIGDRSNVQDGVVLHALESVDAGGKLIEDNVVIHGDNWFAVYVGKNVVLAHRAQVHGPAAVGDDSFVGMNSLVFNSKVGSNCVIEPEAAAIGVTVPDGKYIPAGTVVTSQAEADKLPEITPDYAYYTQNAAVVNVNIGLCRGYKRLA

SEQ ID NO: 267 - protein, 214 aa, synthetic construct
  source 1..214: note = Library of modified or engineered enzymes
MQEITVTVFENIRESPVTPWNPTPKRPVIHPTAYIDPAAVVQGDVTIGANVMVSANASIRSDEGYPIYIGDNSNVQDNVVLHALETVDANGKRIEENVVRVGDKDYAVYIGDNVSLAHQAQVHGPAAVGDNTFIGMQAFVFRSRVGANCVLEPLAAAIGVTVPDGTYVPAGKVVTTQEEADKLPKVTPDHPFATTNAAVVAVNVALAKGYLAQK

SEQ ID NO: 268 - protein, 214 aa, synthetic construct
  source 1..214: note = Library of modified or engineered enzymes
MQEITVLEFSNVTKNEVTSWNPKPVTPVIDPTSYVDPDATVIGDVTIGENVLIAAGATIRADEGKPIYIGDRSSVQDGVVLHALESRDDGGMENGDNVVIHGNTLYAVYVGNNVSLAHQSQVHGPAAVGDDSFVGMNSLVFNSKVGSNCVIEPNAAAIGVTVPDGKYIPAGTVVTSQAEADKLPEITPDYEYYTAVAKVVGVNVALCEAYQELQ

SEQ ID NO: 269 - protein, 235 aa, synthetic construct
  source 1..235: note = Library of modified or engineered enzymes
MTAALLSASAWAAPHWEYSGEAGPANWAKLTPEFGACAGKNQSPINLTGFTQAQLKPLKFNYQADAKSILNNGHTVQVNFKPGNYLELDGQRFELKQFHFHAPSENLIEGKSFPLEAHFVHANAQGELAVLALMFKPGKANPELAKAWQQMPEKAGEETVLKAPINAQDLLPKNLEYYRFSGSLTTPPCSEGVRWLVMKQPVELSQEQIDAFKEIMHHPNNRPLQPLNGRPVLTS

SEQ ID NO: 270 - protein, 214 aa, synthetic construct
  source 1..214: note = Library of modified or engineered enzymes
MQEITVTVYNNIRESPVTSWNPEPKKPEIDPTAYIDPKAVVRGDVKIGKNVMVSANASIRSDEGYPIYIGDNSNVQDNVVLHALETVDEDGNEIEENIVTVGDEKYAVYVGENVSLAHQAQVHGPAAVGDNTFIGMQAFVFRSVVGKNCVLEPLAAAIGVTVPDGTYIPAGKVVTTQEEADKLPKVTPDHPFYKTNEAVVKVNIELAKGYLAQS

SEQ ID NO: 271 - protein, 214 aa, synthetic construct
  source 1..214: note = Library of modified or engineered enzymes
MQEITVTRYENIRESPVTPWNPTPRRPQIHPTAYIDPAAVVQGDVTIGANVMVSPLASIRSDEGYPIVIGDNSNVQDQVVLHALETVDAAGKTLEENVVTVGDEKYAVYVGDNVSLAHQAQVHGPAAVGDNTFIGMQAFVFKSTVGKNCVLAPLAAAIGVTVPDGTYVPAGTVVTTQEEAAKLPKVTPDHPFATTNAAVVAVNVALAAGYRALA

SEQ ID NO: 272 - protein, 257 aa, synthetic construct
  source 1..257: note = Library of modified or engineered enzymes
MRSSAFAVPAVAALAVAGLSAALAFAAQANEPAKPAAAGHHEVYDYDHQEAWQALHDKSQSPIDIVTAGAAAADPAEPRAIEFSHTHGAIDKIEDNGHAVQVDTHATEATIRGRHFKLAQFHFHAQSEHTLDGKHFPLEGHFVFKAQDGRLAVVGVMYEQGKANAVAQEVLDDLKPGKAKPAQPEIDIEGLLPKAHGYYHYLGSLTTPPLTENVEWYVMPTPVTMSKQQIDGFLSHYRRNNRNIQPLNGRPLIRYEG

SEQ ID NO: 273 - protein, 260 aa, synthetic construct
  source 1..260: note = Library of modified or engineered enzymes
MARKPSGHLTIYEQDVNCFWSFIEPIEGTGQSPIDLHTKEIKYDSSLKPLSVKYDPSTAKEISNTGHSFQVTFEDNDNKSVLRGGPLTDSYRLSQFHFHWGSSDEHGSEHVVDGVKYAAELHLVHWNAAKYSSFAEAAHEPDGLAVLGVFLKVGEHNPQLQKVIDALNSIKTKGKRAPFTNFDPSTLLPSSLDYWTYDGSLTTPPLLESVTWIVLKEPISVSSEQMSKFRSLLFTSEGETACCMVDNYRPPQPLKGRQVR

SEQ ID NO: 274 - protein, 214 aa, synthetic construct
  source 1..214: note = Library of modified or engineered enzymes
MQEITVTRFENIRESPVTSWNPTPKKPKIHPTAYVDPLASVIGDVTIGENVMISPHASIRSDEGMPIYIGDNSNVQDGVVLHALETVDDNGNVIEENVVVVGDEKYAVYIGENVSLAHQSQVHGPAIVGDNTFIGMQSFVFKSKVGKNCVLMPLAAAIGVEVPDNKYIPAGKVVTTQEEADKLPEITPDHPYYNTNKAVVYVNVELAKGYLALS

SEQ ID NO: 275 - protein, 214 aa, synthetic construct
  source 1..214: note = Library of modified or engineered enzymes
MQEITVTVYNNIQASPVTPWNPTPKLPEIHPTAYVHPAAVVQGDVTIGENVYIAANAVIRADEGYPIVIGDNSSVQDNVVLHALETVDEDGNVIEENVVKVGDKDYAVYVGKNVVLAHNAQVHGPAAVGDNTFVGMNALVFRSTVGKNCFLAPNAAAIGVTVPDGTYIPAGKVVTTQEEAAKLPKITPDHPGANLNERVVKVNVALAKGYLAQA

SEQ ID NO: 276 - protein, 214 aa, synthetic construct
  source 1..214: note = Library of modified or engineered enzymes
MQEITVTKYENIRESPVTPWNPTPKKPEIHPTAYIDPAAVVQGDVTIGKNVMVSANASIRSDEGYPIKIGDNSNVQDNVVLHALETVDENGKEIEENIVVVGDEKYAVYIGDNVSLAHQAQVHGPAAVGDNTFIGMQAFVFRSRVGKNCVLEPLAAAIGVTVPDGTYIPAGKVVTTQEEADKLPKITPDHPFANTNAAVVKVNVALAKGYLAQA

SEQ ID NO: 277 - protein, 214 aa, synthetic construct
  source 1..214: note = Library of modified or engineered enzymes
MQEITVFIFSNVEKNEVTETNPKPVVPKIDPTSYIDPNATVIGDVTIGENCYIAPFASIRADEGKPIVIGDRSNIQDGVVLHALESVDDNGEIIEENVVLEGDEYYAVYIGKNVSLAHQSQVHGPAKVGDDSFVGMKSLVFNSIVGSNCVIEPNAAAIGVTVPDGKYIPAGTVVTTQEEADSLPEVTPDHAAYTKIAAVVTVNVSLCLAYLGES

SEQ ID NO: 278 - protein, 203 aa, synthetic construct
  source 1..203: note = Library of modified or engineered enzymes
MSAPVIRWTYEGDKGPHFWNQLCEEYEIAKTGKNQSPIDIHMEKVMEVQGAPPLELNYKPTKYTVRRVENSVHLFPKDKEQGLTFNGKRYNLIAFHGHIPSEHTLNEHYFAIEWHLVHMNEAGERLVLGIWMEKELEGSDFGELAEIFPEVFADFGIEKEISLDVSGFLPEERAYFTYQGSLTTPPTFEGVTWIVLRNATSIS

SEQ ID NO: 279 - protein, 382 aa, synthetic construct
  source 1..382: note = Library of modified or engineered enzymes
MVASSLSSLCLVLASLVGQTLATSPCDVDKTSPECDKTGVNRASWGYAASNGPATWAANYPDFCAGDMQSPIDLDSSKAVTMDPGPITMVGYNLKQAGKIENNGHTLGFAFASGSTPYIMGGRLPAGDRFDFVQLHWHWGSDSSKGSEHTMNGKEYPIEVHLVHANTKYYVNGAPSNDNLVMPDGLAVLGIFYEVSTEDNANLTNIVSKVNEVAVEQRRRRKQGRAGSNEVDLDMTLALDSFLPADTTQYYYYQGGLTTPSCNEAVLWTNMKSTQTISEAQLEVFRSMTDSDGITLNNNYRPPQPLNNRTIYTTGTSTTAGSSNMFTELLNTAFTAAVVTGLVGIVAPLFAPPPSQQRSDAASARAEQALRAGRDQWGGYWG

SEQ ID NO: 280 - protein, 214 aa, synthetic construct
  source 1..214: note = Library of modified or engineered enzymes
MQEITVLTFSNVTKNEVTATNPKPTTPVIDPTSYVDPNATVTGDVTIGKNCFIGANAVIRADEGKPIVIGDNSNVQDGVVLHALESVDDGGKVREDNVVVHGNEWYAVYIGKNVSLAHQSQVHGPAYVGDDSFVGMKSLVFKSIVGSNCVIEPEAAAIGVTVPDGKYIPAGTVVTTQEEADKLPEITPDYAFYTQIAEVVKVNVGLCKAYREKA

SEQ ID NO: 281 - protein, 214 aa, synthetic construct
  source 1..214: note = Library of modified or engineered enzymes
MQEITVTRYTNIRPSPVTPWNPEPKLPEIHPTAYIDPLAYVQGDVTIGENVMISANASIRSDEGYPIVIGNNSNVQDNVVLHALETVDENGNRLEENVVKVGDEEYAVYVGDNVSLAHQAQVHGPAAVGDNTFIGMQAFVFRSRVGKNCYLAPLAAALGVTVPDGKYIPAGKVVTTQEEAAKLPEITPDHPFANTNAAVVKVNVALAKGYLAQA

SEQ ID NO: 282 - protein, 309 aa, synthetic construct
  source 1..309: note = Library of modified or engineered enzymes
MFKSSLILLATLSVVLCGDDAKSWGYRNKGRNIVPEKWGEMQPKCLGSVQSPINVDFASTQFDANLGKLNIKKHGNETEQWDVKNNGHSVVFTPVNTDFSFVIYPQKEEFKLLQLHFHWRGSEHFVNGIKYAGELHLVHQSKTNPNQFSVIGFLLQLVNADNLKMKAVIDVLADVTEYEATKKIDNFELNDMVPFEVENFFRYSGSLTTPGCDEFVEWNLADKPVIGLSENQILEFQSLLDNHKYPILSNSRPVQEINDRIVKRSFYPFEAKARTHGASGYSVSGANKFQFTSSVFFTLIASAFCFYSL

SEQ ID NO: 283 - protein, 214 aa, synthetic construct
  source 1..214: note = Library of modified or engineered enzymes
MQEITVLEFSNVTKNEVTPTNPKPVTPVIDPTSYVDPDATVVGDVTIGANVLIWPKAVIRADEGKPIVIGDRSNVQDGVVLHALESVDDGGEIREDNVVLVGDENYAVYVGNNVSLAHQSQVHGPARVGDDSFVGMKSLVFKSDVGSNCVLEPNAAAIGVTVPDGKYIPAGTVVTTQEEAAKLPEVTDDYPFYTAQDAVVEVNVDLCEAYKGQA

SEQ ID NO: 284 - protein, 214 aa, synthetic construct
  source 1..214: note = Library of modified or engineered enzymes
MQEITVDTFSNVTKNEVTSTNPEPVTPVIDPTSYVDPNATVTGDVTIGKNCYIGANAVIRADEGAPIVIGDRSNVQDGVVLHALESVDDEGEIREDNVVVHGDENYAVYIGEDVSLAHQSQVHGPARVGDDSFVGMKSLVFNSTVGENCVIEPEAAAIGVTVPDGKYIPAGTVVTTQAEADKLPEVTPDYAFYTEVAEVVTVNVALCEAHREQK

SEQ ID NO: 285 - protein, 314 aa, synthetic construct
  source 1..314: note = Library of modified or engineered enzymes
MIGRSSLRARLATASAGLVLSAVPVAAPVTAAAAATPVMSIMAGETAEWNHDPASPIGPTHWGELDPAWSACRSVQDQSPIAVTPTREADRPVLLVDYPRTPLVVRNTGHVIEVPAPPGGGGTLLVGGHSYRLLQWHTHVPSEHVVNGHRADLEIHLVHQDEQGEIAVLAVFADVVSLGEAAPRMPAADLLRTTVQAAPSTAGEEIDLDQKVSAAALLGATVEDGEQRRAITNYLSYTGSLTTPPCTGGVRWFLLPGIIGVDPASVQPLHALIASFPGYDGYPDNNRPVQPVGSRMVERRVGWPSVGGVTSGAA

SEQ ID NO: 286 - protein, 224 aa, synthetic construct
  source 1..224: note = Library of modified or engineered enzymes
MSPLCWGYEKDNGAHVWRQTFIAAEGPRQSPIDIQTSKAVPDLTLKPLTLSYDPATSLEILNNGHSFQVTFADDSDSSTLTEGPVSGIYRLKQFHFHWGASDDKGSEHTVDGVKYPAELHLVHWNAVKFKSFGEAALEENGLAVVGVFLKIGKHHPELQKLVDALPAIKHKDTLVKFGSFDPSCLMPTCPDYWTYPGSLTTPPLSESVTWIVLREPISVSPEQL

SEQ ID NO: 287 - protein, 237 aa, synthetic construct
  source 1..237: note = Library of modified or engineered enzymes
MAAPSASKGHDVHWSYEGDNGPANWGKIKPEWAKCSTGNRQSPIDIRDGMKVELDQIQFDYRPSSFSVIDNGHTVQVGVSGGNYITVQNRMYELQQFHFHRPSEERINGKAFEMVIHLVHKDAEGRLAVLAVLLERGAPQPVIQTVWNHLPLEKFETMQPTILLDPAELLPARRDYFTYMGSLTTPPCTEGVLWMVMREPIQASSEQIAIFARLYPMNARPIQETNGRMIWKSKYLS

SEQ ID NO: 288 - protein, 260 aa, synthetic construct
  source 1..260: note = Library of modified or engineered enzymes
MRLSTIFVAGYCPEKWDHQNPITGGEHQSPINIISSQTKYDPNLKPLNISYDPSTSLEILNNGHSFQVTFKDNDNRSVLKGGPLDDVYRLEQFHFHWGKKDAEGSEHTVDGVKYSSELHLVHWNAVKYSSFEEAASKENGLAVLGVFLKVGEHNPKLQKIIDALNSIKTKGKQTTFTNFDPSTLLPSSLDYWTYSGSLTTPPLSECVTWIVLKEPISVSPAQMAKFRSLLFTSEGEKACCMVDNYRPPQPLKGRKVRASF

SEQ ID NO: 289 - protein, 214 aa, synthetic construct
  source 1..214: note = Library of modified or engineered enzymes
MQEITVLFFSNVTKNEVTATNPKPVTPVIDPTSYVHPEATVIGDVTIGENCYIAPFASIRADEGSPIVIGNDSNVQDGVVLHALESVDDGGKLIEDNVVLEGHKNYTVYIGKNVSLAHQSQVHGPARVGDDSFVGMNSFVFNSKVGSNCVIEPNAAAIGVTIPDGKYIPAGTVVTSQAEADNLPEITEDYKYYTQIAAVVNVNVGLCRAYREKA

SEQ ID NO: 290 - protein, 282 aa, synthetic construct
  source 1..282: note = Library of modified or engineered enzymes
MKNVTGHLSARCFQIEDYPWSYDNDLLGGPDFWGLINKHWKLCAIGKMQSPIDIDPNILLYDPNLKPIHIDKHKVSGTLENTGQSLVFRVDKETKHHVNISGGPLAYKYQFHEFYIHFGLHDHLGSEHSIDRYSFPAEIQLYGFNSDLYNNMSEAQEKSQGLVGVSLMVQIGETPNPELRIITSTFNKVIYRGKSAPVKRLSVRSLLPDTKDYVTYEGSTTHPGCWETTVWIILNKPIYITKQELYALRKLMQGSKSHPKAPLGNNARPIQDLHGRTVRTNI

SEQ ID NO: 291 - protein, 214 aa, synthetic construct
  source 1..214: note = Library of modified or engineered enzymes
MQEITVTRYENIRPSPVTPWNPTPRRPRIHPTAYVDPAAVVQGDVTIGANVLVMANAVIRADEGYPIVIGDNSSVQDNVVLHALETVDAAGRRLEENVVRVGDEDYAVYVGANVVLAHNAQVHGPAAVGDNTFVGMNALVFRSRVGANCVLAPNAAAIGVTVPDGTYVPAGLVVTTQEEAARLPRVTPDHPFANLNARVVAVNVALAAGYRALA

SEQ ID NO: 292 - protein, 214 aa, synthetic construct
  source 1..214: note = Library of modified or engineered enzymes
MQEITVTKYENIRPSPVTPWNPEPKLPEIHPTAYIDPAAVVQGDVTIGANVMVSANASIRSDEGYPIVIGDNSNVQDNVVLHALETVDADGKVLEENVVKVGDERYAVYVGDNVSLAHQAQVHGPAAVGDNTFIGMQSFVFRSVVGKNCVLEPLAAAIGVTVPDGTYVPAGKVVTTQEEAAKLPKITPDHPFANTNAAVVAVNVALAKGYLAQS

SEQ ID NO: 293 - protein, 341 aa, synthetic construct
  source 1..341: note = Library of modified or engineered enzymes
MAGSSARALAALVALVLVAVAVIAEPRQQQLKTALLRIEGGPADTFAAAAEAEAAAAKAAEAAAACPWSYEGANGPANWGTICGKIFSECATGMQQSPINIKLLRMHQGPGQSMIGWKIPSDAYNKFVTFGGGGDYLESYDGHSFVVSHADALFPFGGVTYKLQSFHTHTVSEHTIDGEHYDMEMQFVHKTVDGAKFSTGLKGELGQTLIVSVMFQVGKGQGSPHWLRQLAKAVPSVTNESAQVIPLDFTEVAQSVMVGTLPQDARFKDFKPNYNHYYGYTGSLTAPPCTQGVQWLVLANPIYAEAEDIQAFKDLEGDNFRPVQRINGRIVTQRYCGLSCE

SEQ ID NO: 294 - protein, 268 aa, synthetic construct
  source 1..268: note = Library of modified or engineered enzymes
MAFTRLSISLLLSGLILSAGMPAQAAPETVMVPEVTAMALEGKWPADWSYQGENGPAHWGELHPSYSKCARGRVQSPVDLGKATTRSRRSTVRVAFHPIRYEIFNDGRGIRAVPLEAQHPIRIDRHDYTLKHIVFRAPSEHTFQGRHYPLEAQLVYEADDGALAVLATVFSPGHSNPSLAALTRQPLAEGQLDKPMGTRVLLPRRLPHLRLNGSLTTPPCTEGVNWVVFTQPVQATRAQIDAMTRLIGHPNNRPVQPAHRRLMVEEMR

SEQ ID NO: 295 - protein, 267 aa, synthetic construct
  source 1..267: note = Library of modified or engineered enzymes
MKRTSLFAVIGECPQWNYDHQEEWKIVFPQANGDQQSPINIEPSSAVYDSALKPLELKYDPSTSLEILNNGHSFQVTFVDDSDSSTLKDGPITGVYRLKQFHFHWGAADDKGSEHTVDGVKYPAELHLVHWNAKYGSFGEAASKPDGLAVVGVFLKIGKHHPGLQKLTDALYSIRFKGTKAEFSGFNPKCLLPASLDYWTYPGSLTTPPLSESVTWIVLKEPISVSPEQMAKFRSLLFTSEGETACCMVDNYRPLQPLKGRKVRASF

SEQ ID NO: 296 - protein, 214 aa, synthetic construct
  source 1..214: note = Library of modified or engineered enzymes
MQEITVLEFSNITKNEVTSWNPKPKTPVIDPTSYIDPNATVIGDVTIGKNCYIGASAVIRADEGRPIVIGDNSNVQDGVVLHALESVNDDGKVIEDNVVLEGNKYYAVYIGKNVVLAHQSQVHGPAAVGDDSFIGMNSLVFRSIVGSNCVIEPNAAAIGVTVPDNKYIPAGTVVTTQEEADKLPEITEDYAYWNTIAEVVKVNVNLCEAYKNEA

SEQ ID NO: 297 - protein, 214 aa, synthetic construct
  source 1..214: note = Library of modified or engineered enzymes
MQEITVLEYSNVRKNEVTTTNPKPKTPVIDPTSYVDPKATVIGDVTIGENVLIGPFAVIRADEGRPIVIGDRSNVQDGVVLHALESVDDDGKIIEDNVVVEGDEYYAVYIGKNVVLAHQAQVHGPARVGDDSFVGMNALVFNSIVGDNCVIEPNAAAIGVTVPDGKYIPAGTVVTTQEEADTLPEITPDDEYYTKIAEVVKVNVALCEAYREKA

SEQ ID NO: 298 - protein, 214 aa, synthetic construct
  source 1..214: note = Library of modified or engineered enzymes
MQEITVTNYNNIQASPVTSWNPEPKLPEIHPTAYIHPKAVVQGDVTIGANVMISANASIRSDEGYPIVIGDNSNVQDNVVLHALETVDENGNVIKENVVKVGDKDYAVYIGKNVSLAHQAQVHGPAAVGDNTFIGMQAFVFRSNVGKNCVLMPLAAAIGVTIPDGTYIPAGKVVTTQEEADKLPKVTPDHPFYKTNEAVVKVNVELAKGYLAMA

SEQ ID NO: 299 - protein, 214 aa, synthetic construct
  source 1..214: note = Library of modified or engineered enzymes
MQEITVLFFSNVRKNEVTPQNPKPVTPVIDPTSYVDPNATVIGDVTIGENCLIGASAVIRADEGHPIVIGDRSSVQDGVVLHALESVDDGGKIIEDNVVLEGDEYYAVYVGRNVVLAHQSQVHGPAAVGDDSFVGMKSLVFRSIVGSNCVIEPEAAAIGVTVPDGKYIPAGTVVTTQEEADKLPEVTPDHADYTKQAEVVKVNVGLCEAYRELS

SEQ ID NO: 300 - protein, 265 aa, synthetic construct
  source 1..265: note = Library of modified or engineered enzymes
MKNSCATYSVVVMTLSLILVLTISLGYQNPFAMGQDTINDTSIISKQWPNIMSNVNTFVVENVTSPRIDDTAYIHPFAIIIGDCSIGKKVLVAPTAVCRADEGIPIHIGDYSNIQDGVILHALDAVRDGTNVDNKRFSQEGDRLLGNDTRFDEGYAIYLSGNVSLAHDSLIHGPVWIGNNTLIGVKSAVLDSKIGNNVVIRVGSIITGVEIPDNTLVPPGSVLTNQSQVATLPSVIGSPSQNLNQGDLKNSQALATAYDNTNIER

SEQ ID NO: 301 - protein, 214 aa, synthetic construct
  source 1..214: note = Library of modified or engineered enzymes
MQEITVLSFSNVTKNEVTSTNPKPTTPKIDPTSYVDPKATVTGDVTIGKNCLIGPFAVIRADEGAPIVIGDNSNVQDGVVLHALESVDDGGKIIEENVVLYGNKYYAVYIGKNVVLAHQAQVHGPARVGDDSFVGMNSLVFNSIVGSNCVIEPNAAAIGVTVPDNKYIPAGTVVTTQAEADNLPEITPDHPYYTAVEKVVEVNVNLCKAYRNKE

SEQ ID NO: 302 - protein, 214 aa, synthetic construct
  source 1..214: note = Library of modified or engineered enzymes
MQEITVTKYENIQESPVTPWNPTPKRPVIDPTAYVHPAAVVQGDVTIGKNVMISANASIRSDEGYPIYIGDNSNVQDNVVLHALETVDADGNEIEENIVTVGDKKYAVYIGDNVSLAHQAQVHGPAAVGDNTFIGMQAFVFRSNVGKNCVLAPLAAAIGVTVPDGTYVPAGTVVTTQEEADKLPKVTPDHPFANTNAAVVKVNVALAKGYLALS

SEQ ID NO: 303 - protein, 328 aa, synthetic construct
  source 1..328: note = Library of modified or engineered enzymes
MRGCLEKTGEGAIAGILVMAGPPQIQGSNFHSGFIFLKNFIMNSTQHCTLLAISFCFALLLLFACNGANKEQQQSSTSNISKDHPKADTLLGDNEVKAEAPDANSKAEQGYALPQHTDRLAQSPIDIISVKADKTVKEQISFAFHSDINAAKNLGHTIELEFKEGSTCKVNGKDYASRQFHFHTPSEHLVDGITFPMEMHIVNILADSVNTNKPSYLVLAVLFKIGTENKFIKEFFNKIPNKEGEENTLQTGDVRLDDLLSQFTPNDIKSYYTYQGSLTTPPFTESVQWVILKHIVEASEEQIMAIEKMEGNNARHVQAINDRKIYSH

SEQ ID NO: 304 - protein, 263 aa, synthetic construct
  source 1..263: note = Library of modified or engineered enzymes
MSRPGTVLAIFCWNHQEKYDMKKVLAVAAALLALGGVAAEASHWGYEGEGAPEHWGALDEAYKACQAGKNQSPINIEHALKAHHGQLDLAFKPGAQQIVNNGHTIQVNVSAGNTLTLDGDTFTLQQFHFHAPSENEIDGKQFPLEAHFVYKDKDGALVVLALMFQQGKANPQLAQAWQQMPAAIDQVATLNQPVDIKALLPKEFNFYRFSGSLTTPPCSEGVRWLVLDQPVSASAEQIQQFRAVVHHANNRPVQPLHGRVIVD

SEQ ID NO: 305 - protein, 214 aa, synthetic construct
  source 1..214: note = Library of modified or engineered enzymes
MQEITVLDFSNITKNEVTPTNPEPITPVIDPTSYIDPNATVTGDVTIGKNVLIGPNAVIRADEGRPIVVGDRSNVQDGVVLHALESVDDEGEPIEDNVVEKGDELYAVYIGKNVSLAHQSQVHGPARVGDDSFVGMKSLVFKSDVGENCVIEPESAAIGVTIPDGKYIPAGTVVTSQAEAAKLPEVTPDYAYSDTNEAVVKVNVGLCEAYKEQA

SEQ ID NO: 306 - protein, 214 aa, synthetic construct
  source 1..214: note = Library of modified or engineered enzymes
MQEITVTRFENIRPSPVTPWNPTPKRPVIHPTAYVDPAAVVQGDVTIGANVMISALASIRSDEGYPIYIGDNSNVQDNVVLHALETVDADGKRIEENVVKVGDKDYAVYIGDNVSLAHQAQVHGPAAVGDNTFIGMQAFVFRSRIGKNCYLAPLAAAIGVEVPDGTYIPAGKVVTTQEEAAKLPKMTPDHPFYKTNEAVVAVNVALAKGYLALA

SEQ ID NO: 307 - protein, 275 aa, synthetic construct
  source 1..275: note = Library of modified or engineered enzymes
MKRSLVATIFGCYEPNDHQWGYTKDNGPATWAKSFPAANGPRQSPIDIKPSETKYDSSLKPLSLKYDPSTALEILNNGHSFQVTFKDSENKSVLQGGPLEGTYRLEQFHFHWGSSDEHGSEHVVDGVKYASELHVVHWNAKYGDFGEAVKHPDGLAVLGIFLKVGKHHPEFQKLLDALNSIKNKGKQASFTNFDPSVLLPACLDYWTYSGSLTTPPLLESVTWIVLKEPISVSPAQMEQFRSLLFTSEGEKEKRMVDNFRPLQPLMNRTVRSSFR

SEQ ID NO: 308 - protein, 263 aa, synthetic construct
  source 1..263: note = Library of modified or engineered enzymes
MTLSRAFIVGCPQEDHKNWYNQYPIAKGNRQSPINIETKQAQYDSSLKPLTFSYDPSTAKEIVNVGHSFHVNFEDNDNQSVLSGGPLTGSYRLKQFHFHWGASDEHGSEHTVDGLKYPAELHLVHWNAKYGSFSEAASQPDGLAVVGVFLKIGDENPKLQKIIDALESIKTKGKQTRFTNFDPSTLLPSCLDYWTYHGSLTTPPLLESVTWIICKEPISVSPSQMEKFRSLLFTSEGEKECCMVDNYRPLQPLMNRTVRSSFR

SEQ ID NO: 309 - protein, 214 aa, synthetic construct
  source 1..214: note = Library of modified or engineered enzymes
MQEITVTRYENIRPSPVTPWNPEPKRPKIHPTAYIDPAAVVVGDVTIGENVLVMANAVIRADEGYPIVIGDNSVVQDNVVLHALETVDENGNRIEENVVRVGDEDYAVYVGKNVVLAHNAQVHGPAAVGDNTFVGMNALVFRSRVGKDCVLMPNAAAIGVTVPDGTYVPAGLVVTTQEEAAKLPKVTPDHPFANLNARVVKVNVALAKGYLALA

SEQ ID NO: 310 - protein, 214 aa, synthetic construct
  source 1..214: note = Library of modified or engineered enzymes
MQEITVTRYENIRPSPVTPWNPTPKLPKIHPTAYVDPAAVVQGDVTIGANVMVSANASIRSDEGYPIVIGDNSNVQDNVVLHALETVDAAGRRLEENVVRVGDEEYAVYVGDNVSLAHQAQVHGPAAVGDNTFIGMQAFVFRSRVGKNCVLEPLAAAIGVTVPDGRYVPAGTVVTTQAEADKLPKVTPDHPFYKTNAAVVKVNVALAKGYLAQA

SEQ ID NO: 311 - protein, 225 aa, synthetic construct
  source 1..225: note = Library of modified or engineered enzymes
MGSHAHWSYSGASGPEHWASLTPEYGACAGRNQSPVDLAGFIEADLAPIAFHYQAGGTEVVNNGHTVQVNYAPGSAIELDGHRFELKQFHFHAPSENLIDGKSYPLEMHLVHADEAGHLAVVALMFKAGAENAALAKLWKAMPEQPGETVHLAPLVSAEALLPKDRDYYRFNGSLTTPPCSEGVRWLVMKEPVSASAEQIAAFEKRLPHPNNRPLQPTNARLVLK

SEQ ID NO: 312 - protein, 214 aa, synthetic construct
  source 1..214: note = Library of modified or engineered enzymes
MQEITVLFFSNVTKNEVTPTNPKPSTPVIDPTSYVDPNATVTGDVTIGANVLVAANATIRADEGKPIVIGDRSSVQDGVVLHALESVDDEGEIKEDNVVVVGNKNYAVYIGKNVSLAHQSQVHGPAHVGDDSFVGMKSLVFKSDVGSNCVIEPFAAAIGVTVPDGKYIPAGTVVTTQEEADKLPEVTPDYAESNTNEAVIKVNVGLCEAYKNKS

SEQ ID NO: 313 - protein, 260 aa, synthetic construct
  source 1..260: note = Library of modified or engineered enzymes
MAWNRRNFLGSFACFGLSLATGRALAVTSCKPEEQPCWGYHGDEGPDHWGRLHPDWVACAEGSEQSPIALAGEEVKPTAERFALHYQPTTARLSDNVHTVRIDMEPGSQLLLGDRTFSLRQFHFHTPSEHLWSDTADLGELHLVHVADSREIAVLGVALRPDAAQAFPDSFWNWLQAAEAGESLTLDPAGLVPRQGRLVGYRGSFTTPPCTEGVNWLLAMEPMAMGPEEQRWLEQRMGRNARPVQPLGSRTVHTVLREGS

SEQ ID NO: 314 - protein, 214 aa, synthetic construct
  source 1..214: note = Library of modified or engineered enzymes
MQEITVTRYENIRESPVTSWNPEPRRPEIHPTAYVDPAAVVQGDVTIGENVMVSAHASIRSDEGYPIVIGNNSNVQDNVVLHALETVDENGNEIEENIVTVGDKKYAVYVGDNVSLAHQAQVHGPAAVGDNTFIGMQAFVFRSRVGKDCYLAPLAAAIGVTVPDGTYIPAGKVVTTQEEAAKLPKMTPDHPFYKTNAAVVKVNVELAKGYLAQA

SEQ ID NO: 315 - protein, 214 aa, synthetic construct
  source 1..214: note = Library of modified or engineered enzymes
MQEITVTVFNNIQPSPVTPWNPEPKLPEIHPTAYVHPLAYVQGDVKIGENVMISALASIRSDEGYPIVIGNNSNVQDNVVLHALETVDENGNEIEENIVTVGDEKYAVYIGDNVSLAHQAQVHGPAAVGDNTFIGMQAFVFNSIVGKNCVLEPLAAAIGVTIPDGTYIPAGKVVTTQEEADKLPKVTPDHPFANTNAAVVKVNVALAKGYLALS

SEQ ID NO: 316 - protein, 294 aa, synthetic construct
  source 1..294: note = Library of modified or engineered enzymes
MIRKMFCTIIAVALAGLFASSEQLSQQNPLASKSPEPAKTESNTKPAEAAKQEEHAKHWDYTENGPDKWAGLDPQNKLCSEGKMQSPIDITNPKPDQLPEVSIEFPPAVFSMTHNEHVKDIENNGHTIQVDFDEKNTDTLKIGNAKYSLSQFHFHSSSEHTVNGKSFPMEMHLVHKAGDNFAVLGIFIEEGPEDNKAFEPIWSKLPQKGKTEENINIDINQFLPKSRTTYRYEGSLTTPKCGEAVKWIVFAEPIRMSSGQIAKFRSIVKKNNRPTQPLNERVVQTDIIEEKDSK

SEQ ID NO: 317 - protein, 275 aa, synthetic construct
  source 1..275: note = Library of modified or engineered enzymes
MLTSLFLLSALFSTAWSCPKHDNYQSHPHLGRRQIRVDKGREPKDWNYDVSADWATINPEYVLCQSGTHQSPINIAQQDLSTLHKPNFEGYQSVKIPGNFFNWQFAPAWTPHHPEGDVTGLPSFNFDGEEVFNIGWHIHAPSEHLIDGKRSRAEIHMVHVTAEEHEAAVIGIRLAVGPQESAFIKQLGPMIHYNDTAQLEGLEVNLRLAIDEVGGVEEFWTYKGSLTTPPCSEGLRWFLPKQELIVSEQQMVEILAASRFSHRVEQPVWLHDINL

SEQ ID NO: 318 - protein, 261 aa, synthetic construct
  source 1..261: note = Library of modified or engineered enzymes
MRLSVFATICGYQPEHWNDKYKMASGKRQSPIDIQPKDTKYDSSLKPLTISYDPSTSKEILNNGHSFQVTFEDSNNKSVLKGGPLTDSYRLTQFHFHWGASDEHGSEHVVDGAKYSAELHLVHWNSDKYSSFAEAASKPDGLAVLGVFLKVGKEHAELQKLTDALPSIKHKDTLAKFGNFDPSCLMPSCPDYWTYAGSLTTPPLLESVTWIILKEPISVSPSQMAKFRSLLFTSEGEKEKRMVDNFRPLQPLMNRTVRSSF

SEQ ID NO: 319 - protein, 214 aa, synthetic construct
  source 1..214: note = Library of modified or engineered enzymes
MQEITVTKYENIQPSPVTPWNPEPKKPEIHPTAYIHPAAVVIGDVKIGENVMVSANASIRSDEGYPIVIGDNSNVQDNVVLHALETVDENGNEIEENIVTVGDEKYAVYVGKNVSLAHQAQVHGPAAVGDNTFIGMQAFVFRSRVGKNCVLEPLAAAIGVTVPDGTYVPAGKVVTTQEEAAKLPKVTPDHPFYNTNAAVVKVNVALAKGYLALS

SEQ ID NO: 320 - protein, 214 aa, synthetic construct
  source 1..214: note = Library of modified or engineered enzymes
MQEITVDEYSNVTKNEVTPYNPKPTTPVIDPTSYVDPNATVTGDVTIGKNVLIGAFARIRADEGQPIVIGDRSNVQDGVVLHALESVDDDGEVLEDNVVLHGDEDYAVYVGKDVSLAHQSQVHGPARVGDDSFVGMKSLVFNSDVGDNCVIEPFAAAIGVTVPDGKYIPAGTVVTTQEEAAKLPEITPDHAASNAQAAVVEVNVQLNQAYRGQA

SEQ ID NO: 321 - protein, 214 aa, synthetic construct
  source 1..214: note = Library of modified or engineered enzymes
MQEITVTKFENIQPSPVTSWNPEPKLPKIDPTAYIHPLATVVGDVTIGKNVMISANASIRSDEGYPIYIGDNSNVQDNVVLHALETVDENGNRIEENIVVVGDKEYAVYIGKNVSLAHQAQVHGPAAVGDNTFIGMQAFVFRSVVGKNCVLEPLAAALGVTVPDGRYIPAGKVVTTQEEADKLPKITPDHPFATTNAAVVKVNVELAKGYLALK

SEQ ID NO: 322 - protein, 289 aa, synthetic construct
  source 1..289: note = Library of modified or engineered enzymes
MKRISGVLATFCWPEQHNDYLLLHLLYVLKMNSWGYNESNGPATWHEHYPIANGDRQSPIDIKTKEVKYDSSLRPLSIKYDPSTAKEILNNGHSFNVEFEDSQDKSVLKGGPLTGSYRLRQFHFHWGSADDHGSEHTVDGVKYPSELHLVHWNAVKFSSFGEAALEENGLAVIGVFLKLGRHHGEFDKIVDALDSIKTKGKQASFTNFDPSCLLPPCPDYWTYSGSLTTPPLSESVTWIILKQPISVDSEQLAKFRSLLSSSEGEKASFMLSNHRPLQPLKGRKVRSSF

SEQ ID NO: 323 - protein, 214 aa, synthetic construct
  source 1..214: note = Library of modified or engineered enzymes
MQEITVFNYSNVTKNEVTPENPKPTTPEIDPTAFVDPDATVIGDVTIGKNVMISASASIRSDEGKPIVIGDRSNVQDGVVLHALESVDENGKVLEDNVVLEGDEWYAVYVGKNVSLAHQSQVHGPAMVGDDSFIGMQSFVFKSHVGSNCVIEPDAAAIGVTVPDGKYIPAGTVVTTQEEADKLPEVTPDYAYYTTVAAVVEVNVNLAQAYKSKA

SEQ ID NO: 324 - protein, 214 aa, synthetic construct
  source 1..214: note = Library of modified or engineered enzymes
MQEITVTRYENIRPSPVTPWNPTPKRPKIHPTAYIDPAAVVQGDVTIGANVLVMANAVIRADEGYPIVIGDNSSVQDNVVLHALETVDAAGRVLEENVVKVGDERYAVYVGANVVLAHQAQVHGPAAVGDNTFVGMQALVFRSTVGANCVLAPLAAAIGVTVPDGTYVPAGLVVTTQAEAAALPRVTPDHPFADLNARVVAVNVALAAGYRALA

SEQ ID NO: 325 - protein, 214 aa, synthetic construct
  source 1..214: note = Library of modified or engineered enzymes
MQEITVTRFENIRPSPVTPWNPEPKLPEIHPTAYIDPAAVVQGDVTIGENVLVMANAVIRADEGYPIYIGDNSVVQDNVVLHALETVDENGNEIEENVVVVGDKKYAVYIGKNVVIAHNAQVHGPAIVGDNTFVGMNALVFRSEVGKDCYLAPNAAAIGVKVPDGTYIPAGKVVTTQEEAAKLPKMTPDHPGYKLNERVVKVNIALAKGYLALS

SEQ ID NO: 326 - protein, 214 aa, synthetic construct
  source 1..214: note = Library of modified or engineered enzymes
MQEITVTKYNNIRPSPVTPWNPEPKLPEIHPTAYIDPAAVVQGDVTIGENVLVMANAVIRADEGYPIVIGDNSSVQDNVVLHALETVDENGNRIEENIVKVGDEEYAVYIGKNVVIAHLAQVHGPAAVGDNTFIGMLALVFNSIVGKNCVLAPLAAAIGVTIPDGKYIPAGKVVTTQEEADKLPEVTPDHPLYNLNARVVKVNVALAKGYLALS

SEQ ID NO: 327 - protein, 237 aa, synthetic construct
  source 1..237: note = Library of modified or engineered enzymes
MKLIFTVASGQEYDPNHWCRGYPIAKGNRQSPIDIDTKSAKYDSSLKPLTVSYDPATAREIVNVGHSFNVTFDDSQDKSVLRGGPLTGVYRLRQFHFHWGSSDDHGSEHVVDGVKYSAELHLVHWNAKYGSFAEAARHPDGLAVVGVFLKIGREKGEFQILLDALDAIKTKGKQTRFTNFDPSCLFPPCRDYWTYSGSLTTPPLSESVTWIVLKQPIEVDHDQLEKFRTLLFTSEGE

SEQ ID NO: 328 - protein, 253 aa, synthetic construct
  source 1..253: note = Library of modified or engineered enzymes
MITKLFAGSVQRCYEPDNWHRYYPVANGNSIDIEAAETQYDSSLKPLTISYNPATAKEILNVGHFFTVHFEDKDNQAQEKGGPLDGVYRLIQFHFHWGSIDGQGSEHTVDKKKYAAELHLVHWNAKYGDFGEAAQQPDGLAVLGIFLKVGSAKPGLQKVVDALNSIKTKGKSADFTNFDPSTLLPGCLDYWTYDGSLTTPPLLESVTWIVLKEPISVSSEQMSKFRSLLFTSEGEAACCMVDNYRPPQPLKGR

SEQ ID NO: 329 - protein, 297 aa, synthetic construct
  source 1..297: note = Library of modified or engineered enzymes
MDNNLAAAVRIVVEVLLFVFICILIWVVIQSKREEATLQVKLAGAQVQINTATEKLATTEAAFAEAEHKLDKAAKGALWEYEGRFGPDFWGKVFPTCGIGKSQAPLDIRGPFGKAKAKIQVDYKLSGLKLIHNGHTVQVNVAPGSRLLVDGVAYELLQFHFHRPSEEWIEGKPSDMSLHLVHKSADGKLAVLGVLLQAMAADNQGLVPIWTHLPSAEGPEQSFPETNVDPAKLIPSNLAYYQYEGSLTTPPCTEGVTFFILKTKMPISKGQLDAFPIPHSNARPVQPLNGRTIYSSS

SEQ ID NO: 330 - protein, 264 aa, synthetic construct
  source 1..264: note = Library of modified or engineered enzymes
MRIKRLGLSRLGIGLLSITVVGTASAEGVLATEAGPPAQGATLAWQYEGEQGPSHWGTLAPTTASCEKGTHQSPINIRTASHPHGHDGMLIQYRAASGHVGTSHHTVEVDFQSGGTLELSGRSYSLKEFHFHEPSEHQLNGRIYPMEAHLVHRDESGHLVVLAILMELGTETAPLADVWERIPSGKQEEVRDLLFNPQDLLPKDLHHYAYDGSLTTPPCTEGVHWIVLKEPIHITAAHLERFVSLIGHNARPVQPLNEREVDEE

SEQ ID NO: 331 - protein, 214 aa, synthetic construct
  source 1..214: note = Library of modified or engineered enzymes
MQEITVTRFENIRPSPVTPWNPEPRLPRIHPTAYIDPAAVVQGDVTIGANVMVSANASIRSDEGYPIVIGDNSNVQDNVVLHALETRDAAGRELEENVVTVGDEKYAVYVGANVSLAHQAQVHGPAAVGDNTFIGMQAFVFNSRVGANCVLMPLAAAIGVTVPDGTYIPAGTVVTTQEEAAKLPKVTPDHPFATTNAAVVKVNVALAAGYRALA

SEQ ID NO: 332 - protein, 214 aa, synthetic construct
  source 1..214: note = Library of modified or engineered enzymes
MQEITVLVFSNVRKNEVTPWNPKPETPKIDPTSYIDPEATVIGDVTIGKNCYIAASAVIRADEGRPIVIGDRSNVQDGVVLHALESVDDGGKVREENIVLEGGKYYAVYVGKNVVLAHQAQVHGPAWVGDDSFVGMKSLVFKAIVGSNCVLEPEAAAIGVTVPDGKYIPAGTVVTTQAEADKLPEVTPDDAKYTANVAVVNVNVNLAKAYRELA

SEQ ID NO: 333 - protein, 247 aa, synthetic construct
  source 1..247: note = Library of modified or engineered enzymes
MLNSAGIFPKRVTQECYHWDYGKHMEWEKTFPSAAGNRQSPINIQPREAQFDPSLKPLTLKYDPSTSLEILNNGHSFQVTFVDDTDSSTLTGGPITGTYRLKQFHFHWGAADDKGSEHTVDGVKYPCELHLVHWNAVKYASFAEAAAEPDGLAVVGVFLKIGQHHEELQKLVDALPSIKHKDTLVTFGSFDPSCLMPTCPDYWTYSGSLTTPPLSESVTWIIKKQPVEVDHDQLEQFRTLLFTSEGE

SEQ ID NO: 334 - protein, 261 aa, synthetic construct
  source 1..261: note = Library of modified or engineered enzymes
MKSLIAVFGTCHWNYDEQRPEEWHNDYPVANGLRQSPIDIKPAETQYDSTLRPLSFKYDPSTAKEILNNGHSFQVTFDDSSDKSVLSGGPLTGTYRLKQFHFHWGASDEHGSEHTVDGVKYAAELHLVHWNSDKYASFAEAAAKPDGLAVVGVFLKIGEANPALQKLLDALSSIKTKGKQTTFTNFDPSTLLPSSLDYWTYLGSLTVPPLLESVTWIVLKEPISVSPAQLAKFRSLLCSGEGEAACCMVDNYRPPQPLKGR

SEQ ID NO: 335 - protein, 214 aa, synthetic construct
  source 1..214: note = Library of modified or engineered enzymes
MQEITVTRFENIRPSPVTPWNPTPKLPKIHPTAYIDPAAVVQGDVTIGENVLVMANAVIRADEGYPIYIGDNSSVQDNVVLHALETVDENGNRIEENIVTVGDKEYAVYIGKNVVIAHNAQVHGPAIVGDNTFIGMNALVFRSKVGKNCVLEPLAAAIGVTIPDGTYIPAGKVVTTQEEADKLPKVTPDHPFYKLNERVVKVNIALAKGYRALS

SEQ ID NO: 336 - protein, 258 aa, synthetic construct
  source 1..258: note = Library of modified or engineered enzymes
MLRSAPGVFICTNWQEKYDHVAEGSEQSPINIVTDKAVYDSTLKPLELKYDASTALEIVNNGHSVQVKFDDSSDKAVLKGGPLTGPYRLKQFHFHWGKKDDVGSEHTVDGVKYASELHLVHWNAKYGSFGEAASQPDGLAVVGVFLKIGSAKPGLQKVVDALNSIKTKGKSADFTNFDPSTLLPSSLDYWTYDGSLTTPPLLESVTWIVLKEPISVSSEQMSKFRSLLFTSEGEAACCMVDNYRPPQPLKGRQVRASF

SEQ ID NO: 337 - protein, 214 aa, synthetic construct
  source 1..214: note = Library of modified or engineered enzymes
MQEITVLEFSNITKNEVTPWNPEPVTPVIDPTAYIDPQATVIGDVTIGANCYIAASAVIRADEGKPIVIGDRSNVQDGVVLHALESINDGGMVREDNVVEVGDENYAVYVGKNVVLAHQSQVHGPAAVGDDSFVGMKSLVFKSIVGSNCVIEPEAAAIGVTVPDGKYIPAGTVVTTQAEAAKLPEVTPDHAAYSQIAAVVAVNVALCQAYRDQA

SEQ ID NO: 338 - protein, 217 aa, synthetic construct
  source 1..217: note = Library of modified or engineered enzymes
MVLFFLLSSSYLISASTAHGEVEDESEFTYDEGSEKGPKNWGKIKPQWKACSTGKLQSPIDLLDQRVQVLPNLGELKREYKPAPAVIKNRGHDITIKWKGDAGKIKINGTDFKLQQCHWHSPSEHTFNGSRYNLEMHIVHLSAQNKIAVIAILYKYGRPDPFLSRLFHHIKTVGTEERDIGIINPGEIKFGSRKYYRYIGSLTTPPCTEGVIWTVFK

SEQ ID NO: 339 - protein, 214 aa, synthetic construct
  source 1..214: note = Library of modified or engineered enzymes
MQEITVLLFSNVQKNEVTTTNPKPTTPVIDPTSYVDPNATVVGDVTIGKNVLIWATAVIRADEGKPIVIGDRSNVQDGVVLHALESVDDGGKIRTDNVVLHGDELYAVYIGNNVSLAHQAQVHGPAHVGDDSFVGMKSLVFKSKVGSNCVIEPFAAAIGVTIPDGKYVPSGTVVTTQEEAAKLPEITADHAQYTQQAQVVSVNVRLCKAYREQK

SEQ ID NO: 340 - protein, 214 aa, synthetic construct
  source 1..214: note = Library of modified or engineered enzymes
MQEITVTLYENIRPSPVTSWNPTPKRPVIDPTAYIDPAAVVQGDVTIGKNVLVMANAVIRADEGYPIVIGDNSSVQDNVVLHALETVDENGNVIEENVVEVGDKKYAVYVGDNVVLAHLAQVHGPAAVGDNTFVGMLALVFRSRVGKNCVLEHLAAAIGVTVPDNTYVPAGTVVTTQEEAAKLPKMTPDHPHYNLVERVVKVNVELAKGYLALS

SEQ ID NO: 341 - protein, 274 aa, synthetic construct
  source 1..274: note = Library of modified or engineered enzymes
MKLNSFVAIGCTPREQYHWDEVIKGGPNSWAEYFPLANGDKQSPIDIVPGSAKYDSGLKPLTLKYDPSTSLEILNNGHSFQVTFSDDTDSSTLTEGPISGVYRLKQFHFHWGASDDKGSEHTVDGVKYAAELHLVHWNAVKYSSFGEAASKPDGLAVLGVFLKVGKHHGEFEKIVNALGSIKHKDTLATFENFDPSCLMPACPDYWTYDGSLTTPPLLESVTWIVLKEPISVSPSQMAKFRSLLFTSEGEKACCMVDNYRPPQPLKGRQVRASF

SEQ ID NO: 342 - protein, 214 aa, synthetic construct
  source 1..214: note = Library of modified or engineered enzymes
MQEITVTVFENIQPSPVTPWNPEPKRPEIHPTAYIHPAAVVQGDVTIGANVLVMANAVIRADEGYPIVVGDNSAVQDNVVLHALETVDASGKELEENIVTVGDEKYAVYVGDNVVLAHNAQVHGPAAVGDNTFVGMNALVFRSRVGANCVLEHLAAALGVTVPDGRYVPAGRVVTTQEEAARLPAVTPDHPHADLNARVVAVNVALAKGYLALA

SEQ ID NO: 343 - protein, 276 aa, synthetic construct
  source 1..276: note = Library of modified or engineered enzymes
MKKTIMLVPVLFVFIAIFMTCDNKTNNHKDVKHSKETKEEMKKETAKKDCDQVHWSHHKGEHGPENWANLCEGFKDCNGEKQSPIDIKEAVKGEDLKPLEFEYGKTKVNIINNGHTVQFNIDKGSSMMVDGKKYDLLQFHYHATSEHTIKGEYSPLEVHFVHRHADDDFAVLGIMYEEGEANDLFNKYLKHFPADKGEYTSDEKFDLDALLPDNLSYYHYGGSLTTPPCSEVVSWYLLQNPLRASQEQIKDFSEILDKNFRPIQELNGRTIYKFGE

SEQ ID NO: 344 - protein, 264 aa, synthetic construct
  source 1..264: note = Library of modified or engineered enzymes
MKRLASVFTICGWNYQDPEHWHELFPTAKGNHQSPINIETRKTIYDSTLKPLTFSYEAATSRRIVNNGHSFQVEFEDTDNKSVLKGGPLTDRYRLTQFHFHWGSSDDHGSEHTVDGLKYPAELHLVHWNAKYGSFGEAASKPDGLAVVGVFLKIGRENAEFQLVLDALDSIKTKGKQTPFTNFDPSCLFPACRDYWTYSGSLTTPPLSESVTWIVLKQPIEVSPRQLSKFRSLLFTSEGEKEKRMVDNFRPLQPLMNRSVRSSF

SEQ ID NO: 345 - protein, 332 aa, synthetic construct
  source 1..332: note = Library of modified or engineered enzymes
MITRLPAVTAVLAMFVVCSMAHDPWNTDYTSQRGPLFWGQRPEFKMCGLGREQSPINIRRSSTIYQDFPPLAFELKSPIVHSNIENKGSATAAFPLSDIPILSGGPLGNRKYRVYNVHLHFGNYSFRAAEHAFDGVRTTGEFHIVTYDSRYPHIKAALGSGRRGALAVLGVMFEARNVSNIDMGVTNLIELSSNVTYKGDHYMTGIDFSNLVSEVDMGYYYAYNGSLTTPTCNEVVQWMVIDRIHYVLPETADLLLELKTGYRREHSIPIFGNTRPLQPLYGRKVLRSFGPVVTDVHDQGEEIVYSSADLVGPLGRVMLLALAAIATFVIKA

SEQ ID NO: 346 - protein, 242 aa, synthetic construct
  source 1..242: note = Library of modified or engineered enzymes
MKLTSAFIVCGYQNHEPRDWHEVAPSAKGNRQSPINIQWRDSVYDPGLKPLTISYDPATCLHIWNNGYSFLVEFEDSTDKSVIKGGPLENNYRLKQFHFHWGATDDHGSEHTVDGVKYSAELHLVHWNADKFDSFVEAAHEKDGLAVLGVFLKIGEHNAQLQKITDILDSIKEKGKQTRFTNFDPVCLLPPCPDYWTYPGSLTVPPLSESVTWIILKQPINISSQQLAKFRTLLFTSEGETA

SEQ ID NO: 347 - protein, 335 aa, synthetic construct
  source 1..335: note = Library of modified or engineered enzymes
MNNRPIQPLNDRSIWINRIKTEKCEFGWCPPVEEEPEKASKKVEKDDDSKASSKQGKSDKKGKSSGDKKSGKKSGKSNKKEKEPPQWNYASVQRWEDDYSMCGGKKQSPVNANTSKIQSVQGPGAGLVSRMAYSAVGPNAGFQFKNNGKSLVLEGNWGTLRLPDGDYIAKSIKFHFPSEHAVDGVLAAGEMHIVHQRSDATGTDGLAVIAILLRDSDLLGQAGPVGFFDRLGFSSRLPVEGETVILGADTVLDIGAIFAPQLGGKYWHYEGSLTTPPCSETVHWYLMQTPAGINKAMVNNFKSLFPSPANNRPVQGMYGRAIVVTELSVSSKEFD

SEQ ID NO: 348 - protein, 252 aa, synthetic construct
  source 1..252: note = Library of modified or engineered enzymes
MKFAAAVASIVFAVSGAAAIAAPEGAVDWVYGDGDLPEKWSITNEAYGACDAGNMQSPIDLDLANTRGEIEFASSYEETTGELKTGPSKVQVDVAPGMGMISGQHLFSLVQFHFHTPSEHRLHGQRYPLTVHLVHGTATGDFAVLGVMFEEGDENPALARILSGIDGGSKNVAVDVRELVPENIDVYRYMGSLTTPPCTEGVLWLILKQPASISAEQLRLFSQLYPNNARPVQSLNGRPVRDALLIAPGGRP

SEQ ID NO: 349 - protein, 214 aa, synthetic construct
  source 1..214: note = Library of modified or engineered enzymes
MQEITVTKYENIRPSPVTPWNPEPKLPKIHPTAYIDPAAVVQGDVTIGANVMVSANASIRSDEGYPIYIGDNSNVQDNVVLHALETVDENGKPLEENIVKVGDKDYAVYVGDNVSLAHQAQVHGPAAVGDNTFIGMQAFVFRSRVGKNCVLEPLAAAIGVTVPDGTYVPAGTVVTTQEEAAKLPKVTPDHPFYKTNEAVVKVNIALAKGYLALK

SEQ ID NO: 350 - protein, 214 aa, synthetic construct
  source 1..214: note = Library of modified or engineered enzymes
MQEITVLTYSNVTKNEVTSTNPKPTTPVIDPTSYVDPNATVTGDVTIGKNCFIGAFAVIRADEGKPIVIGDRSNVQDGVVLHALESVDDGGEIREDNVVVHGDEYYAVYIGKNVSLAHQAQVHGPAHVGDDSFVGMKSLVFNSIVGDNCVIEPDAAAIGVTVPDGKYIPAGTVVTTQEEAAKLPEITPDHEFYTQIAAVVQVNVDLCKAYRDKK

SEQ ID NO: 351 - protein, 260 aa, synthetic construct
  source 1..260: note = Library of modified or engineered enzymes
MLKEWGYASHNGPDTWVQIFRCARGNNQSPIELKTKDIKHDPSLQPLSVSYDPGTAKEIVNVGHSFHVNFEDSDNRSVLKDGPITGSYRLRQFHFHWGASDDHGSEHVVDGVRYAAELHVVHWNADKYPSFVEAAHEPDGLAVLGVFLKIGEHNPHLQKITDVLYAVKFKGTKAQFTNFNPKCLLPASLDYWTYPGSLTTPPLSECVTWIVLKEPISVSPSQMAKFRSLLFSSEGETACCMVDNYRPPQPLKGRTVRASF

SEQ ID NO: 352 - protein, 214 aa, synthetic construct
  source 1..214: note = Library of modified or engineered enzymes
MQEITVLEFSNITKNEVTPTNPKPSTPVIDPTSYVDPNATVIGDVTIGKNVLIAANAVIRADEGAPIVIGDRSNVQDGVVLHALESVDDDGKILEDNVVEKGDEYYAVYIGKNVHLAHQAQVHGPARVGDDSFVGMKSLVFKSKVGSNCVIEPDAAAIGVTVPDNKYIPAGTVVTTQEEADKLPEVTPDYAYSDTNEAVVTVNVDLNEAYRNQQ

SEQ ID NO: 353 - protein, 214 aa, synthetic construct
  source 1..214: note = Library of modified or engineered enzymes
MQEITVTKYENIRPSPVTSWNPEPKLPKIHPTAYIDPAAVVQGDVTIGENVMISALASIRSDEGYPIYIGNNSNVQDQVVLHALETVDENGNVLEENVVTVGDKKYAVYIGDNVSLAHQAQVHGPAAVGNNTFIGMQSFVFRSVVGENCVLEPLAAAIGVTVPDGTYVPAGTVVTTQEEADKLPKVTPDHPFYNTNAAVVKVNVELAKGYLALK

SEQ ID NO: 354 - protein, 214 aa, synthetic construct
  source 1..214: note = Library of modified or engineered enzymes
MQEITVLNYSNIEKNEVTSTNPKPTTPVIDPTSYIDPNATVTGDVTIGKNCYIGPFAVIRADEGAPIVIGDNSNVQDGVVLHALESVDDGGKIRKDNVVEVGDNNYAVYVGKNVSLAHQSQVHGPARVGDDSFVGMNATVFNSIVGSNCVIEPNAAAIGVTVPDGKYIPAGTVVTTQEEADKLPEITEDYPFYTAVEEVVKVNVALCKAYREKK

SEQ ID NO: 355 - protein, 272 aa, synthetic construct
  source 1..272: note = Library of modified or engineered enzymes
MLSRKPAGHTDIYEQFVCNWKNGGGKLHRSSATDPEGERQSPIDIQTSKVEVDQKLQPLTLTYDPSTSLEILNNGHSVQVTFKDKDNRSVLKGGPLTGPYRLKQFHFHWGKKDDVGSEHTVDGAKFASELHLVHWNAKKYSSFAEAASKSDGLAVLGVFLQVGEHNAQLQKITDILDSIKEKGKQTRFTNFDPLSLLPPCRDYWTYHGSLTVPPLLESVTWIILKQPISVSSQQLAKFRSLLCTSEGEKAVPMLSNHRPPQPLKGRQVRASF

SEQ ID NO: 356 - protein, 333 aa, synthetic construct
  source 1..333: note = Library of modified or engineered enzymes
MTMKLSVLGQLFLVICCLTVVINAMPNPQAQAPSGALVATAESGHFSYDDPNKWKEHNSLCAGEHQSPINIDTRKSRTDKFPPFRFHNYAKGLPENLENNGHTVQLTIDNLIKDLPTISGGGLEGPYEFAQMHFHWGEDEFGSEHKINNKQYAGEVHIVHWNKKYGNFVNATKHNDGLAVLGILIDLQDKENIAFSHIEQFDEIRDASKKNEKLPYSVPLKDLLPSNTASFFRYEGSLTDARCNEDVTGPFLKHQFTSPIIRQLNDDEGEPLSKNVRPTQEEHDRIVTYSGRAMQCGTCSTATADRSSEGRSDSSESGESKEITKSKTRHYGY

SEQ ID NO: 357 - protein, 214 aa, synthetic construct
  source 1..214: note = Library of modified or engineered enzymes
MQEITVTRYENIRPSPVTPWNPEPRLPEIHPTAYIDPAAVVTGDVRIGANVMVSALASIRSDEGYPIVIGDNSNVQDQVVLHALETVDADGKVLEENVVEVGDERYAVYIGDNVSLAHQAQVHGPAAVGDNTFIGMQAFVFRSRVGKDCVLMPLAAAIGVEVPDGRYIPAGKVVTTQEEADKLPKVTPDHPFANTNAAVVAVNVALAKGYLALA

SEQ ID NO: 358 - protein, 214 aa, synthetic construct
  source 1..214: note = Library of modified or engineered enzymes
MQEITVTTYNNIRPSPVTPWNPEPKLPKIHPTAYIDPAAVVQGDVTIGENVLVMANAVIRADEGYPIVIGNNSSVQDNVVLHALETVDEDGKRIEENVVKVGDKDYAVYVGDNVVLAHNAQVHGPAAVGDNTFVGMNALVFHSNVGKNCVLAPNAAAIGVTVPDGKYIPAGKVVTTQEEADKLPEVTPDHPFYDLNARVVKVNVALAKGYLALA

SEQ ID NO: 359 - protein, 267 aa, synthetic construct
  source 1..267: note = Library of modified or engineered enzymes
MAMLNKKRKEWKMRGRVFFALILAMCLSVAGYSLYEKEQNQHDEERIEDVYYSYDEHGPDAANVCERGMMQSPVQITRKDALQNQSPEIEIHYGEGRFEIIKKAHTAEAVSKSGQNYILIDHQQYKLESFHFHLPSEHQVEGQSYEMELHFVHENKNGEQAVMAVFIQEGQANEMVKEIWSRLQDGFSKKDNVSIRLPEFIPKERRAFYYTGSLTTPPCTEGVKWIVFEMPVEFSEEQIGTFHRLFGNNSRQVQPLNGRKIYQLTVR

SEQ ID NO: 360 - protein, 293 aa, synthetic construct
  source 1..293: note = Library of modified or engineered enzymes
MKHCTLLAILLSGLLSAAVETDFSYADQGAWQTLPNSQCGGRRQSPVDLDLRNVTVDKILGQDLACTWQQNKAVIEGDLVNTGRTIELDVRSPHTCRGVPGSPSAHFRLAAVHIHYGSASDQGSEHTINGRTSALEVHMVHFDTRFASLDKAREQPGGIMVAGLLFDEADEAIANPELTKMAVISGTALRSTGGVLASRLNAAPLIEGTGLDKARARFLTYAGSLTTPTCNEVVTWIVAAEPGLVGHQTMHLLRTVTGLGNKTISPNFRQVQPLNGRTITSSFQPACGLHGRC

SEQ ID NO: 361 - protein, 266 aa, synthetic construct
  source 1..266: note = Library of modified or engineered enzymes
MLKNPAGDWSYEQTVHFIRCTDFIPAVILPGGARQSPINIVTSQAVYSPSLKPLELSYEACTSLSITNNGHSVQVEFNDSTDRTVIKGGPLEGPYRLKQFHFHWGARDSRGSEHTVDGARYPSELHLVHWNAKYASFGEAASQPDGLAVVGVFLKIGREKPGLQKVLDALDAIKTKGKQTRFTNFDPSTLLPGCLDYWTYDGSLTTPPLLESVTWIVLKEPISVSSGQMAKFRSLLFTSEGETACCMVDNYRPPQPLKGRQVRASF

SEQ ID NO: 362 - protein, 214 aa, synthetic construct
  source 1..214: note = Library of modified or engineered enzymes
MQEITVLEYSNIRKNEVTPWNPKPSTPVIDPTSYIDPNATVIGDVTIGANVLIGPNAVIRADEGRPIVIGDRSNVQDGVVLHALESVNDEGMEIGDNVVLEGNSYYAVYIGKNVVLAHQSQVHGPAAVGDDSFVGMQSLVFNSIVGSNCVIEPNAAAIGVTVPDGKYIPAGTVVTTQAEADKLPEVTPDHAFYTDVAKVVSVNVNLCRAYKEQS

SEQ ID NO: 363 - protein, 186 aa, synthetic construct
  source 1..186: note = Library of modified or engineered enzymes
MIRKNPSGHLPVIAETAFIDQTAIICGKVIIYDNVFVGPYAVIRADEVNEHGDMEAIVIKRDTNIQDGVVIHSKAGAAVTIGERSSIAHRSIIHGPCWVGDDVFIGFNSVVFNAKIGKGCVIRHNSVVDGLDLPEHFHVPPMTNIGADFDLSSISKVPPEYSAFSESVVSANHELVQGYRRIANEL

SEQ ID NO: 364 - protein, 212 aa, synthetic construct
  source 1..212: note = Library of modified or engineered enzymes
MRILVTACFYPNSEHDGQKWANIKPEWKTCGHGKMQSPIDLSSHRVSLVHDQTWNRDYKPAPAVIVNRGHDIMVSWKEDAGKVTIHQTDYKLVQCHWHSPSEHTVNGTRYDLELHMVHTSAQGKTAVIGVLYKLGRPNEFLAKLLDGIKSVGKEEKDLGIVDPRTIGFHTDKFYRYVGSLTTPPCTEGVIWTVVKKVNTVSMEQLAALREAV

SEQ ID NO: 365 - protein, 214 aa, synthetic construct
  source 1..214: note = Library of modified or engineered enzymes
MQEITVTRYENIRPSPVTSWNPEPKLPKIDPTAYVDPAAVVQGDVTIGKNVMVSANASIRSDEGYPIYIGDNSNVQDNVVLHALETVDADGKRIEKNIVTVGDKEYAVYVGDNVSLAHQAQVHGPAAVGDNTFIGMQSFVFNSTVGKNCYLAPLAAAIGVTIPDGTYIPAGKVVTTQEEAAKLPKITPDHPFANTNAAVVKVNVELAKGYLALK

SEQ ID NO: 366 - protein, 237 aa, synthetic construct
  source 1..237: note = Library of modified or engineered enzymes
MSTPLVRWGYKEDNGAHQWCIFFPEACGKRQSPINIQTSKVVYDPGLRPLNLNYDPSTSLEILNNGHSVQVNFKETDDRSVLSGGPVTGTYRLRQFHFHWGAKDCRGSEHTVAGVKYPSELHLVHWNAVKYESFAEAALEENGLAVIGVFLKLGKHHDELQKLVDALPSIKHKDTLVEFGSFDPSCLMPTCPDYWTYSGSLTTPPLSESVTWIIKKQPVEVDHDQLEQFRSLLFTSE

SEQ ID NO: 367 - protein, 305 aa, synthetic construct
  source 1..305: note = Library of modified or engineered enzymes
MSSTFVVGRPCDTRLPRGSRKEPARVPHGFAAGIATVVSLALLCLAGGCAHAPVPREAGRSVAQSEADYYSDALAPWTYPEGPSWGAACAKQPPPQQSPIDLTRVTTAPWSASSVITQATFDGHDQNVVFQASPGPSVTMAPGVDGSGRAFVYTVAGFHFHYRNEHVIAGNPVYELHIKTVDQHGGVAVFAVLWTADDAAGEDPTLAAAYRSLSAPPDSVVAVDLGRALWRFGQQPFYSYVGSLTTPPCTTGIRWFVLQTPIRTSSASIGRLNAALIARGMPRDNVRTVRPVAQPQPVVYLVTPK

SEQ ID NO: 368 - protein, 214 aa, synthetic construct
  source 1..214: note = Library of modified or engineered enzymes
MQEITVLEFSNITKNEVTPTNPKPTTPVIDPTSYVDPNATVTGDVTIGKNVLIGPNAVIRADEGRPIVIGDNSSVQDGVVLHALESVDDEGKIIEDNVVLYGNKYYAVYIGKNVVLAHQSQVHGPAAVGDDSFVGMNSLVFNSIVGSNCVIEPNAAAIGVTVPDGKYIPAGTVVTTQEEADKLPEVTEDYKFYTQVAKVVTVNVNLCEAYRNQA

SEQ ID NO: 369 - protein, 214 aa, synthetic construct
  source 1..214: note = Library of modified or engineered enzymes
MQEITVTTFTNIRPSPVTPWNPTPKLPKIHPTAYVDPAAVVQGDVTIGENVMISANASIRSDEGYPIYIGNNSNVQDNVVLHALETVDENGKVIEENVVTVGDKKYAVYIGDNVSLAHQAQVHGPAAVGDNTFIGMQAFVFKSNIGKNCVLEPLAAAIGVTVPDNKYIPAGTVVTTQEEAAKLPEVTPDHPFANTNAAVVKVNNALAAGYLALS

SEQ ID NO: 370 - protein, 283 aa, synthetic construct
  source 1..283: note = Library of modified or engineered enzymes
MKLSNVFIAGCTYQEPWDHREWDYSKKGPATWGLINSAWSLCSIGKRQSPIDIELNQLLYDPFLPPLRLSSGGKKLGGTMYNTGRHVSFRPDKAQLVNISGGPLSYSHRLEEIRLHFGSEDSQGSEHLLNGEAFSGEVQLIHYNQELYSNFSEAARKPNGLLIISIFMKVADTSNPFLNRMLNRDTITRISYKNDAYFLMNLNIELLYPESFGFITYQGSMSTPPCYETATWILIDRPINITSLQMHSLRLLSQNLPSQIFLSMSDNSRPLQPLAHRALRGNR

SEQ ID NO: 371 - protein, 214 aa, synthetic construct
  source 1..214: note = Library of modified or engineered enzymes
MQEITVTRYENIRASPVTPWNPTPKLPKIHPTAYVHPLAEVVGDVTIGANVMVSANASIRSDEGYPIVIGDNSNVQDNVVLHALETVDANGNRIEENIVKVGDEEYAVYVGDNVSLAHQAQVHGPAAVGDNTFIGMQAFVFRSVVGKNCYLAPLAAAIGVTVPDNTYIPAGKVVTTQEEADKLPKMTPDHPFYNTNKAVVKVNVALAKGYLALA

SEQ ID NO: 372 - protein, 280 aa, synthetic construct
  source 1..280: note = Library of modified or engineered enzymes
MRSPLAGWTIVFCQKDEYNHLDGILGGPGYWGLINPEWRMCSKGKMQSPIDIDPKVLLYDPNLSAVHLDKHKVSGTLENTGQSLVFRVDKGSRQHVNISGGPLAYRYQFHEIFLHYGLKDSMGSEHRINGYSFPAEIQLYGFNSELYHNMSEAQHKSQGIVGVSLMVQIGETPNPELRILTSQLERVRYRGQSAPIHHLSLRGLLPDTEHYMTYEGSTTHPGCWETTVWVILNKPIYITRQELYALRRLMQGSQSQPKAPLGNNARPVQDLHGRTVRTNI

SEQ ID NO: 373 - protein, 221 aa, synthetic construct
  source 1..221: note = Library of modified or engineered enzymes
MLISRFATGHPVKENDCYQWFGPNGEKGPDKWGKINPKWKVCGEGKLQSPIDLLNQRVQILPNLGKLQKDYKPAPAVLKNRGHDIMVKWKGDAGKLNINGTYYKLVQCHWHTPSEHTINGTKFDMELHAVHKSSKGETAVIGIWYKIGRPDSFLSKLLKNIKSVGDKEIDLGVINPGDIKFGSRKYYRYMGSLTVPPCTEGVIWTIVKKVRTVSREQLRAL

SEQ ID NO: 374 - protein, 224 aa, synthetic construct
  source 1..224: note = Library of modified or engineered enzymes
MKRSLIFAVGTCPQEDWHYNYDEASGRGPSRWGLLKPEWRTCSVGKLQSPIDIGTVQVSSELGDLQRNYRSAPALLRNRTEDVAVIWLGNAGSITINGVVYRVVNCHWHSPSEHTFNGTRLPLEIHIVHRSSQNRIAVVGILYKYGLPDPFLSKLFHSIKSLGKEEKNLGIVNPESIGFQDKKYYRYIGSLTTPPCSEGVVWTVFKKVRTVSREQLKALKDAVD

SEQ ID NO: 375 - protein, 214 aa, synthetic construct
  source 1..214: note = Library of modified or engineered enzymes
MQEITVTRFENIRPSPVTPWNPEPKLPKIHPTAYIDPAAVVQGDVTIGANVMVSANASIRSDEGYPIVIGDNSNVQDNVVLHALETVDENGNVLEENVVTVGDEKYAVYVGDNVSLAHQAQVHGPAIVGDNTFIGMQAFVFRSRVGKNCVLAPLAAAIGVTVPDGTYVPAGKVVTTQEEAAKLPKVTPDHPFANTNAAVVKVNVALAKGYLALA

SEQ ID NO: 376 - protein, 214 aa, synthetic construct
  source 1..214: note = Library of modified or engineered enzymes
MQEITVTKYNNIRASPVTPWNPTPKLPNIHPTAYIDPAAVVQGDVTIGANVMVSANASIRSDEGYPIYIGDNSNVQDNVVLHALETVDENGNPIKENIVKVGDKDYAVYVGDNVSLAHQAQVHGPAAVGDNTFIGMQSFVFASEVGKNCYLAPLAAAIGVKVPDNTYIPAGKVVTTQEEAAKLPKITPDHPFANTNAAVVKVNVALAKGYLAQS

SEQ ID NO: 377 - protein, 214 aa, synthetic construct
  source 1..214: note = Library of modified or engineered enzymes
MQEITVLIYSNVTKNEVTTWNPKPKTPKIDPTSYVDPKATVIGDVTIGKNCMISPFASIRSDEGMPIVIGDNSNVQDGVVLHALETVDTNGKIIEDNVVIKGDKRYAVYVGKNVSLAHQSQVHGPARVGDDSFIGMQSFVFKSIVGSNCVIEPNAAAIGVTVPDNKYIPAGTVVTTQEEADKLPEITEDYKYYNTNDAVVYVNVKLCKAYRNKS

SEQ ID NO: 378 - protein, 214 aa, synthetic construct
  source 1..214: note = Library of modified or engineered enzymes
MQEITVTNFNNIQPSPVTPWNPEPKLPKIHPTAYIHPLAYVQGDVTIGENVLVMANAVIRADEGYPIVIGNNSSVQDNVVLHALETVDENGNRIEENIVKVGDEEYAVYIGDNVVLAHNAQVHGPAAVGDNTFVGMNALVFRSRVGKNCVLEPLAAAIGVTIPDGTYIPAGKVVTTQEEAAKLPKITPDHPFYNLVDRVVKVNVALAKGYLALS

SEQ ID NO: 379 - protein, 227 aa, synthetic construct
  source 1..227: note = Library of modified or engineered enzymes
MRKSLFACTVIGWNYEDQPHWSELDPAYAACATGKEQSPIDIRGARRADLPPLRFEYRSAPLKYVINNGYTIRVNYHDSPGSGNFLIVGDARYQLTQFHFHRPSEEYVHGKPYTMELHLMHQSSDGEVAGVAVLLKAGRANATIQRLWEHMPATEGQEQVLAGVTIDPAGLLPRETGYYVYMGSVTAPPCTEGVTWFVLKTPVEISAEQIAVFARLYPHDVRPLQPL

SEQ ID NO: 380 - protein, 214 aa, synthetic construct
  source 1..214: note = Library of modified or engineered enzymes
MQEITVTRYENIRPSPVTPWNPEPRLPEIHPTAYIDPKAVVQGDVTIGENVLVMANAVIRADEGYPIVIGDNSSVQDNVVLHALETVDENGNRLKENVVTVGDKEYAVYIGKNVVIAHLAQVHGPAAVGDNTFVGMLALVFNSTIGKNCYLAPLAAAIGVTVPDGTYIPAGTVVTTQEEAAKLPKITPDHPFADLNARVVKVNIALAKGYLALA

SEQ ID NO: 381 - protein, 214 aa, synthetic construct
  source 1..214: note = Library of modified or engineered enzymes
MQEITVTVYENIRESPVTSWNPTPRRPEIHPTAYVDPAAVVVGDVRIGANVMVSANASIRSDEGYPIVIGDNSNVQDNVVLHALETVDAAGNRITENIVTVGDEEYAVYVGDNVSLAHQAQVHGPAAVGDNTFIGMQAFVFRSNVGKNCVLEPLAAAIGVTVPDGTYVPAGKVVTTQEEAAKLPKVTPDHPFANTNAAVVAVNVELAKGYLALA

SEQ ID NO: 382 - protein, 214 aa, synthetic construct
  source 1..214: note = Library of modified or engineered enzymes
MQEITVTRYENIRPSPVTPWNPEPKLPKIHPTAYVDPAAVVVGDVTIGANVMVSANASIRSDEGYPIYIGDNSNVQDNVVLHALETVDENGKVIEENVVTVGDKKYAVYIGKNVSLAHQAQVHGPAAVGDNTFIGMQAFVFRSVVGKDCVLEPLAAAIGVTVPDGTYIPAGKVVTTQEEAAKLPKITPDHPFANTNAAVVKVNVALAKGYLALA

SEQ ID NO: 383 - protein, 214 aa, synthetic construct
  source 1..214: note = Library of modified or engineered enzymes
MQEITVFEFSNITKNEVTPTNPKPTTPVIDPTSYIDPNATVTGDVTIGKNVLIGPNAVIRADEGAPIVIGDNSSVQDGVVLHALESVDDEGEIIEDNVVLEGDEYYAVYIGKNVVLAHQAQVHGPAMVGDDSFVGMKALVFKSKVGSNCVIEPEAAAIGVTVPDGKYIPAGTVVTTQAEADKLPEVTDDYPFYTAVEEVVEVNVNLAEAYREQS

SEQ ID NO: 384 - protein, 214 aa, synthetic construct
  source 1..214: note = Library of modified or engineered enzymes
MQEITVMDFSNIVKNEVTPTNPKPTTPVIDPTSYIDPNATVIGDVTIGKNCYIGPFAVIRADEGAPIVIGDDSNVQDGVVLHALESVDAGGKIREDNVVTVGDRSYAVYVGKNVSLAHQSQVHGPARVGDDSFVGMNSLVFNSIVGDNCVIEPGAAAIGVTVPDGKYIPAGTVVTTQAEAAKLPEVTPDHAAYSANAAVVEVNVALSEAYRNLK

SEQ ID NO: 385 - protein, 271 aa, synthetic construct
  source 1..271: note = Library of modified or engineered enzymes
MVKRNILADFSPEGQTHWCYDCFIRPLPPVKWAKLFPKAKGNFQSPINIESRETRYDPSLKPLTLKYDPSTAKLISNSGHSFNVDFDDTEDKSVLRGGPLTGSYRLRQFHLHWGSADDHGSEHAVDGVKYAAELHVVHWNAVKFESFEEAALEENGLAVIGVFLKLGEHNPHLQKITDILYSIKFKDTLAEFTNFNPKCLLPTSLDYWTYSGSLTTPPLLESVTWIVLKEPISVSSEQMAKFRSLLFTSEGETACCMVDNYRPPQPLKGRQ

SEQ ID NO: 386 - protein, 238 aa, synthetic construct
  source 1..238: note = Library of modified or engineered enzymes
MLSKRIFGVATCQPENWHDYYKNANGEKQSPINIVTKETKYDSSLKPLTFKYDPSTAKEIVNVGHSFHVNFEDSENKSVLKGGPLTGTYRLKQFHFHWGSADDKGSEHTVDGVKYPSELHLVHWNAVKFESFAEAALEENGLAVIGVFLKLGEHHKELQKLTDTLPSIKHKDTLANFGSFDPSCLMPTCPDYWTYPGSLTTPPLSESVTWIVLKQPIEVSEEQLAAFRSLLFTSEGEK

SEQ ID NO: 387 - protein, 214 aa, synthetic construct
  source 1..214: note = Library of modified or engineered enzymes
MQEITVLEFSNVTKNEVTSWNPKPSTPVIDPTSYVDPNATVIGDVTIGKNCYIAASAVIRADEGKPIVIGDNSNVQDGVVLHALESVDDGGKIREDNVVIHGDKWYAVYIGKNVSLAHQSQVHGPAYVGDDSFVGMNSLVFKSIVGSNCVIEPNAAAIGVTVPDGKYIPAGTVVTTQAEADKLPEITPDYAFYTQVAAVVKVNVNLCRAYRNQA

SEQ ID NO: 388 - protein, 214 aa, synthetic construct
  source 1..214: note = Library of modified or engineered enzymes
MQEITVLIYSNVTKNEVTSTNPKPVTPVIDPTSYVDPNATVTGDVTIGKNCLIGPSAVIRADEGAPIVIGDRSNVQDGVVLHALESVDDEGKIIEENVVVHGDKYYAVYIGKDVVLAHQAQVHGPARVGDHSFVGMKSLVFNSIVGSNCVIEPNAAAIGVTVPDGKYIPAGTVVTTQEEAAKLPEITPDHEKYTKIAEVVTVNVNLCRAYRNKA

SEQ ID NO: 389 - protein, 274 aa, synthetic construct
  source 1..274: note = Library of modified or engineered enzymes
MIKLSVAFCTGNQRPEWDYHNNGHGKEEWPEEYPSCGGQLQSPIDLHGDILQYDASLTPLQFQGYNVSATEQFTLTNNGHSVQLSLPSDMYLKGLPSRYTATQLHLHWGKKGDLEGSEHQINSEATAAELHIVHYDSEKYSNISEAMNKPQGLAVLGILIEVGETENPAYDHILSRLHEIRYKDQKTSVPGFNIRELLPEQLEEYYRYQGSLTTPPCYQSVLWTLFNRRAQISMGQLEKLQETLSSTESEPSEPLVQNYRVPQPLNQRTVFASF

SEQ ID NO: 390 - protein, 327 aa, synthetic construct
  source 1..327: note = Library of modified or engineered enzymes
MGHTWCNDEEGTRRGRSEGPTPAAAGVRVERMIREGGSRRRAATPHVRCGVLYAVRGVPMSARSWLTASALTVAAVTLIGCAQAAPAETAPTERPVAEPAHWSYDGDSGPESWAGLDDAFQACEAGTDQSPIDLPAAVPAPSTSIELSAEEAEGDVFDSGHAVEIETDGQGETLTFADDDYSLQQLHAHVPSEHTVAGQPAAAELHLVHADADGNLLVLGVLVTEGAASDALTPFIEAASHLADDEEVTLDLAAVLPASLENYEYSGSLTTPPCTEDVQWVVMGTPISMSAEQIGTLAGAHNHNARPTQPLGDRTVVGGAGKVEITG

SEQ ID NO: 391 - protein, 186 aa, synthetic construct
  source 1..186: note = Library of modified or engineered enzymes
MARKNPSGHLPQVSETAFIDPTAIICGKVIIEDYVFIGPYAVIRADELNAAGDMEPIVIGAHSNIQDGVVIHSKSGAAVTIGEFSSIAHRSIVHGPCWIGDRVFIGFNSVLFNCHIQSGCVVRYNAVVDGVTLPENTYIPSTERVGPDSDLSLYRQVDRGALQFSEEVAATNVELVRGYQALRNEF

SEQ ID NO: 392 - protein, 214 aa, synthetic construct
  source 1..214: note = Library of modified or engineered enzymes
MQEITVTRYENIRESPVTPWNPTPKRPKIHPTAYIDPLAYVQGDVTIGENVMVSALASIRSDEGYPIYIGNNSNVQDNVVLHALETVDENGKEIEENIVTVGDEKYAVAIGDNVSLAHQAQVHGPAIVGDNTFIGMQAFVFRSKVGKNCVLEPLAAAIGVTVPDNTYIPAGTVVTTQEEAAKLPKVTPDHPFANTNAAVVKVNVALAKGYLALA

SEQ ID NO: 393 - protein, 200 aa, synthetic construct
  source 1..200: note = Library of modified or engineered enzymes
MNPITSFNPVQRYPKIDKTAFISPFSSVIGDVRIKDNVYVAPNVSIRADEGTPFYIGSNTNLQDGVILHGLLNKFVTVNDKKYSIYIGNQVSIAHDALIHGPCYIGDKVFVGFKAIVYNAIVGKGTVISYNAVVTNGVRIAPNRFVPPGANIDTQEKADALSRVPKDEEEFAREVQRVNQEFPASYHLLFGENRCSCGLS

SEQ ID NO: 394 - protein, 214 aa, synthetic construct
  source 1..214: note = Library of modified or engineered enzymes
MQEITVLDFSNVTKNEVTPTNPKPKTPVIDPTSYIDPNATVIGDVTIGKNVMVWPSAVIRADEGKPIVIGDNSSVQDGVVLHALESVDDGGKVIEDNVVLEGNKRYAVYIGKNVTLAHQSQVHGPAYVGDDSFVGMSSFVFNSKVGSNCVIEPNAAAIGVTVPDGKYIPAGTVVTSQAEADKLPEITDDYPYSNAIAAVVKVNVQLCEAYKAQA

SEQ ID NO: 395 - protein, 214 aa, synthetic construct
  source 1..214: note = Library of modified or engineered enzymes
MQEITVLRFSNIRKNEVTPENPEPETPVIDPTSYIDPNATVIGDVTIGANCYIGPFARIRADEGRPIVIGDRSNVQDGVVLHALESVDAEGEIIEDNVVIEGDELYAVYIGRDVSLAHQSQVHGPARVGDDSFVGMKSLVFKSDVGSNCVIEPFAAAIGVTIPDGKYIPAGTVVTSQAEAEKLPEITEDYPFYTTIEEVVKVNVNLAKAYREQK

SEQ ID NO: 396 - protein, 214 aa, synthetic construct
  source 1..214: note = Library of modified or engineered enzymes
MQEITVMEFSNVTKNEVTSTNPKPKTPVIDPTSYVDPEATVIGDVTIGKNCYIGPFARIRADEGAPIVIGDDSSVQDGVVLHALESVDADGKIIEDNVVLHGDKLYAVHIGKNVSLAHQAQVHGPARVGDDSFVGMNSLVFNSVVGSNCVIEPNAAAIGVTVPDGKYIPAGTVVTTQEEADKLPEITPDYAKSTAIAAVVEVNVALCEAYREQA

SEQ ID NO: 397 - protein, 214 aa, synthetic construct
  source 1..214: note = Library of modified or engineered enzymes
MQEITVAEFSNITKNEVTSWNPKPKTPVIDPTSYVDPNATVIGDVTIGKNCYIAPFASIRADEGTPIVIGDDSNVQDGVVLHALESVDADGKILEDNIVLHGDKRYAVYIGKNVSLAHQSQVHGPAHVGDDSFVGMMSLVFKSKVGNNCVIEPGAAAIGVTVPDGKYIPAGTVVTTQAEADKLPEITPDYAKSNQVAAVVKVNVALCEAYRKQS

SEQ ID NO: 398 - protein, 317 aa, synthetic construct
  source 1..317: note = Library of modified or engineered enzymes
MQTYDSSLRESLLPLPDKKESVRYWLILGGFVTAAAVAVFVIAARSGSHADASVLNSALIAVAPHFEYAEANCDETKCEASEIVQLDTWSWAPTCITGRAQSPIDIVTKEVATAGLLDDAISLSIGSATLVPSNTGHGFQLTSTGGTPSAMFRGEKFNFKQTHWHTPSENTVDGEHAAMEGHFVFQLDDPLWVNTTLNLAVMAVFFELGDCNQHLSAVWDTFPVDRLGTGSGTFSGEILASLLASVLGGGYYQFTGSLTTPPCTEGVAWNVMKKRTTVCQDQVDRLKHALSATANGVDISNRVVQPLHQRVVTQTSR

SEQ ID NO: 399 - protein, 265 aa, synthetic construct
  source 1..265: note = Library of modified or engineered enzymes
MSRLTGKIAVFCYQNPEHWDYDSPIAKGNRQSPIDIDLWSAKYDPGLKPLTFTGYDKKSLRTLLNNGHSVSVQFEDSEDKAVLSGGPLTGVYRLKQFHFHWGAADDKGSEHTVDGVKYPSELHLVHWNAVKFSSFAEAASKPDGLAVVGVFLKIGKEHVELNKLTDALYMVRFKGTKAQFSCFNPKCLLPASRHYWTYPGSLTTPPLSESVTWIVLREPISVSERQMEKFRSLLFTSEDDERIHMVNNFRPLQPLMNRTVRSSFR

SEQ ID NO: 400 - protein, 210 aa, synthetic construct
  source 1..210: note = Library of modified or engineered enzymes
MVPYPYPIVYQNPPVAEVTSVSYPKISRKAVIGTDSMIIGDITIADDVYIGFKNLLRADSGHPYYVGPYTNIQDYVLMHVHPGREHVVVNNQKWGVFLEGMNSVLHHAAVHGPLFIGKNTFIGQHANIYDAVIGRDCVVMHGATVTNGVKIADNRFVAPGQSVWQQSEADKLPPVPEKFKDLNRSIVDHYYRLGKSYGLNTPLAYSYSGG

SEQ ID NO: 401 - protein, 288 aa, synthetic construct
  source 1..288: note = Library of modified or engineered enzymes
MLALTLAILLLLNARAVLSSCAHGTYLLRRAIDDNKPIKLPNFGYGPFDGPTNWHSLSEDNILCGTGRRQSPIDIDDTISQVAAGFVSMDVPIQDVSFLNLRTTVEVILKGSTRINGREFVLEQFHFHTPSEHVLNGEIFVAEVHFVHSNKENPKELAVITLMVQVSADHSTRSLDRVIGEITRISTPGNKVAIPALNIGDITSLVNKQQLFTYTGSLTTPPCTEGVQFFILPQPIPMRATVFNALKSVTGHNARFLQNNNATRPNVLVAGCQVIAAEVWSNATQYSR

SEQ ID NO: 402 - protein, 236 aa, synthetic construct
  source 1..236: note = Library of modified or engineered enzymes
MLAATVPAGTALADEWGYAGDGAPVNWGALSPDFAVCSAGVQQSPVDLVPGIIADGVRPVLDFADVSGVEAERSAHGVTYHVPSGSAQLSLNGRSFDLLQFHFHAASEHWVEGQSYPLEVHFVTASEGDLAVVGVLFERGEAHATVDTLWDAIGEPGDREEIDGPVSLASLLPQDQAAFRYEGSLTTPPCSEIVSWTVFTTPLSVSDAQIDAFVETVGENARPPQPLNRRYVLLDN

SEQ ID NO: 403 - protein, 183 aa, synthetic construct
  source 1..183: note = Library of modified or engineered enzymes
TAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKETS

SEQ ID NO: 404 - protein, 183 aa, synthetic construct
  source 1..183: note = Library of modified or engineered enzymes
TAFIAPNAEVIGDVTIEGNAMISPNASIRADEGMPIYLGKDVNLQDNVQLHALETVDEEGNLIEENLVEVNGKKYAVYLGENVSLGHQAQVHGPAYVGKDTFIGMNAVVFKSRIGNNCVLEPNATVIGVTIPDGRYVKAGTVIRTQADLPKLLDLKEVPEVKKKVDEYHNKLKELIKAKKAAA

SEQ ID NO: 405 - protein, 183 aa, synthetic construct
  source 1..183: note = Library of modified or engineered enzymes
TAYIHPSAQVIGEVEIGANVMVSPMASIRADEGMPIVLGDNANVQDGVQLHALETVNEEGELIEENVVEVDGKKYAVYVGENVSLAHQAQVHGPAIVGKDTFIGMGAKVFKSTVGNGCVLEPGAEVIGVTIPDGRYVPAGTVVRTQEQIPSLREMTPDDPLLAVRDRVIAENLKRAAELKARA

SEQ ID NO: 406 - protein, 183 aa, synthetic construct
  source 1..183: note = Library of modified or engineered enzymes
TAYIAEGAEVIGEVYIGENVMISPNASIRSDEGMPIYIGENANVQDGVELHALETKDEEGNLIEENVVEVNGKKYAVYVGENVSLAHQAQIHGPARVGKDTFIGMGAVVRGSTLGENVLLGEGVVIENVTIEEGTLVEEGTVITKQEDVKKLRKLKPSDKMVKIKKEVLEKNKKLWEKLKEEE

SEQ ID NO: 407 - protein, 183 aa, synthetic construct
  source 1..183: note = Library of modified or engineered enzymes
TAYIHPSATVIGPVRIEEGVMISPNASIRADEGGPIYLGENVNVQDGVTLHCLEVKREEGRVDESVFVEVDGEKYCVYLGEGVSLGHQATIHGPAKVGEDTFIGMGATVFRSTIGEGCVLEPGATVIGVTIPEGRYVPAGKTVTTQAEADALPLITDSYPYRETNENVVAVNLALAEAARAAA

SEQ ID NO: 408 - protein, 183 aa, synthetic construct
  source 1..183: note = Library of modified or engineered enzymes
TAYIHPSAQVIGEVQIGANVMVSPMASIRSDEGMPIYIGDNANVQDGVQLHALEARNEEGVEDESAWVEVNGKRYRVYVGENVSLAHQAQVHGPAAVGKDTFIGMGASVFKSRLGNGCVLEPGATVIGVTIPDGRYLPAGTVLRGRPIEEEIELREVTEELRARHQAVVEANLARAAELKARA

SEQ ID NO: 409 - protein, 183 aa, synthetic construct
  source 1..183: note = Library of modified or engineered enzymes
TAYIHPSAEVIGEVEIGANVMVSPMASIRSDEGMPIYIGDNANVQDGVLLHALEALDEEGEEDEEAYVEVDGKRYRVYLGNNVSLAHQAQVHGPAKVGDDTFIGMGASVFKSILGNGCVLEPNATVIGVTIPDGRYIPAGAVLNVEDAEKTRELPEVTPELRAKRAAVLAANAARYAELRAAA

SEQ ID NO: 410 - protein, 183 aa, synthetic construct
  source 1..183: note = Library of modified or engineered enzymes
TAYIHPSAQVIGDVQIGANVMVSPMASIRSDEGMPIYIGDNANVQDGVQLHALEARDEEGVEDEEAYVEVNGKRYRVYLGENVSLAHQAQVHGPAAVGRDTFIGMGASVFKSRLGNGCVLEPQATVIGVTIPDGRYLPAGAVLEGETAEAVAALPEVTPEMREAVAAQQAAAAAQYAAAKAAA

SEQ ID NO: 411 - protein, 183 aa, synthetic construct
  source 1..183: note = Library of modified or engineered enzymes
TAYIAPSAEVIGEVTIEDDCMISPNASIRADEGMPIYLGNGTNVQDGVTLHGLEVKREEGEEDESAYVEVNGKKYVVYLGDNVSLGHQAQIHGPAKVGDDTFIGMGATVFKSVIGNGCVLEPGATVIGVTIPDGRYVPAGATVTTQAEADALPLMTPDYALYHTNERVVAVNRALAAEARAAA

SEQ ID NO: 412 - protein, 183 aa, synthetic construct
  source 1..183: note = Library of modified or engineered enzymes
TAYIHPSASVIGDVEIGANVMVSPMASIRSDEGMPIHIGDNANVQDGVVLHGLEVKNEEGEEDESQYVEVNGKKYVVYLGKNVSLAHQAQIHGPAKVGDDTFIGMGAFVFKSTLGNNCVLEPGATVIGVTIPDGRYLPAGTTVTGKPLAEDVTVRPVTEEQRNKHKKVVEKNLKLAKLLKELS

SEQ ID NO: 413 - protein, 183 aa, synthetic construct
  source 1..183: note = Library of modified or engineered enzymes
TAYIHPSAQVIGPVQIGANVMVSPMASIRSDEGMPIYIGDNANVQDGVQLHALEARDEEGVEDEAAYVEVNGKRYRVYIGENVSLAHQAQVHGPASVGSDTFIGMGASVFKSRLGNGCVLEPQATVIGVTIPDGRYLPAGTVLLPGRLEDNTPLREVTPEQREAHKAVVTENLARATELKAAL

SEQ ID NO: 414 - protein, 549 aa, synthetic construct
  source 1..549: note = Library of modified or engineered enzymes
TASIAPSATVIGDVEIADNVMISPNASIRSDEGMPIYLGANANIQDNVTLHALETKDEEGNLIEENYVEVNGKKYAVYIGDGSTLPAGLTIKGNGYVEVLAGPGELLVVTEPYKVEITSPEPVLVLRRLSPEVRELLEVSPDSERLLAREADGGTALFAAFDARRAALKAANLAANAAAVAALTASIAPSATVIGDVEIADNVMISPNASIRSDEGMPIYLGANANIQDNVTLHALETKDEEGNLIEENYVEVNGKKYAVYIGDGSTLPAGLTIKGNGYVEVLAGPGELLVVTEPYKVEITSPEPVLVLRRLSPEVRELLEVSPDSERLLAREADGGTALFAAFDARRAALKAANLAANAAAVAALTASIAPSATVIGDVEIADNVMISPNASIRSDEGMPIYLGANANIQDNVTLHALETKDEEGNLIEENYVEVNGKKYAVYIGDGSTLPAGLTIKGNGYVEVLAGPGELLVVTEPYKVEITSPEPVLVLRRLSPEVRELLEVSPDSERLLAREADGGTALFAAFDARRAALKAANLAANAAAVAAL

SEQ ID NO: 415 - protein, 549 aa, synthetic construct
  source 1..549: note = Library of modified or engineered enzymes
TAYIHPSASVIGDVEIGENVMISPNASIRSDEGMPIYLGENVNVQDNVTLHGLEVYTEEGELIEENLVEVNGKRYVVYTGKNVSLGHQAQIHGPAKVGDDTFIGMNATVFKSVIGNNCVLEPNATVIGVTIPDGRYVPAGKTVTTQAEADALPVLTPDYALYHTNERVNAVNLKLAQEANAAATAYIHPSASVIGDVEIGENVMISPNASIRSDEGMPIYLGENVNVQDNVTLHGLEVYTEEGELIEENLVEVNGKRYVVYTGKNVSLGHQAQIHGPAKVGDDTFIGMNATVFKSVIGNNCVLEPNATVIGVTIPDGRYVPAGKTVTTQAEADALPVLTPDYALYHTNERVNAVNLKLAQEANAAATAYIHPSASVIGDVEIGENVMISPNASIRSDEGMPIYLGENVNVQDNVTLHGLEVYTEEGELIEENLVEVNGKRYVVYTGKNVSLGHQAQIHGPAKVGDDTFIGMNATVFKSVIGNNCVLEPNATVIGVTIPDGRYVPAGKTVTTQAEADALPVLTPDYALYHTNERVNAVNLKLAQEANAAA

SEQ ID NO: 416 - protein, 549 aa, synthetic construct
  source 1..549: note = Library of modified or engineered enzymes
TAYIAPGAEVIGEVEIGANVMVSPMASIRSDEGMPIYLGDNTNVQDGVTLHGLEVEDEEGEEDESVYVEVNGKKYRVYIGNNVSLAHQAQVHGPAYVGDDTFIGMGATVFKSRIGNGCVLEPGATVIGVTIPDGRYVPAGKTVTTQAEADALPVLTPDYAMYHTNETVVAVNLALAAAAKAAATAYIAPGAEVIGEVEIGANVMVSPMASIRSDEGMPIYLGDNTNVQDGVTLHGLEVEDEEGEEDESVYVEVNGKKYRVYIGNNVSLAHQAQVHGPAYVGDDTFIGMGATVFKSRIGNGCVLEPGATVIGVTIPDGRYVPAGKTVTTQAEADALPVLTPDYAMYHTNETVVAVNLALAAAAKAAATAYIAPGAEVIGEVEIGANVMVSPMASIRSDEGMPIYLGDNTNVQDGVTLHGLEVEDEEGEEDESVYVEVNGKKYRVYIGNNVSLAHQAQVHGPAYVGDDTFIGMGATVFKSRIGNGCVLEPGATVIGVTIPDGRYVPAGKTVTTQAEADALPVLTPDYAMYHTNETVVAVNLALAAAAKAAA

SEQ ID NO: 417 - protein, 549 aa, synthetic construct
  source 1..549: note = Library of modified or engineered enzymes
TAYIAPTAEVIGDVIIGDNVMISPNASIRSDEGMPIYIGENVNVQDGVTITADRTKDEAGNDIPENWVTVNGKKYAVYLGKNVVLAHNATVNGRTVLGENVLVQENATLTASTLGENVIVQENATLTGVTVAEGKVVEAGKTITTQAEADKLKDLTKDHPLYNKNKEVVAKNLAILEEKKKLETAYIAPTAEVIGDVIIGDNVMISPNASIRSDEGMPIYIGENVNVQDGVTITADRTKDEAGNDIPENWVTVNGKKYAVYLGKNVVLAHNATVNGRTVLGENVLVQENATLTASTLGENVIVQENATLTGVTVAEGKVVEAGKTITTQAEADKLKDLTKDHPLYNKNKEVVAKNLAILEEKKKLETAYIAPTAEVIGDVIIGDNVMISPNASIRSDEGMPIYIGENVNVQDGVTITADRTKDEAGNDIPENWVTVNGKKYAVYLGKNVVLAHNATVNGRTVLGENVLVQENATLTASTLGENVIVQENATLTGVTVAEGKVVEAGKTITTQAEADKLKDLTKDHPLYNKNKEVVAKNLAILEEKKKLE

SEQ ID NO: 418 - protein, 549 aa, synthetic construct
  source 1..549: note = Library of modified or engineered enzymes
TAYIAPTATVIGDVEIADNVMISPNASIRSDEGMPIYIGENANLQDNVVLHALETKDEEGNDIEENWVEVDGKKYAVYIGRRVSLGHQAQIHGPALVGDDTFIGMNAKVFKSRIGNRCVLEPNAQVIGVTIPDGRYVPAGKVVTTQEEADKLPLLTPDYAMYHTNERVNAVNLALAAEARALATAYIAPTATVIGDVEIADNVMISPNASIRSDEGMPIYIGENANLQDNVVLHALETKDEEGNDIEENWVEVDGKKYAVYIGRRVSLGHQAQIHGPALVGDDTFIGMNAKVFKSRIGNRCVLEPNAQVIGVTIPDGRYVPAGKVVTTQEEADKLPLLTPDYAMYHTNERVNAVNLALAAEARALATAYIAPTATVIGDVEIADNVMISPNASIRSDEGMPIYIGENANLQDNVVLHALETKDEEGNDIEENWVEVDGKKYAVYIGRRVSLGHQAQIHGPALVGDDTFIGMNAKVFKSRIGNRCVLEPNAQVIGVTIPDGRYVPAGKVVTTQEEADKLPLLTPDYAMYHTNERVNAVNLALAAEARALA

SEQ ID NO: 419 - protein, 549 aa, synthetic construct
  source 1..549: note = Library of modified or engineered enzymes
TAYIHPTAEVIGDVEIGDNVMISPNASIRADEGMPIVIEENVNVQDGVEITALRSDLPEEEVEKLDLQEVDGKKVRAYFGKGAVLAHGAKILVASTRLKVEPVPGVTVLKQDNAVLRNVLLTEMHGLILEVNAETGSIVIRESSDPALESKAKTWKVTPEDKAKIAAVIAANAAARQEALAAATAYIHPTAEVIGDVEIGDNVMISPNASIRADEGMPIVIEENVNVQDGVEITALRSDLPEEEVEKLDLQEVDGKKVRAYFGKGAVLAHGAKILVASTRLKVEPVPGVTVLKQDNAVLRNVLLTEMHGLILEVNAETGSIVIRESSDPALESKAKTWKVTPEDKAKIAAVIAANAAARQEALAAATAYIHPTAEVIGDVEIGDNVMISPNASIRADEGMPIVIEENVNVQDGVEITALRSDLPEEEVEKLDLQEVDGKKVRAYFGKGAVLAHGAKILVASTRLKVEPVPGVTVLKQDNAVLRNVLLTEMHGLILEVNAETGSIVIRESSDPALESKAKTWKVTPEDKAKIAAVIAANAAARQEALAAA

SEQ ID NO: 420 - protein, 549 aa, synthetic construct
  source 1..549: note = Library of modified or engineered enzymes
TAYIEPNAEVIGDVKIGENVMISPNASIRSDEGMPIVIKENVNVQDGVVINAKLKKNENGEVDESQLNTINGEKVQIYLEKNVQLAHNVTIEDSVVLKENVLLQENVVLKNSTLGEGVVLAENVVIENVTLPENTVVEAGTVIKNQEEVKTLKQLTADSPAIVQLQAVLAKNAALWEELKAAETAYIEPNAEVIGDVKIGENVMISPNASIRSDEGMPIVIKENVNVQDGVVINAKLKKNENGEVDESQLNTINGEKVQIYLEKNVQLAHNVTIEDSVVLKENVLLQENVVLKNSTLGEGVVLAENVVIENVTLPENTVVEAGTVIKNQEEVKTLKQLTADSPAIVQLQAVLAKNAALWEELKAAETAYIEPNAEVIGDVKIGENVMISPNASIRSDEGMPIVIKENVNVQDGVVINAKLKKNENGEVDESQLNTINGEKVQIYLEKNVQLAHNVTIEDSVVLKENVLLQENVVLKNSTLGEGVVLAENVVIENVTLPENTVVEAGTVIKNQEEVKTLKQLTADSPAIVQLQAVLAKNAALWEELKAAE

SEQ ID NO: 421 - protein, 549 aa, synthetic construct
  source 1..549: note = Library of modified or engineered enzymes
TAYIHPSAEVIGDVTIGDNVMISPNASIRADEGMPIYLGDNANVQDGVTLHGLETKDEEGNIIEENLVEVNGKKYAVYVGDNVSLAHQAQIHGPAIVGDDTFIGMGATVRRSILGDGVLLGEGVQIENATLPAGLCLGPGRVIRTPEELVDDCTEEQRAELKKKHAEVVAKNLALHEELKAAATAYIHPSAEVIGDVTIGDNVMISPNASIRADEGMPIYLGDNANVQDGVTLHGLETKDEEGNIIEENLVEVNGKKYAVYVGDNVSLAHQAQIHGPAIVGDDTFIGMGATVRRSILGDGVLLGEGVQIENATLPAGLCLGPGRVIRTPEELVDDCTEEQRAELKKKHAEVVAKNLALHEELKAAATAYIHPSAEVIGDVTIGDNVMISPNASIRADEGMPIYLGDNANVQDGVTLHGLETKDEEGNIIEENLVEVNGKKYAVYVGDNVSLAHQAQIHGPAIVGDDTFIGMGATVRRSILGDGVLLGEGVQIENATLPAGLCLGPGRVIRTPEELVDDCTEEQRAELKKKHAEVVAKNLALHEELKAAA

SEQ ID NO: 422 - protein, 549 aa, synthetic construct
  source 1..549: note = Library of modified or engineered enzymes
TAYIAPNAQVIGEVTIGENVMISPNASIRSDEGMPIYIGENANLQDNVVLHGLEVYTEEGELIEENLVEVDGKKYVVYIGKNVSLGHQAQIHGPAKVGDDTFIGMNAKVFKSVIGNRCVLEPNATVIGVTIPDGRYVPAGKVVTTQAEADALPVLTPDYALYHTNELVNEVNLALAAEGRAAATAYIAPNAQVIGEVTIGENVMISPNASIRSDEGMPIYIGENANLQDNVVLHGLEVYTEEGELIEENLVEVDGKKYVVYIGKNVSLGHQAQIHGPAKVGDDTFIGMNAKVFKSVIGNRCVLEPNATVIGVTIPDGRYVPAGKVVTTQAEADALPVLTPDYALYHTNELVNEVNLALAAEGRAAATAYIAPNAQVIGEVTIGENVMISPNASIRSDEGMPIYIGENANLQDNVVLHGLEVYTEEGELIEENLVEVDGKKYVVYIGKNVSLGHQAQIHGPAKVGDDTFIGMNAKVFKSVIGNRCVLEPNATVIGVTIPDGRYVPAGKVVTTQAEADALPVLTPDYALYHTNELVNEVNLALAAEGRAAA

SEQ ID NO: 423 - protein, 549 aa, synthetic construct
  source 1..549: note = Library of modified or engineered enzymes
TAYIHPSAEVIGDVEIADNVMISPNASIRADEGMPIYLGENTNVQDGVSLHALENSSEEGEEDESNWVEVNGKKYRVYIGNNVSLGHQAQVHGPAIVGDDTFIGMGAKVFKSTIGNGCVLEPGVTVIGVTIPDGRYLEAGTVLRSQADIEKAKPIKEDMPTYKKVKEHKEKLKEEREKLKKERTAYIHPSAEVIGDVEIADNVMISPNASIRADEGMPIYLGENTNVQDGVSLHALENSSEEGEEDESNWVEVNGKKYRVYIGNNVSLGHQAQVHGPAIVGDDTFIGMGAKVFKSTIGNGCVLEPGVTVIGVTIPDGRYLEAGTVLRSQADIEKAKPIKEDMPTYKKVKEHKEKLKEEREKLKKERTAYIHPSAEVIGDVEIADNVMISPNASIRADEGMPIYLGENTNVQDGVSLHALENSSEEGEEDESNWVEVNGKKYRVYIGNNVSLGHQAQVHGPAIVGDDTFIGMGAKVFKSTIGNGCVLEPGVTVIGVTIPDGRYLEAGTVLRSQADIEKAKPIKEDMPTYKKVKEHKEKLKEEREKLKKER

SEQ ID NO: 424 - protein, 549 aa, synthetic construct
  source 1..549: note = Library of modified or engineered enzymes
TAIIAPGATVIGEVHIADGVMISPNASIRADEGMPIYLGEYTNLQDNVVLHALETYDEEGNLIEENLVEVNGKKYAVYVGKNVSLGHQAQLHGPTIVGDDTFIGMNAKVIRSTLGEGVVLEENVVVEGQTLEKGTYLEKGMKLLTPEDLKKAKKIKEEDPVKKKLEAHIKEQKAQAKAAQAAATAIIAPGATVIGEVHIADGVMISPNASIRADEGMPIYLGEYTNLQDNVVLHALETYDEEGNLIEENLVEVNGKKYAVYVGKNVSLGHQAQLHGPTIVGDDTFIGMNAKVIRSTLGEGVVLEENVVVEGQTLEKGTYLEKGMKLLTPEDLKKAKKIKEEDPVKKKLEAHIKEQKAQAKAAQAAATAIIAPGATVIGEVHIADGVMISPNASIRADEGMPIYLGEYTNLQDNVVLHALETYDEEGNLIEENLVEVNGKKYAVYVGKNVSLGHQAQLHGPTIVGDDTFIGMNAKVIRSTLGEGVVLEENVVVEGQTLEKGTYLEKGMKLLTPEDLKKAKKIKEEDPVKKKLEAHIKEQKAQAKAAQAAA

SEQ ID NO: 425 - protein, 549 aa, synthetic construct
  source 1..549: note = Library of modified or engineered enzymes
TAYIHPSAEVIGEVEIGANVMVSPMASIRSDEGMPIKIGDNVNVQDGVVLHGLETKNEEGEEIEENLVEVDGEKYVVYLGKNVSLAHQAQVHGPSIVGDDTFIGMGAKVEGSTLGDGVFLGEGATVTGLTIPAGAVVSPGTVLTTPAQLASLKPLTADDPLLKKKKDVVENNLATAAALKALETAYIHPSAEVIGEVEIGANVMVSPMASIRSDEGMPIKIGDNVNVQDGVVLHGLETKNEEGEEIEENLVEVDGEKYVVYLGKNVSLAHQAQVHGPSIVGDDTFIGMGAKVEGSTLGDGVFLGEGATVTGLTIPAGAVVSPGTVLTTPAQLASLKPLTADDPLLKKKKDVVENNLATAAALKALETAYIHPSAEVIGEVEIGANVMVSPMASIRSDEGMPIKIGDNVNVQDGVVLHGLETKNEEGEEIEENLVEVDGEKYVVYLGKNVSLAHQAQVHGPSIVGDDTFIGMGAKVEGSTLGDGVFLGEGATVTGLTIPAGAVVSPGTVLTTPAQLASLKPLTADDPLLKKKKDVVENNLATAAALKALE

SEQ ID NO: 426 - protein, 549 aa, synthetic construct
  source 1..549: note = Library of modified or engineered enzymes
TAYIHPSASVIGDVEIADNVMISPNASIRADEGMPIKIGPNANVQDGVTLHGLETYDEEGNLIEENYVEVNGERYVVYIGDNVSLGHQAQIHGPAKVGDDTFIGMKATVFKSVIGNNCVLEPGATVIGVTIPDGRYVPAGKVVTTQAEADALPVLTPDYALYHTNERVNAVNLKLAEKARLEATAYIHPSASVIGDVEIADNVMISPNASIRADEGMPIKIGPNANVQDGVTLHGLETYDEEGNLIEENYVEVNGERYVVYIGDNVSLGHQAQIHGPAKVGDDTFIGMKATVFKSVIGNNCVLEPGATVIGVTIPDGRYVPAGKVVTTQAEADALPVLTPDYALYHTNERVNAVNLKLAEKARLEATAYIHPSASVIGDVEIADNVMISPNASIRADEGMPIKIGPNANVQDGVTLHGLETYDEEGNLIEENYVEVNGERYVVYIGDNVSLGHQAQIHGPAKVGDDTFIGMKATVFKSVIGNNCVLEPGATVIGVTIPDGRYVPAGKVVTTQAEADALPVLTPDYALYHTNERVNAVNLKLAEKARLEA

SEQ ID NO: 427 - protein, 549 aa, synthetic construct
  source 1..549: note = Library of modified or engineered enzymes
TAYIAPSAEVIGDVEIGANVMVSPMASIRADEGMPIYIGDNANVQDGVVLHALETYDEEGNLIEEAYVEVDGKKYAVYVGDNVSLAHQAQIHGPAKVGEDTFIGMGAKVVGSTLGKGVFLAEGVVVENATLPEGTILEKGTVVTPSDKELPKAPEELRAKLAAEHKAVVAANIAAAAAAKAAATAYIAPSAEVIGDVEIGANVMVSPMASIRADEGMPIYIGDNANVQDGVVLHALETYDEEGNLIEEAYVEVDGKKYAVYVGDNVSLAHQAQIHGPAKVGEDTFIGMGAKVVGSTLGKGVFLAEGVVVENATLPEGTILEKGTVVTPSDKELPKAPEELRAKLAAEHKAVVAANIAAAAAAKAAATAYIAPSAEVIGDVEIGANVMVSPMASIRADEGMPIYIGDNANVQDGVVLHALETYDEEGNLIEEAYVEVDGKKYAVYVGDNVSLAHQAQIHGPAKVGEDTFIGMGAKVVGSTLGKGVFLAEGVVVENATLPEGTILEKGTVVTPSDKELPKAPEELRAKLAAEHKAVVAANIAAAAAAKAAA

SEQ ID NO: 428 - protein, 549 aa, synthetic construct
  source 1..549: note = Library of modified or engineered enzymes
TAYIAPGAEVIGDVEIGANVMVSPMASIRADEGMPIYVGDNANVQDGVVLHGLETLDEEGNLIEENWVEVDGKKYVVYLGKNVSLAHQAQIHGPAKVGEDTFIGMQALVFKSTIGNGCVLEPGAAVIGVTVPDGRYVPAGAVVTSQAEADALPKMTPDYAYAHTNETVVAVNNALAAGYKAAATAYIAPGAEVIGDVEIGANVMVSPMASIRADEGMPIYVGDNANVQDGVVLHGLETLDEEGNLIEENWVEVDGKKYVVYLGKNVSLAHQAQIHGPAKVGEDTFIGMQALVFKSTIGNGCVLEPGAAVIGVTVPDGRYVPAGAVVTSQAEADALPKMTPDYAYAHTNETVVAVNNALAAGYKAAATAYIAPGAEVIGDVEIGANVMVSPMASIRADEGMPIYVGDNANVQDGVVLHGLETLDEEGNLIEENWVEVDGKKYVVYLGKNVSLAHQAQIHGPAKVGEDTFIGMQALVFKSTIGNGCVLEPGAAVIGVTVPDGRYVPAGAVVTSQAEADALPKMTPDYAYAHTNETVVAVNNALAAGYKAAA

SEQ ID NO: 429 - protein, 549 aa, synthetic construct
  source 1..549: note = Library of modified or engineered enzymes
TAYIHPSATVIGQVNIGANVMVSPMASIRADEGMPITLEDNVNVQDGVLIQNESLKNESGEIDYSKVHPKNKRIESIVLKKNVSLAHQATVYSNTELSEGVFLQEGVVVKNSVIEGRVVLQRGVTVENVYIGEEVVIAEGTVLKGDEDLKKTTLAPLTPEQVAQIQAVIAQNLAAAAAAKAAATAYIHPSATVIGQVNIGANVMVSPMASIRADEGMPITLEDNVNVQDGVLIQNESLKNESGEIDYSKVHPKNKRIESIVLKKNVSLAHQATVYSNTELSEGVFLQEGVVVKNSVIEGRVVLQRGVTVENVYIGEEVVIAEGTVLKGDEDLKKTTLAPLTPEQVAQIQAVIAQNLAAAAAAKAAATAYIHPSATVIGQVNIGANVMVSPMASIRADEGMPITLEDNVNVQDGVLIQNESLKNESGEIDYSKVHPKNKRIESIVLKKNVSLAHQATVYSNTELSEGVFLQEGVVVKNSVIEGRVVLQRGVTVENVYIGEEVVIAEGTVLKGDEDLKKTTLAPLTPEQVAQIQAVIAQNLAAAAAAKAAA

SEQ ID NO: 430 - protein, 549 aa, synthetic construct
  source 1..549: note = Library of modified or engineered enzymes
TAYIAPGAQVIGDVEIADNVMISPNASIRADEGMPIYIGENANLQDNVQLHGLEVYTEEGELIEENFVEVDGKKYVVYIGRRVSLAHQAQVHGPAKVGDDTFIGMNAKVFKSIVGNRCVLEPNATVIGVTIPDGRYVPAGKTVTTQAEADALPVLTPDYALYHTNELVNAVNLALAAEARAAATAYIAPGAQVIGDVEIADNVMISPNASIRADEGMPIYIGENANLQDNVQLHGLEVYTEEGELIEENFVEVDGKKYVVYIGRRVSLAHQAQVHGPAKVGDDTFIGMNAKVFKSIVGNRCVLEPNATVIGVTIPDGRYVPAGKTVTTQAEADALPVLTPDYALYHTNELVNAVNLALAAEARAAATAYIAPGAQVIGDVEIADNVMISPNASIRADEGMPIYIGENANLQDNVQLHGLEVYTEEGELIEENFVEVDGKKYVVYIGRRVSLAHQAQVHGPAKVGDDTFIGMNAKVFKSIVGNRCVLEPNATVIGVTIPDGRYVPAGKTVTTQAEADALPVLTPDYALYHTNELVNAVNLALAAEARAAA

SEQ ID NO: 431 - protein, 549 aa, synthetic construct
  source 1..549: note = Library of modified or engineered enzymes
TAYIHPTARVIGEVTIAAGVMISPGASIRADEGMPIVIGENANVQDGVSLHGLEVYDEEGNLIEENLVEVNGEKYVVYIGENVSLGHQAQIHGPALVGDDTFIGMGAKVTRSILGEGVILEEGAQLTNVIVPDGAYVKSGQVFVSTGEPVVLSELKQTPEQKEKLAAQLAAERAARAAAQAAATAYIHPTARVIGEVTIAAGVMISPGASIRADEGMPIVIGENANVQDGVSLHGLEVYDEEGNLIEENLVEVNGEKYVVYIGENVSLGHQAQIHGPALVGDDTFIGMGAKVTRSILGEGVILEEGAQLTNVIVPDGAYVKSGQVFVSTGEPVVLSELKQTPEQKEKLAAQLAAERAARAAAQAAATAYIHPTARVIGEVTIAAGVMISPGASIRADEGMPIVIGENANVQDGVSLHGLEVYDEEGNLIEENLVEVNGEKYVVYIGENVSLGHQAQIHGPALVGDDTFIGMGAKVTRSILGEGVILEEGAQLTNVIVPDGAYVKSGQVFVSTGEPVVLSELKQTPEQKEKLAAQLAAERAARAAAQAAA

SEQ ID NO: 432 - protein, 549 aa, synthetic construct
  source 1..549: note = Library of modified or engineered enzymes
TAYIHPTAEVIGNVKIGENVMISPNASIRSDEGMPIVIKENANVQDGVVIRADPTKDENGNDIEENWVTVNGEKYAVYLEKNVVLAHNAVVEGRTVLKEGVLVQENAVVRRSTLGEGVILQENAVLEGVTVADGKIVPAGATIRTQAEADTLATLTPDHPLYNLNKVVNAKNLALLKENLAAKTAYIHPTAEVIGNVKIGENVMISPNASIRSDEGMPIVIKENANVQDGVVIRADPTKDENGNDIEENWVTVNGEKYAVYLEKNVVLAHNAVVEGRTVLKEGVLVQENAVVRRSTLGEGVILQENAVLEGVTVADGKIVPAGATIRTQAEADTLATLTPDHPLYNLNKVVNAKNLALLKENLAAKTAYIHPTAEVIGNVKIGENVMISPNASIRSDEGMPIVIKENANVQDGVVIRADPTKDENGNDIEENWVTVNGEKYAVYLEKNVVLAHNAVVEGRTVLKEGVLVQENAVVRRSTLGEGVILQENAVLEGVTVADGKIVPAGATIRTQAEADTLATLTPDHPLYNLNKVVNAKNLALLKENLAAK

SEQ ID NO: 433 - protein, 549 aa, synthetic construct
  source 1..549: note = Library of modified or engineered enzymes
TAIIAPGATVIGEVEIGDNVMISPNASIRSDEGMPIVLGEGANLQDNVELHALEVYDEEGNLIEENYVEVNGKKYAVYIGNNVSLGHQAQIHGPAIVGDDTFIGMNAEVFKSIIGNGCVLEPNARVIGVTIPDGRYVKAGTTITDQAEIPSLKQLKDSDPIKAKVEAHKAALKAERERLLAERTAIIAPGATVIGEVEIGDNVMISPNASIRSDEGMPIVLGEGANLQDNVELHALEVYDEEGNLIEENYVEVNGKKYAVYIGNNVSLGHQAQIHGPAIVGDDTFIGMNAEVFKSIIGNGCVLEPNARVIGVTIPDGRYVKAGTTITDQAEIPSLKQLKDSDPIKAKVEAHKAALKAERERLLAERTAIIAPGATVIGEVEIGDNVMISPNASIRSDEGMPIVLGEGANLQDNVELHALEVYDEEGNLIEENYVEVNGKKYAVYIGNNVSLGHQAQIHGPAIVGDDTFIGMNAEVFKSIIGNGCVLEPNARVIGVTIPDGRYVKAGTTITDQAEIPSLKQLKDSDPIKAKVEAHKAALKAERERLLAER

SEQ ID NO: 434 - protein, 213 aa, synthetic construct
  source 1..213: note = Library of modified or engineered enzymes
QEITVDEFSNIRENPVTPWNPEPSAPVIDPTAYIDPQASVIGEVTIGANVMVSPMASIRSDEGMPIFVGDRSNVQDGVVLHALETINEEGEPIEDNIVEVDGKEYAVYIGNNVSLAHQSQVHGPAAVGDDTFIGMQAFVFKSKVGNNCVLEPRSAAIGVTIPDGRYIPAGMVVTSQAEADKLPEVTDDYAYSHTNEAVVYVNVHLAEGYKETS

SEQ ID NO: 435 - protein, 212 aa, synthetic construct
  source 1..212: note = Library of modified or engineered enzymes
GKVYVKKPVFIPARHIPSDKTIIEPEIDEEAVIEEGAIITGGVIIKGRVYIASGATIRSDEGVPIVIEENSSIQDGALVHADETVDEDGNPIEENIVEVNGKPYAVYIGENVVLEHNATVHGPAAVGKNSLIGEGALVRNSVIGENCVLEEGASAENVTIPAGRYVPAGVTVTTQAAAAALPAVTPDHPLYKRNEELVKENIEKAEKLLAEA

SEQ ID NO: 436 - protein, 212 aa, synthetic construct
  source 1..212: note = Library of modified or engineered enzymes
GSVLVEPSDIQCSPPNKYHKEPRCPTIAKGAYIEKGALIEGDVIIEENVYIESGAIIRSDEGTPIYIGKNSVIQDGALVHADETVDEDGNPIEENIVEVNGKPYAVYIGENVVLEHNAEVHGPAAVGKNSLIGEGALVRNSIIGENCVLEEGASAENVTIPAGRYVPAGKTVTTQAEAAALPKMTPDHPLYKRNEELVKENLEKVKKANAAA

SEQ ID NO: 437 - protein, 212 aa, synthetic construct
  source 1..212: note = Library of modified or engineered enzymes
GVVLVEEEGIRPSPATPRYPEPRAPIIHPSAYVADGALITGEVIIEDNVLIAEGAVIRSDEGRPIYIGKNSSVQDGAVIHADETVDAEGKEIEENIVEVNGKKYAVYIGENVVIEHGATVHGPAKIGENSLIGRGALVENSVIGKNCVLEEGASAIGVTIPEGRYIPAGVTVTTQEEADALPEVTPDHPDYNRVAELVAKNIALAKELNAAR

SEQ ID NO: 438 - protein, 212 aa, synthetic construct
  source 1..212: note = Library of modified or engineered enzymes
PAPVRGHEAVFEDSLHPVTGKKLVTTIAETAYIEEGATISGAVILADNVYVESGATIRSDEGIPIYVGENSAIQDGAVLHADETVDADGNPIPENIVEVNGEPYAVYIGENVVLEHGATVHGPAAVGKNSLIGKNAVVRNSVVGENCVLEEGASAENVTIPAGRYVPAGKKVTTQEEADALPEVTPDHPDYKRNEKLVAENNAKVKAYNAAR

SEQ ID NO: 439 - protein, 212 aa, synthetic construct
  source 1..212: note = Library of modified or engineered enzymes
GVVLTPVSDIRPSAPTPRYKESKAPTIHPSAYIAPGATIVGDVTIAANVYVEAGATIRSDEGVPIYVGANSAVQDGAVLHADETVDENGNPIEENIVEVNGKKYAVYVGENVVLEHGATVHGPAAIGANSLVGEGALVRNSIVGANCVLEPGASAINVTIPAGRYVPAGVTVTTQAAADALPAVTPDHPLANRNAELVAKNVAKAKAAVAAR

SEQ ID NO: 440 - protein, 212 aa, synthetic construct
  source 1..212: note = Library of modified or engineered enzymes
GTVVVATSPIRPSEPTPWRKESRAPTLAPGAYVHPDATVEGAVILEEGALVQGGATIRSDEGVPIYVGRNSVIQDGATLHADETVDEEGNPIPENIVEVDGKPYAVYVGENVVIQHGATIHGPAAVGENSLIGENALVENSVVGKNCVIQEGGAARNVTIPEGRYIPAGKTVTTQAEADALPKVTPDHPYYNKNAALVAENLARRAELLAAR

SEQ ID NO: 441 - protein, 212 aa, synthetic construct
  source 1..212: note = Library of modified or engineered enzymes
MVVLVEEAGLRPSPPTPRHREPRAPTLAEGAWVAPGATIEGEVHIAAGAYIADGATIRSDEGTPIYVGANSVIQDGALLHADETVDLDGKVIEENVVTVNGEPYAVYIGENVVIEHNATIHGPAAVGANSLIGENALVRHSIIGENCVLEEGASAINVTIPAGRYVPAGKTVTTQAEAAALPKVTPDHPNYNKNAALVAENLALNKALVAAA

SEQ ID NO: 442 - protein, 212 aa, synthetic construct
  source 1..212: note = Library of modified or engineered enzymes
MLLVEEEPLIRPSEPTPWRGTRREPTIAEGAYIEPGAVITGDTIIEAGAYIESGAVIRSDEGVPIYIGANSAIQDGAVLHADETVDENGNLIEENVVEVNGKKYAVYVGANVVIEHNAVIHGPAAVGANSLIGEGAVVRNSIVGANCVLEPGASVENVTVPAGRYVPAGVRVTTQAAADALPAVTPDHPLANRNAELVAKNVAKVKAANAAK

SEQ ID NO: 443 - protein, 212 aa, synthetic construct
  source 1..212: note = Library of modified or engineered enzymes
GLKKRKLTPAELRKPDPRYTGPRVSTIGETCLFAPGAVISPGVTLGENVYIESGAVIRSDEGRPIVVGDNSVIQDGAVLHADETVDADGNPIPENIVTVNGQPYAVYVGSNVVIDHGATIHGPAAVGANTLIGEGATVRNSTVGSNCVLEPGASAIGVTIPAGRYVPAGKTVTTQAEADALPAVTPDHPYANRVAELVAKNLAKVKAANAAR

SEQ ID NO: 444 - protein, 212 aa, synthetic construct
  source 1..212: note = Library of modified or engineered enzymes
GVVVAPVSDIQCSPPTPRFPESRCPTLHPSAYIEEGATIIGEVTIGDNVLIEKGATIRSDEGVPIYIGENSSIQDGATLHADETVDEAGNPIEENIVTVNGKPYAVYIGENVVIEHGATVHGPAAIGRNSLIGEGATVRNSIVGENCVLEEGASAIGVTIPAGRYIPAGKVVTTQEEAAALPEVTPDHPNYKRVEELVAKNIALVKALLAAR

SEQ ID NO: 445 - protein, 183 aa, synthetic construct
  source 1..183: note = Library of modified or engineered enzymes
TAYIHPSATVIGDVEIADNAMISPNASIRADEGMPIKIEKNAVVQDNATIEAKPTKDADGNLIEENIVEVNGKKYAVYIGEGAVLQKNATLEGGTIIGKNVLVQENATLTNSTLGENVIVQENATLTGVTIAEGKIVPEGATITTQEEAEKLAPLTPDHPLYNKRAEIVAKALAEREAALAAA

SEQ ID NO: 446 - protein, 183 aa, synthetic construct
  source 1..183: note = Library of modified or engineered enzymes
TAYIHPSAKVIGDVEIGANVMVSPMASIRSDEGMPIKIEENANVQDGVKIEGKRVYSASGELIEENIVEVNGKKYVVYIGENVSLAHQATIIGGTVLKKNVFIQEGAYIENSVLGENVIVQKNATIIGVTIKEGKVVPEGAVITTQEEADKLPKLTPEHPLYNLNAQVVAANIAKAAALKAAE

SEQ ID NO: 447 - protein, 183 aa, synthetic construct
  source 1..183: note = Library of modified or engineered enzymes
TAYIHPTATVIGNVKIGENVMISPEASLRADEGMPIVLEENVNVQDGVVIRGDRVLDEAGNLIEENLVEVNGERYVVYLGKGVVLAHGAVVEGSTVLGEGVLVQEGAVLRRSTLGERVIVQEGAVLEGVTVAPGAVVPAGAVIRTQAEAATLAALTPDHPLYNENARVVAKNLALLEELKALE

SEQ ID NO: 448 - protein, 183 aa, synthetic construct
  source 1..183: note = Library of modified or engineered enzymes
TAYIHPSATVIGDVIIGKNAMISPNASIRSDEGMPIVLEENAIVQDNATITAKPTKTASGELIEENVVEVNGKKYAVYLGENAILQKNATIEGGTVVGKNVLVQENATLTGSTLGENVIVQENATITGVTIAEGAIVPEGATITTQEEAEKLEKLTPDHPLYNLNAELNAKALALRDLLLALS

SEQ ID NO: 449 - protein, 183 aa, synthetic construct
  source 1..183: note = Library of modified or engineered enzymes
TAYIHPSATVIGDVEIGENAMISPNASIRSDEGMPIVLEKNVVVQDNATIEANPVLDENGELIEENVREVNGKKYAVYLEEGVVLQKNATIKGGTVVKKNVLVQENATLTNSTLGENVIVQENATLTGVTVAEGKVVPEGATITTQAEAEKLAPLTPDHPLYNLGAEIRAKALALRELKLALE

SEQ ID NO: 450 - protein, 183 aa, synthetic construct
  source 1..183: note = Library of modified or engineered enzymes
TAYIHPSAKVEGEVEIGANVMVSPMATINSTEGEPIFLGDRVNVQDGVTITCEPVYNADGELDESKLVEIDGKKYCVYVAENVSLAHQSTLTSGTVLKKNVFVQEGAVLKNSTLGENVVVRENAVIEGVTIEEGTVAEEGTVVLTEEDLAKLRPLTPEDPLLEIHKKVVEENIAKAKALKAAA

SEQ ID NO: 451 - protein, 183 aa, synthetic construct
  source 1..183: note = Library of modified or engineered enzymes
TAYIHPSAEVIGDVEIGANVMISPNASIRADEGMPIVLGDNTNVQDGVSLHALETLDEEGNLIEENVVEVDGKKYAVYVGDNVSLAHQAQVHGPAIVGEDTFIGMGAVVRRSVLGKGVILREGATVEGVTIEEGTVVEENTVLTKQEEVKKLKKLTPEHPYYNLNKKVVEQNIKKVQAARAAA

SEQ ID NO: 452 - protein, 183 aa, synthetic construct
  source 1..183: note = Library of modified or engineered enzymes
TAVIHPSATVIGDVTIADNVMISPNASIRADEGMPIVLGENVNLQDNVSLHALETLDEEGNLIEENVVEVNGKKYAVYLGEGVSLAHQAQVHGPAIVGKDTFIGMNAKVSGSILGEGVILQDNATVEGATIAEGKVVPEGAVITTQEEADKLAPLTPDHPYYELVKRTREENLRLRDLLLELE

SEQ ID NO: 453 - protein, 183 aa, synthetic construct
  source 1..183: note = Library of modified or engineered enzymes
TAYIHPSAKVIGDVEIGENVMISPNASIRADEGMPIKLEKNVIVQDNAVIEADRIYDENGELIESAVVTVNGKKYAVYLGENVILQKNATVRGGTVLGKNVLVQENAVLTNSTLGENVIVQENATVTGATLKEGTIVPEGATITTQEEADKLEKLTPDHPLYNLHAALLAAGLALRDLLLALE

SEQ ID NO: 454 - protein, 183 aa, synthetic construct
  source 1..183: note = Library of modified or engineered enzymes
TAYIHPSAEVIGDVTIGANVMVSPMASIRADEGMPIVLGNNVNVQDGVVLHGLEVLNEEGELIEENVVEVDGKKYVVYVGEGVSLAHQATVVGATVLGKGVFVGEGVVLERSILGEGVIVGENAVIKGVTIAEGKVVKEGTTVTTQEEADKLEKLTPDHPYYELNARVVAENIAKARLLKLLE

SEQ ID NO: 455 - protein, 549 aa, synthetic construct
  source 1..549: note = Library of modified or engineered enzymes
TAYIAPGASVIGEVEIGDNVMISPNASLRSDEGMPIKLGDNVNVQDGVTLHGLETKDEEGNLIEENVVEVNGEKYVVYVGKNVVLAHNVTLTSRTVVEDNVYLEENVTLTRSTLGKYVYVEKGVVIEGVTIKEGMYAKEGTVIRTQEDVKSLEMIKDLAKHKAKVQAVIDANLRIHQEALAAATAYIAPGASVIGEVEIGDNVMISPNASLRSDEGMPIKLGDNVNVQDGVTLHGLETKDEEGNLIEENVVEVNGEKYVVYVGKNVVLAHNVTLTSRTVVEDNVYLEENVTLTRSTLGKYVYVEKGVVIEGVTIKEGMYAKEGTVIRTQEDVKSLEMIKDLAKHKAKVQAVIDANLRIHQEALAAATAYIAPGASVIGEVEIGDNVMISPNASLRSDEGMPIKLGDNVNVQDGVTLHGLETKDEEGNLIEENVVEVNGEKYVVYVGKNVVLAHNVTLTSRTVVEDNVYLEENVTLTRSTLGKYVYVEKGVVIEGVTIKEGMYAKEGTVIRTQEDVKSLEMIKDLAKHKAKVQAVIDANLRIHQEALAAA

SEQ ID NO: 456 - protein, 549 aa, synthetic construct
  source 1..549: note = Library of modified or engineered enzymes
TAYIHPTATVIGSVTIADGVMISPYASIRADEGMPIYIGEGANVQDGVQLHGLETRDEEGNLIEENVVEVNGKKYVVYIGKGVSLGHQAQVHGPAIVGDDTFIGMGAQVTGAILPEGLVLAEGVRVESVDLSGYRYLPPGTVIRTQADKERVREDESLAAEVEARRAALAAERAAAEAARAAATAYIHPTATVIGSVTIADGVMISPYASIRADEGMPIYIGEGANVQDGVQLHGLETRDEEGNLIEENVVEVNGKKYVVYIGKGVSLGHQAQVHGPAIVGDDTFIGMGAQVTGAILPEGLVLAEGVRVESVDLSGYRYLPPGTVIRTQADKERVREDESLAAEVEARRAALAAERAAAEAARAAATAYIHPTATVIGSVTIADGVMISPYASIRADEGMPIYIGEGANVQDGVQLHGLETRDEEGNLIEENVVEVNGKKYVVYIGKGVSLGHQAQVHGPAIVGDDTFIGMGAQVTGAILPEGLVLAEGVRVESVDLSGYRYLPPGTVIRTQADKERVREDESLAAEVEARRAALAAERAAAEAARAAA

SEQ ID NO: 457 - protein, 549 aa, synthetic construct
  source 1..549: note = Library of modified or engineered enzymes
TAFIHPSATVIGDVTIGANVMISPMASIRSDEGMPIYVGANANLQDQVTLHALEVFDEEGNLIEENWVEVNGEKYAVYLGDNVSLAHQAQVHGPAIVGEDTFIGMQASVFKSTIGNGCVLEPGAAVIGVTIPDGRYVPAGKVVTSQAEADALPKMTPDYAYYHTNEKVVAVNRALAAGYRAAQTAFIHPSATVIGDVTIGANVMISPMASIRSDEGMPIYVGANANLQDQVTLHALEVFDEEGNLIEENWVEVNGEKYAVYLGDNVSLAHQAQVHGPAIVGEDTFIGMQASVFKSTIGNGCVLEPGAAVIGVTIPDGRYVPAGKVVTSQAEADALPKMTPDYAYYHTNEKVVAVNRALAAGYRAAQTAFIHPSATVIGDVTIGANVMISPMASIRSDEGMPIYVGANANLQDQVTLHALEVFDEEGNLIEENWVEVNGEKYAVYLGDNVSLAHQAQVHGPAIVGEDTFIGMQASVFKSTIGNGCVLEPGAAVIGVTIPDGRYVPAGKVVTSQAEADALPKMTPDYAYYHTNEKVVAVNRALAAGYRAAQ

SEQ ID NO: 458 - protein, 549 aa, synthetic construct
  source 1..549: note = Library of modified or engineered enzymes
TAYIHPSATVIGDVTIGANVMVSPMASIRSDEGMPIYLGDNVNVQDGVSLHGLETVDEEGNVIEENLVEVDGKKYVVYLGDNVSLAHQAVVEGGTVLGENVFLQEGVRVRRSTLGEGVILREGATVEGVTIAPGKVVAAGQTVTTQAAADALPTLTASDPLYSEHATVVAANIAAAAAAKAAATAYIHPSATVIGDVTIGANVMVSPMASIRSDEGMPIYLGDNVNVQDGVSLHGLETVDEEGNVIEENLVEVDGKKYVVYLGDNVSLAHQAVVEGGTVLGENVFLQEGVRVRRSTLGEGVILREGATVEGVTIAPGKVVAAGQTVTTQAAADALPTLTASDPLYSEHATVVAANIAAAAAAKAAATAYIHPSATVIGDVTIGANVMVSPMASIRSDEGMPIYLGDNVNVQDGVSLHGLETVDEEGNVIEENLVEVDGKKYVVYLGDNVSLAHQAVVEGGTVLGENVFLQEGVRVRRSTLGEGVILREGATVEGVTIAPGKVVAAGQTVTTQAAADALPTLTASDPLYSEHATVVAANIAAAAAAKAAA

SEQ ID NO: 459 - protein, 549 aa, synthetic construct
  source 1..549: note = Library of modified or engineered enzymes
TAYIAPSATVIGDVTIGANVMISPMASIRADEGMPIKVGDNANVQDQVTLHALETKDEEGNDIEENWVEVNGEKYAVYVGANVSLAHQAQLHGPCIVGDDTFIGMQARVFKSSIGNGCVLEPQAAVIGVTIPDGRYVPAGAVVTSQAEADALPKLTDDYAYAHTNEKVVAVNLALAKGYLTTPTAYIAPSATVIGDVTIGANVMISPMASIRADEGMPIKVGDNANVQDQVTLHALETKDEEGNDIEENWVEVNGEKYAVYVGANVSLAHQAQLHGPCIVGDDTFIGMQARVFKSSIGNGCVLEPQAAVIGVTIPDGRYVPAGAVVTSQAEADALPKLTDDYAYAHTNEKVVAVNLALAKGYLTTPTAYIAPSATVIGDVTIGANVMISPMASIRADEGMPIKVGDNANVQDQVTLHALETKDEEGNDIEENWVEVNGEKYAVYVGANVSLAHQAQLHGPCIVGDDTFIGMQARVFKSSIGNGCVLEPQAAVIGVTIPDGRYVPAGAVVTSQAEADALPKLTDDYAYAHTNEKVVAVNLALAKGYLTTP

SEQ ID NO: 460 - protein, 549 aa, synthetic construct
  source 1..549: note = Library of modified or engineered enzymes
TAYIHPSAVVIGQVEIGANVMVSPMASIRSDEGMPIKIEANANVQDGVLIQSLVSKENDKELLEKLKKLNNGEYYNIYLEEGVSLAHQATILNSCYLSSGCFLAEGVVLENSVLNDAVFLGRGVTVTNAEVLEPHVFEAGDVITEEKVEPVEIPEELRAAIAAQRAAVIAANLAAAAAAKAAATAYIHPSAVVIGQVEIGANVMVSPMASIRSDEGMPIKIEANANVQDGVLIQSLVSKENDKELLEKLKKLNNGEYYNIYLEEGVSLAHQATILNSCYLSSGCFLAEGVVLENSVLNDAVFLGRGVTVTNAEVLEPHVFEAGDVITEEKVEPVEIPEELRAAIAAQRAAVIAANLAAAAAAKAAATAYIHPSAVVIGQVEIGANVMVSPMASIRSDEGMPIKIEANANVQDGVLIQSLVSKENDKELLEKLKKLNNGEYYNIYLEEGVSLAHQATILNSCYLSSGCFLAEGVVLENSVLNDAVFLGRGVTVTNAEVLEPHVFEAGDVITEEKVEPVEIPEELRAAIAAQRAAVIAANLAAAAAAKAAA

SEQ ID NO: 461 - protein, 549 aa, synthetic construct
  source 1..549: note = Library of modified or engineered enzymes
TAYIHPQANVIGDVEIGANVMVSPMASIRSDEGMPIFVGENANVQDQVTLHALETYDEEGNPIEENIVEVDGKKYAVYLGKNVSLAHQAQIHGPSIVGDDTFIGMQALVFKSVLGNNCVLEPQAAAIGVTIPDGRYIPAGKVVTSQAEADALPEVTPDYAYYHTNEQVVYVNTQLAEGYRAAATAYIHPQANVIGDVEIGANVMVSPMASIRSDEGMPIFVGENANVQDQVTLHALETYDEEGNPIEENIVEVDGKKYAVYLGKNVSLAHQAQIHGPSIVGDDTFIGMQALVFKSVLGNNCVLEPQAAAIGVTIPDGRYIPAGKVVTSQAEADALPEVTPDYAYYHTNEQVVYVNTQLAEGYRAAATAYIHPQANVIGDVEIGANVMVSPMASIRSDEGMPIFVGENANVQDQVTLHALETYDEEGNPIEENIVEVDGKKYAVYLGKNVSLAHQAQIHGPSIVGDDTFIGMQALVFKSVLGNNCVLEPQAAAIGVTIPDGRYIPAGKVVTSQAEADALPEVTPDYAYYHTNEQVVYVNTQLAEGYRAAA

SEQ ID NO: 462 - protein, 549 aa, synthetic construct
  source 1..549: note = Library of modified or engineered enzymes
TAYIHPSAEVIGSVEIGENVMISPNASIRSDEGMPIVIGDNANVQDGVTLHGLETKTEEGELIEENYVEVDGKKYVVYIGENVSLAHQAQVHGPAKVGEDTFIGMGATVTQSILGEGVLLREGAQITGVTLAPGTVVDRGTVLTTQADVASLRKLEPSDPLLKENEEVRKKNLALWEELKKAETAYIHPSAEVIGSVEIGENVMISPNASIRSDEGMPIVIGDNANVQDGVTLHGLETKTEEGELIEENYVEVDGKKYVVYIGENVSLAHQAQVHGPAKVGEDTFIGMGATVTQSILGEGVLLREGAQITGVTLAPGTVVDRGTVLTTQADVASLRKLEPSDPLLKENEEVRKKNLALWEELKKAETAYIHPSAEVIGSVEIGENVMISPNASIRSDEGMPIVIGDNANVQDGVTLHGLETKTEEGELIEENYVEVDGKKYVVYIGENVSLAHQAQVHGPAKVGEDTFIGMGATVTQSILGEGVLLREGAQITGVTLAPGTVVDRGTVLTTQADVASLRKLEPSDPLLKENEEVRKKNLALWEELKKAE

SEQ ID NO: 463 - protein, 549 aa, synthetic construct
  source 1..549: note = Library of modified or engineered enzymes
TAVIAPNAQVIGEVHIGDNVMISPNASIRSDEGMPIYIGENANLQDNVQLHGLEVLDEEGNVIEEALVEVDGKKYVVYIGKNVSLGHQAQIHGPALVGDDTFIGMNAKVFKSRIGNGCVLEPNAQVIGVTIPDGRYVPAGKVVTTQAEADALPVLTPDYAMAHTNERVVAVNLALAAAARAAATAVIAPNAQVIGEVHIGDNVMISPNASIRSDEGMPIYIGENANLQDNVQLHGLEVLDEEGNVIEEALVEVDGKKYVVYIGKNVSLGHQAQIHGPALVGDDTFIGMNAKVFKSRIGNGCVLEPNAQVIGVTIPDGRYVPAGKVVTTQAEADALPVLTPDYAMAHTNERVVAVNLALAAAARAAATAVIAPNAQVIGEVHIGDNVMISPNASIRSDEGMPIYIGENANLQDNVQLHGLEVLDEEGNVIEEALVEVDGKKYVVYIGKNVSLGHQAQIHGPALVGDDTFIGMNAKVFKSRIGNGCVLEPNAQVIGVTIPDGRYVPAGKVVTTQAEADALPVLTPDYAMAHTNERVVAVNLALAAAARAAA

SEQ ID NO: 464 - protein, 549 aa, synthetic construct
  source 1..549: note = Library of modified or engineered enzymes
TAYIHPQATVIGDVTIGANVMVSPMASIRSDEGMPIFVGDNANVQDGVTLHALETYDEEGNPIEENWVEVDGKKYAVYLGDNVSLAHQAQVHGPAAVGEDTFIGMQATVFKSKLGNNCVLEPGAAAIGVTIPDGRYIPAGKVVTSQAEADALPEVTPDYAYYHTNEDVVYVNIALAEGYKKLSTAYIHPQATVIGDVTIGANVMVSPMASIRSDEGMPIFVGDNANVQDGVTLHALETYDEEGNPIEENWVEVDGKKYAVYLGDNVSLAHQAQVHGPAAVGEDTFIGMQATVFKSKLGNNCVLEPGAAAIGVTIPDGRYIPAGKVVTSQAEADALPEVTPDYAYYHTNEDVVYVNIALAEGYKKLSTAYIHPQATVIGDVTIGANVMVSPMASIRSDEGMPIFVGDNANVQDGVTLHALETYDEEGNPIEENWVEVDGKKYAVYLGDNVSLAHQAQVHGPAAVGEDTFIGMQATVFKSKLGNNCVLEPGAAAIGVTIPDGRYIPAGKVVTSQAEADALPEVTPDYAYYHTNEDVVYVNIALAEGYKKLS